(19) United States
(12) Patent Application Publication
(10) Pub. No.: US 2023/0122364 A1
(43) Pub. Date: Apr. 20, 2023

(54) HUMAN MONOCLONAL ANTIBODIES TO SEVERE ACUTE RESPIRATORY SYNDROME CORONAVIRUS 2 (SARS-CoV-2)

(71) Applicant: VANDERBILT UNIVERSITY, Nashville, TN (US)

(72) Inventors: James E. CROWE, Jr., Nashville, TN (US); Seth ZOST, Nashville, TN (US); Robert CARNAHAN, Nashville, TN (US); Pavlo GILCHUK, Nashville, TN (US)

(21) Appl. No.: 17/907,320

(22) PCT Filed: Mar. 26, 2021
(86) PCT No.: PCT/US21/24341

§ 371 Date: Sep. 26, 2022

Related U.S. Application Data
(60) Provisional application No. 63/000,735, filed on Mar. 27, 2020.
(60) Provisional application No. 63/002,896, filed on Mar. 31, 2020.
(60) Provisional application No. 63/023,545, filed on May 12, 2020.
(60) Provisional application No. 63/024,214, filed on May 13, 2020.
(60) Provisional application No. 63/024,248, filed on May 13, 2020.
(60) Provisional application No. 63/027,173, filed on May 19, 2020.
(60) Provisional application No. 63/037,984, filed on Jun. 11, 2020.
(60) Provisional application No. 63/040,246, filed on Jun. 17, 2020.
(60) Provisional application No. 63/142,196, filed on Jan. 27, 2021.

(51) Int. Cl. C07K 16/10; G01N 33/569; A61P 31/14

(52) U.S. Cl. CPC C07K 16/10; G01N 33/56983; A61P 31/14; G01N 2333/165; G01N 2469/10; C07K 2317/52; A61K 2039/545

(57) ABSTRACT

The present disclosure is directed to antibodies binding to and neutralizing tire coronavirus designated SARS-CoV-2 and methods for use thereof.

### PRIORITY CLAIM

This application claims benefit of priority to the following applications, each of which are hereby incorporated by reference in their entirety: U.S. Provisional Application Ser. Nos. 63/000,735 and 63/024,214, filed Mar. 27, 2020, and May 13, 2020, respectively; and U.S. Provisional Application Ser. Nos. 63/002,896, 63/024,248, 63/027,173, 63/037,984, 63/040,246, and 63/142,196, filed Mar. 31, 2020, May 13, 2020. May 19, 2020, Jun. 11, 2020, Jun. 17, 2020, and Jan. 27, 2021, respectively; and U.S. Provisional Application Ser. No. 63/023,545, filed May 12, 2020.

### FEDERAL FUNDING DISCLOSURE

This invention was made with government support under HR0011-18-2-0001 awarded by the Defense Advanced Research Projects Agency (DARPA) and HHS Contract 75N93019C00074 awarded by the National Institutes of Allergy and Infection Disease/National Institutes of Health. The government has certain rights in the invention.

### BACKGROUND

### 1. Field of the Disclosure

The present disclosure relates generally to the fields of medicine, infectious disease, and immunology. More particular, the disclosure relates to human antibodies binding to a novel coronavirus designated SARS-CoV-2 and methods of use therefor.

### 2. Background

An epidemic of a novel coronavirus (SARS-CoV-2) affected mainland China, along with cases in 179 other countries and territories. It was identified in Wuhan, the capital of China's Hubei province, after 41 people developed pneumonia without a clear cause. The virus, which causes acute respiratory disease designated coronavirus disease 2019 (COVID-19), is capable of spreading from person to person. The incubation period (time from exposure to onset of symptoms) ranges from 0 to 24 days, with a mean of 3-5 days, but it may be contagious during this period and after recovery. Symptoms include fever, coughing and breathing difficulties. An estimate of the death rate in February 200 was 2% of confirmed cases, higher among those who require admission to hospital.

As of 10 Feb. 2020, 40,627 cases have been confirmed (6,495 serious), including in every province-level division of China. A larger number of people may have been infected, but not detected (especially mild cases). As of 10 Feb. 2020, 910 deaths have been attributed to the virus since the first confirmed death on 9 January, with 3,323 recoveries. The first local transmission outside China occurred in Vietnam between family members, while the first international transmission not involving family occurred in Germany on 22 January. The first death outside China was in the Philippines, where a man from Wuhan died on 1 February. As of 10 Feb. 2020, the death toll from this virus had surpassed the global SARS outbreak in 2003.

As of early February 2020, there is no licensed vaccine and no specific treatment, although several vaccine approaches and antivirals are being investigated. The outbreak has been declared a Public Health Emergency of International Concern (PHEIC) by the World Health Organization (WHO), based on the possible effects the virus could have if it spreads to countries with weaker healthcare systems. Thus, there is an urgent need to explore the biology and pathology of SARS-CoV-2 and well as the human immune response to this virus.

### SUMMARY

Thus, in accordance with the present disclosure, there is provided a method of detecting COVID-19 infection with SARS-CoV-2 in a subject comprising (a) contacting a sample from said subject with an antibody or antibody fragment having clone-paired heavy and light chain CDR sequences from Tables 3 and 4, respectively; and (b) detecting SARS-CoV-2 in said sample by binding of said antibody or antibody fragment to a SARS-CoV-2 antigen in said sample. The sample may be a body fluid, such as blood, sputum, tears, saliva, mucous or serum, semen, cervical or vaginal secretions, amniotic fluid, placental tissues, urine, exudate, transudate, tissue scrapings or feces. Detection may comprise ELISA, RIA, lateral flow assay or western blot. The method may further comprise performing steps (a) and (b) a second time and determining a change in SARS-CoV-2 antigen levels as compared to the first assay. The antibody or antibody fragment may be encoded by clone-paired variable sequences as set forth in Table 1. The antibody or antibody fragment may be encoded by light and heavy chain variable sequences having at least 70%, 80%, 90% or 95% identity to clone-paired variable sequences as set forth in Table 1, or by light and heavy chain variable sequences having 100% identity to clone-paired sequences as set forth in Table 1. The antibody or antibody fragment may comprise light and heavy chain variable sequences according to clone-paired sequences from Table 2, or light and heavy chain variable sequences having at least 70%, 80%, 90% or 95% identity to clone-paired sequences from Table 2. The antibody or antibody fragment may bind to a SARS-CoV-2 surface spike protein. The antibody fragment may be a recombinant scFv (single chain fragment variable) antibody, Fab fragment, F(ab′)₂ fragment, or Fv fragment.

In another embodiment, there is provided a method of treating a subject infected with SARS-CoV-2 or reducing the likelihood of infection of a subject at risk of contracting SARS-CoV-2, comprising delivering to said subject an antibody or antibody fragment having clone-paired heavy and light chain CDR sequences from Tables 3 and 4, respectively. The antibody or antibody fragment may be encoded by light and heavy chain variable sequences having at least 70%, 80%, 90% or 95% identity to clone-paired variable sequences as set forth in Table 1, or by light and heavy chain variable sequences having 100% identity to clone-paired sequences as set forth in Table 1. The antibody or antibody fragment may comprise light and heavy chain variable sequences according to clone-paired sequences from Table 2, or light and heavy chain variable sequences having at least 70%, 80%, 90% or 95% identity to clone-paired sequences from Table 2. The antibody fragment may be a recombinant scFv (single chain fragment variable) antibody, Fab fragment, F(ab′)₂ fragment, or Fv fragment. The antibody may be a chimeric antibody or a bispecific antibody. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment may bind to a SARS-CoV-2 antigen such as a surface spike protein. The antibody or antibody fragment may be administered prior to infection or after infection. The subject may be of age 60 or older, may be immunocompromised, or may suffer from a respiratory and/or cardiovascular disorder. Delivering may comprise antibody or antibody fragment administration, or genetic delivery with an RNA or DNA sequence or vector encoding the antibody or antibody fragment.

In yet another embodiment, there is provided a monoclonal antibody, wherein the antibody or antibody fragment is characterized by clone-paired heavy and light chain CDR sequences from Tables 3 and 4, respectively. The antibody or antibody fragment may be encoded by light and heavy chain variable sequences having at least 70%, 80%, 90% or 95% identity to clone-paired variable sequences as set forth in Table 1, or by light and heavy chain variable sequences having 100% identity to clone-paired sequences as set forth in Table 1. The antibody or antibody fragment may comprise light and heavy chain variable sequences according to clone-paired sequences from Table 2, or light and heavy chain variable sequences having at least 70%, 80%, 90% or 95% identity to clone-paired sequences from Table 2. The antibody fragment may be a recombinant scFv (single chain fragment variable) antibody, Fab fragment. F(ab′)2 fragment, or Fv fragment. The antibody may be a chimeric antibody, is bispecific antibody, or is an intrabody. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment may bind to a SARS-CoV-2 surface spike protein.

A hybridoma or engineered cell encoding an antibody or antibody fragment wherein the antibody or antibody fragment is characterized by clone-paired heavy and light chain CDR sequences from Tables 3 and 4, respectively. The antibody or antibody fragment may be encoded by light and heavy chain variable sequences having at least 70%, 80%, 90% or 95% identity to clone-paired variable sequences as set forth in Table 1, or by light and heavy chain variable sequences having 100% identity to clone-paired sequences as set forth in Table 1. The antibody or antibody fragment may comprise light and heavy chain variable sequences according to clone-paired sequences from Table 2, or light and heavy chain variable sequences having at least 70%, 80%, 90% or 95% identity to clone-paired sequences from Table 2. The antibody fragment may be a recombinant scFv (single chain fragment variable) antibody, Fab fragment, F(ab′)₂ fragment, or Fv fragment. The antibody may be a chimeric antibody, a bispecific antibody, or an intrabody. The antibody may bean IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. The antibody or antibody fragment may bind to a SARS-CoV-2 surface spike protein.

In still yet another embodiment, there is provided a vaccine formulation comprising one or more antibodies or antibody fragments characterized by clone-paired heavy and light chain CDR sequences from Tables 3 and 4, respectively. The at least one of said antibodies or antibody fragments may be encoded by light and heavy chain variable sequences according to clone-paired sequences from Table 1, by light and heavy chain variable sequences having at least 70%, 80%, or 90% identity to clone-paired sequences from Table 1, or by light and heavy chain variable sequences having at least 95% identity to clone-paired sequences from Table 1. The at least one of said antibodies or antibody fragments may comprise light and heavy chain variable sequences according to clone-paired sequences from Table 2, or may comprise light and heavy chain variable sequences having at least 70%, 80$, 90% or 95% identity to clone-paired sequences from Table 2. The at least one of said antibody fragments is a recombinant scFv (single chain fragment variable) antibody, Fab fragment, F(ab′)₂ fragment, or Fv fragment. The at least one of said antibodies may a chimeric antibody, a bispecific antibody or an intrabody. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, LALA PC, N297, GASD/ALIE, DHS YTE or LS mutation or glycan modified to alter (eliminate or enhance) FcR interactions such as enzymatic or chemical addition or removal of glycans or expression in a cell line engineered with a defined glycosylating pattern. The antibody or antibody fragment may bind to a SARS-CoV-2 antigen surface spike protein.

In a further embodiment, there is provided a vaccine formulation comprising one or more expression vectors encoding a first antibody or antibody fragment as described herein. The expression vector(s) may be Sindbis virus or VEE vector(s). The vaccine may be formulated for delivery by needle injection, jet injection, or electroporation. The vaccine formulation may further comprise one or more expression vectors encoding for a second antibody or antibody fragment, such as a distinct antibody or antibody fragment as described herein.

In yet a further embodiment, there is provided a method of protecting the health of a subject of age 60 or older, an immunocompromised, subject or a subject suffering from a respiratory and/or cardiovascular disorder that is infected with or at risk of infection with SARS-CoV-2 comprising delivering to said subject an antibody or antibody fragment having clone-paired heavy and light chain CDR sequences from Tables 3 and 4, respectively. The antibody or antibody fragment may be encoded by light and heavy chain variable sequences having at least 70%, 80%, 90% or 95% identity to clone-paired variable sequences as set forth in Table 1, or by light and heavy chain variable sequences having 100% identity to clone-paired sequences as set forth in Table 1. The antibody or antibody fragment may comprise light and heavy chain variable sequences according to clone-paired sequences from Table 2, or light and heavy chain variable sequences having at least 70%, 80%, 90% or 95% identity to clone-paired sequences from Table 2. The antibody fragment may be a recombinant scFv (single chain fragment variable) antibody, Fab fragment, F(ab′)₂ fragment, or Fv fragment. The antibody may an IgG, or a recombinant IgG antibody or antibody fragment comprising an Fe portion mutated to alter (eliminate or enhance) FcR interactions, to increase half-life and/or increase therapeutic efficacy, such as a LALA, LALA PG. N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The said antibody or antibody fragment may be administered prior to infection or after infection. The antibody or antibody fragment may bind to a SARS-CoV-2 antigen such as a surface spike protein. Delivering may comprise antibody or antibody fragment administration, or genetic delivery with an RNA or DNA sequence or vector encoding the antibody or antibody fragment. The antibody or antibody fragment may improve the subject's respiration as compared to an untreated control and/or may reduce viral load as compared to an untreated control.

In still yet a further embodiment, there is provided a method of determining the antigenic integrity, correct conformation and/or correct sequence of a SARS-CoV-2 surface spike protein comprising (a) contacting a sample comprising said antigen with a first antibody or antibody fragment having clone-paired heavy and light chain CDR sequences from Tables 3 and 4, respectively; and (b) determining antigenic integrity, correct conformation and/or correct sequence of said antigen by detectable binding of said first antibody or antibody fragment to said antigen. The sample may comprise recombinantly produced antigen or a vaccine formulation or vaccine production batch. Detection may comprise ELISA, RIA, western blot, a biosensor using surface plasmon resonance or biolayer interferometry, or flow cytometric staining. The first antibody or antibody fragment may be encoded by clone-paired variable sequences as set forth in Table 1, by light and heavy chain variable sequences having at least 70%, 80%, or 90% identity to clone-paired variable sequences as set forth in Table 1, or by light and heavy chain variable sequences having at least 95% identity to clone-paired sequences as set forth in Table 1. The first antibody or antibody fragment may comprise light and heavy chain variable sequences according to clone-paired sequences from Table 2, may comprise light and heavy chain variable sequences having at least 70%, 80% or 90% identity to clone-paired sequences from Table 2, or may comprise light and heavy chain variable sequences having at least 95% identity to clone-paired sequences from Table 2. The first antibody fragment may be a recombinant scFv (single chain fragment variable) antibody. Fab fragment, F(ab′)₂ fragment, or Fv fragment. The method may further comprise performing steps (a) and (b) a second time to determine the antigenic stability of the antigen over time.

The method may further comprise (c) contacting a sample comprising said antigen with a second antibody or antibody fragment having clone-paired heavy and light chain CDR sequences from Tables 3 and 4, respectively; and (d) determining antigenic integrity of said antigen by detectable binding of said second antibody or antibody fragment to said antigen. The second antibody or antibody fragment may be encoded by clone-paired variable sequences as set forth in Table 1, by light and heavy chain variable sequences having at least 70%, 80%, or 90% identity to clone-paired variable sequences as set forth in Table 1, or by light and heavy chain variable sequences having at least 95% identity to clone-paired sequences as set forth in Table 1. The second antibody or antibody fragment may comprise light and heavy chain variable sequences according to clone-paired sequences from Table 2, may comprise light and heavy chain variable sequences having at least 70%, 80% or 90% identity to clone-paired sequences from Table 2, or may comprise light and heavy chain variable sequences having at least 95% identity to clone-paired sequences from Table 2. The second first antibody fragment may be a recombinant scFv (single chain fragment variable) antibody. Fab fragment, F(ab′)₂ fragment, or Fv fragment. The method may further comprise performing steps (c) and (d) a second time to determine the antigenic stability of the antigen over time.

Also provided is human monoclonal antibody or antibody fragment, or hybridoma or engineered cell producing the same, wherein said antibody binds to a SARS-CoV-2 antigen surface spike protein. The use of the word “a” or “an” when used in conjunction with the term “comprising” in the claims and/or the specification may mean “one.” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. IMGT/DomainGapAlign results of COV2-2196 heavy and light chains. Key interacting residues and their corresponding residues in germline genes are colored in red.

FIG. 2. Identification of critical residues for COV2-21% and COV2-2130 through deep mutational scanning coupled with resistant variant selection. Antibody neutralization as measured by FRNT against reference strains and SARS-CoV-2 variants of concern. Neutralization assays were performed in duplicate and repeated twice, with results shown from one experimental replicate. Error bars denote the range for each point. Mutations compared to the WA-1 reference strain are denoted. B.1.1.7-OXF contains 69-70 and 144-145 deletion and the following substitutions: N501Y, A570D, D614G, P681H, and T716I.

FIGS. 3A-B. Identification of putative public clonotype members genetically similar to COV2-21% in the antibody variable gene repertoires of virus-naïve individuals. Antibody variable gene sequences from healthy individuals with the same sequence features as COV2-2196 heavy chain and light chain are aligned. Sequences from three different donors as well as cord blood included sequences with the features of the public clonotype. The sequence features and contact residues used in COV2-2196 are highlighted in red boxes below each multiple sequence alignment. (SEQ ID NOS: 1050-1054)

FIGS. 4A-E. Functional characteristics of neutralizing SARS-CoV-2 mAbs. FIG. 4A. Heatmap of mAb neutralization activity, hACE2 blocking activity, and binding to either trimeric S2P_{ecto} protein or monomeric SRBD. MAbs are ordered by neutralization potency (highest at the top, lowest at the bottom). Dashed lines indicate the 13 antibodies with a neutralization IC₅₀ value lower than 150 ng/mL for wt virus. IC₅₀ values are visualized for viral neutralization and hACE2 blocking, while EC₅₀ values are visualized for binding. A recombinant form of the cross-reactive SARS-CoV SRBD mAb CR3022 is shown as a positive control, while the anti-dengue mAb 2D22 is shown as a negative control. Data are representative of at least 2 independent experiments, each performed in technical duplicate. No inhibition indicates an IC₅₀ value of >10,000 ng/mL, while no binding indicates an EC₅₀ value of >10,000 ng/mL. FIGS. 4B-D. Correlation of hACE2 blocking, S2P_{ecto} trimer binding, or SRBD binding of mAbs with their neutralization activity. R2 values are shown for linear regression analysis of log-transformed values. Purple circles indicate mAbs with a neutralization IC₅₀ value lower than 150 ng/mL. FIG. 4E. Correlation of hACE2 blocking and S2P_{ecto} trimer binding. R² values are shown for linear regression analysis of log-transformed values.

FIGS. 5A-B. Epitope mapping of mAbs by competition-binding analysis and synergistic neutralization by a pair of mAbs. FIG. 4A. Left: biolayer interferometry-based competition binding assay measuring the ability of mAbs to prevent binding of reference mAbs COV2-2196 and rCR3022 to RBD fused to mouse Fc (RBD-mFc) loaded onto anti-mouse Fc biosensors. Values in squares are % of binding of the reference mAb in the presence of the competing mAb relative to a mock-competition control. Black squares denote full competition (<33% of binding relative to no-competition control), while white squares denote no competition (>67% of binding relative to no-competition control). Right: biolayer interferometry-based competition binding assay measuring the ability of mAbs to prevent binding of hACE2. Values denote % binding of hACE2, normalized to hACE2 binding in the absence of competition. Red color denotes competition of mAb with hACE2. FIG. 4B. Competition of neutralizing mAb panel with reference mAbs COV2-2130, COV2-2196, or rCR3022. Reference mAbs were biotinylated and binding of reference mAbs to trimeric S2P_{ecto} was measured in the presence of saturating amounts of each mAb in a competition ELISA. ELISA signal for each reference mAb was normalized to the signal in the presence of the non-binding anti-dengue mAb 2D22. Black denotes full competition (<25% binding of reference mAb), grey denotes partial competition (25-60% binding of reference mAb), and white denotes no competition (>60% binding of reference mAb).

FIG. 6. SARS-CoV-2 neutralization curves for mAb panel. Neutralization of authentic SARS-CoV-2 by human mAbs. Mean±SD of technical duplicates is shown. Data represent one of two or more independent experiments

FIG. 7. Inhibition curves for mAb inhibition of S2P_{ecto} binding to hACE2. Blocking of hACE2 binding to S2P_{ecto} by anti-SARS-CoV-2 neutralizing human mAbs. Mean±SD of triplicates of one experiment is shown. Antibodies CR3022 and 2D22 served as controls.

FIG. 8. ELISA binding of anti-SARS-CoV-2 neutralizing human mAbs to trimeric SRBD, S2P_{ecto}, or SARS-CoV S2P_{ecto} antigen. Mean±SD of triplicates and representative of two experiments are shown. Antibodies CR3022 and 2D22 served as controls.

### DESCRIPTION OF ILLUSTRATIVE EMBODIMENTS

As discussed above, SARS-CoV-2 is a major health concern with active cases increasing daily. Therefore, understanding the biology of this virus and the nature and extent of the human immune response to the virus is of paramount importance. The inventors have identified the sequences of human antibodies to SARS-CoV-2. Those sequences and uses for such antibodies are disclosed herein.

These and other aspects of the disclosure are described in detail below.

### I. Coronavirus 2019 (SARS-CoV-2)

SARS-CoV-2 is a contagious virus that causes the acute respiratory disease designated coronavirus disease 2019 (COVID-19), a respiratory infection. It is the cause of the ongoing 2019-20 coronavirus outbreak, a global health emergency. Genomic sequencing has shown that it is a positive-sense, single-stranded RNA coronavirus.

During the ongoing outbreak, the virus has often been referred to in common parlance as “the coronavirus”, “the new coronavirus” and “the Wuhan coronavirus”, while the WHO recommends the designation “SARS-CoV-2”. The International Committee on Taxonomy of Viruses (ICTV) announced that the official name for the virus is SARS-CoV-2.

Many early cases were linked to a large seafood and animal market in the Chinese city of Wuhan, and the virus is thought to have a zoonotic origin. Comparisons of the genetic sequences of this virus and other virus samples have shown similarities to SARS-CoV (79.5%) and bat coronaviruses (96%). This finding makes an ultimate origin in bats likely, although an intermediate host, such as a pangolin, cannot be ruled out. The virus could be a recombinant virus formed from two or more coronaviruses.

Human-to-human transmission of the virus has been confirmed. Coronaviruses are primarily spread through close contact, in particular through respiratory droplets from coughs and sneezes within a range of about 6 feet (1.8 m). Viral RNA has also been found in stool samples from infected patients. It is possible that the virus can be infectious even during the incubation period.

Animals sold for food were originally suspected to be the reservoir or intermediary hosts of SARS-CoV-2 because many of the first individuals found to be infected by the virus were workers at the Huanan Seafood Market. A market selling live animals for food was also blamed in the SARS outbreak in 2003; such markets are considered to be incubators for novel pathogens. The outbreak has prompted a temporary ban on the trade and consumption of wild animals in China. However, some researchers have suggested that the Huanan Seafood Market may not be the original source of viral transmission to humans.

With a sufficient number of sequenced genomes, it is possible to reconstruct a phylogenetic tree of the mutation history of a family of viruses. Research into the origin of the 2003 SARS outbreak has resulted in the discovery of many SARS-like bat coronaviruses, most originating in the Rhinolophus genus of horseshoe bats. SARS-CoV-2 falls into this category of SARS-related coronaviruses. Two genome sequences from Rhinolophus sinicus published in 2015 and 2017 show a resemblance of 80% to SARS-CoV-2. A third virus genome from Rhinolophus affinis. “RaTG13” collected in Yunnan province, has a 96% resemblance to SARS-CoV-2.^{[28][29]} For comparison, this amount of variation among viruses is similar to the amount of mutation observed over ten years in the H3N2 human influenza virus strain.

SARS-CoV-2 belongs to the broad family of viruses known as coronaviruses; “nCoV” is the standard term used to refer to novel coronaviruses until the choice of a more specific designation. It is a positive-sense single-stranded RNA (+ssRNA) virus. Other coronaviruses are capable of causing illnesses ranging from the common cold to more severe diseases such as Middle East respiratory syndrome (MERS) and Severe acute respiratory syndrome (SARS). It is the seventh known coronavirus to infect people, after 229E, NL63, OC43, HKU1, MERS-CoV, and SARS-CoV.

Like SARS-CoV, SARS-CoV-2 is a member of the subgenus Sarbecovirus (Beta-CoV lineage B). Its RNA sequence is approximately 30,000 bases in length. By 12 January, five genomes of SARS-CoV-2 had been isolated from Wuhan and reported by the Chinese Center for Disease Control and Prevention (CCDC) and other institutions; the number of genomes increased to 28 by 26 January. Except for the earliest GenBank genome, the genomes are under an embargo at GISAID. A phylogenic analysis for the samples is available through Nextstrain.

Publication of the SARS-CoV-2 genome led to several protein modeling experiments on the receptor binding protein (RBD) of the spike (S) protein of the virus. Results suggest that the S protein retains sufficient affinity to the Angiotensin converting enzyme 2 (ACE2) receptor to use it as a mechanism of cell entry. On 22 January, a group in China working with the full virus and a group in the U.S. working with reverse genetics independently and experimentally demonstrated human ACE2 as the receptor for SARS-CoV-2.

To look for potential protease inhibitors, the viral 3C-like protease M(pro) from the ORF1a polyprotein has also been modeled for drug docking experiments. Innophore has produced two computational models based on SARS protease, and the Chinese Academy of Sciences has produced an unpublished experimental structure of a recombinant SARS-CoV-2 protease. In addition, researchers at the University of Michigan have modeled the structures of all mature peptides in the SARS-CoV-2 genome using I-TASSER.

The first known human infection occurred in early December 2019. An outbreak of SARS-CoV-2 was first detected in Wuhan, China, in mid-December 2019, likely originating from a single infected animal. The virus subsequently spread to all provinces of China and to more than two dozen other countries in Asia, Europe, North America, and Oceania. Human-to-human spread of the virus has been confirmed in all of these regions. On 30 Jan. 2020, SARS-CoV-2 was designated a global health emergency by the WHO.

As of 10 Feb. 2020 (17:15 UTC), there were 40.645 confirmed cases of infection, of which 40.196 were within mainland China. Initially, nearly all cases outside China occurred in people who either traveled from Wuhan, or were in direct contact with someone who traveled from the area. Later, spread from travelers to other countries resulted in transmission in many countries in the world. While the proportion of infections that result in confirmed infection or progress to diagnosable SARS-CoV-2 acute respiratory disease remains unclear, the total number of deaths attributed to the virus was over 19,000 as of 25 Mar. 2020.

The basic reproduction number (R-zero) of the virus has been estimated to be between 1.4 and 3.9. This means that, when unchecked, the virus typically results in 1.4 to 3.9 new cases per established infection. It has been established that the virus is able to transmit along a chain of at least four people.

In January 2020, multiple organizations and institutions began work on creating vaccines for SARS-CoV-2 based on the published genome. In China, the Chinese Center for Disease Control and Prevention is developing a vaccine against the novel coronavirus. The University of Hong Kong has also announced that a vaccine is under development there. Shanghai East Hospital is also developing a vaccine in partnership with the biotechnology company Stemirna Therapeutics.

Elsewhere, three vaccine projects are being supported by the Coalition for Epidemic Preparedness Innovations (CEPI), including projects by the biotechnology companies Moderna and Inovio Pharmaceuticals and another by the University of Queensland. The United States National Institutes of Health (NIH) is cooperating with Moderna to create an RNA vaccine matching a spike of the coronavirus surface; Phase I clinical trials began in March 2020. Inovio Pharmaceuticals is developing a DNA-based vaccination and collaborating with a Chinese firm in order to speed its acceptance by regulatory authorities in China, hoping to perform human trials of the vaccine in the summer of 2020. In Australia, the University of Queensland is investigating the potential of a molecular clamp vaccine that would genetically modify viral proteins to make them mimic the coronavirus and stimulate an immune reaction.

In an independent project, the Public Health Agency of Canada has granted permission to the International Vaccine Centre (VIDO-InterVac) at the University of Saskatchewan to begin work on a vaccine. VIDO-InterVac aims to start production and animal testing in March 2020, and human testing in 2021. The Imperial College Faculty of Medicine in London is now at the stage of testing a vaccine on animals.

COVID-19 acute respiratory disease is a viral respiratory disease caused by SARS-CoV-2. It was first detected during the 2019-20 Wuhan coronavirus outbreak. Symptoms may include fever, dry cough, and shortness of breath. There is no specific licensed treatment available as of March 2020, with efforts focused on lessening symptoms and supporting functioning.

Those infected may either be asymptomatic or have mild to severe symptoms, like fever, cough, shortness of breath. Diarrhoea or upper respiratory symptoms (e.g., sneezing, runny nose, sore throat) are less frequent. Cases of severe infection can progress to severe pneumonia, multi-organ failure, and death. The time from exposure to onset of symptoms is estimated at 2 to 10 days by the World Health Organization, and 2 to 14 days by the US Centers for Disease Control and Prevention (CDC).

Global health organizations have published preventive measures individuals can take to reduce the chances of SARS-CoV-2 infection. Recommendations are similar to those previously published for other coronaviruses and include: frequent washing of hands with soap and water; not touching the eyes, nose, or mouth with unwashed hands; and practicing good respiratory hygiene.

The WHO has published several testing protocols for SARS-CoV-2. Testing uses real time reverse transcription-polymerase chain reaction (rRT-PCR). The test can be done on respiratory or blood samples. Results are generally available within a few hours to days.

Research into potential treatments for the disease were initiated in January 2020. The Chinese Center for Disease Control and Prevention started testing existing pneumonia treatments in coronavirus-related pneumonia in late January. There has also been examination of the RNA polymerase inhibitor remdesivir, and interferon beta. In late January 2020, Chinese medical researchers expressed an intent to start clinical testing on remdesivir, chloroquine, and lopinavir/ritonavir, all of which seemed to have “fairly good inhibitory effects” on SARS-CoV-2 at the cellular level in exploratory research. On 5 Feb. 2020, China started patenting use of remdesivir for the disease.

Overall mortality and morbidity rates due to infection with SARS-CoV-2 are unknown, both because the case fatality rate may be changing over time in the current outbreak, and because the proportion of infections that progress to diagnosable disease remains unclear. However, preliminary research into SARS-CoV-2 acute respiratory disease has yielded case fatality rate numbers between 2% and 3%, and in January 2020 the WHO suggested that the case fatality rate was approximately 3%. An unreviewed Imperial College preprint study among 55 fatal cases noted that early estimates of mortality may be too high as asymptomatic infections are missed. They estimated a mean infection fatality ratio (the mortality among infected) ranging from 0.8% when including asymptomatic carriers to 18% when including only symptomatic cases from Hubei province.

Early data indicates that among the first 41 confirmed cases admitted to hospitals in Wuhan, 13 (32%) individuals required intensive care, and 6 (15%) individuals died. Of those who died, many were in unsound health to begin with, exhibiting conditions like hypertension, diabetes, or cardiovascular disease that impaired their immune systems. In early cases of SARS-CoV-2 acute respiratory disease that resulted in death, the median time of disease was found to be 14 days, with a total range from six to 41 days.

### II. Monoclonal Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V₁ and V_{L} together forms a single antigen-binding site. For the structure and properties of the different classes of antibodies, see, e.g., Basic and Clinical Immunology, 8th edition, Daniel P. Stites, Abba 1. Terr and Tristram G. Parslow (eds.), Appleton & Lange, Norwalk. Conn., 1994, page 71, and Chapter 6.

The L chain from any vertebrate species can be assigned to one of two clearly distinct types, called kappa and lambda based on the amino acid sequences of their constant domains (C). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda. Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63.74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

### A. General Methods

It will be understood that monoclonal antibodies binding to SARS-CoV-2 will have several applications. These include the production of diagnostic kits for use in detecting and diagnosing SARS-CoV-2 infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce SARS-CoV-2-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells identified as responding to infection or vaccination because of plasmablast aor activated B cell markers, or memory B cells labelled with the antigen of interest, can be sorted physically using paramagnetic bead selection or flow cytometric sorting, then RNA can be isolated from the single cells and antibody genes amplified by RT-PCR. Various single-cell RNA-seq methods are available to obtain antibody variable genes from single cells. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes from single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Two main categories of SARS-CoV-2 antigens are the surface spike (S) protein and the internal proteins, especially the nucleocapsid (N) protein. Antibodies to the S protein will be useful for prophylaxis, or therapy, or diagnostics, or for characterizing vaccines. S protein antibodies will have additional binding specificity with that protein, with particular antibodies binding to the full-length ectodomain of the SARS-CoV-2 S protein (presented as a monomer or oligomer such as a timer; with our without conformation stabilizing mutations such as introduction of prolines at critical sites (“2P mutation”)) and (a) anti-S protein antibodies that binds to the receptor binding domain (RBD), (b) anti-S protein antibodies that bind to domains other than the RBD. Some of the subset that bind to domains other than the RBD bind to the N terminal domain (NTD), while others bind to an epitope other than the NTD or RBD), and (c) S protein antibodies may further be found to neutralize SARS-CoV-2 by blocking binding of the SARS-CoV-2 S protein to its receptor, human angiotensin-converting enzyme 2 (hACE2), with others that neutralize but do not block receptor binding. Finally, antibodies can cross-react with both SARS-CoV-2 S protein and the S protein of other coronaviruses such as SARS-CoV, MERS-CoV, HCoV-229E, HCoV-OC43, HCoV-NL63 and/or HCoV-HKU 1, as well as cross-neutralize both SARS-CoV-2 and these other coronaviruses.

Another specificity will be antibodies that bind to N antibodies (or other internal targets) that will have primarily diagnostics uses. For example, antibodies to N or other internal proteins of SARS-CoV-2 that specifically recognize SARS-CoV-2 or that cross-reactively recognize SARS-CoV-2 and other coronaviruses such as SARS-CoV, MERS-CoV, HCoV-229E, HCoV-OC43, HCoV-NL63 and/or HCoV-HKU1.

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies. Harlow and Lane (Cold Spring Harbor Press. Cold Spring Harbor. N.Y.). Cross-blocking can be measured in various binding assays such as ELISA, biolayer interferometry, or surface plasmon resonance. Other methods include alanine scanning mutational analysis, peptide blot analysis (Reineke, Methods Mol. Biol. 248: 443-63, 2004), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer Prot. Sci. 9: 487-496, 2000). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that am not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring, Analytical Biochemistry 267: 252-259 (1999); Engen and Smith, Anal. Chem. 73: 256A-265A (2001). When the antibody neutralizes SARS-CoV-2, antibody escape mutant variant organisms can be isolated by propagating SARS-CoV-2 in vitro or in animal models in the presence of high concentrations of the antibody. Sequence analysis of the SARS-CoV-2 gene encoding the antigen targeted by the antibody reveals the mutation(s) conferring antibody escape, indicating residues in the epitope or that affect the structure of the epitope allosterically.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see U.S. Patent Publication 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening. MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-SARS-CoV-2 antibody, the above-described binding methodology is performed in two orientations: In a first orientation, the reference antibody is allowed to bind to the SARS-CoV-2 antigen under saturating conditions followed by assessment of binding of the test antibody to the SARS-CoV-2 molecule. In a second orientation, the test antibody is allowed to bind to the SARS-CoV-2 antigen molecule under saturating conditions followed by assessment of binding of the reference antibody to the SARS-CoV-2 molecule. If, in both orientations, only the first (saturating) antibody is capable of binding to SARS-CoV-2, then it is concluded that the test antibody and the reference antibody compete for binding to SARS-CoV-2. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 199W 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 3 and 4, respectively. Such antibodies may be produced by the clones discussed below in the Examples section using methods described herein.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences and the amino acid sequences.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.: Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425: Sneath, P. H. A. and Sokal, R. R. (1973) Numerical Taxonomy-—the Principles and Practice of Numerical Taxonomy, Freeman Press, San Francisco, Calif.: Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2. CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen,” J. Exp. Med. 168(3): 1099-1109; Tao, M. H. et al. (1989) “Studies Of Aglycosylated Chimeric Mouse-Human IgG. Role of Carbohydrate in The Structure And Effector Functions Mediated By The Human IgG Constant Region.” J. Immunol. 143(8): 2595-2601; Routledge, E. G. et al. (1995) “The Effect of Aglycosylation on The Immunogenicity of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21: Shields. R. L. et al. (2002) “Lack of Fucose on Human IgG N-Linked Oligosaccharide Improves Binding to Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fe region to be combined with a binding domain (e.g., an antibody variable domain) and can be assessed using various assays (e.g., Fc binding assays, ADCC assays, CDC assays, etc.).

For example, one can generate a variant Fe region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fe region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fe receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fe region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab. C_{H}2, and C_{H}3 domains). Typically unfolding of the Fab domain produces the strongest peak. The DSC profiles and relative stability of the Fe portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H}C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986). According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998), doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see. e.g., U.S. Pat. No. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005: 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fe region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCI publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen
(b) a second Fab molecule which specifically binds to a second antigen, and wherein the variable domains VL and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).
   The antibody may not comprise both modifications mentioned under i) and ii). The constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

### F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3 ξ-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

### G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

### H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

### I. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

### J. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Active/Passive Immunization and Treatment/Prevention of SARS-CoV-2 Infection

### A. Formulation and Administration

The present disclosure provides pharmaceutical compositions comprising anti-SARS-CoV-2 virus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Active vaccines are also envisioned where antibodies like those disclosed are produced in vivo in a subject at risk of SARS-CoV-2 infection. Such vaccines can be formulated for parenteral administration. e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example, by nasal drops, inhalation, by nebulizer, or via intrarectal or vaginal delivery. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

2. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase. GnTIII, or other glycosyltransferases) by the methods described herein, is relative to the ADCC mediated by the same antibody produced by the same type of non-engineered host cells.

3. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-viral effect.

### IV. Antibody Conjugates

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ²phosphorus, rhenium¹⁸⁶, rheniumi¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. Immunodetection Methods

In still further embodiments, the present disclosure concerns immunodetection methods for binding, purifying, removing, quantifying and otherwise generally detecting SARS-CoV-2 and its associated antigens. While such methods can be applied in a traditional sense, another use will be in quality control and monitoring of vaccine and other virus stocks, where antibodies according to the present disclosure can be used to assess the amount or integrity (i.e., long term stability) of antigens in viruses. Alternatively, the methods may be used to screen various antibodies for appropriate/desired reactivity profiles.

Other immunodetection methods include specific assays for determining the presence of SARS-CoV-2 in a subject. A wide variety of assay formats are contemplated, but specifically those that would be used to detect SARS-CoV-2 in a fluid obtained from a subject, such as saliva, blood, plasma, sputum, semen or urine. In particular, semen has been demonstrated as a viable sample for detecting SARS-CoV-2 (Purpura et al., 2016; Mansuy et al., 2016; Barzon et al., 2016; Gornet et al., 2016; Duffy et al., 2009; CDC, 2016; Halfon et al., 2010; Elder et al. 2005). The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of SARS-CoV-2 antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing SARS-CoV-2, and contacting the sample with a first antibody in accordance with the present disclosure, as the case may be, under conditions effective to allow the formation of immunocomplexes.

These methods include methods for purifying SARS-CoV-2 or related antigens from a sample. The antibody will preferably be linked to a solid support, such as in the form of a column matrix, and the sample suspected of containing the SARS-CoV-2 or antigenic component will be applied to the immobilized antibody. The unwanted components will be washed from the column, leaving the SARS-CoV-2 antigen immunocomplexed to the immobilized antibody, which is then collected by removing the organism or antigen from the column.

The immunobinding methods also include methods for detecting and quantifying the amount of SARS-CoV-2 or related components in a sample and the detection and quantification of any immune complexes formed during the binding process. Here, one would obtain a sample suspected of containing SARS-CoV-2 or its antigens and contact the sample with an antibody that binds SARS-CoV-2 or components thereof, followed by detecting and quantifying the amount of immune complexes formed under the specific conditions. In terms of antigen detection, the biological sample analyzed may be any sample that is suspected of containing SARS-CoV-2 or SARS-CoV-2 antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to SARS-CoV-2 or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, for example, with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the SARS-CoV-2 or SARS-CoV-2 antigen is added to the wells. After binding and washing to remove non-specifically bound immune complexes, the bound antigen may be detected. Detection may be achieved by the addition of another anti-SARS-CoV-2 antibody that is linked to a detectable label. This type of ELISA is a simple “sandwich ELISA.” Detection may also be achieved by the addition of a second anti-SARS-CoV-2 antibody, followed by the addition of a third antibody that has binding affinity for the second antibody, with the third antibody being linked to a detectable label.

In another exemplary ELISA, the samples suspected of containing the SARS-CoV-2 or SARS-CoV-2 antigen are immobilized onto the well surface and then contacted with the anti-SARS-CoV-2 antibodies of the disclosure. After binding and washing to remove non-specifically bound immune complexes, the bound anti-SARS-CoV-2 antibodies are detected. Where the initial anti-SARS-CoV-2 antibodies are linked to a detectable label, the immune complexes may be detected directly. Again, the immune complexes may be detected using a second antibody that has binding affinity for the first anti-SARS-CoV-2 antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween. or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween). After incubation with the labeled antibody, and subsequent to washing to remove unbound material, the amount of label is quantified. e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated. e.g., using a visible spectra spectrophotometer.

In another embodiment, the present disclosure contemplates the use of competitive formats. This is particularly useful in the detection of SARS-CoV-2 antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample. Here, the inventor proposes the use of labeled SARS-CoV-2 monoclonal antibodies to determine the amount of SARS-CoV-2 antibodies in a sample. The basic format would include contacting a known amount of SARS-CoV-2 monoclonal antibody (linked to a detectable label) with SARS-CoV-2 antigen or particle. The SARS-CoV-2 antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

### D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### E. Immunodetection Kits

In still further embodiments, the present disclosure concerns immunodetection kits for use with the immunodetection methods described above. As the antibodies may be used to detect SARS-CoV-2 or SARS-CoV-2 antigens, the antibodies may be included in the kit. The immunodetection kits will thus comprise, in suitable container means, a first antibody that binds to SARS-CoV-2 or SARS-CoV-2 antigen, and optionally an immunodetection reagent.

In certain embodiments, the SARS-CoV-2 antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the SARS-CoV-2 or SARS-CoV-2 antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### F. Vaccine and Antigen Quality Control Assays

The present disclosure also contemplates the use of antibodies and antibody fragments as described herein for use in assessing the antigenic integrity of a viral antigen in a sample. Biological medicinal products like vaccines differ from chemical drugs in that they cannot normally be characterized molecularly; antibodies are large molecules of significant complexity and have the capacity to vary widely from preparation to preparation. They are also administered to healthy individuals, including children at the start of their lives, and thus a strong emphasis must be placed on their quality to ensure, to the greatest extent possible, that they are efficacious in preventing or treating life-threatening disease, without themselves causing harm.

The increasing globalization in the production and distribution of vaccines has opened new possibilities to better manage public health concerns but has also raised questions about the equivalence and interchangeability of vaccines procured across a variety of sources. International standardization of starting materials, of production and quality control testing, and the setting of high expectations for regulatory oversight on the way these products are manufactured and used, have thus been the cornerstone for continued success. But it remains a field in constant change, and continuous technical advances in the field offer a promise of developing potent new weapons against the oldest public health threats, as well as new ones—malaria, pandemic influenza, and HIV, to name a few—but also put a great pressure on manufacturers, regulatory authorities, and the wider medical community to ensure that products continue to meet the highest standards of quality attainable.

Thus, one may obtain an antigen or vaccine from any source or at any point during a manufacturing process. The quality control processes may therefore begin with preparing a sample for an inmunoassay that identifies binding of an antibody or fragment disclosed herein to a viral antigen. Such immunoassays are disclosed elsewhere in this document, and any of these may be used to assess the structural/antigenic integrity of the antigen. Standards for finding the sample to contain acceptable amounts of antigenically correct and intact antigen may be established by regulatory agencies.

Another important embodiment where antigen integrity is assessed is in determining shelf-life and storage stability. Most medicines, including vaccines, can deteriorate over time. Therefore, it is critical to determine whether, over time, the degree to which an antigen, such as in a vaccine, degrades or destabilizes such that is it no longer antigenic and/or capable of generating an immune response when administered to a subject. Again, standards for finding the sample to contain acceptable amounts of antigenically intact antigen may be established by regulatory agencies.

In certain embodiments, viral antigens may contain more than one protective epitope. In these cases, it may prove useful to employ assays that look at the binding of more than one antibody, such as 2, 3, 4, 5 or even more antibodies. These antibodies bind to closely related epitopes, such that they are adjacent or even overlap each other. On the other hand, they may represent distinct epitopes from disparate parts of the antigen. By examining the integrity of multiple epitopes, a more complete picture of the antigen's overall integrity, and hence ability to generate a protective immune response, may be determined.

Antibodies and fragments thereof as described in the present disclosure may also be used in a kit for monitoring the efficacy of vaccination procedures by detecting the presence of protective SARS-CoV-2 antibodies. Antibodies, antibody fragment, or variants and derivatives thereof, as described in the present disclosure may also be used in a kit for monitoring vaccine manufacture with the desired immunogenicity.

### G. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods for Example 2

Expression and purification of recombinant receptor binding domain (RBD) of SARS-CoV-2 spike protein. The DNA segments correspondent to the S protein RBD (residues 319-528) was sequence optimized for expression, synthesized, and cloned into the pTwist-CMV expression DNA plasmid downstream of the IL-2 signal peptide (MYRMQLLSCIALSLALVTNS) (Twist Bioscience). A three amino acid linker (GSG) and a His-tag were incorporated at the C-terminus of the expression constructs to facilitate protein purification. Expi293F cells were transfected transiently with the plasmid encoding RBD, and culture supernatants were harvested after 5 days. RBD was purified from the supernatants by nickel affinity chromatography with HisTrap Excel columns (GE Healthcare Life Sciences). For protein production used in crystallization trials, 5 μM kifunensine was included in the culture medium to produce RBD with high mannose glycans. The high mannose glycoproteins subsequently were treated with endoglycosidase F1 (Millipore) to obtain homogeneously deglycosylated RBD.

Isolation or generation of authentic SARS-CoV-2 viruses, including viruses with variant residues. The UK B.1.1.7-OXF isolate was obtained from a nasopharyngeal swab from an infected individual in Kent. England. The clinical studies to obtain specimens after written informed consent were approved by John Radcliffe Hospital in Oxford, U.K. The sample was diluted in DMEM with 2% FBS and passed through a 0.45 μm filter before adding to monolayers of Vero-hACE2-TMPRSS2 cells (a gift of A. Creanga and B. Graham). Two days later, supernatant was harvested to establish a passage zero (p0) stock. The 2019n-CoV/USA_WA1/2019 isolate of SARS-CoV-2 was obtained from the U.S. Centers for Disease Control (CDC) and passaged on Vero E6 cells. Individual point mutations in the spike gene (D614G and E484K/D614G) were introduced into an infectious cDNA clone of the 2019n-CoV/USA_WA1/2019 strain as described previously⁶⁷. Nucleotide substitutions were introduced into a subclone puc57-CoV-2-F6 containing the spike gene of the SARS-CoV-2 wild-type infectious clone⁶⁸. The full-length infectious cDNA clones of the variant SARS-CoV-2 viruses were assembled by in vitro ligation of seven contiguous cDNA fragments following the previously described protocol⁶⁸. In vitro transcription then was performed to synthesize full-length genomic RNA. To recover the mutant viruses, the RNA transcripts were electroporated into Vero E6 cells. The viruses from the supernatant of cells were collected 40 h later and served as p0 stocks. All virus stocks were confirmed by sequencing.

Focus reduction neutralization test. Serial dilutions of mAbs or serum were incubated with 10² focus-forming units (FFU) of different strains or variants of SARS-CoV-2 for 1 h at 37° C. Antibody-virus complexes were added to Vero-hACE2-TMPRSS2 cell monolayer cultures in 96-well plates and incubated at 37° C. for 1 h. Subsequently, cells were overlaid with 1% (w/v) methylcellulose in MEM supplemented with 2% FBS. Plates were harvested 20 h later by removing overlays and fixed with 4% PFA in PBS for 20 min at room temperature. Plates were washed and sequentially incubated with an oligoclonal pool of anti-S mAbs and HRP-conjugated goat anti-human IgG in PBS supplemented with 0.1% saponin and 0.1% bovine serum albumin. SARS-CoV-2-infected cell foci were visualized using TrueBlue peroxidase substrate (KPL) and quantitated on an ImmunoSpot microanalyzer (Cellular Technologies).

Multiple sequence alignments. The inventors searched for antibody variable gene sequences originating with the same features as those encoding COV2-2196 and retrieved the matching sequences from the repertoires of each individual examined. They searched for similar sequences in the publicly available large-scale antibody sequence repertoires for three healthy individuals and cord blood repertoires (deposited at SRP174305). The search parameters for the heavy chain were sequences with IGHV1-58 and IGHJ3 with the P99. D108, and F110 residues. Additionally, the search parameters for the light chain were sequences with Y92 and W98 residues. Sequences from a matching clonotype that belonged to each individual were aligned with either ClustalO (heavy chains) or with MUSCLE (light chains). Then, LOGOs plots of aligned sequences were generated using WebLogo.

Data and materials availability: The crystal structures reported in this paper have been deposited to the Protein Data Bank under the accession numbers 7L7D, 7L7E. Sequence Read Archive deposition for the aligned human antibody gene repertoire data set is deposited at the NCBI: PRJNA511481. All other data are available in the main text or the supplementary materials.

### Example 2—Result and Discussion

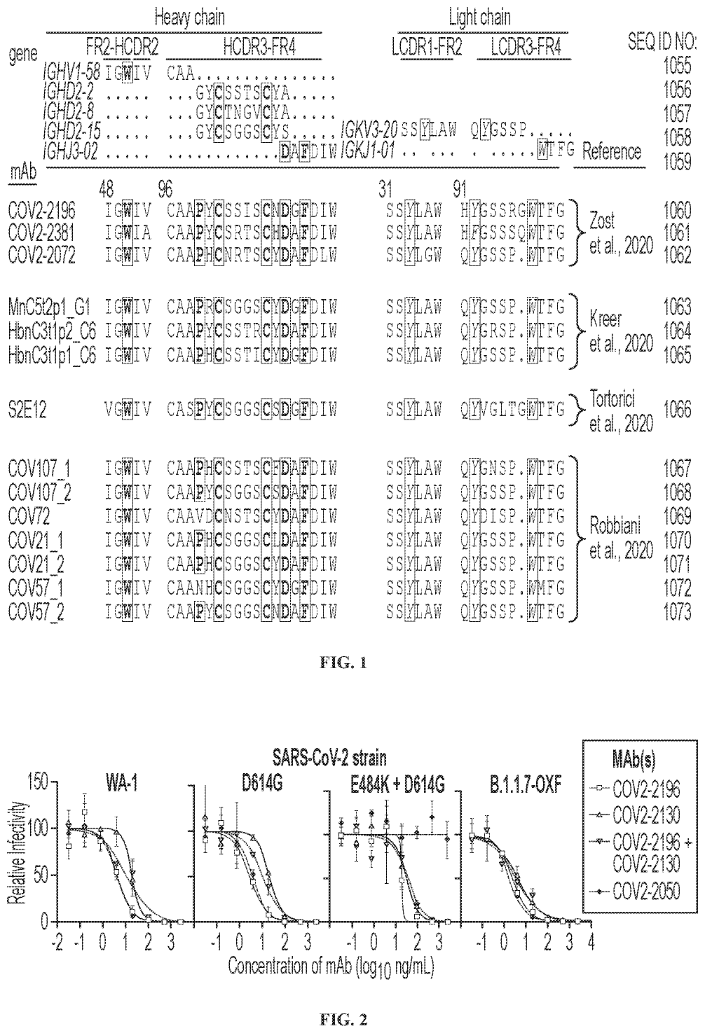

Structural analysis of COV2-2196 in complex with RBD reveals how COV2-2196 recognizes the receptor-binding ridge on the RBD. One of the major contact residues, F486, situates at the center of the binding site, interacting extensively with the hydrophobic pocket (residue P99 of heavy chain and an “aromatic cage” formed by 5 aromatic side chains) between COV2-2196 heavy/light chains via a hydrophobic effect and van der Waals interactions. A hydrogen bond (H-bond) network, constructed with 4 direct Ab-Ag H-bonds and 16 water-mediated H-bonds, surround residue F486 and strengthen the Ab-Ag interaction. Importantly, for all residues except one (residue P99 of the heavy chain) that interact extensively with the epitope, they are encoded by germline sequences (IGHV1-58*01 and IGHJ3*02 for the heavy chain, IGKV3-20*01 and IGKJ1*01 for the light chain) (FIG. 1) or only their backbone atoms are involved in the Ab-Ag interactions, such as heavy chain N107 and G99 and light chain S94. The inventors noted another antibody in the literature, S2E12, that is encoded by the same IGHV/IGHJ and IGKV/IGKJ recombinations, with similar but most likely different IGHD genes to those of COV2-2196 (IGHD2-15 vs IGHD2-2)³⁸. A comparison of the cryo-EM structure of S2E12 in complex with S protein (PDB 7K4N) suggests that the mAb S2E12 likely uses nearly identical Ab-Ag interactions as those of COV2-2196, although variations in conformations of interface residue side-chains can be seen. For example, for light chain residue Y92, the phenyl ring in the crystal structure is perpendicular to that ring in the EM structure as fitted.

The inventors searched genetic databases to determine if these structural features are present in additional SARS-CoV-2 mAbs isolated by others and found additional members of the clonotype (FIG. 1). Two other studies reported the same or a similar clonotype of antibodies isolated from multiple COVID-19 convalescent patients^{4,38}, and one study found three antibodies with the same IGHV1-58 and IGKV3-20 pairing, without providing information on D or J gene usage³⁹. All of these antibodies are reported to bind SARS-CoV-2 RBD avidly and to neutralize virus with high potency^{1,4,38,39}. So far, there are only two atomic resolution structures of antibodies encoded by these V_{H}(D_{H})J_{H} and V_{K}-J_{K} recombinations available, the structure for COV2-2196 and that for S2E12³⁸.

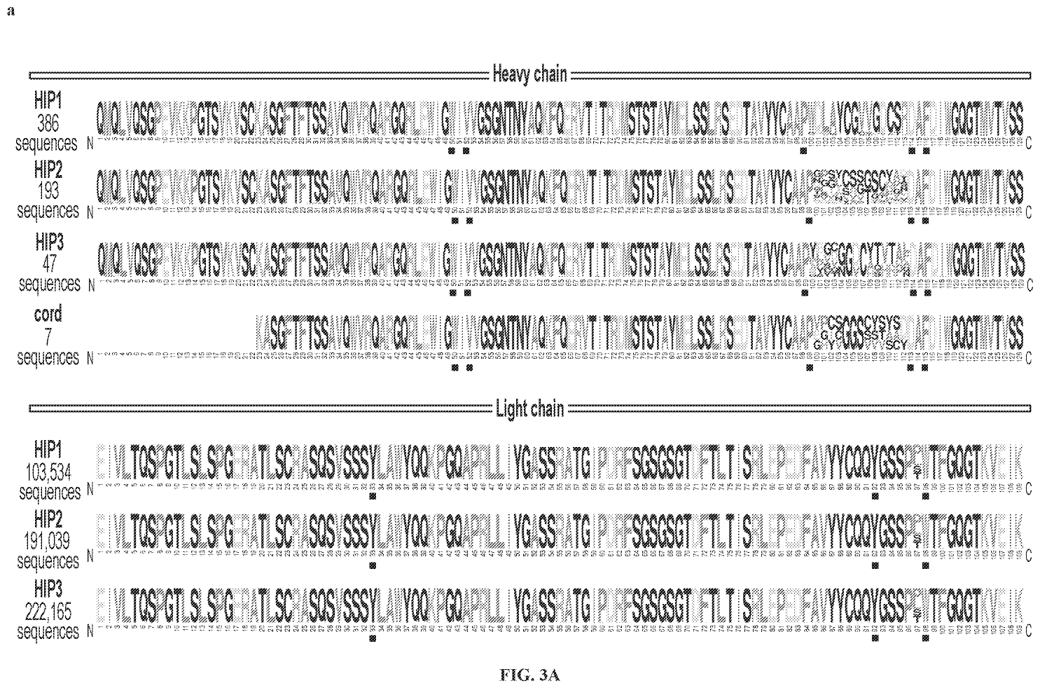

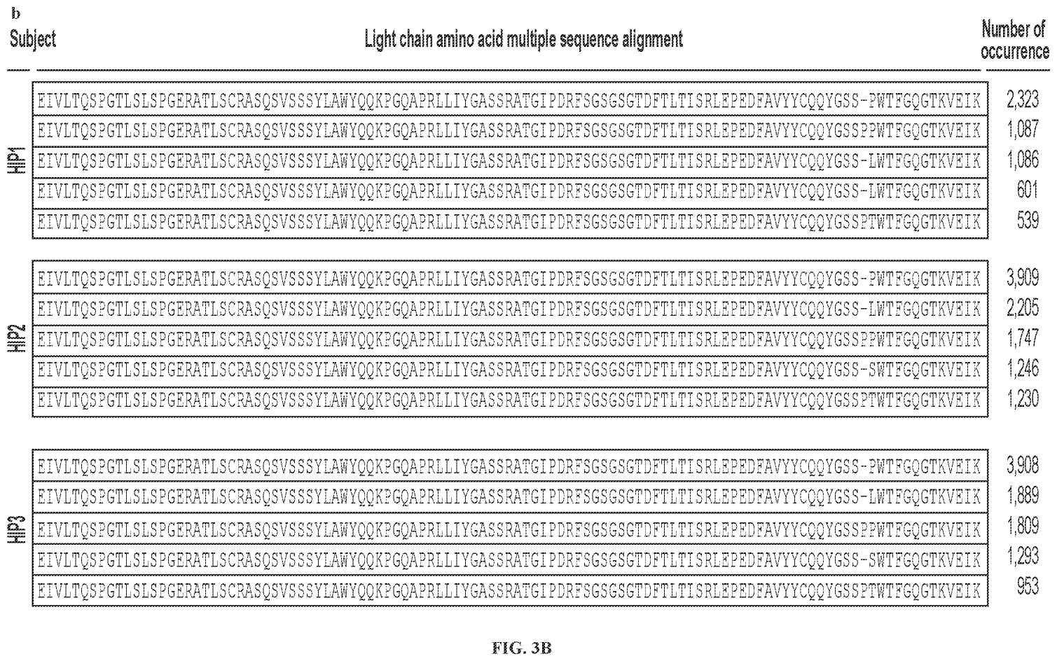

The inventors next determined whether they could identify potential precursors of this public clonotype in the antibody variable gene repertoires of circulating B cells from SARS-CoV-2-naïve individuals. The inventors searched for the V(D)J and VJ genes in previously described comprehensive repertoire datasets originating from 3 healthy human donors, without a history of SARS-CoV-2 infection, and in datasets from cord blood collected prior to the COVID-19 pandemico. A total of 386, 193, 47, or 7 heavy chain sequences for this SARS-CoV-2 reactive public clonotype was found in each donor or cord blood repertoire, respectively (FIG. 3A). Additionally, the inventors found 516,738 human antibody sequences with the same light chain V-J recombination (IGKV3-20-IGKJ1*01). A total of 103.534, 191,039, or 222,165 light chain sequences was found for this public clonotype in each donor respectively. Due to the large number of sequences, the top five abundant sequences were aligned from each donor. Multiple sequence alignments were generated for each donor's sequences using ClustalOmega, and logo plots were generated. The top 5 sequences with the same recombination event in each donor were identical, resulting in the same logo plots (FIGS. 3A-B).

The inventors noted that 8 of the 9 common residues important for binding in the antibody were encoded by germline gene sequences, and all were present all 14 members of the public clonotype listed here from four different antibody-discovery teams (FIG. 1).

Recently, viral variants with increased transmissibility and potential antigenic mutations have been reported in clinical isolates⁴⁸⁻⁵¹. The inventors tested whether some of the variant residues in these rapidly emerging strains would abrogate the activity of these potently neutralizing antibodies. They tested a viral isolate from a nasal sample obtained at Oxford in the United Kingdom (a B.1.1.7 virus designated UK B.1.1.7-OXF), which contains B.1.1.7 lineage defining spike gene changes including the 69-70 and 144-145 deletions in the NTD, and substitutions at N501Y, A570D, D614G, and P681H⁴⁹. The inventors also tested isogenic D614G and E484K variants in the WA-1 strain background (2019n-CoV/USA_WA1/2019, [WA-1]), all prepared as authentic SARS-CoV-2 viruses and used in focus reduction neutralization tests⁴³. The E484K mutation was of special interest, since this residue is located within 4.5 Å of each of the mAbs in the complex of Fabs and RBD, albeit at the very periphery of the Fab footprints, is present in emerging lineages B.1.351 (501Y.V2)⁵⁰ and P.1 (501Y.V3)⁵¹, and has been demonstrated to alter the binding of some monoclonal antibodies^{52,53} as well as human polyclonal serum antibodies⁵⁴. Variants containing E484K also have been shown to be neutralized less efficiently by convalescent serum and plasma from SARS-CoV-2 survivors^{55,56}. For COV2-2196, COV2-2130, and COV2-2050 (a third neutralizing antibody the inventors included for comparison as it interacts with the residue E484), they found virtually no impact of the D614G mutation or the suite of mutations present in the UK B.1.1.7-OXF strain; if anything, the inventors observed a trend toward slightly improved (2- to 3-fold reduction in IC₅₀ values) against the latter circulating virus (FIG. 2). However, they did observe effects on neutralization with the D614G/E484K virus. COV2-2050 completely lost neutralization activity, consistent with our previous study defining E484K as a mutation abrogating COV2-2050 binding⁴¹.

Discussion. These structural analyses define the molecular basis for the frequent selection of a public clonotype of human antibodies sharing heavy chain V-D-J and light chain V-J recombinations that target the same region of the SARS-CoV-2 S RBD. Germline antibody gene-encoded residues in heavy and light chains play a vital role in antigen recognition, suggesting that few somatic mutations are required for antibody maturation of this clonotype. An IGHD2-gene-encoded disulfide bond provides additional restraint for the HCDR3 to adopt a conformation with shape and chemical complementarity to the antigenic site on RBD. It appears that three different IGHD2 genes (IGHD2-2, IGHD2-8, and IGHD2-15) encode portions of the HCDR3 that can function in the context of this clonotype. The inventors suggest that this occurrence of common germline gene-encoded antibodies with preconfigured structural features enabling high specificity and potent neutralizing activity is an unanticipated and beneficial feature of the primary human immune response to SARS-CoV-2. The selection of B cells from this public clonotype enabled rapid isolation of ultra-potent neutralizing antibodies that resist escape and possibly could account in part for the remarkable efficacy of S protein-based vaccines that is being observed in the clinic. One might envision an opportunity to elicit serum neutralizing antibody titers with even higher neutralization potency using domain- or motif-based vaccine designs for this antigenic site to prime human immune responses to elicit this clonotype.

The recent emergence of variant virus lineages with increased transmissibility and altered sequences in many known sites of neutralization is concerning for the capacity of SARS-CoV-2 to evade current antibody countermeasures in development and testing. The inventors tested the activity of the antibodies and the cocktail of both and found sustained activity against several important variants, including a virus containing the E484K mutation and a B.1.1.7 virus with multiple S gene variations. The genetic and structural basis for this broad activity is revealed in the crystal structures and DMS studies the inventors present here.

### REFERENCES FOR EXAMPLES 1 AND 2

1. Zost, S. J. et al. Potently neutralizing and protective human antibodies against SARS-CoV-2. Nature 584, 443-449, doi:10.1038/s41586-020-2548-6 (2020).
2. Yuan, M. et al. Structural basis of a shared antibody response to SARS-CoV-2. Science 369, 1119-1123, doi:10.1126/science.abd2321 (2020).
3. Nielsen, S. C. A. et al. Human B Cell Clonal Expansion and Convergent Antibody Responses to SARS-CoV-2. Cell Host Microbe 28, 516-525 e515, doi:10.1016/j.chom.2020.09.002 (2020).
4. Robbiani, D. F. et al. Convergent antibody responses to SARS-CoV-2 in convalescent individuals. Nature 584, 437-442, doi:10.1038/s41586-020-2456-9 (2020).
5. Brouwer. P. J. M. et al. Potent neutralizing antibodies from COVID-19 patients define multiple targets of vulnerability. Science 369, 643-650, doi:10.1126/science.abc5902 (2020).
6. Zhou, P. et al. A pneumonia outbreak associated with a new coronavirus of probable bat origin. Nature 579, 270-273, doi:10.1038/s41586-020-2012-7 (2020).
7. Zhu, N. et al. A Novel Coronavirus from Patients with Pneumonia in China, 2019. N Engl J Med 382, 727-733, doi:10.1056/NEJMoa2001017 (2020).
8. Hoffmann, M. et al. SARS-CoV-2 Cell Entry Depends on ACE2 and TMPRSS2 and Is Blocked by a Clinically Proven Protease Inhibitor. Cell 181, 271-280 e278. doi:10.1016/j.cell.2020.02.052 (2020).
9. Letko, M., Marzi, A. & Munster, V. Functional assessment of cell entry and receptor usage for SARS-CoV-2 and other lineage B betacoronaviruses. Nat Microbiol 5, 562-569, doi:10.1038/s41564-020-0688-y (2020).
10. Wahba, L. et al. An Extensive Meta-Metagenomic Search Identifies SARS-CoV-2-Homologous Sequences in Pangolin Lung Viromes. mSphere 5, doi:10.1128/mSphere.00160-20 (2020).
11. Walls. A. C. et al. Tectonic conformational changes of a coronavirus spike glycoprotein promote membrane fusion. Proc Natl Acad Sci USA 114, 11157-11162, doi:10.1073/pnas.1708727114 (2017).
12. Algaissi, A. et al. SARS-CoV-2 S1 and N-based serological assays reveal rapid seroconversion and induction of specific antibody response in COVID-19 patients. Sci Rep 10, 16561, doi:10.1038/s41598-020-73491-5 (2020).
13. Long, Q. X. et al. Antibody responses to SARS-CoV-2 in patients with COVID-19. Nat Med 26, 845-848, doi:10.1038/s41591-020-0897-1 (2020).
14. Piccoli, L. et al. Mapping Neutralizing and Immunodominant Sites on the SARS-CoV-2 Spike Receptor-Binding Domain by Structure-Guided High-Resolution Serology. Cell, doi:10.1016/j.cell.2020.09.037 (2020).
15. Cao, Y. et al. Potent Neutralizing Antibodies against SARS-CoV-2 Identified by High-Throughput Single-Cell Sequencing of Convalescent Patients' B Cells. Cell 182, 73-84 e16, doi:10.1016/j.cell.2020.05.025 (2020).
16. Hansen, J. et al. Studies in humanized mice and convalescent humans yield a SARS-CoV-2 antibody cocktail. Science 369, 1010-1014, doi:10.1126/science.abd0827 (2020).
17. Ju, B. et al. Human neutralizing antibodies elicited by SARS-CoV-2 infection. Nature 584, 115-119, doi:10.1038/s41586-020-2380-z (2020).
18. Liu, L. et al. Potent neutralizing antibodies against multiple epitopes on SARS-CoV-2 spike. Nature 584, 450-456, doi:10.1038/s41586-020-2571-7 (2020).
19. Rogers, T. F. et al. Isolation of potent SARS-CoV-2 neutralizing antibodies and protection from disease in a small animal model. Science 369, 956-963, doi:10.1126/science.abc7520 (2020).
20. Shi, R. et al. A human neutralizing antibody targets the receptor-binding site of SARS-CoV-2. Nature 584, 120-124, doi:10.1038/s41586-020-2381-y (2020).
21. Weitkamp, J. H. et al. Infant and adult human B cell responses to rotavirus share common immunodominant variable gene repertoires. J Immunol 171, 4680-4688, doi:10.4049/jimmunol.171.9.4680 (2003).
22. Benton, D. J. et al. Receptor binding and priming of the spike protein of SARS-CoV-2 for membrane fusion. Nature, doi:10.1038/s41586-020-2772-0 (2020).
23. Wrapp, D. et al. Cryo-EM structure of the 2019-nCoV spike in the prefusion conformation. Science 367, 1260-1263, doi:10.1126/science.abb2507 (2020).
24. Wrobel, A. G. et al. SARS-CoV-2 and bat RaTG13 spike glycoprotein structures inform on virus evolution and furin-cleavage effects. Nat Struct Mol Biol 27, 763-767, doi:10.1038/s41594-020-0468-7 (2020).
25. Zost, S. J. et al. Rapid isolation and profiling of a diverse panel of human monoclonal antibodies targeting the SARS-CoV-2 spike protein. Nat Med 26, 1422-1427, doi:10.1038/s41591-020-0998-x (2020).
26. Tian, C. et al. Immunodominance of the VH1-46 antibody gene segment in the primary repertoire of human rotavirus-specific B cells is reduced in the memory compartment through somatic mutation of nondominant clones. J Immunol 180, 3279-3288, doi:10.4049/jimmunol.180.5.3279 (2008).
27. Wu, X. et al. Focused evolution of HIV-1 neutralizing antibodies revealed by structures and deep sequencing. Science 333, 1593-1602, doi:10.1126/science.1207532 (2011).
28. Zhou, T. et al. Structural Repertoire of HIV-1-Neutralizing Antibodies Targeting the CD4 Supersite in 14 Donors. Cell 161, 1280-1292, doi:10.1016/j.cell.2015.05.007 (2015).
29. Huang, C. C. et al. Structural basis of tyrosine sulfation and VH-gene usage in antibodies that recognize the HIV type 1 coreceptor-binding site on gp120. Proc Natl Acad Sci USA 101, 2706-2711, doi:10.1073/pnas.0308527100 (2004).
30. Williams. W. B. et al. HIV-1 VACCINES. Diversion of HIV-1 vaccine-induced immunity by gp41-microbiota cross-reactive antibodies. Science 349, aab1253, doi:10.1126/science.aab1253 (2015).
31. Joyce, M. G. et al. Vaccine-Induced Antibodies that Neutralize Group 1 and Group 2 Influenza A Viruses. Cell 166, 609-623, doi:10.1016/j.cell.2016.06.043 (2016).
32. Pappas, L. et al. Rapid development of broadly influenza neutralizing antibodies through redundant mutations. Nature 516, 418-422, doi:10.1038/nature13764 (2014).
33. Sui, J. et al. Structural and functional bases for broad-spectrum neutralization of avian and human influenza A viruses. Nat Struct Mol Biol 16, 265-273, doi:10.1038/nsmb.1566 (2009).
34. Wheatley, A. K. et al. H5N1 Vaccine-Elicited Memory B Cells Are Genetically Constrained by the IGHV Locus in the Recognition of a Neutralizing Epitope in the Hemagglutinin Stem. J Immunol 195, 602-610, doi:10.4049/jimmunol.1402835 (2015).
35. Bailey, J. R. et al. Broadly neutralizing antibodies with few somatic mutations and hepatitis C virus clearance. JCI Insight 2, doi:10.1172/jci.insight.92872 (2017).
36. Giang, E. et al. Human broadly neutralizing antibodies to the envelope glycoprotein complex of hepatitis C virus. Proc Natl Acad Sci USA 109, 6205-6210, doi:10.1073/pnas. 114927109 (2012).
37. Rappuoli, R., Bottomley, M. J., D'Oro, U., Finco, O. & De Gregorio. E. Reverse vaccinology 2.0: Human immunology instructs vaccine antigen design. J Exp Med 213, 469-481. doi:10.1084/jem.20151960 (2016).
38. Tortorici, M. A. et al. Ultrapotent human antibodies protect against SARS-CoV-2 challenge via multiple mechanisms. Science, doi:10.1126/science.abe3354 (2020).
39. Kreer, C. et al. Longitudinal Isolation of Potent Near-Germline SARS-CoV-2-Neutralizing Antibodies from COVID-19 Patients. Cell 182, 843-854 e812, doi:10.1016/j.cell.2020.06.044 (2020).
40. Soto, C. et al. High frequency of shared clonotypes in human B cell receptor repertoires. Nature 566, 398-402, doi:10.1038/s41586-019-0934-8 (2019).
41. Greaney, A. J. et al. Complete mapping of mutations to the SARS-CoV-2 spike receptor-binding domain that escape antibody recognition. Cell Host Microbe, doi:10.1016/j.chom.2020.11.007 (2020).
42. Starr, T. N. et al. Deep mutational scanning of SARS-CoV-2 receptor binding domain reveals constraints on folding and ACE2 binding. Cell 182, 1295-1310 e1220, doi:10.1016/j.cell.2020.08.012 (2020).
43. Case, J. B. et al. Neutralizing antibody and soluble ACE2 inhibition of a replication-competent VSV-SARS-CoV-2 and a clinical isolate of SARS-CoV-2. Cell Host Microbe 28, 475-485 e475, doi:10.1016/j.chom.2020.06.021 (2020).
44. Klimstra, W. B. et al. SARS-CoV-2 growth, furin-cleavage-site adaptation and neutralization using serum from acutely infected hospitalized COVID-19 patients. J Gen Virol 101, 1156-1169, doi:10.1099/jgv.0.001481 (2020).
45. Sawatzki, K. et al. Ferrets not infected by SARS-CoV-2 in a high-exposure domestic setting. bioRxiv, 2020.2008.2021.254995, doi:10.1101/2020.08.21.254995 (2020).
46. Baum, A. et al. REGN-COV2 antibodies prevent and treat SARS-CoV-2 infection in rhesus macaques and hamsters. Science 370, 1110-1115, doi:10.1126/science.abe2402 (2020).
47. Li, Q. et al. The impact of mutations in SARS-CoV-2 spike on viral infectivity and antigenicity. Cell 182, 1284-1294 e1289, doi:10.1016/j.cell.2020.07.012 (2020).
48. Galloway, S. E. et al. Emergence of SARS-CoV-2 B.1.1.7 Lineage—United States, Dec. 29, 2020-Jan. 12, 2021. MMWR Morb Mortal Wkly Rep 70, 95-99. doi:10.15585/mmwr.mm7003e2 (2021).
49. Leung. K., Shum, M. H., Leung, G. M., Lam, T. T. & Wu, J. T. Early transmissibility assessment of the N501Y mutant strains of SARS-CoV-2 in the United Kingdom, October to November 2020. Euro Surveill 26, doi:10.2807/1560-7917.ES.2020.26.1.2002106 (2021).
50. Tegally, H. et al. Emergence and rapid spread of a new severe acute respiratory syndrome-related coronavirus 2 (SARS-CoV-2) lineage with multiple spike mutations in South Africa. medRxiv, 2020.2012.2021.20248640, doi:10.1101/2020.12.21.20248640 (2020).
51. Voloch, C. M. et al. Genomic characterization of a novel SARS-CoV-2 lineage from Rio de Janeiro, Brazil. medRxiv, 2020.2012.2023.20248598, doi:10.1101/2020.12.23.20248598 (2020).
52. Liu, Z. et al. Landscape analysis of escape variants identifies SARS-CoV-2 spike mutations that attenuate monoclonal and serum antibody neutralization. bioRxiv, 2020.2011.2006.372037, doi:10.1101/2020.11.06.372037 (2021).
53. Weisblum, Y. et al. Escape from neutralizing antibodies by SARS-CoV-2 spike protein variants. Elife 9. doi:10.7554/eLife.61312 (2020).
54. Greaney, A. J. et al. Comprehensive mapping of mutations to the SARS-CoV-2 receptor-binding domain that affect recognition by polyclonal human serum antibodies. bioRxiv, 2020.2012.2031.425021, doi:10.1101/2020.12.31.425021 (2021).
55. Wibmer, C. K. et al. SARS-CoV-2 501Y.V2 escapes neutralization by South African COVID-19 donor plasma. bioRxiv, 2021.2001.2018.427166, doi:10.1101/2021.01.18.427166 (2021).
56. Andreano, E. et al. SARS-CoV-2 escape in vitro from a highly neutralizing COVID-19 convalescent plasma. bioRxiv, 2020.2012.2028.424451, doi:10.1101/2020.12.28.424451 (2020).
57. Huo, J. et al. Neutralization of SARS-CoV-2 by destruction of the prefusion spike. Cell Host Microbe 28,445-454 e446, doi:10.1016/j.chom.2020.06.010 (2020).
58. Kabsch, W. Xds. Acta Crystallogr D Biol Crystallogr 66, 125-132, doi:10.1107/50907444909047337 (2010).
59. Winn, M. D. et al. Overview of the CCP4 suite and current developments. Acta Crystallogr D Biol Crystallogr 67, 235-242, doi:10.1107/S0907444910045749 (2011).
60. McCoy, A. J. et al. Phaser crystallographic software. J Appl Crystallogr 40, 658-674. doi:10.1107/50021889807021206 (2007).
61. Adams, P. D. et al. PHENIX: a comprehensive Python-based system for macromolecular structure solution. Acta Crystallogr D Biol Crystallogr 66, 213-221, doi:10.1107/S0907444909052925 (2010).
62. Emsley. P. & Cowtan, K. Coot: model-building tools for molecular graphics. Acta Crystallogr D Biol Crystallogr 60, 2126-2132. doi:10.1107/S0907444904019158 (2004).
63. Schrodinger, LLC. The PyMOL Molecular Graphics System, Version 1.8 (2015).
64. Cornwell, O., Radford. S. E., Ashcroft, A. E. & Ault, J. R. Comparing hydrogen deuterium exchange and fast photochemical oxidation of proteins: a structural characterisation of wild-type and deltaN6 beta2-microglobulin. J Am Soc Mass Spectrom 29, 2413-2426, doi:10.1007/s13361-018-2067-y (2018).
65. Starr, T. N. et al. Prospective mapping of viral mutations that escape antibodies used to treat COVID-19. bioRxiv, doi:10.1101/2020.11.30.405472 (2020).
66. Otwinowski, J., McCandlish, D. M. & Plotkin, J. B. Inferring the shape of global epistasis. Proc Natl Acad Sci USA 115, E7550-E7558, doi:10.1073/pnas.1804015115 (2018).
67. Plante, J. A. et al. Spike mutation D614G alters SARS-CoV-2 fitness. Nature, doi:10.1038/s41586-020-2895-3 (2020).
68. Xie, X. et al. An infectious cDNA clone of SARS-CoV-2. Cell Host Microbe 27, 841-848 e843, doi:10.1016/j.chom.2020.04.004 (2020).

### Example 3—Materials and Methods for Example 4

Antibodies. The human antibodies studied in this paper were isolated from blood samples from two subjects in North America with previous laboratory-confirmed symptomatic SARS-CoV-2 infection that was acquired in China. The original clinical studies to obtain specimens after written informed consent were previously described¹ and had been approved by the Institutional Review Board of Vanderbilt University Medical Center and the Research Ethics Board of the University of Toronto. The subjects (a 56-year-old male and a 56-year-old female) are a married couple and residents of Wuhan, China who traveled to Toronto, Canada, where PBMCs were obtained by leukopheresis 50 days after symptom onset. The antibodies were isolated using diverse tools for isolation and cloning of single antigen-specific B cells and the antibody variable genes encoding monoclonal antibodies¹.

Cell culture. Vero E6 (CRL-1586, American Type Culture Collection (American Type Culture Collection, ATCC), Vero CCL81 (ATCC), HEK293 (ATCC), and HEK293T (ATCC) were maintained at 37° C. in 5% CO₂ in Dulbecco's minimal essential medium (DMEM) containing 10% (vol/vol) heat-inactivated fetal bovine serum (FBS), 10 mM HEPES pH 7.3, 1 mM sodium pyruvate, 1× non-essential amino acids, and 100 U/mL of penicillin-streptomycin. Vero-furin cells were obtained from T. Pierson (NIH) and have been described previously². Expi293F cells (ThermoFisher Scientific. A1452) were maintained at 37° C. in 8% CO₂ in Expi293F Expression Medium (ThermoFisher Scientific, A1435102). ExpiCHO cells (ThermoFisher Scientific, A29127) were maintained at 37° C. in 8% CO₂ in ExpiCHO Expression Medium (ThermoFisher Scientific, A2910002). Mycoplasma testing of Expi293F and ExpiCHO cultures was performed on a monthly basis using a PCR-based mycoplasma detection kit (ATCC, 30-1012K).

Viruses. SARS-CoV-2 strain 2019 n-CoV/USA_WA1/2020 was obtained from the Centers for Disease Control and Prevention (a gift from Natalie Thornburg). Virus was passaged in Vero CCL81 cells and titrated by plaque assay on Vero E6 cells. All work with infectious SARS-CoV-2 was approved by the Washington University School of Medicine or UNC-Chapel Hill Institutional Biosafety Committees and conducted in approved BSL3 facilities using appropriate powered air purifying respirators and personal protective equipment.

Recombinant antigens and proteins. A gene encoding the ectodomain of a prefusion conformation-stabilized SARS-CoV-2 spike (S2P_{ecto}) protein was synthesized and cloned into a DNA plasmid expression vector for mammalian cells. A similarly designed S protein antigen with two prolines and removal of the furin cleavage site for stabilization of the prefusion form of S was reported previously³. Briefly, this gene includes the ectodomain of SARS-CoV-2 (to residue 1,208), a T4 fibritin trimerization domain, an AviTag site-specific biotinylation sequence, and a C-terminal 8×-His tag. To stabilize the construct in the prefusion conformation, the inventors included substitutions K986P and V987P and mutated the furin cleavage site at residues 682-685 from RRAR to ASVG. This recombinant spike 2P-stabilized protein (designated here as S2P_{ecto}) was isolated by metal affinity chromatography on HisTrap Excel columns (GE Healthcare), and protein preparations were purified further by size-exclusion chromatography on a Superose 6 Increase 10/300 column (GE Healthcare). The presence of trimeric, prefusion conformation S protein was verified by negative-stain electron microscopy¹. For electron microscopy with S and Fabs, the inventors expressed a variant of S2P_{ecto} lacking an AviTag but containing a C-terminal Twin-Strep-tag, similar to that described previously³. Expressed protein was isolated by metal affinity chromatography on HisTrap Excel columns (GE Healthcare), followed by further purification on a StrepTrap HP column (GE Healthcare) and size-exclusion chromatography on TSKgel G4000SW_{XL} (TOSOH). To express the SRBD subdomain of SARS-CoV-2 S protein, residues 319-541 were cloned into a mammalian expression vector downstream of an IL-2 signal peptide and upstream of a thrombin cleavage site, an AviTag, and a 6×-His tag. RBD protein fused to mouse IgG1 Fc domain (designated RBD-mFc), was purchased from Sino Biological (40592-V05H). For epitope mapping by alanine scanning, SARS-CoV-2 RBD (residues 334-526) or RBD single mutation variants were cloned with an N-terminal CD33 leader sequence and C-terminal GSSG linker, AviTag, GSSG linker, and 8×HisTag. Spike proteins were expressed in FreeStyle 293 cells (Thermo Fisher) and isolated by affinity chromatography using a HisTrap column (GE Healthcare), followed by size exclusion chromatography with a Superdex200 column (GE Healthcare). Purified proteins were analyzed by SDS-PAGE to ensure purity and appropriate molecular weights.

Electron microscopy (EM) stain grid preparation, imaging and processing of SARS-CoV-2 S2P_{ecto} protein or S2P_{ecto}/Fab complexes. To perform EM imaging, Fabs were produced by digesting recombinant chromatography-purified IgGs using resin-immobilized cysteine protease enzyme (FabALACTICA, Genovis). The digestion occurred in 100 mM sodium phosphate, 150 mM NaCl pH 7.2 for ˜16 hrs at RT. In order to remove cleaved Fe and intact IgG, the digestion mix was incubated with CaptureSelect Fc resin (Genovis) for 30 min at RT in PBS buffer. If needed, the Fab was buffer exchanged into Tris buffer by centrifugation with a Zeba spin column (Thermo Scientific).

For screening and imaging of negatively-stained (NS) SARS-CoV-2 S2P_{ecto} protein in complex with human Fabs, the proteins were incubated for ˜1 hr and approximately 3 μL of the sample at concentrations of about 10 to 15 μg/mL was applied to a glow discharged grid with continuous carbon film on 400 square mesh copper EM grids (Electron Microscopy Sciences). The grids were stained with 0.75% uranyl formate (UF). Images were recorded on a Gatan US4000 4k×4k CCD camera using an FEI TF20 (TFS) transmission electron microscope operated at 200 keV and control with SerialEM⁵. All images were taken at 50,000× magnification with a pixel size of 2.18 Å/pix in low-dose mode at a defocus of 1.5 to 1.8 μm. Total dose for the micrographs was ˜25 to 38 e⁻/Å². Image processing was performed using the cryoSPARC software package⁶. Images were imported, and particles were CTF estimated. The images then were denoised and picked with Topaz⁷. The particles were extracted with a box size of 256 pixels and binned to 128 pixels. 2D class averages were performed and good classes selected for ab-initio model and refinement without symmetry. For EM model docking of SARS-CoV-2 S2P_{ecto} protein, the closed model (PDB: 6VXX) was used in Chimera⁸ for docking to the EM map. All images were made with Chimera.

MAb production and purification. Sequences of mAbs that had been synthesized (Twist Bioscience) and cloned into an IgG1 monocistronic expression vector (designated as pTwist-mCis_G1) were used for mammalian cell culture mAb secretion. This vector contains an enhanced 2A sequence and GSG linker that allows simultaneous expression of mAb heavy and light chain genes from a single construct upon transfection⁹. The inventors previously described microscale expression of mAbs in 1 mL ExpiCHO cultures in 96-well plates¹. For larger scale mAb expression, they performed transfection (1 to 300 mL per antibody) of CHO cell cultures using the Gibco™ ExpiCHO™ Expression System and protocol for 50 mL mini bioreactor tubes (Corning) as described by the vendor. Culture supernatants were purified using HiTrap MabSelect SuRe (Cytiva, formerly GE Healthcare Life Sciences) on a 24-column parallel protein chromatography system (Protein BioSolutions). Purified mAbs were buffer-exchanged into PBS, concentrated using Amicon® Ultra-4 50 KDa Centrifugal Filter Units (Millipore Sigma) and stored at 4° C. until use.

ELISA binding assays. Wells of 96-well microtiter plates were coated with purified recombinant SARS-CoV-2 S protein or SARS-CoV-2 SRBD protein at 4° C. overnight. Plates were blocked with 2% non-fat dry milk and 2% normal goat serum in DPBS containing 0.05% Tween-20 (DPBS-T) for 1 hr. The bound antibodies were detected using goat anti-human IgG conjugated with HRP (horseradish peroxidase) (Southern Biotech) and TMB (3,3′,5,5′-tetramethylbenzidine) substrate (Thermo Fisher Scientific). Color development was monitored, 1N hydrochloric acid was added to stop the reaction, and the absorbance was measured at 450 nm using a spectrophotometer (Biotek). For dose-response assays, serial dilutions of purified mAbs were applied to the wells in triplicate, and mAb binding was detected as detailed above. Half-maximal effective concentration (EC₅₀) values for binding were determined using Prism v8.0 software (GraphPad) after log transformation of mAb concentration using sigmoidal dose-response nonlinear regression analysis.

RBD minimal ACE2-binding motif peptide binding ELISA. Wells of 384-well microtiter plates were coated with 1 μg/mL streptavidin at 4° C. overnight. Plates were blocked with 0.5% BSA in DPBS containing 0.05% Tween-20 (DPBS-T) for 1 hr. Plates were washed 4× with 1×PBST and 2 μg/mL biotinylated-ACE2 binding motif peptide (cat. #LT5578, from LifeTein, LLC) was added to bind streptavidin for 1 hr at RT. Purified mAbs were diluted in blocking buffer, added to the wells, and incubated for 1 hr at RT. The bound antibodies were detected using goat anti-human IgG conjugated with HRP (horseradish peroxidase) (cat. #2014-05. Southern Biotech) and TMB (3,3′,5,5′-tetramethylbenzidine) substrate (ThermoFisher Scientific). Color development was monitored, 1N hydrochloric acid was added to stop the reaction, and the absorbance was measured at 450 nm using a spectrophotometer (Biotek). For dose-response assays, serial 3-fold dilutions starting at 10 μg/mL concentration of purified mAbs were applied to the wells in triplicate, and mAb binding was detected as detailed above.

Analysis of binding of antibodies to variant RBD proteins with alanine or arginine point mutations. Biolayer light interferometry (BLI) was performed using an Octet RED96 instrument (ForteBio; Pall Life Sciences) and wild-type RBD protein or a mutant RBD protein with a single amino acid change at defined positions to alanine or arginine. Binding of the RBD proteins were confirmed by first capturing octa-His-tagged RBD wild-type or mutant protein from a 10 μg/mL (≈200 nM) solution onto Penta-His biosensors for 300 sec. The biosensor tips then were submerged in binding buffer (PBS/0.2% Tween 20) for a 60 see wash, followed by immersion in a solution containing 150 nM of mAb for 180 sec (association), followed by a subsequent immersion in binding buffer for 180 sec (dissociation). Response for each RBD mutant protein was normalized to that of the wild-type RBD protein.

Focus reduction neutralization test (FRNT). Serial dilutions of mAbs were incubated with 10² FFU of SARS-CoV-2 for 1 hr at 37° C. The mAb-virus complexes were added to Vero E6 cell culture monolayers in 96-well plates for 1 hr at 37° C. Subsequently, cells were overlaid with 1% (w/v) methylcellulose in Minimum Essential Medium (MEM) supplemented to contain 2% heat-inactivated FBS. Plates were fixed 30 hrs later by removing overlays and fixed with 4% PFA in PBS for 20 min at room temperature. The plates were incubated sequentially with 1 μg/mL of rCR3022 anti-S antibody¹⁰ and horseradish-peroxidase (HRP)-conjugated goat anti-human IgG in PBS supplemented with 0.1% (w/v) saponin (Sigma) and 0.1% bovine serum albumin (BSA). SARS-CoV-2-infected cell foci were visualized using TrueBlue peroxidase substrate (KPL) and quantitated on an ImmunoSpot 5.0.37 Macro Analyzer (Cellular Technologies). Data were processed using Prism software version 8.0 (GraphPad).

Generation of S protein pseudotyped lentivirus. Suspension 293 cells were seeded and transfected with a third-generation HIV-based lentiviral vector expressing luciferase along with packaging plasmids encoding for the following: SARS-CoV-2 spike protein with a C-terminal 19 amino acid deletion, Rev, and Gag-pol. Medium was changed 16 to 20 hrs after transfection, and the supernatant containing virus was harvested 24 hrs later. Cell debris was removed by low-speed centrifugation, and the supernatant was passed through a 0.45 μm filter unit. The pseudovirus was pelleted by ultracentrifugation and resuspended in PBS for a 100-fold concentrated stock.

Pseudovirus neutralization assay. Serial dilutions of mAbs were prepared in a 384-well microtiter plate and pre-incubated with pseudovirus for 30 minutes at 37° C., to which 293 cells that stably express human ACE2 were added. The plate was returned to the 37° C. incubator, and then 48 hrs later luciferase activity measured on an EnVision 2105 Multimode Plate Reader (Perkin Elmer) using the Bright-Glo™ Luciferase Assay System (Promega), according to manufacturer's recommendations. Percent inhibition was calculated relative to pseudovirus-alone control. IC₅₀ values were determined by nonlinear regression using the Prism software version 8.1.0 (GraphPad). The average IC₅₀ value for each antibody was determined from a minimum of 3 independent experiments.

MAb quantification. Quantification of purified mAbs was performed by UV spectrophotometry using a NanoDrop spectrophotometer and accounting for the extinction coefficient of human IgG.

Competition-binding analysis through biolayer interferometry. Anti-mouse IgG Fc capture biosensors (FortéBio 18-5089) on an Octet HTX biolayer interferometry instrument (FortéBio) were soaked for 10 minutes in 1× kinetics buffer (Molecular Devices 18-1105), followed by a baseline signal measurement for 60 seconds. Recombinant SARS-CoV-2 RBD fused to mouse IgG1 (RBD-mFc, Sino Biological 40592-V05H) was immobilized onto the biosensor tips for 180 seconds. After a wash step in 1× kinetics buffer for 30 seconds, the reference antibody (5 μg/mL) was incubated with the antigen-containing biosensor for 600 seconds. Reference antibodies included the SARS-CoV human mAb CR3022 and COV2-2196. After a wash step in 1× kinetics buffer for 30 seconds, the biosensor tips then were immersed into the second antibody (5 μg/mL) for 300 seconds. Maximal binding of each antibody was normalized to a buffer-only control. Self-to-self blocking was subtracted. Comparison between the maximal signal of each antibody was used to determine the percent binding of each antibody. A reduction in maximum signal to <33% of un-competed signal was considered full competition of binding for the second antibody in the presence of the reference antibody. A reduction in maximum signal to between 33 to 67% of un-competed was considered intermediate competition of binding for the second antibody in the presence of the reference antibody. Percent binding of the maximum signal >67% was considered absence of competition of binding for the second antibody in the presence of the reference antibody.

High-throughput ACE-2 binding inhibition analysis. Wells of 384-well microtiter plates were coated with purified recombinant SARS-CoV-2 S2P_{ecto} protein at 4° C. overnight. Plates were blocked with 2% non-fat dry milk and 2% normal goat serum in DPBS-T for 1 hr. Purified mAbs from microscale expression were diluted two-fold in blocking buffer starting from 10 μg/mL in triplicate, added to the wells (20 μL/well), and incubated for 1 hr at ambient temperature. Recombinant human ACE2 with a C-terminal FLAG tag protein was added to wells at 2 μg/mL in a 5 L/well volume (final 0.4 μg/mL concentration of ACE2) without washing of antibody and then incubated for 40 min at ambient temperature. Plates were washed, and bound ACE2 was detected using HRP-conjugated anti-FLAG antibody (Sigma) and TMB substrate. ACE2 binding without antibody served as a control. The signal obtained for binding of the ACE2 in the presence of each dilution of tested antibody was expressed as a percentage of the ACE2 binding without antibody after subtracting the background signal. Half-maximal inhibitory concentration (IC₅₀) values for inhibition by mAb of S2P_{ecto}, protein binding to ACE2 was determined after log transformation of antibody concentration using sigmoidal dose-response nonlinear regression analysis (Prism software, GraphPad Prism version 8.0). ACE2 blocking assay using biolayer interferometry biosensor. Anti-mouse IgG biosensors on an Octet HTX biolayer interferometry instrument (FortéBio) were soaked for 10 minutes in 1× kinetics buffer, followed by a baseline signal measurement for 60 seconds. Recombinant SARS-CoV-2 RBD fused to mouse IgG1 (RBD-mFc, Sino Biological 40592-V05H) was immobilized onto the biosensor tips for 180 seconds. After a wash step in Ix kinetics buffer for 30 seconds, the antibody (5 μg/mL) was incubated with the antigen-coated biosensor for 600 seconds. After a wash step in 1× kinetics buffer for 30 seconds, the biosensor tips then were immersed into the ACE2 receptor (20 μg/mL) (Sigma-Aldrich SAE0064) for 300 seconds. Maximal binding of ACE2 was normalized to a buffer-only control. Percent binding of ACE2 in the presence of antibody was compared to ACE2 maximal binding. A reduction in maximal signal to <30% was considered ACE2 blocking.

High-throughput competition-binding analysis. Wells of 384-well microtiter plates were coated with purified recombinant SARS-CoV-2 S protein at 4° C. overnight. Plates were blocked with 2% BSA in DPBS containing 0.05% Tween-20 (DPBS-T) for 1 hr. Micro-scale purified unlabeled mAbs were diluted ten-fold in blocking buffer, added to the wells (20 μL/well) in quadruplicates, and incubated for 1 hr at ambient temperature. A biotinylated preparation of a recombinant mAb based on the variable gene sequence of the previously described mAb CR3022¹² and also newly identified mAbs COV2-2096, -2130, and -2196 that recognized distinct antigenic regions of the SARS-CoV-2 S protein were added to each of four wells with the respective mAb at 2.5 μg/mL in a 5 μL/well volume (final 0.5 μg/mL concentration of biotinylated mAb) without washing of unlabeled antibody and then incubated for 1 hr at ambient temperature. Plates were washed, and bound antibodies were detected using HRP-conjugated avidin (Sigma) and TMB substrate. The signal obtained for binding of the biotin-labeled reference antibody in the presence of the unlabeled tested antibody was expressed as a percentage of the binding of the reference antibody alone after subtracting the background signal. Tested mAbs were considered competing if their presence reduced the reference antibody binding to less than 41% of its maximal binding and non-competing if the signal was greater than 71%. A level of 40-70% was considered intermediate competition.

Binding analysis of mAbs to alanine or arginine RBD mutants. Biolayer light interferometry was performed using an Octet RED96 instrument (FortéBio; Pall Life Sciences). Binding was confirmed by first capturing octa-His-tagged RBD mutants 10 μg/mL (≈200 nM) onto Penta-His biosensors for 300 s. The biosensors then were submerged in binding buffer (PBS/0.2% TWEEN 20) for a wash for 60 see followed by immersion in a solution containing 150 nM of mAbs for 180 sec (association), followed by a subsequent immersion in binding buffer for 180 see (dissociation). Response for each RBD mutant was normalized to that of wild-type RBD.

Mouse experiments using human hACE2-transduced mice. Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Virus inoculations were performed under anesthesia that was induced and maintained with ketamine hydrochloride and xylazine, and all efforts were made to minimize animal suffering.

BALB/c mice were purchased from Jackson Laboratories (strain 000651). Female mice (10-11-week-old) were given a single intraperitoneal injection of 2 mg of anti-Ifnar1 mAb (MAR1-5A³³, Leinco) one day before intranasal administration of 2.5×10⁸ PFU of AdV-hACE2. Five days after AdV transduction, mice were inoculated with 4×10⁵ PFU of SARS-CoV-2 by the intranasal route. Anti-SARS-CoV-2 human mAbs or isotype control mAbs were administered 24 hours prior to SARS-CoV-2 inoculation. Weights were monitored on a daily basis, and animals were sacrificed at days 5 or 7 post-infection, and tissues were harvested.

Measurement of viral burden. Tissues were weighed and homogenized with zirconia beads in a MagNA Lyser instrument (Roche Life Science) in 1 ml of DMEM media supplemented with 2% heat-inactivated FBS. Tissue homogenates were clarified by centrifugation at 10,000 rpm for 5 min and stored at −80° C. RNA was extracted using MagMax mirVana Total RNA isolation kit (Thermo Scientific) and a Kingfisher Flex 96 well extraction machine (Thermo Scientific). TaqMan primers were designed to target a conserved region of the N gene using SARS-CoV-2 (MN908947) sequence as a guide (L Primer: ATGCTGCAATCGTGCTACAA (SEQ ID NO: 1); R primer: GACTGCCGCCTCTGCTC (SEQ ID NO: 2); probe: /56-FAM/TCAAGGAAC/ZEN/AACATTGCCAA/3IABkFQ/). To establish an RNA standard curve, the inventors generated concatenated segments of the N gene in a gBlocks fragment (IDT) and cloned this into the PCR-II topo vector (Invitrogen). The vector was linearized, and in vitro T7-DNA-dependent RNA transcription was performed to generate materials for a quantitative standard curve.

Cytokine and chemokine mRNA measurements. RNA was isolated from lung homogenates at 7 dpi as described above. cDNA was synthesized from DNase-treated RNA using the High-Capacity cDNA Reverse Transcription kit (Thermo Scientific) with the addition of RNase inhibitor, following the manufacturer's protocol. Cytokine and chemokine expression was determined using TaqMan Fast Universal PCR master mix (Thermo Scientific) with commercial primers/probe sets specific for IFNγ (IDT: Mm.PT.58.41769240), IL-6 (Mm.PT.58.10005566), CXCL10 (Mm.PT.58.43575827), CCL2 (Mm.PT.58.42151692 and results were normalized to GAPDH (Mm.PT.39a.1) levels. Fold change was determined using the 2^{-ΔΔCt} method comparing anti-SARS-CoV-2 specific or isotype control mAb-treated mice to naïve controls.

Mouse experiments using wild-type mice. Animal studies were carried out in accordance with the recommendations in the Guide for the Care and Use of Laboratory Animals of the National Institutes of Health. The protocols were approved by the Institutional Animal Care and Use Committee at the UNC Chapel Hill School of Medicine (NIH/PHS Animal Welfare Assurance Number is D16-00256 (A3410-01)). Virus inoculations were performed under anesthesia that was induced and maintained with ketamine hydrochloride and xylazine, and all efforts were made to minimize animal suffering.

Mouse adapted SARS-CoV-2 (MA-SARS-CoV-2) virus. The virus was generated as described previously¹⁴. Virus was propagated in Vero E6 cells grown in DMEM with 10% Fetal Clone II and 1% Pen/Strep. Virus titer was determined by plaque assay. Briefly, virus was serial diluted and inoculated onto confluent monolayers of Vero E6 cells, followed by agarose overlay. Plaques were visualized on day 2 post-infection after staining with neutral red dye.

Wild-type mice. 12-month-old BALB/c mice from Envigo were used in experiments. Mice were acclimated in the BSL3 for at least 72 hours prior to start of experiments. At 6 hours prior to infection, mice were prophylactically treated with 200 μg of human monoclonal antibodies via intraperitoneal injection. The next day, mice were anesthetized with a mixture of ketamine and xylazine and intranasally infected with 10 PFU of MA-SARS-CoV-2 diluted in PBS. Daily weight loss was measured, and at two days post-infection mice were euthanized by isoflurane overdose prior to tissue harvest.

Plaque assay of lung tissue homogenates. The lower lobe of the right lung was homogenized in 1 mL PBS using a MagnaLyser (Roche). Serial dilutions of virus were titered on Vero E6 cell culture monolayers, and virus plaques were visualized by neutral red staining at two days after inoculation. The limit of detection for the assay is 100 PFU per lung.

Quantification and statistical analysis. The descriptive statistics mean±SEM or mean±SD were determined for continuous variables as noted. Technical and biological replicates are described in the figure legends. In the mouse studies, analysis of weight change and viral burden in vivo were determined by two-way ANOVA and Mann-Whitney tests, respectively. Statistical analyses were performed using Prism v8.0 (GraphPad).

### REFERENCES FOR EXAMPLE 3

1. Zost S J, G. P., Chen R E, Case J B, Reidy J X, Trivette A, Nargi R S, Sutton R E, Suryadevara N, Chen E C, Binshtein E, Shrihari S, Ostrowski M, Chu H Y, Didier J E, MacRenaris K W, Jones T, Day S, Myers L, Lee F E-H, Nguyen D C, Sanz I, Martinez D R, Baric R S, Thackray L B, Diamond M S, Carnahan R H, Crowe J E Jr., Rapid isolation and profiling of a diverse panel of human monoclonal antibodies targeting the SARS-CoV-2 spike protein. bioRxiv 2020.05.12.091462; doi: https://doi.org/10.1101/2020.05.12.091462 (2020)
2. Mukherjee, S. et al. Enhancing dengue virus maturation using a stable furin overexpressing cell line. Virology 497, 33-40, doi:10.1016/j.virol.2016.06.022 (2016).
3. Wrapp, D. et al. Cryo-EM structure of the 2019-nCoV spike in the prefusion conformation. Science 367, 1260-1263, doi:10.1126/science.abb2507 (2020).
4. Ohi, M., Li, Y., Cheng. Y. & Walz, T. Negative staining and image classification—Powerful tools in modern electron microscopy. Biol Proced Online 6, 23-34, doi:10.1251/bpo70 (2004).
5. Mastronarde, D. N. Automated electron microscope tomography using robust prediction of specimen movements. J Struct Biol 152, 36-51, doi:10.1016/j.jsb.2005.07.007 (2005).
6. Punjani, A., Rubinstein, J. L., Fleet, D. J. & Brubaker, M. A. cryoSPARC: algorithms for rapid unsupervised cryo-EM structure determination. Nat Methods 14, 290-296, doi:10.1038/nmeth.4169 (2017).
7. Bepler, T., Noble, A. J., and Berger, B. Topaz-Denoise: general deep denoising models for cryoEM. bioRxiv. doi:10.1101/838920 (2019).
8. Pettersen. E. F. et al. UCSF Chimera—a visualization system for exploratory research and analysis. J Comput Chem 25, 1605-1612, doi:10.1002/jcc.20084 (2004).
9. Chng, J. et al. Cleavage efficient 2A peptides for high level monoclonal antibody expression in CHO cells. MAbs 7, 403-412, doi:10.1080/19420862.2015.1008351 (2015).
10. Yuan. M. et al. A highly conserved cryptic epitope in the receptor binding domains of SARS-CoV-2 and SARS-CoV. Science 368, 630-633, doi:10.1126/science.abb7269 (2020).
11. Ianevski, A., He. L., Aittokallio, T. & Tang, J. SynergyFinder: a web application for analyzing drug combination dose-response matrix data. Bioinformatics 33, 2413-2415, doi:10.1093/bioinformatics/btx162 (2017).
12. ter Meulen, J. et al. Human monoclonal antibody as prophylaxis for SARS coronavirus infection in ferrets. Lancet 363, 2139-2141, doi:10.1016/S0140-6736(04)16506-9 (2004).
13. Sheehan, K. C. et al. Blocking monoclonal antibodies specific for mouse IFN-alpha/beta receptor subunit 1 (IFNAR-1) from mice immunized by in vivo hydrodynamic transfection. J Interferon Cytokine Res 26, 804-819 (2006).
14. Dinnon K H, et al. A mouse-adapted SARS-CoV-2 model for the evaluation of COVID-19 medical countermeasures. bioRxiv 2020.05.06.081497; doi: https://doi.org/10.1101/2020.05.06.081497 (2020).

### Example 4—Results

The S protein of SARS-CoV-2 is the molecular determinant of viral attachment, fusion, and entry into host cells³. The cryo-EM structure of a prefusion-stabilized trimeric S protein ectodomain (S2P_{ecto}) for SARS-CoV-2 reveals similar features to that of the SARS-CoV S protein⁴. This type I integral membrane protein and class I fusion protein possesses an N-terminal subunit (S1) that mediates binding to receptor and a C-terminal subunit (S2) that mediates virus-cell membrane fusion. The S1 subunit contains an N-terminal domain (SNTD) and a receptor-binding domain (SRBD). SARS-CoV-2 and SARS-CoV, which share approximately 78% sequence identity in their genomes¹ both use human angiotensin-converting enzyme 2 (hACE2) as an entry receptor⁵⁻⁷. Previous studies of human immunity to other high-pathogenicity zoonotic betacoronaviruses including SARS-CoV⁸⁻¹² and Middle East respiratory syndrome (MERS)¹³⁻²² showed that Abs to the viral surface spike (S) glycoprotein mediate protective immunity. The most potent S protein-specific mAbs appear to neutralize betacoronaviruses by blocking attachment of virus to host cells by binding to the region on SRBD that directly mediates engagement of the receptor. It is likely that human Abs have promise for use in modifying disease during SARS-CoV-2 infection, when used for prophylaxis, post-exposure prophylaxis, or treatment of SARS-CoV-2 infection²³. Many studies including randomized controlled trials evaluating convalescent plasma and one trial evaluating hyperimmune immunoglobulin are ongoing, but it is not yet clear whether such treatments can reduce morbidity or mortality.

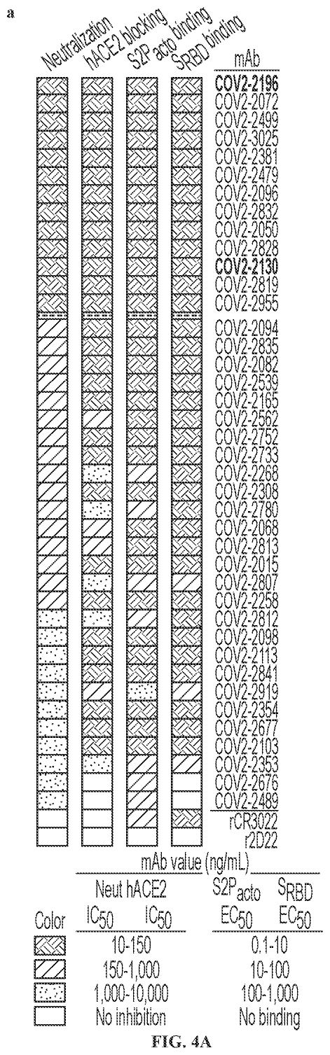

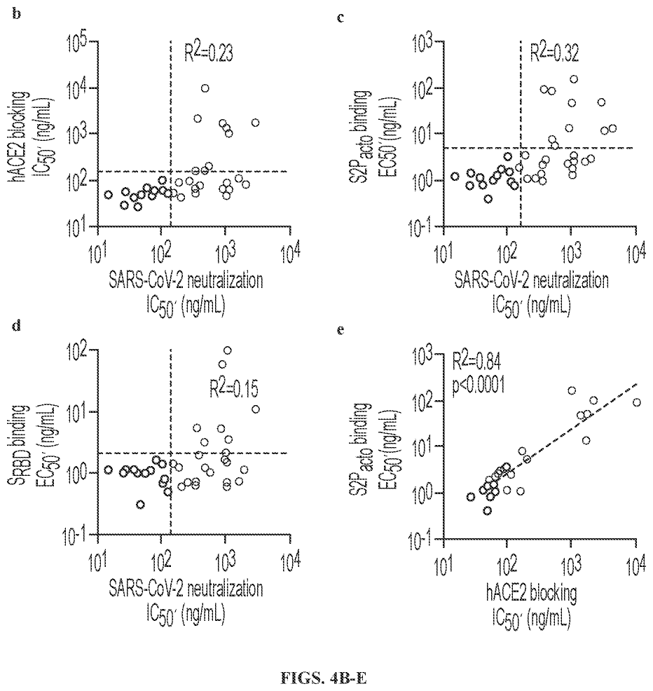

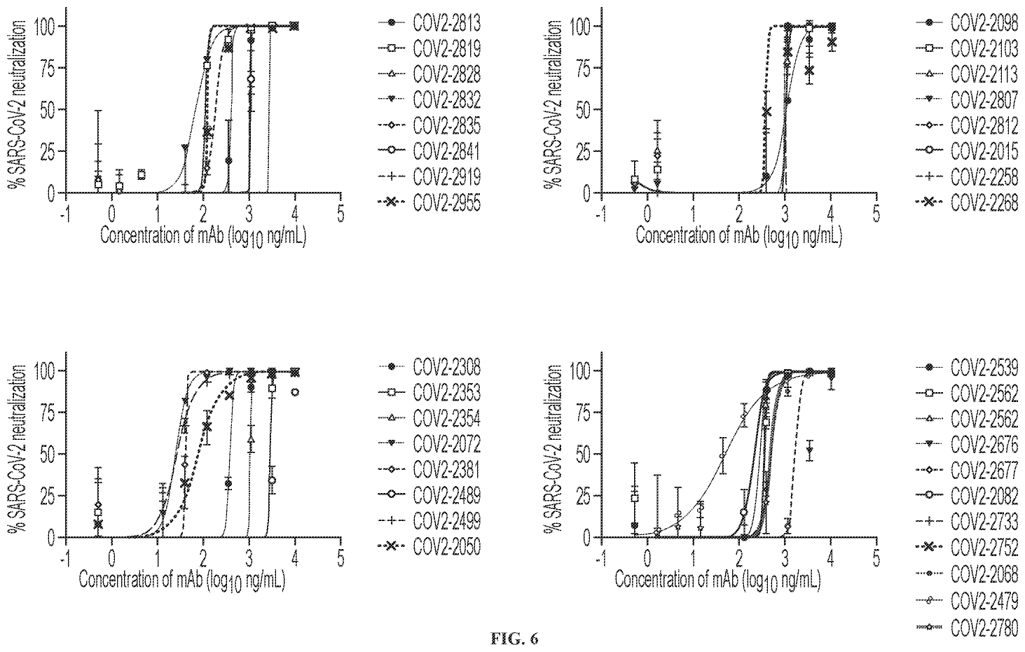

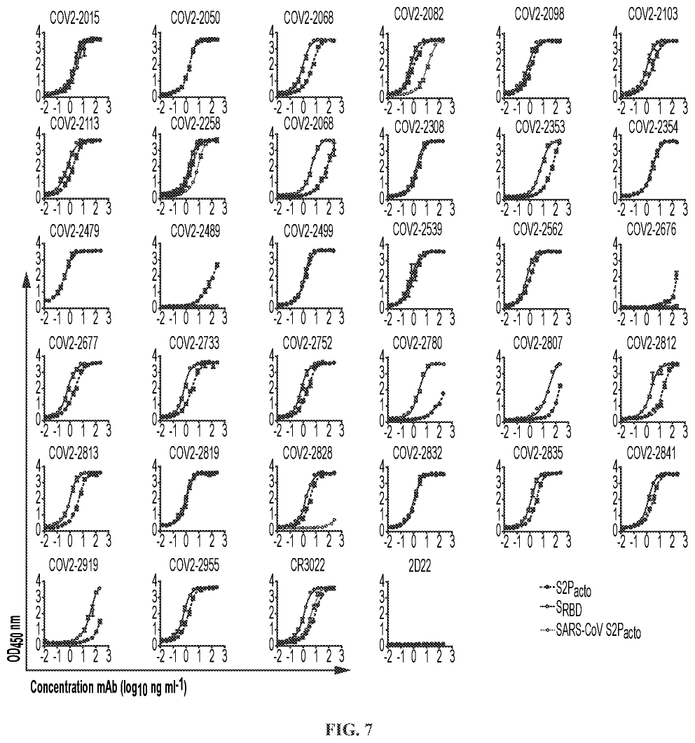

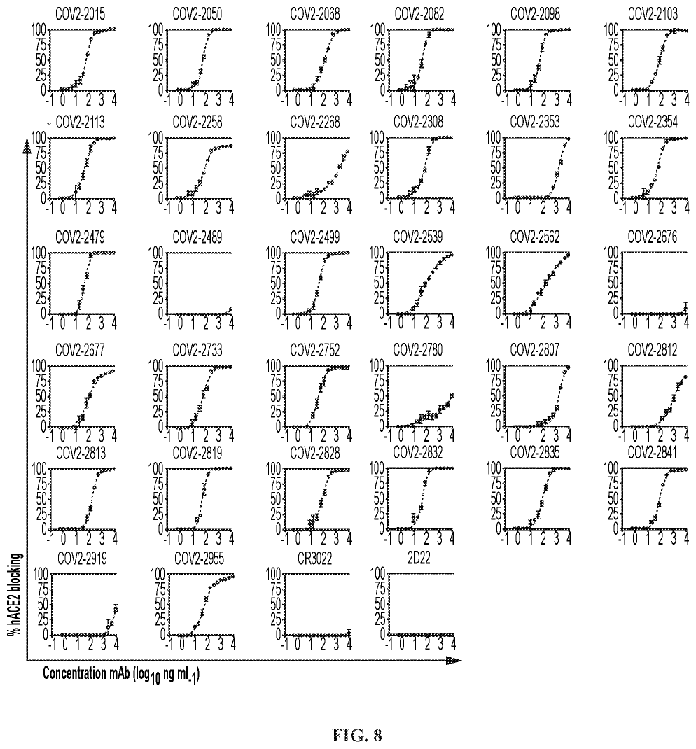

The inventors isolated a large panel of SARS-CoV-2 S protein-reactive mAbs from the B cells of two individuals who were previously infected with SARS-CoV-2 in Wuhan China²⁵. A subset of those antibodies bound to the receptor-binding domain of S (SRBD) and exhibited neutralizing activity in a rapid screening assay with authentic SARS-CoV-2²⁵. Here, the inventors defined the antigenic landscape of SARS-CoV-2 and determined which sites of SRBD are the target of protective mAbs. They tested a panel of 40 anti-S human mAbs previously pre-selected by a rapid neutralization screening assay in a quantitative focus reduction neutralization test (FRNT) with SARS-CoV-2 strain WA1/2020. These assays revealed the panel exhibited a range of half-maximal inhibitory concentration (IC₅₀) values, from 15 to over 4,000 ng/mL (visualized as a heatmap in FIG. 4A, values shown in Table B. and full curves shown in FIG. 6). The inventors hypothesized that many of these SRBD-reactive mAbs neutralize virus infection by blocking SRBD binding to hACE2. Indeed, most neutralizing mAbs that were tested inhibited the interaction of hACE2 with trimeric S protein directly (FIG. 4A, FIG. 7). Consistent with these results, these mAbs also bound strongly to a trimeric S ectodomain (S2P_{ecto}) protein or monomeric SRBD (FIG. 4A, FIG. 8). The inventors evaluated whether S2P_{ecto} or SRBD binding or hACE2-blocking potency predicted binding neutralization potency independently, but none of these measurements correlated with neutralization potency (FIGS. 4B-D). However, each of the mAbs in the highest neutralizing potency tier (IC₅₀<150 ng/mL) also revealed strongest blocking activity against hACE2 (IC₅₀<150 ng/mL) and exceptional binding activity (EC₅₀<2 ng/mL) to S2P_{ecto} trimer and SRBD (FIG. 4E).

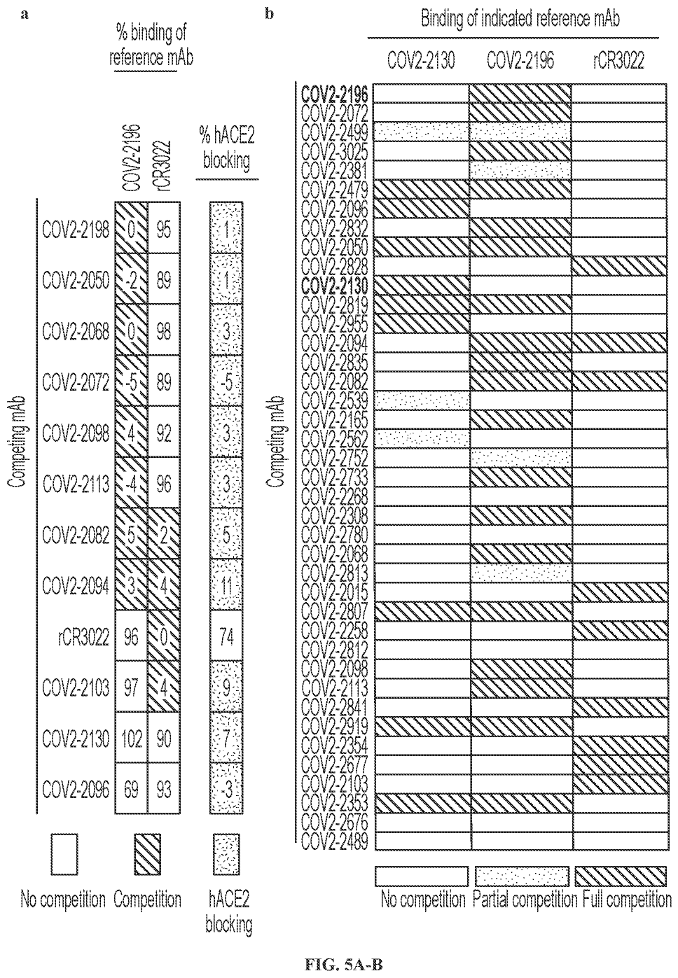

The inventors next defined the major antigenic sites on SRBD for neutralizing mAbs by competition-binding analysis. They first used a biolayer interferometry-based competition assay with a minimal SRBD domain to screen for mAbs that competed for binding with the potently neutralizing mAb COV2-2196 or a recombinant version of the previously described SARS-CoV mAb CR3022, which recognizes a conserved cryptic epitope^{10,26}. The inventors identified three major groups of competing mAbs (FIG. 5A). The largest group of mAbs blocked COV2-2196 but not rCR3022, while some mAbs were blocked by rCR3022 but not COV2-2196. Two mAbs, including COV2-2130, were not blocked by either reference mAb. Most mAbs competed with hACE2 for binding, suggesting that they bound near the hACE2 binding site of the SRBD. The inventors used COV2-2196, COV2-2130, and rCR3022 in an ELISA-based competition-binding assay with trimeric S2P_{ecto} protein and also found that SRBD contained three major antigenic sites, with some mAbs likely making contacts in more than one site (FIG. 5B). Most of the potently neutralizing mAbs directly competed for binding with COV2-2196.

Previous structural studies have defined the interaction between the SRBD and hACE2²⁸. Most of the interacting residues in the SRBD are contained within a 60-amino-acid linear peptide that defines the hACE2 recognition motif.

Here, the inventors defined the antigenic landscape for a large panel of highly potent mAbs against SARS-CoV-2. These detailed studies and the screening studies that identified this panel of mAbs from a larger panel of hundreds²⁵ demonstrate that although diverse human neutralizing antibodies are elicited by natural infection with SARS-CoV-2, only a small subset of those mAbs are of high potency (IC₅₀<50 ng/mL against live SARS-CoV-2 virus), and therefore, suitable for therapeutic development. Biochemical and structural analysis of these potent mAbs defined three principal antigenic sites of vulnerability to neutralization by human mAbs elicited by natural infection with SARS-CoV on the SRBD. Moreover, as SARS-CoV-2 continues to circulate, population immunity elicited by natural infection may start to select for antigenic variants that escape from the selective pressure of neutralizing antibodies, reinforcing the need to target multiple epitopes of S protein in vaccines or immunotherapeutics.

The common S gene variants across the globe reported to date are located at positions D614G, V483A, L5F, Q675H, H655Y and S939F³⁰, far away from the amino acid variants at residues 486 or 487 identified in the inventors' mutation studies for the lead mAbs studied here. Rationally-selected therapeutic cocktails like the one described here might offer even greater resistance to SARS-CoV-2 escape. These studies set the stage for preclinical evaluation and development of the identified mAbs as candidates for use as COVID-19 immunotherapeutics in humans.

### REFERENCES FOR EXAMPLE 4

1. Zhou, P., et al. A pneumonia outbreak associated with a new coronavirus of probable bat origin. Nature 579, 270-273 (2020).
2. Zhu, N., et al. A novel coronavirus from patients with pneumonia in China, 2019. N Engl J Med 382, 727-733 (2020).
3. Pillay, T. S. Gene of the month: the 2019-nCoV/SARS-CoV-2 novel coronavirus spike protein. J Clin Pathol (2020).
4. Wrapp, D., et al. Cryo-EM structure of the 2019-nCoV spike in the prefusion conformation. Science 367, 1260-1263 (2020).
5. Wan, Y., Shang, J., Graham, R., Baric, R. S. & Li, F. Receptor recognition by the novel Coronavirus from Wuhan: an Analysis Based on Decade-Long Structural Studies of SARS Coronavirus. J Virol 94(2020).
6. Hoffmann, M., et al. SARS-CoV-2 cell entry depends on ACE2 and TMPRSS2 and is blocked by a clinically proven protease inhibitor. Cell 181, 271-280 e278 (2020).
7. Li, W., et al. Angiotensin-converting enzyme 2 is a functional receptor for the SARS coronavirus. Nature 426, 450-454 (2003).
8. Sui, J., et al. Potent neutralization of severe acute respiratory syndrome (SARS) coronavirus by a human mAb to S1 protein that blocks receptor association. Proc Natl Acad Sci USA 101, 2536-2541 (2004).
9. ter Meulen, J., et al. Human monoclonal antibody as prophylaxis for SARS coronavirus infection in ferrets. Lancet 363, 2139-2141 (2004).
10. ter Meulen, J., et al. Human monoclonal antibody combination against SARS coronavirus: synergy and coverage of escape mutants. PLoS Med 3, e237 (2006).
11. Zhu, Z., et al. Potent cross-reactive neutralization of SARS coronavirus isolates by human monoclonal antibodies. Proc Natl Acad Sci USA 104, 12123-12128 (2007).
12. Rockx, B., et al. Structural basis for potent cross-neutralizing human monoclonal antibody protection against lethal human and zoonotic severe acute respiratory syndrome coronavirus challenge. J Virol 82, 3220-3235 (2008).
13. Chen, Z., et al. Human neutralizing monoclonal antibody inhibition of Middle East respiratory syndrome coronavirus replication in the common marmoset. J Infect Dis 215, 1807-1815 (2017).
14. Choi, J. H., et al. Characterization of a human monoclonal antibody generated from a B-cell specific for a prefusion-stabilized spike protein of Middle East respiratory syndrome coronavirus. PLoS One 15, e0232757 (2020).
15. Niu, P., et al. Ultrapotent human neutralizing antibody repertoires against Middle East respiratory syndrome coronavirus from a recovered patient. J Infect Dis 218, 1249-1260 (2018).
16. Wang, L., et al. Importance of neutralizing monoclonal antibodies targeting multiple antigenic sites on the Middle East respiratory syndrome coronavirus spike glycoprotein to avoid neutralization escape. J Virol 92(2018).
17. Wang, N., et al. Structural definition of a neutralization-sensitive epitope on the MERS-CoV S1-NTD. Cell Rep 28, 3395-3405 e3396 (2019).
18. Zhang, S., et al. Structural definition of a unique neutralization epitope on the receptor-binding domain of MERS-CoV spike glycoprotein. Cell Rep 24, 441-452 (2018).
19. Corti, D., et al. Prophylactic and postexposure efficacy of a potent human monoclonal antibody against MERS coronavirus. Proc Natl Acad Sci USA 112, 10473-10478 (2015).
20. Jiang, L., et al. Potent neutralization of MERS-CoV by human neutralizing monoclonal antibodies to the viral spike glycoprotein. Sci Transl Med 6, 234ra259 (2014).
21. Tang, X. C., et al. Identification of human neutralizing antibodies against MERS-CoV and their role in virus adaptive evolution. Proc Natl Acad Sci USA 111, E2018-2026 (2014).
22. Ying, T., et al. Exceptionally potent neutralization of Middle East respiratory syndrome coronavirus by human monoclonal antibodies. J Virol 88, 7796-7805 (2014).
23. Jiang, S., Hillyer. C. & Du, L. Neutralizing antibodies against SARS-CoV-2 and other human coronaviruses. Trends Immunol 41, 355-359 (2020).
24. Valk, S. J., et al. Convalescent plasma or hyperimmune immunoglobulin for people with COVID-19: a rapid review. Cochrane Database Syst Rev 5, CD013600((2020).
25. Zost, S. J., et al. Rapid isolation and profiling of a diverse panel of human monoclonal antibodies targeting the SARS-CoV-2 spike protein. bioRxiv, 2020.2005.2012.091462 (2020).
26. Yuan. M., et al. A highly conserved cryptic epitope in the receptor binding domains of SARS-CoV-2 and SARS-CoV. Science 368.630-633 (2020).
27. Ianevski, A., He, L., Aittokallio, T. & Tang, J. SynergyFinder: a web application for analyzing drug combination dose-response matrix data. Bioinformatics 33, 2413-2415 (2017).
28. Lan, J., et al. Structure of the SARS-CoV-2 spike receptor-binding domain bound to the ACE2 receptor. Nature 581, 215-220 (2020).
29. Dinnon, K. H., et al. A mouse-adapted SARS-CoV-2 model for the evaluation of COVID-19 medical countermeasures. bioRxiv. 2020.2005.2006.081497 (2020).
30. Laha, S., et al. Characterizations of SARS-CoV-2 mutational profile, spike protein stability and viral transmission. bioRxiv, 2020.2005.2003.066266 (2020).

TABLE A
Activity Data

| | BINDING ASSAY RESULTS | | | | NEUTRALIZATION ASSAY RESULTS | | | | | |
|---|---|---|---|---|---|---|---|---|---|---|
| | ELISA-Purified IgG | | | | (Yes/No qualitative test, or IC50 value (ng/mL) | | | | | |
| | (OD 450 nm) | | | | | SARS-CoV-2 | | | Nano-luciferase | |
| | SARS- | | | SARS- | | xCelligence neutralization | | | virus reduction | |
| | CoV-2 | SARS- | SARS- | CoV | | test (cell impedence) | | SARS-CoV- | test | |
| Clone ID | Spike | CoV-2 | CoV-2 | Spike | hACE2 | | Estimated | reduction | SARS- | |
| (COV2-xxxx) | trimer | RBD | NTD | trimer? | blocking | Qualitative | IC50 | test 2 focus | CoV-2 | SARS |
| 2050 | 4.3 | 4.3 | 0.12 | 0.11 | Yes | Yes | nt | 25 | <50 | nt |
| 2046 | 4.3 | 4.2 | 0.09 | 0.09 | Yes | Yes | nt | 171 | <100 | nt |
| 2113 | 4.4 | 4.4 | 0.4 | 0.1 | Yes | Yes | nt | 427 | <300 | nt |
| 2132 | 4.4 | 4.4 | 0.15 | 0.09 | Yes | Yes | nt | 673 | <300 | nt |
| 2098 | 4.3 | 4.2 | 0.15 | 0.09 | Yes | Yes | nt | 776 | <100 | nt |
| 2068 | 4.3 | 4.3 | 0.1 | 0.1 | Yes | Yes | nt | 864 | <300 | nt |
| 2082 | 4.3 | 4.3 | 0.11 | 3.9 | Yes | Yes | nt | 302 | <200 | >1,000 |
| 2103 | 4.3 | 4.3 | 0.12 | 0.53 | Yes | Yes | nt | >1,000 | nt | nt |
| 2961 | 3.60 | 3.70 | NT | 0.10 | Yes | Yes | nt | 30 | nt | nt |

TABLE B
Neutralization IC₅₀, hACE2 blocking IC₅₀, and EC₅₀ values
for binding to S2P_{ecto} or SRBD antigens for mAb panel

| | | | | | SARS-CoV |
|---|---|---|---|---|---|
| | Neutral- | hACE2 | S2P_{ecto} | SRBD | S2P_{ecto} |
| | ization | blocking | binding | binding | binding |
| | IC₅₀, | IC₅₀, | EC₅₀, | EC₅₀, | EC₅₀, |
| MAb | ng/mL | ng/mL | ng/mL | ng/mL | ng/mL |
| COV2-2015 | 892 | 68 | 2.2 | 5.1 | 2.7 |
| COV2-2050 | 80 | 63 | 1.7 | 1.6 | — |
| COV2-2068 | 478 | 166 | 7.4 | 1.2 | — |
| COV2-2082 | 204 | 43 | 1.1 | 0.6 | 19.9 |
| COV2-2098 | 1,029 | 48 | 1.3 | 0.7 | — |
| COV2-2103 | 1,969 | 79 | 2.9 | 1.1 | — |
| COV2-2113 | 1,041 | 60 | 1.8 | 0.6 | — |
| COV2-2258 | 989 | 76 | 2.5 | 1.6 | 11.2 |
| COV2-2268 | 371 | 2,198 | 91.2 | 5.3 | — |
| COV2-2308 | 394 | 75 | 2.7 | 1.9 | — |
| COV2-2353 | 2,891 | 1,750 | 48.0 | 10.9 | — |
| COV2-2354 | 1,105 | 67 | 2.6 | 3.5 | — |
| COV2-2489 | 4,378 | — | 13.0 | — | — |
| COV2-2479 | 48 | 50 | 0.4 | 0.3 | — |
| COV2-2499 | 27 | 57 | 1.4 | 1.1 | — |
| COV2-2539 | 274 | 98 | 1.1 | 0.7 | — |
| COV2-2562 | 348 | 154 | 1.0 | 0.6 | — |
| COV2-2676 | 3,247 | — | 12.0 | — | — |
| COV2-2677 | 1,618 | 107 | 2.4 | 0.7 | — |
| COV2-2733 | 356 | 71 | 2.4 | 0.6 | — |
| COV2-2752 | 349 | 53 | 1.9 | 0.7 | — |
| COV2-2780 | 478 | 10,000 | 86.0 | 3.1 | — |
| COV2-2807 | 907 | 1,753 | 13.0 | 55.1 | — |
| COV2-2812 | 1,020 | 1,387 | 45.4 | 2.1 | — |
| COV2-2813 | 555 | 206 | 5.3 | 1.0 | — |
| COV2-2819 | 114 | 60 | 0.9 | 0.8 | — |
| COV2-2828 | 100 | 100 | 3.2 | 1.4 | 338 |
| COV2-2832 | 70 | 47 | 1.3 | 1.1 | — |
| COV2-2835 | 190 | 91 | 3.4 | 1.2 | — |
| COV2-2841 | 1,065 | 91 | 3.3 | 1.5 | — |
| COV2-2919 | 1,091 | 1,000 | 153.0 | 90.3 | — |
| rCR3022 | — | — | 10.2 | 1.1 | 5.2 |
| r2D22 | — | — | — | — | — |

TABLE 1
NUCLEOTIDE SEQUENCES FOR ANTIBODY VARIABLE
REGION

| Clone | Seq ID | Chain | Variable Sequence Region |
|---|---|---|---|
| COV2- | 3 | heavy | GAGGTGCAGCTGGTGGAGTCTGGGGGAGGCTTGGTCCAGCCTGGGGGGTCCCTGAGACTCTCC |
| 2171 | | | TGTGTAGCCTCTGGATTCACCTTTAGTTTCTATTGGATGAGCTGGGTCCGCCAGGCTCCAGGA |
| | | | AAGGGGCTCGAGTGGGTGGCCAACATAAAGCAAGATGGAGGTGAGAAATACTATGTGGACTCT |
| | | | GTGAAGGGCCGATTCACCATCTCCAGAGACAACGCCAAGAACTCACTGTATCTGCAAATGAAC |
| | | | AGCCTGAAGCCGAGGACACGGCTGTGTATTACTGTGCGAGACTGTCTGCAGCAGCTGGGAC |
| | | | TTTGACTACTGGGGCCAGGGAACCCTGGTCACCGTCTCCTCA |
| | 4 | light | TCCTATGAGCTGACTCAGCCACCCTCAGTGTCCGTGTCCCCAGGACAGACAGCCAGCATCACC |
| | | | TGCTCTGGAGATAAATTGGGGGATAAATATGCTTGTTGGTACCAACAGAGGCCAGGCCAGTCC |
| | | | CCTGTGCTGGTCATCTATCAAGATAGTAAGCGGCCCTCAGGGATCCCTGAGCGATTCTCTGGC |
| | | | TCCAACTCTGGGAACACAGCCACTCTGACCATCAGCGGGACCCAGGCTATGGATGAGGCTGAC |
| | | | TATTACTGTCAGGCGTGGGACAGCAGCACTGGAGTCTTCGGAACTGGGACCAAGGTCACCGTC |
| | | | CTA |
| COV2- | 5 | heavy | GAGGTGCAGCTGGTGGAGTCTGGGGGAGGTGTGGTACGGCCTGGGGGGTCCCTGAGACTCTCC |
| 2173 | | | TGTGCAGCCTCTGGATTCACCTTTGATGATTATGGCATGAGCTGGGTCCGCCAAGCTCCAGGG |
| | | | AAGGGGCTGGAGTGGGTCTCTGCTATTAATTGGAATGGTGGTAGCACAGGTTATGCAGACTCT |
| | | | GTAAAGGGCCGATTCACCATCTCCAGAGATAATGCCAAGAACTCCCTGTATCTGCAAATGAAC |
| | | | AGTCTGAGAGCCGAGGACACGGCCTTGTATCACTGTGCGAGACGGCGTAGCTCGTCCCGGTAT |
| | | | AGCAGTGGCTGGTATATGTACTAGTACTACATGGACGTGTGGGGCAAAGGGACCACGGTCACC |
| | | | GTCTCCTCA |
| | 6 | light | GACATCCAGATGACCCAGTCTCCTTCCACCCTGTCTGCATCTGTAGGAGACAGAGTCACCATC |
| | | | ACTTGCCGGGCCAGTCAGAGTGTTAGTACCTGGTTGGCCTGGTATCAGCAGAAACCAGGGAAA |
| | | | GCCCCTAACCTCCTGATCTATGAGGCGTCTAGTTTAGAAAGTGGGGTCCCATCAAGGTTCAGC |
| | | | GGCAGTGGATCTGGGACAGAATTCACTCTCACCATCAGCAGCCTGCAGCCTGATGATTTTGCA |
| | | | ACTTATTACTGCCAACAGTATAATACTTATTCGGGGACGTTCGGCCAAGGGACCAAGGTGGAA |
| | | | ATCAAA |
| COV2- | 7 | heavy | CAGATCACCTTCAAGGAGTCTGGTCCTACGCTGGTGAAACCCACAGAGACCCTCACGCTGACC |
| 2177 | | | TGCACCTTCTCTGGGTTCTCAGTCAGCACTAGTGGAGAGGGTGTGGGCTGGATCCGTCAGCCC |
| | | | CCAGGAAAGGCCCTGGAGTGGCTTGCAGTCATTTATTGGGATGATGATAAGCGCTACAGCCCA |
| | | | TCTCTGAAGAGCAGGCTCACCATCACCAGGGACACCTCCAAAAACCAGGTGGTCCTTACAATG |
| | | | ACCAACATGGACCCTGTGGACACAGCCACATATTACTGTGCACACAGGCTTTGGTTCAGGGAT |
| | | | GCTTTTGATATTTGGGGCCAAGGGACAACGGTCACCGTCTCCTCA |
| | 8 | light | GACATCCAGATGACCCAGTCTCCATCGTCCCTGTCTGCATCTGTAGGAGACAGAGTCACAATC |
| | | | ACTTGCCGGGCACGTCAGAGCATTAGCAACTATTTAAATTGGTATCAGCAGAAACCAGGGAAA |
| | | | GCCCCTAAGCTCCTGATCTATGCTGCATCCAGTTTGCACAGTGGGGTCCCATCAAGGTTCAGT |
| | | | GGCAGTGGATCTGGGACAGATTTCACTCTCACCATCAGCAGTCTGCAACCTGAAGATTTTGCA |
| | | | ACTTACTACTGTCAACAGACTTACAGTACCTTCTGGACGTTCGGCCAAGGGACCAACGTGGAA |
| | | | ATCAAA |
| COV2- | 9 | heavy | GAGGTGCAGCTGGTGGAGTCTGGGGGAGGCTTGGTCCAGCCTGGGGGGTCCCTAAGACTCTCC |
| 2178 | | | TGTGCAGCCTCTGGATTCACCTTTAGTACCTATTGGATGACCTGGGTCCGCCAGGCTCCAGGG |
| | | | AAGGGGCTGGAGTGGGTGGCCAACATAAAGCAAGATGGAAGTGAGAAATACTATGTGGACTCT |
| | | | GTTAAGTACCGATTCACCATCTCCAGAGACAACGCCAAGAACTCACTGTATCTGCAAATGAAC |
| | | | AGCCTGAGAGCCGAGGACACGGCTGTGTATTACTGTGCGAGAGTCGGGAGCAGCAGCTTTTAC |
| | | | TTTGACTACTGGGGCCAGGGAACCCTGGTCACCGTCTCCTCA |
| | 10 | light | TCCTATGAACTGACTCAGCCACCCTCAGTGTCCGTGTCCCCAGGACAGACAGCCAGCATCACC |
| | | | TGCTCTGGAGATAAATTGGGGGATAAATATGCTTGCTGGTATCAGCAGAAGCCAGGCCAGTCC |
| | | | CCTGTGGTGGTCATCTATCAAGATAGCAAGCGGCCCTCAGGGATCCCTGAGCGATTCTCTGGC |
| | | | TCCAACTCTGGGAACACAGCCACTCTGACCATCAGCGGGACCCAGGCTATGGATGAGGCTGAC |
| | | | TATTACTGTCAGGCGTGGGACAGCAGCACTGCGGTATTCGGCGGAGGGACCAAGCTGACCGTC |
| | | | CTA |
| COV2- | 11 | heavy | CAGCTACAGCTGCAGGAGTCGGGCCCAGGACTGGTGAAGCCTTCGGAGACCCTGTCCCTCACC |
| 2179 | | | TGCACTGTCTCTGGTGGCTCCATCAGCAGTGGAACTTACTACTGCGGCTGGATCCGCCAGCCC |
| | | | CCAGGGAAGGGGCTGGAGTGGATTGGGAGTACATATTATGGTGGGAGCACCCTCTACAACCCG |
| | | | TCCCTCAGGGGTCGAGTCACCATATCCGTAGACACGTCCAAGAACCAGTTTTCCCTGAAGCTG |
| | | | AGCTCTGTGACCGCCGCAGACACGGCTGTGTATTACTGTGCGAGACGGGGTAATTACTATGAT |
| | | | AGTAAGAACTGGTTCGACCCCTTGGGGCCAGGGAACCCTTGGTCACCGTCTcCTCA |
| | 12 | light | GAAATAGTGATGACGCAGTCTCCAGCCACCGTGTCTGTGTCTCCAGGGGAAAGAGCCACCCTC |
| | | | TCCTGCAGGGCCAGTCAGAGTGTTAGCAGCAACTTAGCCTGGTACCAGCAGAAACCTGGCCAG |
| | | | GCTCCCAGGCTCCTCATCTATGGTGCATCCACCAGGGCCACTGGTATCCCAGCCAGGTTCAGT |
| | | | GCCAGTGGGTCTGGGACAGAGTTCACTCTCACCATCAGCAGCCTGCAGTCTGAAGATTTTGCA |
| | | | GTTTATTACTGTCAGCAGTATAATAACTGGCCACCTATGTACACTTTTGGCCAGGGGACCAAG |
| | | | GTGGAGATCAAA |
| COV2- | 13 | heavy | CAGGTGCAGCTGGTGGAGTCTGGGGGAGGCGTGGTCCAGCCTGGGAGGTCCCTGAGAGTCTCC |
| 2181 | | | TGTGCAGCGTCTGGATTCACCTTCAGTAGCCATGGCATGCACTGGGTCCGCCAGGCTCCAGGC |
| | | | AAGGGGCTGGAGTGGGTGTCAGTTATATGGTATGATGGAAGTAATAAATACTATGCAGACTCC |
| | | | GTGAAGGGCCGATTCACCATCTCCAGAGACAATTCCAAGAACACGCTGTCTCTGCAAATGAAC |
| | | | AGCCTGAGAGCCGAAGACACGGCTGTGTATTACTGTGCGAGAGAGAGCGCGGACATATCATCT |
| | | | CGTCTTGACTACTGGGGCCGGGGAACCCTGGTCACCGTCTCCTCA |
| | 14 | light | TCCTATGAGCTGACACAGCCACCCTCGGTGTCAGTGCCCCCAGGACAAACGGCCAGGATCACC |
| | | | TGCTCTGGAGATGCATTGCCAACAAAATATGCTTATTGGTACCAGCAGAAGTCAGGCCAGGCC |
| | | | CCTGTGCTGGTCATCTATGACGACAGCAAACGACCCTCCGGGATCCCTGAGAGATTCTCTGGC |
| | | | TCCAGCTCAGGGACAATGGCCACCTTGACTATCAGTGGGGCCCAGGTGGAGGATGAAGCTGAC |
| | | | TACTACTGTTACTCAACAGACAGCAGTGGTAATGTCTTCGGAACTGGGACCAAGGTCACCGTC |
| | | | CTA |
| COV2- | 15 | heavy | CAGGTGCAGCTGGTGGAGTCTGGGGGAGGCGTGGTCCAGCCTGGGAGGTCCCTGAGAGTCTCC |
| 2183 | | | TGTGCAGCCTCTGGATTCACCTTCAGTAGCTATGCTATGCACTGGGTCCGCCAGGCTCCAGGC |
| | | | AAGGGGCTGGAGTGGGTGGCAGGTATATCATATGATGGAAGCAATAAATACTACGCAGACTCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTGTATTACTGTGCGAGAGCGGACACTATGGTTCGGGGA |
| | | | ACTTATTTTGAGTACTGGGGCCAGGGAACCCTGGTCACCGTCCCTCA |
| | 16 | light | CAGTCTGCCCTGACTCAGCCTGCCTCCGTGTCTGGGTCTCCTGGACAGTCGATCACCATCTCC |
| | | | TGCACTGGAACCAGCAGTGACGTTGGTGGTTATAACTATGTCTCTTGGTACCAACAACACCCA |
| | | | GGCAAAGCCCCCAAACTCATGATTTATGATGTCAGTAATCGGCCCTCAGGGGTTTCTAATCGC |
| | | | TTCTCTGGCTCCAAGTCTGGCAACACGGCCTCCCTGACCATCTCTGGGCTCCAGGCTGAGGAC |
| | | | GAGGCTGATTATTGCTGCAGCTCATATACAAGCAGCAGAGCTGTGCTATTCGGCGGAGGGACC |
| | | | AAGCTGACCGTCCTA |
| COV2- | 17 | heavy | CAGATCACCTTGAAGGAGTCTGGTCCTACGCTGGTAAAACCCACACAGACCCTCACGCTGACC |
| 2184 | | | TGCACCTTCTCTGGGTTCTCACTCAGCACTAGTGGAGTGGGTGTGGCCTGGATCCGTCAGCCC |
| | | | CCAGGAAAGGCCCTGGAGTGGCTTGCTCTCATTTATTGGGATGATGATAAGCGCTACAGCCCA |
| | | | TCTCTGAAGAGCAGGCTCACCATCACCAAGGACACCTCCAAAAACCAGGTGGTCCTTACAATG |
| | | | ACCAACATGGACCCTGTGGACACAGCCACATATTACTGTGCACACAGACTTCCAACGCCCCAA |
| | | | CTGCTACCATCTTTTGAAAACTGGTTCGACCCCTGGGGCCAGGGAACCCTGGTCACCGTCTCC |
| | | | TCA |
| | 18 | light | CAGTCTGTGCTGACTCAGCCACCCTCGGTGTCTGAAGCCCCCAGGCAGAGGGTCACCATCTCC |
| | | | TGTTCTGGAGGCAGCTCCAACATCGGAAATAATGCTGTAAACTGGTACCAGCAGCTCCCAGGA |
| | | | AAGGCTCCCAAACTCCTCATCTATTATGATGATCTGCTGCCCTCAGGGGTCTCTGACCGATTC |
| | | | TCTGGCTCCAAGTCTGGCACCTCAGCCTCCCTGGCCATCAGTGGGCTCCAGTCTGAAGATGAG |
| | | | GCTGATTATTACTGTGCATCATGGGATGACAGCCTGATTGGTCCGGTATTCGGCGGAGGGACC |
| | | | AAGCTGACCGTCCTA |
| COV- | 19 | heavy | GAGGTGCAGCTGGTGGAGTCCGGGGGAGGCTTAGTTCAGCCTGGGGGGTCCCTGAGACTCTCC |
| 2185 | | | TGTGCAGTCTCTGGATTCACCTTCAGTAGCTACTGGATGCACTGGGTCCGCCAAGCTCCAGGG |
| | | | AAGGGGCTGGTGTGGGTCTCACGTATTAATAGTGATGGGAGTAGCACAAGCTACGCGGACTCC |
| | | | GTGAAGGGCCGATTCACCATCTCCAGAGACAACGCCAAGAACACGCTGTATCTGCAAATGAAC |
| | | | AGTCTGAGAGCCGAGGACACGGCTGTGTATTACTGTGCAAGAGAAGTGGAGCAGCTGGCTCAT |
| | | | ATGGTTGACTACTGGGGCCAGGGAACCCTGGTCACCGTCTCCTCA |
| | 20 | light | TCCTATGAGCTGACACAGCCACCCTCGGTGTCAGTGTCCCCAGGACAGACGGCCAGGATCACC |
| | | | TGCTCTGGAGATGCATTGCCAAACCAATATGCTTATTGGTACCAGCAGAAGCCAGGCCAGGCC |
| | | | CCTGTGCTGGTGATATATAAAGACAGTGAGAGGCCCTCAGGGATCCCTGAGCGATTCTCTGGC |
| | | | TCCAGCTCAGGGACAACAGTCACGTTGACCATCAGTGGAGTCCAGGCAGAAGACGAGGCTGAC |
| | | | TATTACTGTCAATCAGCAGACAGCAGTGGTACATCTTGGGTGTTCGGCGGAGGGACCAAGCTG |
| | | | ACCGTCCTA |
| COV2- | 21 | heavy | CAGGTGCAGCTGGTGGAGTCTGGGGGAGGCGTGGTCCAGCCTGGGAGGTCCCTGAGACTCTCC |
| 2186 | | | TGTGCAGCCTCTGGATTCACCTTCAGTAGCTATGCTATGCACTGGGTCCGCCAGGCTCCAGGC |
| | | | AAGGGGCTGGAGTGGGTGGCAGTTATATCATATGATGGAATCAATAAATACTACGCAGACGCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTGTATTACTGTGCGAGGCCACGAAGTGGGAGCTACTAC |
| | | | GCCTACTTTGACTACTGGGGCCAGGGAACCCTGGTCACCGTCTCCTCA |
| | 22 | light | GACATCCAGATGACCCAGTCTCCATCCTCACTGTCTGCATCTGTAGGAGACAGAGTCACCATC |
| | | | ACTTGTCGGGCGAGTCAGGGCATTAGCAATTATTTAGCCTGGTTTCAGCAGAAACCAGGGAAA |
| | | | GCCCCTAAGTCCCTGATCTATGCTGCATCCAGTTTACAAAGTGGGGTCCCATCAAAGTTCAGC |
| | | | GGCAGTGGATCTGGGACAGATTTCACTCTCACCATCAGCAGCCTGCAGCCTGAAGATGTTGCA |
| | | | ACTTATTACTGCCAACAGTATAATAGTCACCCTCCCACTTTCGGCGGAGGGACCAAGGTGGAG |
| | | | ATCAAA |
| COV2- | 23 | heavy | CAGGTGCAGCTGGTGGAGTCTGGGGGAGGCGTGGTCCAGCCTGGGAGGTCCCTGAGACTCTCC |
| 2187 | | | TGTGCAGCCTCTGGATTCACCTTCAGTTACTATCCTATGCACTGGCTCTGGGTCCGCCAGGCT |
| | | | CCAGGCAAGGGGCTGGAGTGGGTGGCAGTTACATCATATGATGGAACCAATAAATACTACGCA |
| | | | GACTCCGTGAAGGGCCGATTCACCATCTCCAGAGACAATTCCAAGAACACTCTGTATCTGCAA |
| | | | ATGAACAGCCTGAGAGCTGAGGACACGGCTGTGTATTACTGTGCGAGAGGGGGAGCTACTAAC |
| | | | TTTGACTACTGGGGCCAGGGAACCCTGGTCACCGTCTCCTCA |
| | 24 | light | TCCTATGTGCTGACTCAGCCACCCTCGGTGTCAGTGGCCCCAGGAAAGACGGCCAACATTACC |
| | | | TGTGGGGGAAACAACATTGGAAGAAAAAGTGTGCACTGGTACCAGCAGAAGTCAGGCCAGGCC |
| | | | CCTGTGCTGGTCGTCTATGATGATAGCGACCGGCCCTCAGGGATCCCTGAGCGATTCTCTGGC |
| | | | TCCAACTCTGGGAACACGGCCACCCTGACCATCAGCAGGGTCGAAGCCGGGGATGAGGCCGAC |
| | | | TATTACTGTCAGGTGTGGGATAGTAGTAGTGATCATCCGGAGTGGGTGTTCGGCGGAGGGACC |
| | | | AAGCTGACCGTCCTA |
| COV2- | 25 | heavy | GAAGCGCAGCTGGTGGAGTCTGGGGGAGGCTTGGTACAGCCTGGCAGGTCCCTGAGACTCTCC |
| 2189 | | | TGTGCAGCCTCTGGATTCACCTTTGATGATTCTGCCATGCACTGGGTCCGGCAAGCTCCAGGG |
| | | | AAGGGCCTGGAGTGGGTCTCAGGTATTAGTTGGAATAGTGGTAACGTAGGCTATGCGGACTCT |
| | | | GTGAAGGGCCGATTCACCATCTCCAGAGACAACGCCAAGAACTCCCTGTATCTGCAAATGAAC |
| | | | AGTCTGAGAGCTGAGGACACGGCCTTGTATTACTGTACAAAAGCCTCTCGATATTGTAGTAGT |
| | | | ACCATCTGCTATTGGAACTGGTTCGACCCCTGGGGCCAGGGAACCCTGGTCACCGTCTCCTCA |
| | 26 | light | GACATCCAGATGACCCAGTCTCCATCCTCCCTGTCTGCATCTGTAGGAGACAGAGTCACCATC |
| | | | ACTTGCCGGGCAAGTCAGAGCATTAGCAGCTATTTAAATTGGTATCAGCAGAAACCAGGGAAA |
| | | | GCCCCTAAGCTCCTGATCTATGGTGCATCCAGTTTGCAAACTGGGGTCCCATCAAGGTTCAGT |
| | | | GGCAGTGGATCTGGGACAGATTTCACTCTCACCATCAGAAGTCTGCAACCTGAAGATTTTGCA |
| | | | AGTTACTACTGTCAACAGAGTTACAGTACCCCCACTTTCGGCGGAGGGACCAAGGTGGAGATC |
| | | | AAA |
| COV2- | 27 | heavy | GAGGTGCAGCTGGTGGAGTCTGGGGGAGGCTTGGTACAGCCTGGAGGGTCCCTGAGACTCTCC |
| 2190 | | | TGTGCAGCCTCTGGATTCACCTTCAGTAGTTATGAAATGAACTGGGTCCGCCAGGCTCCAGGG |
| | | | AAGGGGCTGGAGTGGGTTTCATACATTAGTAGTAGTGGTAGTGCCATATACTACGCAGACTCT |
| | | | GTGAAGGGCCGATTCACCATCTCCAGAGACAACGCCAAGAACTCACTGTATCTGCAAATGAAC |
| | | | AGCCTGAGAGTCGAGGACACGGCTGTTTATTACTGTGCGAGAGAAGCGCGGTCACGATATTTT |
| | | | GACTGGTTACCCTCGTACTACTTTGACTACTGGGGCCAGGGAACCCTGGTCACCGTCTCCTCA |
| | 28 | light | CAGTCTGCCCTGACTCAGCCTGCCTCCGTGTCTGGGTCTCCTGGACAGTCGATCACCATCTCC |
| | | | TGCACTGGAACCAGCAGTGACATTGGTGGTTATAACTATGTCTCCTGGTACCAACAACACCCA |
| | | | GGCAAAGCCCCCAAACTCCTGATTTATGATGTCAGTAATCGGCCCTCAGGGGTTTCTACTCGC |
| | | | TTCTCTGGCTCCAAGTCTGGCAACACGGCCTCCCTGACCATCTCTGGGCTCCAGGCTGAGGAC |
| | | | GAGGCTGATTATTACTGCAGCTCATATACAAGCAGCAGCACCCATGTGGTGTTCGGCGGAGGG |
| | | | ACCAAGCTGACCGTCCTA |
| COV2- | 29 | heavy | CAGGTGCAGGTGGTGCAATCTGGGTCTGAGTTGAAGAAGCCTGGGGCCTCAGTGAAGGTTTCC |
| 2192 | | | TGCAAGGCTTCTGGATACACCTTCACTACCTATGCTATGAATTGGGTGCGACAGGCCCCTGGA |
| | | | CAAGGACTTGAGTGGATGGGATGGATCAACACCAACACTGGGAACCCAACGTATGCCCAGGGC |
| | | | TTCACAGGACGGTTTGTCTTCTCCTTGGACACCTCTGTCAACACGGCATTTCTGCATATCGGC |
| | | | AGCCTAAAGGCTGAGGACACTGCCGTGTATTACTGTGCGAGAGATCAGGACAGTGGCTACCCA |
| | | | ACTTACTACTACTACTACATGGACGTCTGGGGCAAAGGGACCACGGTCACCGTCTCCTC |
| | 30 | light | GATATTGTGATGACCCAGACTCCACTCTCTCTGTCCGTCACCCCTGGACAGCCGGCCTCCATC |
| | | | TCCTGCAAGTCTAGTCAGAGCCTCCTGCATAGTGATGGAAAGACCTATTTGTATTGGTACCTG |
| | | | CAGAAGCCAGGCCAGTCTCCACAGCTCCTGATCTATGAAGTTTCCAACCGGTTCTCTGGAGTG |
| | | | CCAGATAGGTTCAGTGGCAGCGGGTCAGGGACAGATTTCACACTGAAAATCAGCCGGGTGGAG |
| | | | GCTGAGGATGTTGGGGTTTATTACTGCATGCAAAGTATACAGCCTCCTCTCACTTTCGGCGGA |
| | | | GGGACCAAGGTGGAGATCAAA |
| COV2- | 31 | heavy | CAGGTGCAGCTGGTGGAGTCTGGGGGAGGCGTGGTCCAGCCTGGGAGGTCCCTGAGACTCTCC |
| 2193 | | | TGTGCAGCCTCTGGATTCACCTTCAGTACCTATGCTATGCACTGGGTCCGCCAGGCTCCAGGC |
| | | | AAGGGGCTGGCGTGGGTGGCACTTATATCATATGATGGATATAATAAATACTACGCAGACTCC |
| | | | GTGAGGGGCCGATTCACCATCTCCAGAATCAATTCCAAGAACACGCTGTCTCTGCAGATGAAC |
| | | | AGCCTGAGAGCTGAGGACACGGCTGTCTATTACTGTGCGAGAGGGTCAGCTGGAAACTACTAC |
| | | | TACGGTATGGACGTCTGGGGCCAGGGGACCACGGTCACCGTCTCCTCA |
| | 32 | light | GACATCCAGATGACCCAGTCTCCATCCTCCCTGTCTGCATCTGTAGGAGACAGAGTCACCATC |
| | | | ACTTGCCGGGCAAGTCAGACCATTACCAACTATTTAAATTGGTATCAGCTGAAATCAGGGAGA |
| | | | GCCCCCAAGCTCCTGATCTATGCTGCATCCAGTTTGCAAAGTGGGGTCCCATCAAGGTTCAGT |
| | | | GGCAGTGGATCTGGGACAGATTTCACTCTCACCATCAGCAGTCTGCAACCTGAAGATTTTGCA |
| | | | ACTTACTACTGTCAACAGAGTTACAGTACGCCGTACACTTTTGGCCAGGGGACCAAGCTGGAG |
| | | | ATCAAA |
| COV2- | 33 | heavy | CAGGTGCATCTGGTGGAGTCTGGGGGAGGCGTGGTCCAGCCTGGGAGGTCCCTGAGACTCTCC |
| 2195 | | | TGTGCAGCCTCTGGATTCACCTTCAGTAACTATGGCATGCACTGGGTCCGCCAGGCTCCAGGC |
| | | | AAGGGGCTGGAGTGGGTGGCAGTTATCTCAAATGATGAATTTAATAAATTCTATGCAAACTCC |
| | | | GTGAAGGGCCGATTCACCATCTCCAGAGACAATTCGAAGAACACGGTGTATTTGCAATTGAAC |
| | | | AGTCTGAGAACTGAGGACACGGCTCGATATTACTGTGCGAAAGGGGGGGATGGCAGTGGCTGG |
| | | | GCCTGGGACGGTGATAACCCCCCAACGGACTACTGGGGCCAGGGAACCCTGGTCATCGTCTCT |
| | | | TCA |
| | 34 | light | GACATCGTGATGACCCAGTCTCCGGACTTCCTGGCTGTGTCTCTGGGCGAGAGGGCCACCATC |
| | | | AGCTGCAAGTCCAGTCAGAGTGTTTTATACACCCCCAAGAATAAGAACTACTTAGCTTGGTAC |
| | | | AAGCAGAAACCAGGACAGCCTCCTAAGGTGCTCATTTACTGGGCATCTACCCGGGAATCCGGG |
| | | | GTCCCTGACCGATTCAGTGGCAGCGGGTCTGGGACAGATTTCACTCTCATCATCAGCAGCCTG |
| | | | CAGGCTGAGGATGCGGCAGTTTATTACTGTCAGCAATATTATACTGCTCCTCTCACTTTCGGC |
| | | | GGTGGGACCAGGGTGGAGATCAA |
| COV2- | 37 | heavy | GAGGTGCAGCTGGTGCAGTCTGGAGCAGAGGTGAAAAAGCCCGGGGAGTCTCTGAAGATCTCC |
| 2197 | | | TGTAAGGGTTCTGGATACAGCTTTACCAGCTACTGGATCGGCTGGGTGCGCCAGATGCCCGGG |
| | | | AAAGGCCTGGAGTGGATGGGGATCATCTATCCTGGTGACTCTGATACCAGATACAGCCCGTCC |
| | | | TTCCAAGGCCAGGTCACCATCTCAGCCGACAAGTCCATCAGCACCGCCTACCTGCAGTGGAGC |
| | | | AGCCTGAAGGCCTCGGACACCGCCATGTATTACTGTGCGAGACCCGACTATAGCAGTGGCTGG |
| | | | TTTAGCTACTGGTACTTCGATCTCTGGGGCCGTGGCACCCTGGTCACTGTCTCCTCA |
| | 38 | light | GAAATTGTGTTGACGCAGTCTCCAGGCACCCTGTCTTTGTCTCCAGGGGAAAGAGCCACCCTC |
| | | | TCCTGCAGGGCCAGTCAGAGTGTTAGCAGCAACTTCTTAGCCTGGTACCAGCAGAAACCTGGC |
| | | | CAGGCTCCCAGGCTCCTCATCTATGGTGCATCCAGCAGGGCCACTGGCATCCCAGACAGGTTC |
| | | | AGTGGCAGTGGGTCTGGGACAGACTTCACTCTCACCATCAGCAGACTGGAGCCTGAAGATTTT |
| | | | GCAGTGTATTACTGCCAGCAGTATGGTCGCTCACCGATCACCTTCGGCCAAGGGACACGACTG |
| | | | GAGATTAAA |
| COV2- | 39 | heavy | CAGGTCCAGCTGGTACAGTCTGGGGCTGAGGTGAAGAAGCCTGGGGCCTCAGTGAAGGTCTCC |
| 2199 | | | TGCAAGGTTTCCGGATACACCCTCACTGAACTATCCATACACTGGGTGCGACAGGCTCCTGGA |
| | | | AAAGGGCTTGAGTGGATGGGAGGTTTTGATCCTGAAGATGCTGAAACAATCTACGCACAGCAG |
| | | | TTCCAGGGCAGAGTCACCATGACCGAGGACACATCTACAGACACAGCCTACATGGAGCTGAGC |
| | | | AGCCTGAAATCTGAGGACACGGCCCTGTATTACTGTGCAACAGGGTTCGCGGTGTTTGGGAGG |
| | | | GCAGCAGTTCCCTACTGGGGCCAGGGAACCCTGGTCACCGTCTCCTCA |
| | 40 | light | TCCTATGAGCTGACTCAGCCACCCTCAGTGTCCGTGTCCCCAGGACAGACAGCCAGCATCACC |
| | | | TGCTTTGGAGATAAATTGGGGGATAAATATGCTTGCTGGTTTCAGCAGAAGCCAGGCCAGTCC |
| | | | CCTGTGCTGATCATCTATCAAGGTGCCAAGCGGCCCTCAGGGATCCCTGAGCGATTCTCTGGC |
| | | | TCCAACTCTGGGAACACAGCCACTCTGACCATCAGCGGGACCCAGGCTATGGATGAGGCTGAC |
| | | | TATTACTGTCAGGCGTGGGACAGCAGCACTGTGGTTTTCGGCGGAGGGACCAAGCTGACCGTC |
| | | | CTA |
| COV2- | 41 | light | CAGGTGCAGGTGGTGGAGTCTGGGGGAGGCGTGGTCCAGCCTGGGAGGTCCCTGAGACTCTCC |
| 2200 | | | TGTGCAGCCTCTGGATTCACGTTCAGTAGCTATGGCATGCACTGGGTCCGCCAGGCTCCAGGC |
| | | | AAGGGGCTGGAGTGGGTGGCAGTTATATCATATGATGGAAGTAATAAATACTATGCAGACTCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTGTATTACTGTGCGAAAGATCTCACAATTGTAGTAATA |
| | | | CCAGCTGCCCCAAATTTTGACTACTGGGGCCAGGGAACCCTGGTCACCGTCTCCTCA |
| | 42 | light | CAGTCTGCCCTGACTCAGCCTGCCTCCGTGTCTGGGTCTCCTGGACAGTCGATCACCATCTCC |
| | | | TGCACTGGAACCAGCAGTGACGTTGGTGGTTATAACTATGTCTCCTGGTACCAACAACACCCA |
| | | | GGCAAAGCCCCCAAACTCATGATTTATGATGTCAGTAATCGGCCCTCAGGGGTTTCTAATCGC |
| | | | TTCTCTGGCTCCAGGTCTGGCAACACGGCCTCCCTGACCATCTCTGGGCTCCAGGCTGAGGAC |
| | | | GAGGCTGATTATTATTGCAGGTCATATACAAGCAGCAGCACTCCTGTGGTGTTCGGCGGAGGG |
| | | | ACCAAGCTGACCGTCCTA |
| COV2- | 43 | heavy | GAGGTGCAGCTGGTGCAGTCTGGAGCAGAGGTGAAAAAGCCCGGGGAGTCTCTGAAGATCTCC |
| 2207 | | | TGTAAGGGTTCTGGATACAGCTTTACCAGTCACTGGATCGGCTGGGTGCGCCAGATGCCCGGG |
| | | | AAAGGCCTGGAGTGGATGGGGATCATCTATCCTGGTGACTCTGATACCAGATATAGCCCGTCC |
| | | | TTCCAAGGGCAGGTCACCATCTCAGCCGACAAGTCCATCAGCACCGCCTACCTGCAGTGGAGC |
| | | | AGCCTGAAGGCCTCGGACACCGCCATGTATTACTGTGCGAGTGCCCTCAGAGAGCGAGGCGTA |
| | | | CAGCTGTGGTCAGTTTGGGGCCAGGGAACCCTGGTCACCGTCTCCTCA |
| | 44 | light | CAGTCTGTGCTGACGCAGCCGCCGTCAGTGTCTGGGGGCCCAGGGCAGAGGGTCACCATCTCC |
| | | | TGCACTGGGAGCAGCTCCAACATCGGGGCAGGTTATGATGTACACTGGTACCAGCAGCTTCCA |
| | | | GGAACAGCCCCCAAACTCCTCATCTTTATTAACTCCAATCGGCCCTCAGGGGTCCCTGACCGC |
| | | | TTCTCTGGCTCCAAGTCTGGCACCTCAGCCTCCCTGGCCATCACTGGGCTCCAGGCTGAGGAT |
| | | | GAGGCTGATTATTACTGCCAGTCCTATGACAGCAGCCTGGGTGCCTTGTLCGGCGGAGGGACC |
| | | | AAGCTGACCGTCCTA |
| COV2- | 45 | heavy | CAGGTGCAGCTGGTGGAGTCTGGGGGAGGCGTGGTCCAGCCTGGGAGGTCCCTGAGACTCTCC |
| 2210 | | | TGTGCAGCCTCTGGATTCACCTTCAGTAGCTATGCTATGCACTGGGTCCGCCAGGCTCCAGGC |
| | | | AAGGGGCTGGAGTGGGTGGCAGTTATATCATATGATGGAAGCAATAAATACTACGCAGACTCC |
| | | | GTGAAGGGCCGATTCACCATCTCCAGAGACAATTCCAAGAACACTCTTTATCTGCAAATGAAC |
| | | | AGCCTGAGAGTTGAGGACACGGCTGTGTATTACTGTGCGAGAGATCAAGAATGGTTCAGGGAG |
| | | | TTATTCCTTTTTGACTACTGGGGCCAGGGAACCCTGGTCACCGTCTCCTCA |
| | 46 | light | GACATCCAGATGACCCAGTCTCCATCTTCCGTGTCTGCATCTGTAGGAGACAGAGTCACCATC |
| | | | ACTTGTCGGGCGAGTCAGGGTATTAGCAGCTGGTTAGCCTGGTATCAGCAGAAACCAGGGAAA |
| | | | GCCCCTAAGCTCCTGATCTATGATGCATCCAGTTTGCAAAGTGGGGTCCCATCAAGGTTCAGC |
| | | | GGCAGTGGATCTGGGACAGATTTCACTCTCACCATCAGCAGCCTGCAGCCTGAAGATTTTGCA |
| | | | ACTTACTATTGTCAACAGGCTAACAGTTTCCCTCCGACTTTCGGCCCTGGGACCAAAGTGGAT |
| | | | ATCAAA |
| COV2- | 47 | heavy | CAGGTGCAGUTGGTGGAGTCCGGGGGAGGCGTGGTCCAGCCTGGGAGGTCCCTGAGACTCTCC |
| 2211 | | | TGTGCAGCCTCTGGATTCACCTTCAGTACCTATGGCATGCACTGGGTCCGCCAGGCTCCAGGC |
| | | | AAGGGGCTGGAGTGGGTGGCAGTTATATCATATGATGGAAGTAATAAATACTATGCAGACTCC |
| | | | GTGAAGGGCCGATTCACCATCTCCAGAGACAATTCCAAGAACACACTGTATCTGCAAATGAAC |
| | | | AGCCTGAGAGCTGAGGACACGGCTATGTATTACTGTGCGAAAGATGGGAGTATAGCAGCAGCT |
| | | | GACTACTGGGGCCAGGGAACCCTGGTCACCGTCCTC |
| | 48 | light | GACATCGTGATGACCCAGTCTCCAGACTCCCTGGCTGTGTCTCTGGGCGAGAGGGCCACCATC |
| | | | AACTGCAAGTCCAGCCAGAGTGTCTTACACAGCTCCAACAACAAGGACTCCTTAGTTTGGTAC |
| | | | CAGCAGAAACCAGGACAGCCTCCTAAGCTGCTCATTTACTGGGCATCTAGCCGGGAATCCGGG |
| | | | GTCCCTGACCGATTCAGTGGCAGCGGGTCTGGGACAGATTTCACTCTCACCATCAGCAGCCTG |
| | | | CAGGCTGAAGATGTGGCAGTTTATTACTGTCAGCAATATTATAGTACTCCTTGGACGTTCGGC |
| | | | CAAGGGACCAAGGTGGAAATCAAA |
| COV2- | 49 | heavy | GAGGTGCAGCTGGTGGAGTCTGGGGGAGGCCTGGTCAAGCCTGGGGGGTCCCTGAGACTCTCC |
| 2212 | | | TGTGCAGCCTCTGGATTCACCTTCAGTAGTTATAGCATGAACTGGGTCCGCCAGGCTCCAGGG |
| | | | AAGGGGCTGGAGTGGGTCTCATCCATAAGTAATAGTAATAGTTTCATATACTACGCAGACTCA |
| | | | ATGAAGGGCCGATTCACCATCTCCAGAGACAACGCCAAGAACTCACTGTATCTGCAAATGAAC |
| | | | AGCCTGAGAGCCGAGGACACGGCTGTGTATTACTGTGCGCGAGTTAACGGCAACTCAAACTGG |
| | | | AACTTTGGGTCTTACTACTACTACTACATGGACGTCTGGGGCAAAGGGACCACGGTCACCGTC |
| | | | TCCTCA |
| | 50 | light | GAAATTGTGTTGACACAGTCTCCAGCCATTTTGTCTTTGTCTCCAGGGGAAAGAGCCACCCTC |
| | | | TCCTGCAGGGCCAGTCAGAGTGTTAGTAGCTACTTAGCCTGGTACCAACAGAAGCCTGGCCAG |
| | | | GCTCCCAGGCTCCTCATCTATGATACATCCAACAGGGCCACTGGCATCCCAGCCAGGTTCAGT |
| | | | GGCAGTGGGTCTGGGACAGACTTCACTCTCACTATCAGCAGCCTAGAGCCTGAAGATTTTGCA |
| | | | TTTTATTACTGTCAGCAGCGTGGCAACTGGTGGACGTTCGCCCAAGGGACCAAGGTGGAAATC |
| | | | AAA |
| COV2- | 51 | heavy | GAGGTGCAGCTGGTGGAGTCTGGGGGAGGCCTGGTCAAGCCTGGGGGGTCCCTGAGACTCTCC |
| 2215 | | | TGTGCAGCCTCTGGATTCACTTTCAGTGGCTATAGCATGAACTGGGTCCGCCAGGCTCCAGGG |
| | | | AAGGGGCTGGAGTGGGTCTCATCCATTAGTAGTAGTAGTAGTTACATATACTACGCAGACTCA |
| | | | GTGAAGGGCCGATTCACCATCTCCAGAGACAACGCCAAGAACTCACTGTATCTGCAAATGAAC |
| | | | AGCCTGAGGGCCGAGGACACGGCTGTGTATTACTGTGCGAGATGGCTACAGTTAAGATCAGAC |
| | | | TACTATTACTTCGGTATGGACGTCTGGGGCCAAGGGACCACGGTCACCGTCTCCTCA |
| | 52 | light | GAAATAGTGATGACGCAGTCTCCAGCCACCCTGTCTGTGTCTCCAGGGGAAAGAGCCACCCTC |
| | | | TCCTGCAGGGCCAGTCAGAGTGTTAGCAACAACTTAGCCTGGTACCAGCACAAACCTGGCCAG |
| | | | GCTCCCAGGCTCCTCATCTATGGTGCATCCACCAGGGCCACTGGTATCCCAGCCAGGTTCAGT |
| | | | GGCAGTGGGTCTGGGACAGAGTTCACTCTCACCATCAGCAGCCTACAGTCTGAAGATTTTGCA |
| | | | GTTTATTTCTGTCAGCAGTGTTATAACTGGCCTCCGTGGACGTTCGGCCAAGGGACCAAGGTG |
| | | | GAAATCAAA |
| COV2- | 53 | heavy | CAGGTGCAGCTGCAGGAGTCGGGCCCAGGACTGGTGAAGCCTTCGGAGACCCTGTCCCTCACC |
| 2222 | | | TGCACTGTCTCTGGTGGCTCCATCAGTAGTTACTACTGGAGCTGGATCCGGCAGCCCCCAGGG |
| | | | AAGGGACTGGAGTGGATTGGTTATATCTATTACAGTGGGAGCACCAACTACAACCCCTCCCTC |
| | | | AAGAGTCGAGTCACCATATCAGTAGACATGTCCAAGAACCAGTTCTCCCTGAAGCTGAGGTCT |
| | | | GTGACCGCTGCGGACACGGCCGTGTATTACTGTGCGAGAGCCCCGAGGGAAAGGCTCCAATGG |
| | | | GGGGAGTACTACTTCGACTACTGGGGCCAGGGAACCCTGGTCACCGTCTCCTCA |
| | 54 | light | CAGTCTGCCCTGACTCAGCCTGCCTCCGTGTCTGGGTCTCCTGGACAGTCGATCACCATCTCC |
| | | | TGCACTGGAACCAGCAGTGATGTTGGGAGTTATAACCTTGTCTCCTGGTACCAACAGCATGCA |
| | | | GGCAAAGCCCCCAAACTCATGATTTATGAGGTCATTAAGCGGCCCTCAGGGGTTTCTAATCGC |
| | | | TTCTCTGGCTCCAAGTCTGGCAACACGGCCTCCCTGACAATCTCTGGGCTCCAGGCTGAGGAC |
| | | | GAGGCTGATTATTACTGCTGCTCATATGCAGTTAGTACCACTTATGTTATATTCGGCGGAGGG |
| | | | ACCAAGCTGACCGTCCTA |
| COV2- | 55 | heavy | GAGGTGCAGCTGGTGCAGTCTGGAGCAGAGGTGAAAAAGCCCGGGGAGTCTCTGAAGATCTCC |
| 2226 | | | TGTAAGGGTTCTGGATACAGCTTTACCAACTCCTGGATCGGCTGGGTGCGCCAGATGCCCGGG |
| | | | AAAGGCCTGGAGTGGATGGGGATCATCTATCCTGGTGACTCTGATACCAGATACAGCCCGTCC |
| | | | TTCCAAGGCCAGGTCACCATCTCAGCCGACAAGTCCATCAGCACCGCCTACCTGCAGTGGAGC |
| | | | AGCCTGAAGGCCTCGGACACCGCCATCTATTACTGTGCGACACATCGTTGTAGTGGTGGTTTG |
| | | | TGCTACTTAGCCTACTGGGGCCAGGGAACCCTGGTCACCGTCTCCTCA |
| | 56 | light | CAGCCTGTGCTGACTCAGCCACCTTCTGCATCAGCCTCCCTGGGAGCCTCGGTCACACTCACC |
| | | | TGCACCCTGAGCAGCGGCTACAGTAATTATAAAGTGGACTGGTACCAGCAGAGACCAGGGAAG |
| | | | GGCCCCCGGTTTGTGATGCGAGTGGGCACTGGTGGGATTGTGGGATCCAAGGGGGATGGCATC |
| | | | CCTGATCGCTTCTCAGTCTTGGGCTCAGGCCTGAATCGGTACCTGACCATCAAGAACATCCAG |
| | | | GAAGAGGATGAGAGTGACTACCACTGTGGGGCAGACCATGGCAGTGGGAGCAACTTCGTTTTT |
| | | | GTGGTTTTCGGCGGAGGGACCAAGCTGACCGTCCTA |
| COV2- | 57 | heavy | CAGGTGCAGCTGGTGGAGTCTGGGGGAGGCGTGGTCCAGCCTGGGAGGTCCCTGAGACTCTCC |
| 2227 | | | TGTGCAGCGTCTGGATTCACCTTCAGTAGCTATGGCATGCACTGGGTCCGCCAGGCTCCAGGC |
| | | | AAGGGGCTGGAGTGGGTGGCAGTTATATGGTATGATGGAAGTAAAAAAGACTATGCAGACTCC |
| | | | GTGAAGGGCCGATTCACCATCrCCAGAGACAATTCCAAGAACACGCTGTATCTGCAAATGAAC |
| | | | AGCCTGAGAGCCGAGGACACGGCTGTGTATTACTGTGCGAGAGATCAATCTCAGGGCGCTTAT |
| | | | ATTTTGACTGGTTATAGGGGCTACGGTATGGACGTCTGGGGCCAGGGGACCACGGTCACCGTC |
| | | | TCCTCA |
| | 58 | light | GATATTGTGATGACTCAGTCTCCACTCTCCCTGCCCGTCACCCCTGGAGAGCCGGCCTCCATC |
| | | | TCCTGCAGATCTAGTCAGAGCCTCCTGCATAGTAATGGATACAACTATTTGGATTGGTACTTG |
| | | | CAGAAGCCAGGGCAGTCTCCACAGTTCCTGATCTATTTGGGTTCTAATCGGGCCTCCGGGGTC |
| | | | CCTGACAGGTTCAGTGGCAGTGGATCAGGCACAGATTTTATACTGAAAATCAGCAGAGTGGAG |
| | | | GCTGAGGATGTTGGGGTTTATTACTGCATGCAAGCTCTTCAAACTCCATTCACTTTCGGCCCT |
| | | | GGGACCAAAGTGGATATCAAA |
| COV2- | 59 | heavy | CAGGTGCAGCTACAGCAGTGGGGCGCAGGACTGTTGAAGCCTTCGGAGACCCTGTCCCTCACC |
| 2228 | | | TGCGCTGTCTATGGTGGGTCCTTCAGTGGTCACTACTGGAGCTGGATCCGCCAGCCCCCAGGG |
| | | | AAGGGACTGGAGTGGATTGGGGAAATCAATCACAGTGGAAGCACCAACTACAACCCGTCCCTC |
| | | | AAGAGTCGAGTCACCATATCAGTAGACACGTCCAAGAACCAGTTCTCCCTGAAGCTGAGCTCT |
| | | | GTGACCGCCGCGGACACGGCTGTGTATTACTGTGCGAGACCCCCCCAAGCAGCTCGTATTCAT |
| | | | TACTACTACTACATGGACGTCTGGGGCAAAGGGACCACGGTCACCGTCTCCTCA |
| | 60 | light | GAAATAGTGATGACGCAGTCTCCAGCCACCCTGTCTGTGTCTCCAGGGGAAAGAGCCACCCTC |
| | | | TCCTGCAGGGCCAGTCAGAGTGTTAGCAGCAACTTAGCCTGGTACCAGCAGAAACCTGGCCAG |
| | | | GCTCCCAGGCTCCTCATCTATGGTGCATCCACCAGGGCCACTGGTATCCCAGCCAGGTTCAGT |
| | | | GGCAGTGGGTCTGGGACAGAGTTCACTCTCACCATCAGCAGCCTGCAGTCTGAAGATTTTGCA |
| | | | GTTTATTACTGTCAGCAGTATAATTACTGGCCTCCCCTCACTTTCGGCGGAGGGACCAAGGTG |
| | | | GAGATCAAA |
| COV2- | 61 | heavy | GAAGTGCAGCTGGTGGAGTCTGGGGGAGGCTTGGTACAGCCTGGCAGGTCCCTGAGACTCTCC |
| 2231 | | | TGTGCAGCCTCTGGATTCACCTTTGATGATTATGCCATGAACTGGGTCCGGCAACCTCCAGGG |
| | | | AAGGGCCTGGAGTGGGTCTCAGGTATTAGTTGGAATAGTGATAGCATAGGCTATGCGGACTCC |
| | | | GTGAAGGGCCGATTCACCATCTCCAGAGACAACGCCAAGAACTCCCTGTATCTGCAAATGAAC |
| | | | AGTCTGAGAGCTGAGGACACGGCCATGTATTACTGTGCAAAAGGAAGGGGTGCTGGTTATACT |
| | | | TCCTACATGGACGTCTGGGGCAAAGGGACCACGGTCACCGTCTCCTCA |
| | 62 | light | TCCTATGTGCTGACTCAGCCACCCTCGGTGTCAGTGGCCCCAGGAAAGACGGCCAGGATTACC |
| | | | TGTGAGGGAAACAACATTGGAAGTAAAAGTGTGCACTGGTACCAGCAGAAGCCAGGCCAGGCC |
| | | | CCTGTGCTGGTCGTCTATGATGATAGCGGCCGGCCCTCAGGGATCCCTGAACGATTCTCTGGT |
| | | | TCCAACTCTGGGAACACGGCCACCCTGACCATCAGCAGGGTCGAAGCCGGGGATGAGGCCGAC |
| | | | TATTTCTGTCAGGTGTGGGATAGTAGTAGTGATCATCATGTGGTTTTCGGCGGAGGGACCAAG |
| | | | CTGACCGTCCT |
| COV2- | 63 | heavy | CCTACACGACGCTCTTCCGATCTGGGGGTCACTGGATCGGCTGGGTGCGCCAGATGCCCGGGA |
| 2233 | | | AAGGCCTGGAGTGGATGGGGATCATCTATCCTGGTGACTCTGATACCAGATATAGCCCGTCCT |
| | | | TCCAAGGGCAGGTCACCATCTCAGCCGACAAGTCCATCAGCACCGCCTACCTGCAGTGGAGCA |
| | | | GCCTGAAGGCCTCGGACACCGCCATGTATTACTGTGCGAGTGCCCTCAGAGAGCGAGGCGTAC |
| | | | AGCTGTGGTCAGTTTGGGGCCAGGGAACCCTGGTCACCGTCTCCTCAG |
| | 64 | light | CAGTCTGTGCTGACGCAGCCGCCCTCAGTGTCTGGGGCCCCAGGGCAGAGGGTCACCATCTCC |
| | | | TGCACTGGGAGCAGCTCCAACATCGGGGCAGGTTATGATGTACACTGGTACCAGCAGCTTCCA |
| | | | GGAACAGCCCCCAAACTCCTCATCTTTATTAACTCCAATCGGCCCTCAGGGGTCCCTGACCGC |
| | | | TTCTCTGGCTCCAAGTCTGGCACCTCAGCCTCCCTGGCCATCACTGGGCTCCAGGCTGAGGAT |
| | | | GAGGCTGATTATTACTGCCAGTCCTATGACAGCAGCCTGGGTGCCTTGTTCGGCGGAGGGACC |
| | | | AAGCTGACCGTCCTA |
| COV2- | 65 | heavy | GAAGTGCAGCTGGTGGAGTCTGGGGGAGGCTTGGTACAGCCTGGCAGGTCCCTGAGACTCTCC |
| 2046 | | | TGTGCAGCCTCTGGATTCACCTTTGATGATTATGCCATGCACTGGGTCCGGCAAGCTCCAGGG |
| | | | AAGGGCCTGGAGTGGGTCTCAGGTATTAGTTGGAATAGTGGTAGCATAGCCTATACGGACTCT |
| | | | GTGAAGGGCCGATTCACCATCTCCAGAGATAACGCCAAGAACTCCCTGTATCTGCAAATGAAC |
| | | | AGTCTGAGAGCTGAGGACACGGCCTTGTATTACTGTGCAAAAGCACACTCGACTGGACACCAA |
| | | | TACTAGTACGGTATGGACGTCTGGGGCCAAGGGACCACGGTCACTGTCTCCTCA |
| | 66 | light | GACATCCAGATGACCCAGTCTCCATCCTCCTTGTCTGCATCTGTAGGAGACAGAGTCACCATC |
| | | | ACTTGCCGGGCAAGTCAGAGCATTAGCAGCTTTTTAAATTGGTATCAGCAGAAACCAGGGAAA |
| | | | GCCCCTAAGCTCCTGATCTATGCTGCATTCAATTTGCAAAGTGGGGTCCCATCAAGGTTCAGT |
| | | | GGCAGTGGATCTGGGACAGATTTCACTCTCACCATCAGCAGTCTGCAATCTGAAGATTTTGCA |
| | | | ACTTACTACTGTCAACAGAGTTACAATACCCCTTACACTTTTGGCCAGGGGACCAAGCTGGAG |
| | | | ATCAAA |
| COV2- | 67 | hEavy | GAAGTGCAGCTGGTGGAGTCTGGGGGAGGCTTGGTACAGCCTGGCAGGTCCCTGAGACTCTCC |
| 2047 | | | TGTGCAGCCTCTGGATTCACCTTTGATGATTATGCCATGCACTGGGTCCGGCAAGCTCCAGGG |
| | | | AAGGGCCTGGAGTGGGTCTCAGGTATTAGTTGGAATAGTGGTAGCATAGGCTATGCGGACTCT |
| | | | GTGAAGGGCCGATTCACCATCTCCAGAGACAACGCCAAGAACTCCCTATATCTGCAAATGAAC |
| | | | AGTCTGAGAGCTGAGGACACGGCCTTGTATTACTGTGCAAAAGTGTCGTCCATTACTAGCCTA |
| | | | TTGGGATACTACTTTGACTCCTGGGGCCAGGGAACCCTGGTCACCGTCTCCTCA |
| | 68 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTACTTAGCCTGGTACCAACAGAAACCTGGCCAG |
| | | | GCTCCCAGGCTCCTCATCTATGATGCATCCAACAGGGCCACTGGCATCCCAGCCAGGTTCAGT |
| | | | GGCAGTGGGTCTGGGACAGACTTCACTCTCACCATCAGCAGCCTAGAGCCTGAAGATTTTGCA |
| | | | GTTTATTACTGTCAGCACCGTAGCAACTGGCCTCCGAGGCTCACTTTCGGCGGAGGGACCAAG |
| | | | GTGGAGATCAAA |
| COV2- | 69 | heavy | CAGGTCCAGGTGGTTCAATCTGGGGCTGAGGTGAAGAAGCCTGGGTCCTCGGTGAAGGTCTCC |
| 2048 | | | TGCAAGACTTCTGGAGACACCTCCAGCAGTTATACTGTCGGCTGGGTGCGACAGGCCCCTGGA |
| | | | CAAGGGCTTGAGTGGATGGGAAGGATCATCCCTATCCTTGGTATAGCATACTCCGCACAGAAG |
| | | | TTCCAGGGCAGACTCACGATTACCGCGGACAAATCCACGAGCACATCCTACATGGAGCTGAGC |
| | | | AGCCTGAGATCTGAGGACACGGCCGTTTATTACTGTGCGAGAGGGGTGGAGCTGCTACTCCG |
| | | | GGTTGGTTCGACCCCTGGGGCCAGGGAACCCTGGTCACCGTCTCCTCA |
| | 70 | light | GAAATAGTGATGACGCAGTCTCCAGCCACCCTGTCTGTGTCTCCAGGGGAAAGAGTCACCCTC |
| | | | TCCTGCAGGGCCAGTCAGAGTGTTAGCAGCAACTTAGCCTGGTACCAGCAGAAACCTGGCCAG |
| | | | GCTCCCAGGCTCCTCATCTATGGTGCATCCACCAGGGCCACTGGTATCCCAGCCAGGTTCAGT |
| | | | GGCGGTGGGTCTGGGACAGAGTTCACTCTCACCATCAGCAGCCTGCAGTCTGAAGATTTTGCA |
| | | | GTTTATTACTGTCAGCAGTATAATAACTTCCTCACTTTCGGCGGAGGGACCAAGGTAGAGATC |
| | | | AAA |
| COV2- | 71 | heavy | GAGGTGCAGCTGGTGGAGTCCGGGGGAGGCTTAGTTCAGCCTGGGGGGTCCCTGAGACTCTCC |
| 2049 | | | TGTGCAGCCTCTGGATTCACCTTCAGTAGCTACTGGATGCACTGGGTCCGCCAAGTTCCAGGG |
| | | | AAGGGGCTGGTGTGGGTCTCACGTATTAATAGTGATGGGAGTAGCACAAGCTACGCGGACTCC |
| | | | GTGAAGGGCCGATTCACCATCTCCAGAGACAACGCCAAGAACACGCTGTATCTGGAAATGAAC |
| | | | AGTCTGAGAGCCCAGGACACGGCTGTTTATTACTGTGCAGGTTCCCCGTGGCTACGAGGCGAC |
| | | | ATTGACTACTGGGGCCAGGGAACCCTGGTCACCGTCTCCTCA |
| | 72 | light | AATTTTATGCTGACTCAGCCCCACTCTGTGTCGGAGTCTCCGGGGAAGACGGTAACCATCTCC |
| | | | TGCACCGGCAGCAGTGGCAGCATTGCCAGCAACTATGTGCAGTGGTACCAGCAGCGCCCGGGC |
| | | | AGTGCCCCCACCACTGTGATCTATGAGGATAACCAAAGGCCCTCTGGGGTCCCTGATCGGTTC |
| | | | TCTGGCTCCATCGACAGCTCCTCCAACTCTGCCTCCCTCACCATCTCTGGGCTGATGACTGAG |
| | | | GACGAGGCTGACTACTACTGTCAGTCTTATGATGGCAGCAATCATGCTGTGGTATTCGGCGGA |
| | | | GGGACCAAGCTGACCGTCCTA |
| COV2- | 73 | heavy | CAGGTGCAGCTGGTGCAGTCTGGGGCTGAGGTGAAGAAGCCTGGGGCCTCAGTGAAGGTCTCC |
| 2050 | | | TGCAAGGCTTCTGGATACACCTTCACCGACTACTATATGCACTGGGTGCGACAGGCCCCTGGA |
| | | | CAAGGGCTTGAGTGGATGGGATGGATCAACCCTAACAGTCGTGGCACAAACTATGCACAGAAG |
| | | | TTTCAGGGCAGGGTCACTATGACCAGGGACACGTCCATCAGCACAGTCTACATGGAGCTGAGC |
| | | | AGGCTGACATCTGACGACACGGCCGTCTATTACTGTGCGAGAGTGGTGGTCCTCGGCTATGGC |
| | | | CGCCCAAACAATTACTATGATGGTAGGAATGTGTGGGACTACTGGGGCCAGGGAACCCTGGTC |
| | | | ACCGTCTCCTCA |
| | 74 | light | CAGTCTGTGCTGACTCAGCCACCCTCAGCGTCTGGGACCCCCGGGCAGAGGGTCATCATCTCT |
| | | | TGTTCTGGAAGCAGCTCCAACATCGGAAGTAATACTGTAAAGTGGTATCATCAGCTCCCAGGA |
| | | | ACGGCCCCCAAACTCCTCATCTGTAGTAATAATCAGCGGCCCTCAGGGGTCCCTGACCGATTC |
| | | | TCTGGCTCCAAGTGTGACACCTCAGCCTCCCTGGCCATCAGTGGGCTCCAGTCTGAGGATGAG |
| | | | GCTGATTATTACTGTGCAGCATGGGATGACAGCCTGAATGCTTTGGTATTCGGCGGAGGGACC |
| | | | AAGCTGACCGTCCTA |
| COV2- | 75 | heavy | CAGGTCACCTTGAGGGAGTCTGGTCCTGCGCTGGTGAAACCCACACAGACCCTCTCACTGACC |
| 2051 | | | TGCACCTTCTCTGGGTTCTCACTCGGCACTAGTGGAATGTGTGTGAGCTGGATCCGTCAGCCC |
| | | | CCAGGGAAGGCCCTGGAGTGGCTTGCACGCATTGATTGGGATGATGATAAATACTACAGCACA |
| | | | TCTCTGAAGACCAGGCTCACCATCTCCAAGGACACCTCCAAAAACCAGGTGGTCCTTACAATG |
| | | | ACCAACATGGACCCTGTGGACACAGCCACGTATTACTGTGCACGGGGGGTGGTTACTTATGAC |
| | | | TATTGGGGCCAGGGAACCCTGGTCACCGTCTCCTCA |
| | 76 | light | GACATCCAGATGACCCAGTCTCCATCCTCCCTGTCTGCATCTGTAGGAGACAGAGTGACCATC |
| | | | ACTTGCCGGGCAAGTCAGAGCATTGCCGGCTATTTAAACTGGTATCAGCAGAAACCAGGGAAA |
| | | | GCCCCTAAGCTCCTGATCTATGGTACAACCAGTTTGCAAAGTGGGGTCCCAGTAAGGTTCAGT |
| | | | GGCAGTGGATCTGGGACAGATTTCACTCTCACCATCAGCAGTCTGCAACCTGAAGATTTTGCA |
| | | | ACTTACTACTGTCAACAGAGTTACAGTACCCCTGGCACCTTCGGCCAAGGGACACGACTGGAG |
| | | | ATTAAA |
| COV2- | 77 | heavy | CAGGTCCAGCTGGTGCAATCAGGGGCTGAGGTGAAGAAGCCTGGGTCCTCGGTGAAGGTCTCC |
| 2054 | | | TGCAAGGCTTCTGGAGACACCTTCAGCAGCTATACTATCAACTGGGTGCGACAGGCCCCTGGA |
| | | | CAAGGGCTTGAGTGGATGGGAAGGATCATCCCTATCCTTGGTATACCAAACTACGCACAGAAA |
| | | | TTCCAGGGCAGAGTCACCATTACCGCGGACAAATCCACGAGCACAGCCTTCATGGAGCTGAGC |
| | | | AGCCTGAGATCTGAGGACACGGCCGTCTATTACTGTGCGAGAGGGAGGGGCTACAGTAACTAC |
| | | | GGGGCCTCCTACTACATGGACGTCTGGGGCAAAGGGACCACGGTCACCGTCTCCTCA |
| | 78 | light | GACATCCAGATGACCCAGTCTCCATCCTCCCTGTCTGCATCTGTAGGAGACAGAGTCACCATC |
| | | | ACTTGCCAGGCGAGTCAGGACATTAACCACTATTTAAATTGGTATCAGCAGAAACCAGGGAAA |
| | | | GCCCCTAAGCTCCTGATCTACGATGCATCCAATTTGGAAACCGGGGTCCCATCAAGGTTCAGT |
| | | | GGAAGTGGATCTGGGACAGATTTTACTTTCACCATCACCAGCCTGCAGCCTGAAGATGTTGCA |
| | | | ACATATTACTGTCAACAGTCTGATAATCTCCCCATGTACACTTTTGGCCAGGGGACCAAGCTG |
| | | | GAGATCAAA |
| COV2- | 79 | heavy | GAAGTGCAGCTGGTGGAGTCTGGGGGAGGCTTGGTACAGCCTGGCAGGTCCCTGAGACTCTCC |
| 2055 | | | TGTGCAGCCTCTGGATTCACCTTTGATGATTATGCCATGAACTGGGTCCGGCAACCTCCAGGG |
| | | | AAGGGCCTGGAGTGGGTCTCAGGTATTAGTTGGAATAGTGATAGCATAGGCTATGCGGACTCC |
| | | | GTGAAGGGCCGATTCACCATCTCCAGAGACAACGCCAAGAACTCCCTGTATCTGCAAATGAAC |
| | | | AGTCTGAGAGCTGAGGACACGGCCATGTATTACTGTGCAAAAGGAAGGGGTGCTGGTTATACT |
| | | | TCCTACATGGACGTCTGGGGCAAAGGGACCACGGTCACCGTCTCCTCA |
| | 80 | light | TCCTATGTGCTGACTCAGCCACCCTCGGTGTCAGTGGCCCCAGGAAAGACGGCCAGGATTACC |
| | | | TGTGAGGGAAACAACATTGGAAGTAAAAGTGTGCACTGGTACCAGCAGAAGCCAGGCCAGGCC |
| | | | CCTGTGCTGGTCGTCTATGATGATAGCGGCCGGCCCTCAGGGATCCCTGAACGATTCTCTGGT |
| | | | TCCAACTCTGGGAACACGGCCACCCTGACCATCAGCAGGGTCGAAGCCGGGGATGAGGCCGAC |
| | | | TATTTCTGTCAGGTGTGGGATAGTAGTAGTGATCATCATGTGGTTTTCGGCGGAGGGACCAAG |
| | | | CTGACCGTCCT |
| COV2- | 83 | heavy | CAGGTGCAGCTGGCGCAGTCTGGGGCTGAGGTGAAGAAGCCTGGGGCCTCAGTGAAGGTCTCC |
| 2064 | | | TGCAAGGCTGCTGGATACACCTTCACCAGTTATGATATCAACTGGGTGCGACAGGCCACTGGA |
| | | | CAAGGGCTTGAGTGGATGGGGTGGATGAACTCTAACAGTGGTAACGCAGGCTATGCACAGAAG |
| | | | TTCCAGGGCAGAGTCACTATGACCAGGGACACCTCCACAAGTACAGCCTACATGGAGTTGAGC |
| | | | AGCCTGACATCTGATGACACGGCCGTGTATTATTGTGCGAGAATGCGCACCGGCTGGCCCACA |
| | | | CATGGCCGCCCGGATGACTTCTGGGGGCGGGGAACCCTGGTCACCGTCTCCTCA |
| | 84 | light | CAGTCTGTGCTGACTCAGCCACCCTCAGCGTCTGGGACCCCCGGGCAGAGGGTCACCATCTCT |
| | | | TGTTCTGGAAGCAACTCCAACATCGGAAGTTATACTGTAAACTGGTACCAGCAGTTCCCAGGA |
| | | | ACGGCCCCCAAACTCCTCATTTATGATAATAATCAGCGGACCTCAGGGGTCCCTGACCGATTC |
| | | | TCTGGCTCCAAGTCTGGCACCTCAGCCTCCCTGGCCATCAGTGGGCTCCAGTCTGAGGATGAG |
| | | | GCTAATTATTACTGTTTAGTATGGGATGACAGCCTGAATGGCCTGGTATTCGGCGGAGGGACC |
| | | | AAGCTGACCGTCCTA |
| COV2- | 85 | heavy | GAAGTGCAGCTGGTGGAGTCTGGAGGAGGCTTGGTCCAGCCGGGGGGGTCCCTGAGACTCTCC |
| 2068 | | | TGTGCAGCCTCTGGGTTCACCGTCAGTAGCAACTACATGAGCTGGGTCCGCCAGGCTCCAGGG |
| | | | AAGGGGCTGGAGTGGGTCTCAGTTATTTATCCCGGTGGTAGCGCATTCTACGLAGACTCCGTG |
| | | | AAGGGCCGATTCACCATCTCCAGACACAATTCCAACAACACACTGTGTCTTCAAATGAACAGC |
| | | | CTGCGAACTGAGGACACGGCCGTGTATTATTGTGCGAGATCTTACGATATTTTGACTGGTTAT |
| | | | AGAGATGCTTTTGATATCTGGGGCCAAGGGACAATGGTCACCGTCTCTTC |
| | 86 | light | CAGTCTGTGCTGACGCAGCCGCCCTCAGTGTCTGGGGCCCCAGGGCAGAGGGTCACCATCTCC |
| | | | TGCACTGGGAGCAGCTCCAACATCGGGTCAGGTTCTGATGTACACTGGTACCAGCAGCTTCCA |
| | | | GGAACAGCCCCCAAACTCCTCATCTATGGCAACACCAATCGGCCCTCAGGGGTCCCTGACCGA |
| | | | TTCTCTGGCTCCAAGTCTGGCACCTCAGCCTCCCTGGCCATCACTGGGCTCCAGGCTGAGGAT |
| | | | GAGGCTGATTATTACTGCCAGTCCTATGACAGCAGGCTGAGTGGTTTTGTGGTATTCGGCGGA |
| | | | GGGACCAAGCTGACCGTCCTA |
| COV2- | 87 | heavy | GAGGTGCAGCTGGTGGAGTCTGGGGGAGGCTTGGTCCAGCCTGGGGGGTCCCTGAGACTCTCC |
| 2069 | | | TGTGCAGCCTCTGGACTCACCGTCAGTAGCAACTACATGAGTTGGGTCCGCCAGGCTCCAGGG |
| | | | AAGGGGCTGGAGTGCGTCTCAGTTATTTATGCCGGTGGTAATACATACTACGCAGACTCCGTG |
| | | | AAGGGCAGATTCACCATCTCCAGAGACAATTCCAAGAACACGCTGTATCTTCAAATGAACAGC |
| | | | CTGAGAGCCGAGGACACGGCTGTGTATTACTGTGCGAGAGGCGATGGTGGTTATTACTCACCC |
| | | | TTTGACTACTGGGGCCAGGGAACCCTGGTCACCGTCTCCTCA |
| | 88 | light | GACATCCAGATGACCCAGTCTCCATCCTCCCTGTCTGCATCTGTAGGAGACAGAGTCACCATC |
| | | | ACTTGCCGGGCAAGTCAGAGCATTAGCAGCTATTTAAATTGGTATCAGCAGAAACCAGGGAAA |
| | | | GCCCCTAAGGTCCTGATCTATGCTGCATCCACTATGCAAAGTGGGGTCCCATCAAGGTTCAGG |
| | | | GGCAGTGGCTCTGGGACAGATTTCACTCTCACCATCAGCAGTCTGCAACTTGAGGATTTTGCA |
| | | | ACTTACTACTGTCAACAGAGTTACAGTACCCCTCAGACGTTTGGCCAAGGGACCAAGGTGGAA |
| | | | ATCAAA |
| COV2- | 89 | heavy | CAGGTGCAGTTACAGCAGTGGGGCGCAGGGCTGTTGAAGCCTTCGGAGACCCTGTCCCTCACC |
| 2070 | | | TGCGCTGTCTCTGGTGGGTCCTTCAGTGCTTACTACTGGAGCTGGATCCGCCAGCCCCCAGGG |
| | | | AAGGGGCTGGAGTGGATTGGGGAAATCAATCATAGTGGAAGCACCAACTACAACCCGTCCCTC |
| | | | AGGAGTCGAGTCACCATATCAGTAGACACGTCCAAGAACCAATTCTCCCTGAAGCTGAGCTCT |
| | | | GTGACCGCCGCGGACACGGCTGTGTATTACTGTGCGAGAGTGGGTTATTCCCAAGGGTACTAC |
| | | | TACTACTACATGGACGTCTGGGGCAAAGGGACCACGGTCACCGTCTCCTCA |
| | 90 | light | GACATCCAGATGACCCAGTCTCCATCCTCCCTGTCTGCATCTGTAGGAGACAGAGTCACCATC |
| | | | ACTTGCCGGGCAAGTCAGAGCATTAGCAACTATTTAAATTGGTATCAGCAGAAACCGGGGAAA |
| | | | GCCCCTAAGCTCCTGATCTATGCTGCATCCAGTTTGCAAAGTGGGGTCCCCTCAAGGTTCAGT |
| | | | GGCAGTGGATCTGGGACAGATTTCTCTCTCACCATCAGCAGTCTGCAACCTGAAGATTTTGCA |
| | | | ACTTACTCCTGTCAACAGAGTTACACTACCCTCCTCACTTTCGGCGGAGGGACCAAGGTGGAG |
| | | | ATCAAA |
| COV2- | 93 | heavy | CAGGTCCAGCTGGTGCAGTCTGGGGCTGAGGTGAAGAAGCCTGGGTCCTCGGTGAAGGTCTCC |
| 2078 | | | TGCAAGGCTTCTGGAGGCACCTTCAGCAGCTATAGTATCACCTGGGTGCGACAGGCCCCTGGA |
| | | | CAAGGGCTTGAGTGGATGGGAAGGATCATCCCTGTCCTTGGTATAGCAAACTACGCACAGAAG |
| | | | TTCCAGGACAGAGTCACGATTACGGCGGACAAATCCACGAGCACAGCCTACATGGAGCTGAGC |
| | | | AGCCTGAGATCTGAGGACACGGCCGTCTATTACTGTGCGAGAGTGGGCGTGAGTGGTTTTAAA |
| | | | AGTGGCTCGAACTGGTACTTCGATCTCTGGGGCCGTGGCACCCTGGTCACTGTCTCCTCA |
| | 94 | light | CAGTCTGTGCTGACGCAGCCGCCCTCAGTGTCTGGGGCCCCAGGGCAGAGGGTCACCCTCTCC |
| | | | TGCACTGGGAGCAACTCCAACATCGGGGCAGGTTATGATGTACACTGGTACCAGCAGCTTCCA |
| | | | GGAACAGCCCCCAAACTCCTCATCTATGGTAATAGCAATCGGCCCTCCGGGGTCCCTGACCGA |
| | | | TTCTCTGGCTCCAAGTCTGGCACCTCAGCCTCCCTGGCCATCACTGGGCTCCAGGCTGAGGAT |
| | | | GAGGCTGATTATTACTGCCAGTCCTATGACAGCAGCCTGAGTGATTCGGTGTTCGGCGGAGGG |
| | | | ACCAAGGTGACCGTCCTA |
| COV2- | 97 | heavy | CAGGTGCAGCTGGTGCAGTCTGGGGCTGAGGTGAAGAAGCCTGGGGCCTCAGTGAAGGTTTCC |
| 2081 | | | TGCAAGGCATCTGGATACACCTTCACCAGCTACTATATGCACTGGGTGCGACAGGCCCCTGGA |
| | | | CAAGGGCTTGAGTGGATGGGAATAATCAACCCTGGTGGTGGTAGTACAACCTACGCACAGAAG |
| | | | TTCCAGGGCAGAGTCACCATGACCAGTGACACGTCCACGAGCACAGTCTACATGGAGCTGAGC |
| | | | AGCCTGAGATCTGAGGACACGGCCATGTATTACTGTGCGAGAGGGGCAATTCCCCCAAATAGC |
| | | | AGAGCCGAAATTGACTACTGGGGCCAGGGAACCCTGGTCACCGTCTCCTCA |
| | 98 | light | GAAATAGTGATGACGCAGTGTCCAGCCACCCTGTCTGTGTCTCCAGGGGAAAGAGTCACCCTC |
| | | | TCCTGCAGGGCCAGTCAGAGTGTTAGCAGCAACTTAGCCTGGTGCCAGCAGAAACCTGGCCAG |
| | | | GCTCCCAGGCTCCTCATCTATGGTGCATCCACCAGGGCCACTGGTATCCCAGCCAGGTTCAGT |
| | | | GGCAGTGGGTCTGGGACAGAGTTCACTCTCACCATCAGCAGCCTGCAGTCTGAAGATTTTGCA |
| | | | GTTTATTACTGTCAGCAATATTATAACTGGCCGCLCACTTTCGGCGGAGGGACCAAGGTGGAG |
| | | | ATCAAA |
| COV2- | 99 | heavy | GAGGTGCAGCTGGTGGAGTCTGGGGGAGGTGTGGTACGGCCTGGGGGGTCCCTGAGACTCTCC |
| 2082 | | | TGTGCAGCCTCTGGATTCATCTTTGATGATTATGACATGACCTGGGTCCGCCAAGCTCCAGGG |
| | | | AAGGGGCTGGAGTGGGTCTCTGGTATTAGTTGGAATGGTGGTAACACAGGTTATGCAGACTCT |
| | | | GTGAAGGGCCGATTCACCATCTCCAGAGACAACGCCAAGAACTCCCTGTATCTGCAAATGAAC |
| | | | AGTCTGAGAGCCGAGGACACGGCCTTGTATCACTGTGCAGTGATTATGTCTCCAATCCCCCGT |
| | | | TATAGTGGCTACGATTGGGCGGGTGGTGCTTTTGATATCTGGGGCCAAGGGACAATGGTCACC |
| | | | GTCTCTTCA |
| | 100 | light | TCTTCTGAGCTGACTCAGGACCCTGCTGTGTCTGTGGCCTTGGGACAGACAGTCAGGATCACA |
| | | | TGCCAAGGAGACAGCCTCAGAAGCTATTATGCAAGCTGGTACCAGCAGAAGCCAGGACAGGTC |
| | | | CCTATACTTGTCATCTATGATAAAAACAACCGGCCCTCAGGGATCCCAGACCGATTCTCTGGC |
| | | | TCCAGCTCAGGAAACACAGCTTCCTTGACCATCACTGGGGCTCAGGCGGAAGATGAGGCTGAC |
| | | | TATTACTGTAACTCCCGGGACAGCAGTGGTAACGCCGTGGTGTTCGGCGGAGGGACCAAGCTG |
| | | | ACCGTCCTA |
| COV2- | 101 | heavy | CAGGTGCAGCTGGTGGAGTCTGGGGGAGGCGTGGTCCAGCCTGGGAGGTCCCTGAGACTCTCC |
| 2083 | | | TGTGCAGCCTCTGGATTCACCTTCAGTAACTATGGCATGCACTGGGTCCGCCAGGCTCCAGGC |
| | | | AAGGGGCTGGAGTGGGTGGCAGTTATGTCATATGATGGAAGTAATAAATACTATGCAGACTCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTATATTACTGTGCGAAAAATTTAGGACCCTATTGTAGT |
| | | | GGTGGTACCTGCTATTCCTTAGTTGGTGACTACTGGGGCCAGGGAACCCTGGTCACCGTCTCC |
| | | | TCA |
| | 102 | light | GACATCCAGATGACCCAGTCTCCATCCTCCCTGTCTGCATCTGTAGGAGACAGAGTCACCATC |
| | | | ACTTGCCAGGCGAGTCAGGACATTAGCAACTATTTAAATTGGTATCAGCAGAAACCAGGGAAA |
| | | | GCCCCTAAGCTCCTGATCTACGATGCATCCAATTTGGAAACAGGGGTCCATCAAGGTTTCAGT |
| | | | GGAAGTGGATCTGGGACAGATTTTACTTTCACCATCAGCAGCCTGCAGCCTGAAGATATTGCA |
| | | | ACATATTACTGTCAACAGTATGCTAATCTCCCATTCACTTTCGGCCCTGGGACCAAAGTGGAT |
| | | | ATCAAA |
| COV2- | 107 | heavy | GAAGTGCAACTGGTGGAGTCTGGGGGAGGCTTGGTACAGCCTGGCAGGTCCCTGAGACTCTCC |
| 2097 | | | TGTGCAGCCTCTGGATTCACCTTTGATGATTATGCCATGCATTGGGTCCGGCAAGCTCCAGGG |
| | | | AAGGGCCTAGAGTGGGTCTCAGGTATCAGTTGGAACAGTGGTACCATACGCTATGCGGACTCT |
| | | | GTGAAGGGCCGATTCATCATCTCCAGAGACAACGCCAAGAACTCCTTGTATCTGCAAATGAAC |
| | | | AGTCTGAGACCTGAGGACACGGCCTTGTATTACTGTGCAAAAGATATAATACGTCAGGGCGAA |
| | | | GACGGTATGGACGTCTGGGGCCAAGGGACCACGGTCACCGTCTCCTCA |
| | 108 | light | GACATCCAGATGACCCAGTCTCCATCCTCCCTGTCTGCATCTGTAGGAGACAGAGTCACCATC |
| | | | ACTTGCCGGGCAAGTCAGAACATTGCCAGCTATTTGAATTGGTATCAGCAGAAACCAGGGAAA |
| | | | GCCCCTAAGCTCCTGATCTATGCTGCATCCAGTTTGCAAAGTGGGGTCCCATCAAGGTTCAGT |
| | | | GGCAGTGGATCGGGGACAGATTTCACTCTCACCATCAGCAGTCTGCAACCTGAAGAGTTTGCA |
| | | | ACTTACTACTGTCAACAGAGTTACAGTACCCCGTGGACGTTCGGCCAAGGGACCAAGGTGGAA |
| | | | ATCAAA |
| COV2- | 109 | heavy | GAGGTGCAACTGTTGGAGTCTGGGGGAGGCTTGATACAGCCTGGGGGGTCCCTGAGACTCTCC |
| 2098 | | | TGTGCAGCCTCTGGATTCACCTTTAGCAACTATGCCATGTCCTGGGTCCGCCAGGCCCCAGGG |
| | | | AAGGGGCTGGAGTGGGTCTCAGGTATTATTAGTACCAGTGGTGGTGCCACATACAACGCAGAC |
| | | | TCCGTGAGGGGCCGGTTCACCACCTCCAGGGACAATTCCAAGAACATACTGTATCTGCAAATG |
| | | | AACAGCCTCAGAGGCGAGGACACGGCCGTTTATTACTGTGTGAAAGGTCTCTTTGACTGGTTC |
| | | | CCGCTCTGGGGCCAAGGGACAATGGTCACCGTCTCTTCA |
| | 110 | light | GACATAGTGATGACGCAGTCTCCAGCCACCCTGTCTGTGTCTCCAGGGGAAAGAGCCATCCTC |
| | | | TCCTGCAGGGCCAGTCAGAGTGTTAGAAGCAATTTAGCCTGGTACCAGCAGAAACCTGGCCAG |
| | | | GCTCCCAGGCTCCTCATCTCTGGTGCATCCACCAGGGCCACTGCTATCCCAGCCAGGTTCAGT |
| | | | GGCAGTGGGTCTGGGACAGAGTTCACTCTCACCATCACCAGCCTGCAGTCTGAAGATTGTGCA |
| | | | GTTTATTACTGTCACCAGTATAATAACTGGCCTCAGACGTTCGGCCAAGGGACCAAGGTGGAA |
| | | | ATCAAA |
| COV2- | 111 | heavy | GAGGTGCAGCTGGTGGAGTCTGGGGGAGGCTTGGTCCAGCCTGGGGGTCCCTGAGACTCTCC |
| 2103 | | | TGTGCAGCCTCTGGATTCACCTTTAGTAGGCATTGGATGACCTGGGTCCGCCAGGCTCCAGGG |
| | | | AAGGGGCTGGAGTGGGTGGCCAACATAAAGCAAGATGGAAGTGAGAAATACTATGTGGACTCT |
| | | | GTGAAGGGCCGACTCACCATCTCCAGAGACAACGCCAAGAACTCACTGTATCTGCAAATGAAC |
| | | | AGCCTGAGAGCCGAGGACACGGCTGTGTATTACTGTGCGAGACTGGGGTTCTATTACGGTGGA |
| | | | GCCGACTACTGGGGCCAGGGAACCCTGGTCACCGTCTCCTCA |
| | 112 | light | AATTTTATGCTGACTCAGCCCCACTCTGTGTCGGAGTCTCCGGGGAAGACGGTAACCATCTCC |
| | | | TGCACCGGCAGCAGTGGCAGCATTGCCAGCAACTATGTGCAGTGGTACCAGCAGCGCCCGGGC |
| | | | AGTGCCCCCACCACTGTGATCTCTGAGGATAACCAAAGACCCTCTGGGGTCCCTGATCGGTTC |
| | | | TCTGGCTCCATCGACAGCTCCTCCAACTCTGCCTCCCTCACCATCTCTGGACTGAAGACTGAG |
| | | | GACGAGGCTGACTACTACTGTCAGTCTTATGATGGCATCAATCGGGCATGGGTGTTCGGCGGA |
| | | | GGGACCAAGCTGACCGTCCTA |
| COV2- | 113 | heavy | AACGTGCAATTAGTGGAGTCTGGGGGAGGCTTGGTTCAGCCTGGCGGGTCCCTGAGACTCTCC |
| 2108 | | | TGTGCAGCCTCTGGATTCACCTTTCATCATTATGCCATGCACTGGGTCCGACAAGCTCCAGGG |
| | | | AAGGGCCTGGAGTGGGTCTCAGGTATTAGTGGGAGTAGTGATTACAGAGCCTATGCGGACTCT |
| | | | CTGAAGGGCCGATTCACCATCTCCAGAGACTACGCCAAGAACTCCCTGTGGCTGCAAATGAAC |
| | | | AGTCTGACATCTGAGGACACGGCCTTCTATTACTGTGCAAAGGGCGTTGACTATGGCGGCAAA |
| | | | CTTGCCTACTTTGACTCCTGGGGCCAGGGAACCCTGGTCACCGTCTCCTCA |
| | 114 | light | GATATTGTGATGACTCAGTCTCCACTCTCCCTGCCCGTCACCCCTGGAGAGCCGGCCTCCATC |
| | | | TCCTGCAGGTCTAGTCAGAGCCTCCTGCATAGTCTTGGATACAACTCTTTGAGTTGGTACCTG |
| | | | CAGAAGCCAGGGCAGTCTCCACACCTCCTGATCTATTTGGGCTCTAATCGGGCCTCCGGGGTC |
| | | | CCTGACAGGTTCAGTGGCAGTGGATCAGCCACAGACTTTACACTGAAAATCAGCAGATTGGAG |
| | | | GCTGAGGATGTTGGCGTTTATTACTGCATGCAAGCTCTACAAACTCCCCTCACCTTCGGCCAA |
| | | | GGGACACGACTGGAGATTAAA |
| COV2- | 115 | heavy | CAGGTGCAGCTGGTGGAGTCTGGGGGAGGCGTGGTCCAGCCTGGGAGGTCCCTGAGACTCTCC |
| 2110 | | | TGTGCAGCCTCTGGATTCAGCTTCAGTAGCTATGTTATGAACTGGGTCCGCCAGGCTCCAGGC |
| | | | AAGGGGCTGGAGTGGGTGGCAGTTATATCATATGATGGAAGCAGTAAATACTACGCAGACTCC |
| | | | GTGAAGGGCCGATTCACCATCTCCAGAGACAATTCCAAGAATACACTGTATCTGCAAATGAAC |
| | | | AGCCTGAGAGCTGAGGACACGGCTGTGTATTACTGTGCGAGAGACATTGATAGTGGCTACGAT |
| | | | CCTACCCCCGTTTTTGACTACTGGGGCCAGGGAACCCTGGTCACCGTCTCCTCA |
| | 116 | light | GACATCCAGATGACCCACTCTCCATCCTCCCTGTCTGCATGTGTAGGAGACAGAGTCACCATC |
| | | | ACTTGCCGGGCAAGTCAGAGCATTAGCAGCTATTTAAATTGGTATCAGCAGAAACCAGGGAAA |
| | | | GGCCCTAAGCTCCTGATCTATGCTGCATCCAGTTTGCAAAGTGGGGTCCCATCAAGGTTCAGT |
| | | | GGCAGTGGATCTGGGACAGACTTCACTCTCACCATCAGCAGTCTGCAACCTGAAGATTTTGCA |
| | | | ACTTACTACTGTCAACAGAGTTACAGTTCCCTTTCGATCACCTTCGGCCAAGGGACACGACTG |
| | | | GAGATTAAA |
| COV2- | 117 | heavy | GAGGTGCAGCTGGTGGAGTCTGGGGGAGGCTTGGTACAGCCTGGGGGGTCCCTGAGACTCTCC |
| 2111 | | | TGTGCAGCCTCTGGATTCACCTTCAGTAGCTACGACCTGCACTGGGTCCGCCAAGGTACAGGA |
| | | | AAACGTCTGGAGTGGGTCTCAGCTATTGGTACTGCTGGTGACACATACTATCTAGGCTCCGTG |
| | | | AAGGGCCGATTCACCATCTCCAGAGAAAATGCCAAGAACTCCTTGTATCTTCAAATGAACAGC |
| | | | CTGAGAGCCGGGGACACGGCTGTGTATTACTGTGCAAGAGTCCTCTATGATAGTAGTGGTTTT |
| | | | TACAACTGGTTCGACCCCTGGGGCCAGGGAACCCTGGTCACCGTCTCCTCA |
| | 118 | light | GACATCCAGATGACCCAGTCTCCATCCTCCCTGTCTGCATCTGTTGGAGACAGAGTCACCATC |
| | | | ACTTGCCGGGCAAGTCAGAGCATTAGCAGCTATTTAAATTGGTATCAGCAGAAACCAGGGAAA |
| | | | GCCCCTAAGCTCCTGATCTATGCTGCATCCAGTTTGGAAAGTGGGGTCCCATCAAGGTTCAGT |
| | | | GGCAGTGGATCTGGGACAGATTTCACTCTCACCATCAGCAGTCTGCAACCTGAAGATTTTGCA |
| | | | ACTTACTACTGTCAACAGAGTTACGAAATCCCTCCGTGGACGTTCGGCCAAGGGACCAAGGTG |
| | | | GAAATCAAA |
| COV2- | 119 | heavy | GAGGTGCAGCTGGTGGAGTCTGGAGGAGGCTTGATCCAGCCTGGGGGGTCCCTGAGACTCTCC |
| 2113 | | | TGTGCAGCCTCTGAGGTCACCGTCAGTAGCAACTACATGAGCTGGGTCCGCCAGGCTCCAGGG |
| | | | AAGGGGCTGGAGTGGGTCTCACTTATTTATAGCGGTGGTACTACATACTACGCAGACTCCGTG |
| | | | AAGGGCCGATTCACCATCTCCAGAGACAATTCCAAGAACACCCTGTATCTTCAAATGAACAGC |
| | | | CTGAGAGCCGAGGACACGGCCGTGTATTACTGTGCGAGAGACTTTCTACGGTGGCACGACCTC |
| | | | TGGGCCAGGGAACCCTGGCACCGTCTCCTCA |
| | 120 | light | GACATCCAGATGACCCAGTCTCCATCCTCCCTGTCTGCATCTGTAGGAGACAGAGTCACCATC |
| | | | ACTTGCCAGGCGAGTCAGGACATTAACAACTATTTAAATTGGTATCAGCAGAAACCAGGGAAA |
| | | | GCCCCTAAGCTCCTGATCTACGATGCATCCAATTTGGAAACAGGGGTCCCATTAAGGTTCAGT |
| | | | GGAAGTGGATCTGGGACAGATTTTATTTTCACCATCAGCAGCCTGCAGCCTGAAGATATTGCA |
| | | | ACATATTACTGTCAACAGTATGATAATCTCCCTCCAGTTTTCGGCGGAGGGACCAAGGTGGAG |
| | | | ATCAAA |
| COV2- | 121 | heavy | CAGGTCCAGCTGGTGCAATCAGGGGCTGAGGTGAAGAAGCCTGGGTCCTCGGTGAAGGTCTCC |
| 2114 | | | TGCAAGGCTTCTGGAGACACCTTCAGCAGCTATACTATCAACTGGGTGCGACAGGCCCCTGGA |
| | | | CAAGGGCTTGAGTGGATGGGAAGGATCATCCCTATCCTTGGTATACCAAACTACGCACAGAAA |
| | | | TTCCAGGGCAGAGTCACCATTACCGCGGACAAATCCACGAGCACAGCCTTCATGGAGCTGAGC |
| | | | AGCCTGAGATCTGAGGACACGGCCGTCTATTACTGTGCGAGAGGGAGGGGCTACAGTAACTAC |
| | | | GGGGCCTCCTACTACATGGACGTCTGGGGCAAAGGGACCACGGTCACCGTCTCCTCA |
| | 122 | light | CAGTCTGTGCTGACGCAGCCGCCGTCAGTGTCTGGGGGCCCAGGGCAGAGGGTCACCATCTCC |
| | | | TGCACTGGGAGCAGCTCCAACATCGGGGCAGGTTATGATGTACACTGGTACCAGCAGCTTCCA |
| | | | GAAACAGCCCCCAAACTCCTCATCTATGCTAACAGCAATCGGCCCTCAGGGGTCCCTGACCGA |
| | | | TTCTCTGGCTCCAAGTCTGGCACCTCAGCCTCCCTGGCCATCACTGGGCTCCAGGCTGAGGAT |
| | | | GAGGCTGATTATTACTGCCAGTCCTATGACAGCAGCCTGAGTGGTTCGGTATTCGGCGGAGGG |
| | | | ACCAAGCTGACCGTCCTA |
| COV2- | 123 | heavy | CAGCTGCAGCTACAGGAGTCGGGCCCAGGACTGGTGAAGCCTTCGGAGACCCTATCCCTCACC |
| 2128 | | | TGCACTGTCTCTGGTGGCTCCATCAGCAGTAGTAGTTACTACTGGGGCTGGATCCGCCAGCCC |
| | | | CCAGGGAACGGGCTGGAGTGGATTGGGAGTATCTATTATAGTGGGAGCACCTACTACAACCCG |
| | | | TCCCTCAAGGGTCGAGTCTCCATATCCGTAGACACGTCCAAGAACCAGTTCTCCCTGAAGCTG |
| | | | AGCTCTGTGACCGCCGCAGACACGGCTGTCTATTACTGTGCGAGAATCTTAGTAATTTTTACT |
| | | | TTAAACTGGTTCGACCCCTGGGGCCAGGGAACCCTGGTCACCGTCTCCTCA |
| | 124 | light | AATTTTATGGTGACTCAGCCCCAGTCTGTGTCGGAGTGTCCGGGGAAGACGGTAACCATCTCC |
| | | | TGCACCGGCAGCAGTGGCAGCATTGCGAGCAACTATGTGCAGTGGTAGCAGCAGGGCCCGGGC |
| | | | AGTGCCCCCACCACTGTGATCTATGAGGATAACCAAAGACCCTCTGGGGTCCCTGATCGGTTC |
| | | | TCTGGCTCCATCGACAGCTCCTCCAACTCTGCCTCCCTCACCATGTCTGGAGTGAAGACTGAG |
| | | | GACGAGGCTGACTACTACTGTCAGTCTTATGATAGCGGCAATCCCATATTCGGCGGAGGGACC |
| | | | AAGCTGACCGTCCTA |
| COV2- | 127 | heavy | GAGGTGCAGCTGGTGGAGTCTGGAGGAGGCTTGATCCAGCCTGGGGGGTCCCTGAGACTCTCC |
| 2132 | | | TGTGCAGCCTCTGAGGTCACCGTCAGTAGCAACTACATGAGCTGGGTCCGCCAGGCTCCAGGG |
| | | | AAGGGGCTGGAGTGGGTCTCACTTATTTATAGCGGTGGTACTACATACTACGCAGACTCCGTG |
| | | | AAGGGCCGATTCACCATCTCCAGAGACAATTCCAAGAACACCCTGTATCTTCAAATGAACAGC |
| | | | CTGAGAGCCGAGGACACGGCCGTGTATTACTGTGCGAGAGACTTTCTACGGTGGCACGACCTC |
| | | | TGGGGCCAGGGAACCCTGGTCACCGTCTCCTCA |
| | 128 | light | GACATCCAGATGACCCAGTCTCCATCCTCCCTGTCTGCATCTGTAGGAGACAGAGTCACCATC |
| | | | ACTTGCCAGGCGAGTCAGGACATTAACAACTATTTAAATTGGTATCAGCAGAAACCAGGGAAA |
| | | | GCCCCTAAGCTCCTGATCTACGATGCATCCAATTTGGAAACAGGGGTCCCATTAAGGTTCAGT |
| | | | GGAAGTGGATCTGGGACAGATTTTACTTTCACCATCAGCAGCCTGCAGCCTGAAGATATTGCA |
| | | | ACATATTACTGTCAACAGTATGATAATCTCCCTCCAGTTTTCGGCGGAGGGACCAAGGTGGAG |
| | | | ATCAAA |
| COV2- | 129 | heavy | CAGGTGCAGCTGCAGGAGTCGGGCCCAGGACTGGTGAAGCCTTCGGAGACCCTGTCCCTCACC |
| 2137 | | | TGCACTGTCTCTGGTGGCTCCGTCAGCAGTGGTAGTTACTACTGGAGCTGGATCCGGCAGCCC |
| | | | CCAGGGAAGGGACTGGAGTGCATTGGGTATATCTATTACAGTGGGAGCTCCAACTACAACCCC |
| | | | TCCCTCAAGAGTCGAGTCACCATATCAGTAGACACGTCCAAGAACCAGTTCTCCCTGAAGATG |
| | | | AGCTCTGTGACCGCTGCGGACACGGCCGTATATTACTGTGCGGGGAGCCCTGTCCCTCCCACG |
| | | | ATTGTGGGAGCTTCGTACTGGGGCCAGGGAACCCTGGTCACCGTCTCCTCA |
| | 130 | light | AATTTTATGCTGACTCAGCCCCACTCTGTGTCGGAGTCTCCGGGGAAGACGGTAACCTTCTCC |
| | | | TGCACCGGCAGCAGTGGCAGCATTGCCAGCAACTATGTGCAGTGGTACCAGCAGCGCCCGGGC |
| | | | AGTGCCCCCACCACTGTGATCTATGAGGATAACCAAAGACCCTCTGGGGTCCCTGATCGATTC |
| | | | TCTGGCTCCATCGACAGCTCCTCCAACTCTGCCTCCCTCACCATCTCTGGACTGAAGACTGAG |
| | | | GACGAGGCTGACTACTACTGTCAGTCTTATGATGGCATCAATCGGTGGCTGGTATTCGGCGGA |
| | | | GGGACCAAGCTGACCGTCCTA |
| COV2- | 131 | heavy | GAGGTGCAGCTGGTGCAGTCTGGAGCAGAGGTGAAAAAGCCCGGGGAGTCTCTGAAGATCTCC |
| 2142 | | | TGTAAGGGTTCTGGATACAGCTTTACCAGCTACTGGATCGACTGGGTGCGCCAGATGCCCGGG |
| | | | AAAGGCCTGGAGTGGATGGGGATCATTTATCCTGGTGACTCTGATACCAGATATAGCCCGTCC |
| | | | TTCCAAGGCCAGGTCACCATCTCAGCCGACAAGTCCACCAGCACCGCCTACCTGCAGTGGAGC |
| | | | AGCTTGAAGGCCTCGGACACCGCCATGTATTACTGTGCGAGACGTGGAGAAGCAGCTGGTATT |
| | | | TGGTACTTCGATCTCTGGGGCCGTGGCACCCTGGTCACTGTCTCCTCA |
| | 132 | light | CAGCCTGTGCTGACTCAGCCACCTTCTGCATCAGCCTCCCTGGGAGCCTCGGTCACACTCACC |
| | | | TGCACCCTGAGCAGCGGCTACAGTAATTATAAAGTGGACTGGTACCAGCAGAGACCAGGGAAG |
| | | | GGCCCCCGGTTTGTGATGCGAGTGGGCACTGGTGGGATTGTGGGATCCAAGGGGGATGGCATC |
| | | | CCTGATCGCTTCTCAGTCTTGGGCTCAGGCCTGAATCGGTATCTGACAATCAAGAACATCCAA |
| | | | GAAGAGGATGAGAGTGACTACCACTGTGGGGCAGACCATGGCAGTGGGAGCAACTTCGAGTAT |
| | | | GTGGTATTCGGCGGAGGGACCAAGCTGACCGTCCTA |
| COV2- | 133 | heavy | GAGGTGCAGCTGGTGGAGTCTGGGGGAGGCTTGGTCCAGCCTGGGGGGTCCCTGAGACTCTCC |
| 2143 | | | TGTGCAGCCTCTGGATTCACCGTCAGTAGCAACTACATGAGCTGGGTCCGCCAGGCTCCAGGG |
| | | | AAGGGGCTGGAGTGGGTCTCAGTTATTTATAGCGCTGGTAGCACATACTACGCAGACTCCGTG |
| | | | AAGGGCAGATTCAGCATCTCCAGAGACAAGTCCAAGAACACGCTGTATCTTCAAATGAACAGC |
| | | | CTGAGAGCCGAGGACACGGCTGTATATTACTGTGCGAAAGAAGGTGGATCGGGGAGCCTCCGC |
| | | | TACTACTACTACGGTATGGACGTCTGGGGCCAAGGGACCACGGTCACCGTCTCCTCA |
| | 134 | light | CAGTCTGTGGTGACTCAGCCACCCTCAGCGTCTGGGACCCCCGGGCAGAGGGTCACCATCTCT |
| | | | TGTTCTGGAAGCAGCTCCAACATCGGATATAATATTGTAAACTGGTACCAGCAGCTCCCAGGA |
| | | | ACGGCCCCCAAACTCCTCATCTATAGTAATAATCAGAGGCCCTCAGGGGTCCCTGACCGATTC |
| | | | TCTGGCTCCAAGTCTGGCACCTCAGCCTCCCTGTCCATCAGTGGGCTCCAGTCTGAGGATGAG |
| | | | GCTGATTATTACTGTGCAGCATGGGATGACAGCCTGAATGGTTATGTCTTCGGAACTGGGACC |
| | | | AAGGTCACCGTCCTA |
| COV2- | 135 | heavy | CAGGTGCAGCTGGTGGAGTCTGGGGGAGGCGTGGTCCAGCCTGGGAGGTCCCTGAGACTCTCC |
| 2145 | | | TGTGCAGCCTCTGGATTCACCTTCAGTACGTATGCTATGCACTGGGTCCGCCAGGCTCCAGGC |
| | | | AAGGGGCTGGAGTGGGTGGCAGTTATATCATATGATGGAAGCAATAAATACTACGCAGACTCC |
| | | | GTGAAGGGCCGATTCACCATCTCCAGAGACAATTCCAAGAACACACTATATCTGCAAATGATC |
| | | | GuCCTGAGAGCTGAGGACACGGCTGTGTATTACLGTGCGAGAGATTGGGCACCTACGTACTAC |
| | | | GATATGCCGAGTGCTTTTGATATCTGGGGCCAAGGGACAATGGTCACCGTCTCTTCA |
| | 136 | light | TCCTATGTGCTGACTCAGCCACCCTCGGTGTCAGTGGCCCCAGGAAAGACGGCCAGGATTACC |
| | | | TGTGGGGGAAACAACATTGGAAATAAAGGTGTGCATTGGTACCAGCAGAAGCCAGGCCAGGCC |
| | | | CCTGTGCTGGTCGTCGATGATGATAGCGACCGGCCCTCAGGGATCCCTGAGCGATTCTCTGGC |
| | | | TCCAACTCTGGGAACACGGCCACCCTGATCATCAGCAGTGTCGAAGTCGGGGATGAGGCCGAC |
| | | | TTTTACTGTCAGGTGTGGGATAGTAGTAGTGATCATCCGGGGGTGTTCGGCGGAGGGACCAAG |
| | | | CTGACCGTCCTA |
| COV2- | 137 | heavy | GAGGTGCAGCTGGTGGAGTCTGGGGGAGGCTTGGTACAGCCTGGAGGGTCCCTGAGACTCTCC |
| 2146 | | | TGTGAAGCCTCTGGATTCACCTTCAGTAGTTCTGAAATCAACTGGGTCCGCCAGGCTCCAGGG |
| | | | AAGGGGCTGGAGTGGGTTTCACACATTAGTAGTAGTGGTAGTATCATATACTACGCAGACTCT |
| | | | GTGAAGGGCCGATTCACCATCTCCAGAGACAACGCCAAGAACTCACTGTATCTGCAAATGAAC |
| | | | AGCCTGAGAGCCGAGGACACGGCTGTTTATTACTGTGCGAGGAGATCTTATAGAAGCAGCTGG |
| | | | TACTACTACTACGGTATGGACGTCTGGGGCCAAGGGACCACGGTCACCGTCTCCTCA |
| | 138 | light | GACATCCAGTTGACCCAGTCTCCATCTTTCCTGTCTGCTTCTGTAGGAGACAGAGTCACCATC |
| | | | ACTTGCCGGGCCAGTCAGGGCATTAGCAGTTATTTAGCCTGGTATCAGCAAAAACCAGGGAAA |
| | | | GCCCCTAAGCTCCTGATCTATGCTGCATCCACTTTGCAAAGTGGGGTCCCATCAAGGTTCAGC |
| | | | GGCAGTGGATCTGGGACAGAATTCACTCTCACAATCAGCAGCCTGCAGCCTGAAGATTTTGCA |
| | | | ACTTATTACTGTCAACAGCTTAATAGTTACCCCGTGACGTTCGGCCAAGGGACCAAGGTGGAA |
| | | | ATCAAA |
| COV2- | 139 | heavy | CAGGTGCAGCTGGCGGAGTCTGGGGGAGGCGTGGTCCAGCCTGGGAGGTCCCTGAGACTCTCC |
| 2147 | | | TGTGCAGCCTCTGGATTCACCTTCAGTAGCTATGCTATGCACTGGGTCCGCCAGGCTCCAGGC |
| | | | AAGGGGCTGGAATGGGTGGCAGTTATATCATATGATGGAAGCAATAAATACTACGCAGACTCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTGTATTACTGrGCGAGAAGCACGAGTGGGAGCTACTAC |
| | | | TACGGTATGGACGTCTGGGGCCAAGGGACCACGGTCACCGTCTCCTCA |
| | 140 | light | CAGTCTGCCCTGACTCAGCCTGCCTCCGTGTCTGGGTCTCCTGGACAGTCGATCACCATCTCC |
| | | | TGCACTGGAACCAGCAGTGACGTTGGTGATTATAACTATGTCTCCTGGTACCAACAACACCCA |
| | | | GGCAAAGCCCCCAAACTCATGATTTATGATGTCAGTAATCGGCCCTCAGGGGTTTCTAATCGC |
| | | | TTCTCTGGCTCCAAGTCTGGCAACACGGCCTCCCTGACCATCTCTGGGCTCCAGGCTGAGGAC |
| | | | GAGGCTGAATATTACTGCAGCTCATATACAAGCAGCAGCACTCTACTTTATGTCTTCGGAACT |
| | | | GGGACCAAGGTCACCGTCCTA |
| COV2- | 141 | heavy | CAGGTGCAGCTGGTGCAGTCTGGGGCTGAGGTGAGGAAGCCTGGGTCCTCGGTGAAGGTCTCC |
| 2151 | | | TGCAAGGCTTCTGGAGGCACCTTCAGCAGCTATGCTATCAGCTGGGTGCGGCAGGCCCCTGGA |
| | | | CAAGGGCTTGAGTGGATGGGAGGGATCATCCCTGTCTTTGGTACAGCAAACTACGCACAGAAG |
| | | | TTCCAGGGCCGAGTCACGATTACCGCGGACAAATCCACGAGCACAGCCTTCATGGAGCTGAAC |
| | | | AGCCTGAGATCTGAGGACACGGCCGTCTATTACTGTGCGAGAATTGGGAGCTACCCTGAATAC |
| | | | TTCCAGCACTGGGGCCAGGGCACCCTGGTCACCGTCTCCTCA |
| | 142 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTTCTTAGCCTGGTACCAACAGAAACCTGGCCAG |
| | | | GCTCCCAGGCTCCTCATCTATGATGCATCCAACAGGGCCACTGGCATCCCAGCCAGGTTCAGT |
| | | | GGCAGTGGGTCTGGGACAGACTTCACTCTCACCATCAGCAGCCTAGAGCCTGAAGATTTTGCA |
| | | | GTTTATTACTGTCACTACCGTAGCAACTGGCCTCCGGTTCTCACTTTCGGCGGAGGGACCAAG |
| | | | GTGGAGATC |
| COV2- | 143 | heavy | CAGGTGCAGCTGGTGCAGTCTGGGGCTGAGGTGAAGAAGCCTGGGTCCTCGGTGAAGGTCTCC |
| 2153 | | | TGCAAGGCTTCTGGAGGCACCTTCAGCAGCTATGCTATCATCTGGGTGCGACAGGCCCCTGGA |
| | | | CAAGGGCTTGAGTGGATGGGAGGGATCATCCCTATCTTTGGTACAACAAACTACGCACAGAAG |
| | | | TTCCAGGGCAGAGTCACGATTACCGCGGACGAATCCACGAGCACAGCCTACGTGGAACTGAGC |
| | | | AGCCTGAGATCTGAGGACACGGCCGTGTATTATTGTGCGAGAATAGGCCATTTTGATAGTAGT |
| | | | GGTTATTACTTAGACTACTGGGGCCAGGGAACCCTGGTCACCGTCTCCTCA |
| | 144 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTTCTTAGCCTGGTACCAACAGAAACCTGGCCAG |
| | | | GCTCCCAGGCTCCTCATCTATGATGCATCCAACAGGCCCACTGGCATCCCAGCCAGGTTCACT |
| | | | GGCAGTGGGTCTGGGACAGACTTCACTCTCACCATCAGCAGCCTAGAGCCTGAAGATTTTGCA |
| | | | GTTTATTACTGTCAGCACCGTACCAACTGGCCTCCCTTATTCACTTTCGGCCCTGGGACCAAA |
| | | | GTGGATATCAAA |
| COV2- | 145 | heavy | CAGGTGCAGCTGGTGGAGTCTGGGGGAGGCGTGGTCCAGCCTGGGAGGTCCCTGAGACTCTCC |
| 2155 | | | TGTGCAGCCTCTGGATTCACCTTCAGTAGTTATGCTCTGTTCTGGGTCCGCCAGGCTCCAGGC |
| | | | AAGGGGCTGGAGTGGGTGGCAGTTATTTCATATGATGGAAATAATAAATACTACGCAGACTCC |
| | | | GTGAGGGGCCGATTCACCATCTCCAGAGACAATTCCAAGAACACGCTGTATCTGCAAATGAAC |
| | | | AGCCTGAGACCTGAGGACACGGCTGTGTATTACTGTGCGAGACCATATACTGGGAGCTACAAG |
| | | | AGCTACATGGACGTCTGGGGCAAAGGGACCACGGTCACCGTCTCCTCA |
| | 146 | light | GACATCGTGATGACCCAGTCTCCAGACTCCCTGGCTGTGTCTCTGGGCGAGAGGGCCACCATC |
| | | | AACTGCAAGTCCAGCCAGAGTGTTTTATACAGCTCCAACAATAAGAACTCCTTAGCTTGGTAC |
| | | | CAGCAGAAACCAGGACAGCCTCCTAAGCTGCTCATTTACTGGGCATCTACCCGGGAATCCGGG |
| | | | GTCCCTGACCGATTCAGTGGCAGUGGGTCTGGGACAGATTTCACTCTCACCATCAGCAGCCTG |
| | | | CAGGCTGAAGATGTGGCAGTATATTACTGTCAGCAATATTATAGTATTTCTTGGACGTTCGGC |
| | | | CAAGGGACCAAGGTGGAAATCAAA |
| COV2- | 147 | heavy | CAGGTGCAGCTGCAGGAGTCGGGCCCAGGACTGGTGAAGCCTTCACAGACCCTGTCCCTCACC |
| 2158 | | | TGCACTGTCTCTGGTGGCTCCATCAGCAGTGGTGGTTACTTCTGGAGCTGGATCCGCCAGCAC |
| | | | CCAGGGAAGGGCCTGGAGTGGATTGGGTCCATCTATTACAGTGGGAGCACCTACTACAACCCG |
| | | | TCCCTCAGGAGTCGAATTACCATATCAGTAGACACGTCTAAGAACCAGTTCTCCCTGAAGCTG |
| | | | AGCTCTGTGACTGCCGCGGACACGGCCGTGTATTACTGTGCGAGGGGGGGTTCGGGGAGTTAT |
| | | | TCTCTTTTTGACTACTGGGGCCAGGGAACCCTGGTCACCGTCTCCTCA |
| | 148 | light | GACATCCAGATGACCCAGTCTCCATCCTCCCTGTCTGCATCTGTAGGAGACAGAGTCACCATC |
| | | | ACTTGCCAGGCGAGTCAGGACATTACCAACTATTTAAATTGGTATCAGCAGAAACCAGGGAAA |
| | | | GCCCCTAAGCTCCTGATCTACGATGCATCCAATTTGGAAACAGGGGTCCCATCAAGGTTCAGT |
| | | | GGAAGTGGATCTGGGACAGATTTTACTTTCACCATCAGCAGCCTGCAGCCTGAAGATTTTGCA |
| | | | ACATATTACTGTCAACAGTATGATAATCTCTACTCTGTACACTTTGGCCAGGGGACCAAGCTG |
| | | | GAGATCAAA |
| COV2- | 149 | heavy | CAGGTGCAGCTGGTGGAGTCCGGGGGAGGCGTGGTCCAGCCTGGGAGGTCCCTGAGACTCTCC |
| 2161 | | | TGTGCAGCCTCTGGATTCACCTTCAGTAGGCATGCTATGCACTGGGTCCGCCAGGCTCCAGGC |
| | | | AAGGGGCTGGAGTGGGTGGCAGTTATATCATATGATGGAAGCAATAAATACTATGCAGACTCC |
| | | | GTGAAGGGCCGATTCGCCATCTCCAGAGACAATTCCAAGAACACGCTGTATCTGCAAATGAAC |
| | | | AGCCTGAGACCTGAGGACACGGCTGTATATTACTGTGCGAGAGATCCGAGCCCGTTAGTGCTT |
| | | | ATTACTTCAATTGACTACTGGGGCCAGGGAACCCTGGTCACCCTCTCCTCA |
| | 150 | light | TCCTATGAGCTGACACAGCCACCCTCGGTGTCAGTGTCCCCAGGACAGACGGCCAGGATCACC |
| | | | TGCTCTGGAGATGCATTGCCAAGGCAATATACTTATTGGTACCAGCAGAAGCCAGGCCAGGCC |
| | | | CCTGTGTTGGTGATATATAAAGACAGTGAGAGGCCCTCAGGGATCCCTGAGCGATTCTCTGGC |
| | | | TCCAGCTCAGGGACAACAGTCACGTTGACCATCAGTGGAGTCCAGGCAGAAGACGAGGCTGAC |
| | | | TATTACTGTCAATCAGCAGACACCATTGGTACTTATTGGGTATTCGGCGGAGGGACCAAGCTG |
| | | | ACCGTCCT |
| COV2- | 151 | heavy | GAGGTGCAGCTGGTGGAGTCTGGGGGAGGCTTGGTCCAGCCTGGGGGGTCCCTGAGACTCTCC |
| 2162 | | | TGTGCAGCCTCTGGATTCACCTTTAGTAGCTATTGGATGACCTGGGTCCGCCAGGCTCCAGGG |
| | | | AAGGGGCTGGAGTGGGTGGCCAACATAAAGCAAGATGGAAGTGAGAAATACTATGTGGACTCT |
| | | | GTGAAGGGCCGATTCACCATCTCCAGAGACAACGCCAAGAACTCACTGTCTCTGCAAATGAAC |
| | | | AGCCTGAGAGTCGAGGACACGGCTGTGTATTATTGTGTGAGACTGGGGGTCAGCAGCTGGTAC |
| | | | TTTGACTACTGGGGCCAGGGAACCCTGGTCACCGTGTCCTCA |
| | 152 | light | TCCTATGAGCTGACTCAGCCACCCTCAGTGTCCGTGTCCCCAGGACAGACAGCCAGCATCACC |
| | | | TGCTCTGGAGATAAATTGGGGGATAAATATGCTTGCTGGTATCAGCAGAAGCCAGGCCAGTCC |
| | | | CCTGTGCTGGTCATCTATCAAGATACCAAGCGGCCCTCAGGGATCCCTGAGCGATTCTCTGGC |
| | | | TCCAACTCTGGGAACACAGCCACTCTGACCATCAGCGGGACCCAGGCTATGGATGAGGCTGAC |
| | | | TATTACTGTCAGGCGTGGGGCAGTAGCAGGGGAGTGTTCGGCGGAGGGACCAAGCTGACCGTC |
| | | | CTA |
| COV2- | 153 | heavy | CAGGTGCAGCTGGTGCAGTCTGGGGCTGAGGTGAAGAAGCCTGGGTCCTCGGTGAAGGTCTCC |
| 2164 | | | TGCAAGGCTTCTGGAGGCACCTTCAGCAGCTATGCTATCAGCTGGGTGCGACAGGCCCCTGGA |
| | | | CAAGGGCTTGAGTGGATGGGAGGGATCATCCCTATCTTTGGTGCAGCAAACTACGCACAGAAC |
| | | | TTCCAGGGCAGAGTCACGATTACCGCGGACGAATCCACGAGCACAGGCTACATGCAACTGAGC |
| | | | AGCCTGAGATTTGAGGACACGGCCGTGTATTACTGTGCGAGAACGTCTCACTATGATAGTAGT |
| | | | GGTTCCTATTTTGAATACTGGGGCCAGGGAACCCTGGTCACCGTCTCCTCA |
| | 154 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTACTTAGCCTGGTACCAACAGAAACCTGGCCAG |
| | | | GCTCCCAGGCTCCTCATCTATGATGCATCCAACAGGGCCACTGGCATCCCAGCCAGGTTCAGT |
| | | | GGCAGTGGGTCTGGGACAGACTTCACTCTCACCATCAGCAGCCTAGACCCTGAAGATTTTGCA |
| | | | GTTTATTACTGTCACAAGCGTAGCAACTGGCCTCCTTCGCTCACTTTCGGCGGAGGGACCAAG |
| | | | GTGGAGATCAA |
| COV2- | 771 | hEavy | GAGGTGCAGCTGGTGGAGTCTGGGGGAGACTTGGTACAGCCTGGGGGGTCCGTGAGACTCTCC |
| 2000 | | | TGTGCAGCCTCTGGATTCACCTTTCGCAGCTATGCCATGAGCTGGGTCCGCCAGGCTCCAGGG |
| | | | AAGGGGCTGGAGTGGGTCTCAGCTATTAGTGATAATGCTTATAGCACATACTACGCAGACTCC |
| | | | GTGAAGGGCCGGTTCACCATCTCCAGAGACAATTCCAAGAACACTCTGTATCTGCAAATGAAC |
| | | | AGCCTGAGAGCCGAGGACACGGCCATATATTACTGTGCGAAGAATCTTTATAGTGGAAACTCC |
| | | | CCATTTGACTACTGGGGCCAGGGAACCCTGGTCACCGTCTCCTCA |
| | 772 | light | NNNTNTGTGCTGACTCAGCCACCCTCAGTGTCCGTGTCCCCAGGACAGACAGCCAGCATCACC |
| | | | TGCTCTGGAGATAAATTGGGGGATAAATATGCTTGCTGGTATCAGCAGAAGCCAGGCCAGTCC |
| | | | CCTGTGCTGGTCATCTATCAAGATAGCAAGCGGCCCTCAGGGATCCCTGAGCGATTCTCTGGC |
| | | | TCCAACTCTGGGAACACAGCCACTCTGACCATCAGCGGGACCCAGGCTATGGATGAGGCTGAC |
| | | | TATTACTGTCAGGCGTGGGACAGCAGCACTGCCGTCTTCGGAACTGGGACCAAGGTCACCGTC |
| | | | CT |
| COV2- | 773 | hEavy | GAGGTGCAGCTGGTGGAGTCTGGGGGAGGCGTGGTCCAGCCTGGGAGGTCCCTGAGACTCTCC |
| 2001 | | | TGTGCAGCGTCTGGATTCACCTTCAGTAGTTATGGCATGCATTGGGTCCGCCAGGCTCCAGGC |
| | | | AAGGGGCTGGAGTGGGTGGCAGTTATATGGCATGATGGAAGTAAGAAATACTATGCAGACTCC |
| | | | GTGAAGGGCCGATTCACCATCTCCAGAGACAATTCCAAGAACACGCTGTATCTGCAAATGAAC |
| | | | AGCCTGAGAGCCGAGGACACGGCTGTGTATTACTGTGCGAGAGATCAAGGTGGATATGATTAC |
| | | | GTTTGGGGGAGTTATCGATATACATTTTACGTCTTTGACTACTGGGGCCAGGGAACCCTGGTC |
| | | | ACCGTCTCCTCA |
| | 774 | light | NANTNTGTGCTGACTCAGGACCCTGCTGTGTCTGTGGCCTTGGGACAGACAGTCAGGATCACA |
| | | | TGCCAAGGAGACAGCCTCAGAAACTATTATGCCAGCTGGTACCAGCAGAAGCCAGGACAGGCC |
| | | | CCTGTAGTTGTCATGTATGGTAAAAACAACCGGCCCTCAGGGATCCCAGACCGAGTCTCTGGC |
| | | | TCCAGCTCAGGAAACACAGCTTCCTTGACCATCACTGGGGCTCAGGCGGAAGATGAGGCTGAC |
| | | | TATTATTGTAACTCCCGGGACAGCAGTGGTAACCATCTGATATTCGGCGGAGGGACCAAGCTG |
| | | | ACCGTCCT |
| COV2- | 775 | heavy | GAGGTGCAGCTGGTGGAGTCTGGGGGAGGCTTGGTCCAGCCTGGGGGGTCCCTGAGACTCTCC |
| 2002 | | | TGTGCAGCCTCTGGATTCACCTTTAGTTTCTATTGGATGAGCTGGGTCCGCCAGGCTCCAGGG |
| | | | AAGGGGCTGGAGTGGGTGGCCAACATAAAGCAAGATGGAAGTGAGAAATACTATGTGGACTCT |
| | | | GTGAAGGGCCGATTCAGCATCTCCAGAGACAACGCCAAGAACTCACTGTATCTGCAAATGAAC |
| | | | AGCCTGAGAGCCGAGGACACGGCTGTGTATTACTGTGCGAGAGTTGGTAGCAGCAGCTGGTAT |
| | | | TTTGACTACTGGGGCCAGGGAACCCTGGTCACCGTCTCCTCA |
| | 776 | light | NNNNTTGTGCTGACTCAGCCACCCTCAGTGTCCGTGTCCCCAGGACAGACAGCCAGCATCACC |
| | | | TGCTCTGGAGATAAATTGGGGGATAAATATGCTTGCTGGTATCAGCAGAAGCCAGGCCAGTCC |
| | | | CCTGTGCTGGTCATCTATCAAGATATCAAGCGGCCCTCAGGGATCCCTGAGCGATTCTCTGGC |
| | | | TCCAACTCTGGGAACACAGCCACTCTGACCATCAGCGGGACCCAGGCTATGGATGAGGCTGAC |
| | | | TATTACTGTCAGGCGTGGGACAGCAACACTGGAGTGTTCGGCGGAGGGACCAAGCTGACCGTC |
| | | | CT |
| COV2- | 777 | heavy | GAGGTGCAGCTGGTGCAGTCTGGGGCTGAGGTGAAGAAGCCTGGGGCCTCAGTGAAGGTCTCC |
| 2003 | | | TGCAAGGCTTCTGGATACACCCTCACCAGGTATGATATCCACTGGGTGCGACAGGCCACTGGA |
| | | | CAAGGGCTTGAGTGGATGGGATGGTTGAACCCTAACGGTGGCAACACAGGCTATGCACAGAAG |
| | | | TTCCAGGGCAGAGTCACCATGACCAGAAACACCGCCATAAGCACAGCCTACATGGAGCTGAGC |
| | | | AGCCTGAGATCTGAGGACGCGGCCGTGTATTACTGTGCGAGGGGTCAGTGGGAACTAGACGCT |
| | | | TGGTACTTCGATCTCTGGGGCCGTGGCACCCTGGTCACCGTCTCCTCA |
| | 778 | light | CAGTNTGTGCTGACTCAGGACCCTGCTGTGTCTGTGGCCTTGGGACAGACAGTCAGGATCACA |
| | | | TGCCAAGGAGACAGCTTCAGAAGCTATTATGCAAGCTGGTACCAGCAGAAGCCAGGACAGGCC |
| | | | CCTGTACTTGTCATCTATGGTAAAAACAACCGGCCCTCAGGGATCCCAGACCGATTCTCTGGC |
| | | | TCCAGCTCAGGAAACACAGCTTCCTTGACCATCACTGGGGCTCAGGCGGAAGATGAGGCTGAC |
| | | | TATTATTGTAACTCCCGGGACACCAGTGGTAACCATCTAGATGTGGTATTCGGCGGAGGGACC |
| | | | AAGCTGACCGTCCT |
| COV2- | 779 | heavy | GAGGTGCAGCTGGTGCAGTCTGGGGCTGAGGTGAAGAAGCCTGGGGCCTCACTGAAGGTCTCC |
| 2004 | | | TGCAAGGCTTCTGGATACACCTTCACCCGTTATGATATCAACTGGGTGCGACAGGCCACTGGA |
| | | | CAAGGGCTTGAGTGGATGGGATGGATGAACCCTAACAGTGATAACACAGGCTATGCACAGAAG |
| | | | TTCCAGGACAGAGTCACCATGACCAGGAACACCTCCATAAGCACAGTCTACATGGAGCTGAGC |
| | | | AGCCTGAGATCTGAGGACACGGCCGTGTATTACTGTGCGAGGGGTCAGTGGGAGCTAGACGTT |
| | | | TGGTACTTCGATCTCTGGGGCCGTGGCACCCTGGTCACCGTCTCCTCA |
| | 780 | light | CAGNNTGTGCTGACTCAGGACCCTGCTGTGTCTGTGGCCTTGGGACAGACACTCAGGATCACA |
| | | | TGCCAAGGAGACAGCCTCAGAAGCTATTATGCAAGCTGGTACCAGCAGAAGCCAGGACAGGCC |
| | | | CCTGTACTTGTCATCTATGGTAAAAACAACCGGCCCTCAGGGATCCCAGACCGATTCTCTGGC |
| | | | TCCAGCTCAGGAAACACAGCTTCCTTGACCATCACTGGGGCTCAGGCGGAAGATGAGGCTGAC |
| | | | TATTACTGTAACTCCCGGGACAGCAGCGGTCACCATCTAGATGTGGTATTCGGCGGAGGGACC |
| | | | AAGCTGACCGTCCT |
| COV2- | 781 | heavy | CAGGTGCAGCTGCAGGAGTCGGGCCCAGGACTGGTGAAGCCTTCGGAGACCCTGTCCCTCACC |
| 2005 | | | TGCACTGTCTCTGGTGGCTCCATCAGTAGTTACTACTGGAGCTGGATCCGGCAGCCCCCAGGG |
| | | | AAGGGACTGGAGTGGATTGGGTGTATCTATTACAGTGGGCGCACCAACTACAGCCCCTCCCTC |
| | | | AAGAGTCGAGTCACCATATCAGTAGACACGTCCAAGAACCTGTTCTCCCTGAAGCTGAGCTCT |
| | | | GTGACCGCTGCGGACACGGCCGTGTATTACTGTGCGAGAGGAGGCCGCCCTGGGGCTGAAGGA |
| | | | CCTTATGATGCTTTTGATATCTGGGGCCAAGGGACAATGGTCACCGTCTCCTCA |
| | 782 | light | CAGNNTGTGCTGACTCAGGACCCTGCTGTGTCTGTGGCCTTGGGACAGACAGTCAGGATCACA |
| | | | TGCCAAGGAGACAACCTCAGAAGGTATTATGCAAGCTGGTACCAGCAGAAGCCAGGACAGGCC |
| | | | CCTGTACTAGTCATCTATGGTAAAAACAACCGGCCCTCAGGGATCCCAGACCGATTCTCTGGC |
| | | | TCCAGCTCAGGAAACACAGCTTCCTTGACCATCACTGGGGCTCAGGCGGAAGATGAGGCTGAC |
| | | | TATTACTGTAACTCCCGGGACAGCAGTGGTAACCATGTGGTATTCGGCGGAGGGACCACGCTG |
| | | | ACCGTCCT |
| COV2- | 783 | heavy | CAGGTGCAGCTGGTGCAATCTGGGTCTGAGTTGAAGAAGCCTGGGGCCTCAGTGAAGGTTTCC |
| 2954 | | | TGCAAGGCTTCTGGATACACCTTCAGTGACTATGCTATGAATTGGGTGCGACAGGCCCCTGGA |
| | | | CAAGGACTTGAGTGGATGGGATGGATGAAGTCTAACAGTGGTAACACAGGCTATGCACAGAAG |
| | | | TTCCAGGGCAGAGTCACTATGACCAGGAACACCTCCATAAGTACAGCCTACATGGAGTTGACC |
| | | | AGCCTGAGATCTGAGGACACGGCCGTGTATTACTGTGCGAGAATGCGCAGTGGCTGGCCCACA |
| | | | CATGGCCGCCCGGATGACCACTGGGGCCGGGGAGCCCTGGTCACCGTCTCCTCAG |
| | 784 | light | GACATCCAGATGACCCAGTCTCCATCCTCCCTGTCTGCATCTGTGGGAGACAGAGTCACCATC |
| | | | ACTTGCCGGGCAAGTCAGAGCATTATCAGCTATTTAAATTGGTATCACCAGAAACCGGGGAAA |
| | | | GCCCCTAAACTCCTGATCTATGCTGCATCCAGTTTGCAAAGTGGGGTCCCATCAAGGTTCAGT |
| | | | GGCAGTGGATCTGGGACAGATTTTACTTTCACCATCAGCAGCCTGCAGCCTGAAGATATTGCA |
| | | | ACATATTACTGTCAACAGTCTGATAATCTCCCCATGTACACTTTTGGCCAGGGGACCAAGCTG |
| | | | GAGATCAAAC |
| COV2- | 785 | heavy | CAGGTGCAGCTGGTGGAGTCTGGGGGAGGCGTGGTCCAGCCTGGGAGGTCCCTGAGACTCTCC |
| 2956 | | | TGTGCAGCCTCTGGATTCACCTTCGTTACCTCTGGCATACACTGGGTCCGCCAGGCTCCAGGC |
| | | | AAGGGGCTGGAGTGGGTGGCAGTTATATCATATGATGGAAGTAATAAATACTATGCAGACTCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTGTATTACTGTGCGAAAGGAGGGCCAAACAAGGAAGTA |
| | | | CTATATTTCGGGGAGTTATTGGACTACGGTATGGACGTCTGGGGCCAAGGGACCACGGTCACC |
| | | | GTCTCCTCA |
| | 786 | light | GACATCGTGATGACCCAGTCTCCAGACTCCCTGGCTGTGTCTCTGGGCGAGAGGGCCACCATC |
| | | | AACTGCAAGTCCAGCCAGAGTGTTTTATACAGTTCCGAAATTAAGAACTACTTAGCTTGGTAT |
| | | | CAGCAGAAACCAGGACAGCCTCCTAAGCTGCTCATTTACTGGGCATCTACCCGGGAATTCGGG |
| | | | GTCCCTGACCGATTCAGTGGCAGCGGGTCTGGGACAGATTTCACTCTCACCATCAGCAGCCTG |
| | | | CAGGCTGAAGATGTGGCAGTTTATTACTGTCAGCAATATTATAGTGGTCCCCTGGACACTTTT |
| | | | GGCCAGGGGACCAAGCTGGAGATCAAAC |
| COV2- | 787 | heavy | CAGGTCCAGCTTGTGCAGTCTGGGGCTGAGGTGAAGAAGCCTGGGGCCTCAGTGAAGGTTTCC |
| 2957 | | | TGCAAGGTCTCTGGATACACCTTCACTGGCTATGTTGTACATTGGGTGCGCCAGGCCCCCGGA |
| | | | CAAGACCTTGAGTGGATGGGATGGATCAACACTGGCTACGGCAACACAAAATATTCACAGAAG |
| | | | TTCCAGGGCAGGGTCACTATTAGCTGGGACACATCCGCGACGACAGCCTACATGGAGCTGAGC |
| | | | AACCTGAAATCTGAGGACAAGGCTGTTTATTATTGTGCGAGTATGAACCGGATGTCAGAGCAA |
| | | | ACTTACTACGGAATGGACGTCTGGGGCCAGGGGACCACGGTCACCGTCTCC |
| | 788 | light | GACATCCAGATGACCCAGTCTCCATCCTCCCTGTCTGCATCTGTAGGAGACAGAGTCACCATC |
| | | | ACTTGCCAGGCGAGTCAGGACATTAGCAACTATTTAAATTGGTATCAGCAGAAACCAGGGAAA |
| | | | GCCCCTAAGCTCCTGATCTACGATGCATCCAATTTGGAAACAGGGGTCCCATCAAGGTTCAGT |
| | | | GGAAGTGGATCTGGGACAGATTTTACTTTCACCATCAGCAGCCTGCAGCCTGAAGATATTGCA |
| | | | ACATATTACTGTCAACAGTATGATAAGCTCACLCTCGGCGGAGGGACCAAGGTGGAGATCAAA |
| | | | C |
| COV2- | 789 | heavy | CAGGTGCAGCTACAGCAGTGGGGCGCAGGACTGTTGAAGCCTTCGGAGACCCTGTCCCTCACC |
| 2958 | | | TGCGCTGTCTATGGTGGGTCCTTCAGTGGTTACTACTGGAGCTGGATCCGCCAGCCCCCAGGG |
| | | | AAGGGGCTGGAGTGGATTGGGGAAATCAATCATAGTGGAAGCACCAACTACAATCCGTCCCTC |
| | | | AAGAGTCGAGTCACCATATCAGTGGACACGTCCAAGAACCACTTCTCCCTGAAAATGAACTCT |
| | | | GTGACCGCCGCGGACACGGCTGTGTATTACTGTGCGAGATGTCGCCAGATGGGGAACTTCTAC |
| | | | TACTACTACATGGACGTCTGGGGCAAGGGGACCACGGTCACCGTCTCC |
| | 790 | light | CAGTCTGTGCTGACGCAGCCGCCCTCAGTGTCTGGGGTCCCAGGGCAGAGGGTCACCGTCTCC |
| | | | TGCACTGGGAGCAGCTCCAACATCGGGGCAGGTTTTGATGTATATTGGTACCAGCAGTTTCTA |
| | | | GGAACAGCCCCCAAACTCCTCATCTATGGCAACAACAATCGGCCCTCAGGCGTCCCTGACCGA |
| | | | TTCTCTGCCTCCAAGTCTGGCACCTCAGCCTCCCTGGCCATCACTGGGCTCCAGGCTGAAGAT |
| | | | GAGGCTGATTATTACTGCCAGTCCTTTGACATCGGCCGGGGTGGTTGGATTTTCGGCGGAGGG |
| | | | ACCAAGCTGACCGTCCTAG |
| COV2- | 791 | heavy | CAGGTGCACCTACAGGAGTCGGGCCCAGGACTGGTGAAGCCTTCGGAGACCCTGTCCCTCACC |
| 2959 | | | TGCACTGTCTCTGGTGGCTCCATCAATAATTATTACTGGAGCTGGATCCGGCAGCCCCCGGGG |
| | | | AAGGGACTGGAGTGGATTGGGGAAATCCATTACAGTGGGAGCACCAGCTACAGCCCCTCCCTC |
| | | | AAGAGTCGACTCAGCATATCAGTAGACAGGTCCAAGAACCAGTTCTCCCTGAAGCTGGCCTCT |
| | | | GTGACCGCTGCAGACACGGCCGTGTATTACTGTGTGAGGGATAATTACTTTGATAATAGTGGT |
| | | | CATCCTGTGTATCCGGTTCCCTGGTTCGACCCCTGGGGCCAGGGAACCCTGGTCACCGTCTCC |
| | | | TCAG |
| | 792 | light | CAGTCTGTGCTGACGCAGCCGCCCTCAGTGTCTGGGGTCCCAGGGCAGAGGGTCACCGTCTCC |
| | | | TGCACTGGGAGCAGCTCCAACATCGGGGCAGGTTTTGATGTATATTGGTACCAGCAGTTTCTA |
| | | | GGAACAGCCCCCAAACTCCTCATCTATGGCAACAACAATCGGCCCTCAGGCGTCCCTGACCGA |
| | | | TTCTCTGCCTCCAAGTCTGGCACCTCAGCCTCCCTGGCCATCACTGGGCTCCAGGCTGAAGAT |
| | | | GAGGCTGATTATTACTGCCAGTCCTTTGACATCGGCCGGGGTGGTTGGATTTTCGGCGGAGGG |
| | | | ACCAAGCTCACCGTCC |
| COV2- | 793 | heavy | CAGGTTCAGGTGGTGCAGTGTGGAGCTGAGGTAAAGAAGCCTGGGGCCTCAGTGAAGGTCTCC |
| 2960 | | | TGCAAGGCTTCTGGTTACACCTTTAAGAACTATGGGATCAGTTGGGTGCGACAGGCCCCTGGA |
| | | | CAAGGGCTTGAGTGGATGGGATGGATCAGCGCTTACACAGGAAACACAAACTATGCACAGAAG |
| | | | TTCCAGGGCAGAATGACCATGACCACAGACACATCCACGGGAACAGGTTATATGGAACTGAGG |
| | | | AGCCTGAGATCTGACGACACGGCCGTGTATTACTGTGCGAGAGTACAACGACGTCGACTTGAC |
| | | | TACTGGGGCCAGGGAACCCTGGTCATCGTCTCGTGAG |
| | 794 | light | GATATTGTGGTGACCCAGACTCCACTCTCTCTGTCCGTCACCCCTGGACAGCCGGCCTCCATC |
| | | | TCCTGCAAGTCTAGTGAGACCCTCCTGCATAGTGATGGAAAGACCTATTTGTCTTGGTATCTG |
| | | | CAGAAGCCAGGCCAGCCTCCACAGCTCCTGATCTATGAAGTTTCCAACCGGTTCTCTGGAGTG |
| | | | CCAGACAGGTTCAGTGGCAGCGGGTCAGGAACAGATTTCACACTTAAAATCGGCCGGGTGGAG |
| | | | GCTGAGGATGTTGGGCTTTATTACTGCATGCAAAGTATACAGCTCGCCTTCGGCCAAGGGACA |
| | | | CGACTGGAAATTGAAC |
| COV2- | 795 | heavy | GAGGTGCAGCTGGTGGAGTCTGGGGGAGGCGTGGTCCAGCCTGGGAGGTCCCTGAGACTCTCC |
| 2150 | | | TGTGCAGCCTCTGGATTCACCTTCAGTACGTATGCTATGCACTGGGTCCGCCAGGCTCCAGGC |
| | | | AAGGGGCTGGAGTGGGTGGCAGTTATATCATATGATGGAAGCAATAAATACTACGCAGACTCC |
| | | | GTGAAGGGCCGATTCACCATCTCCAGAGACAATTCCAAGAACACACTATATCTGCAAATGATC |
| | | | GGCCTGAGAGCTGAGGACACGGCTGTGTATTACTGTGCGAGAGATTGGGCACCTACGTACTAC |
| | | | GATATGCCGAGTGCTTTTGATATCTGGGGCCAAGGGACAATGGTCACCGTCTCTTCA |
| | 796 | light | TCCTATGAGCTGACACAGCCACCCTCGGTGTCAGTGGCCCCAGGAAAGACGGCCAGGATTACC |
| | | | TGTGGGGGAAACAACATTGGAAATAAAGGTGTGCATTGGTACCAGCAGAAGCCAGGCCAGGCC |
| | | | CCTGTGCTGGTCGTCGATGATGATAGCGACCGGCCCTCAGGGATCCCTGAGCGATTCTCTGGC |
| | | | TCCAACTCTGGGAACACGGCCACCCTGATCATCAGCAGTGTCGAAGTCGGGGATGAGGCCGAC |
| | | | TTTTACTGTCAGGTGTGGGATAGTAGTAGTGATCATCCGGGGGTGTTCGGCGGAGGGACCAAG |
| | | | CTGACCGTCCTA |
| COV2- | 797 | heavy | GAGGTGCAGCTGGTGGAGTCTGGGGGAGGCGTGGTCCAGCCTGGGAGGTCCCTGAGACTCTCC |
| 2159 | | | TGTGCAGCCTCTGGATTCACCTTCAGTAGCTATGCTATGCACTGGGTCCGCCAGGCTCCAGGC |
| | | | AAGGGGCTGGAATGGGTGGCAGTTATATCATATGATGGAAGCAATAAATACTACGCAGACTCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTGTATTACTGTGCGAGAAGCACGAGTGGGAGCTACTAC |
| | | | TACGGTATGGACGTCTGGGGCCAAGGGACCACGGTCACCGTCTCCTCA |
| | 798 | light | CAGTCTGCCCTGACTCAGCCTGCCTCCGTGTCTGGGTCTCCTGGACAGTCGATCACCATCTCC |
| | | | TGCACTGGAACCAGCAGTGACGTTGGTGATTATAACTATGTCTCCTGGTACCAACAACACCCA |
| | | | GGCAAAGCCCCCAAACTCATGATTTATGATGTCAGTAATCGGCCCTCAGGGGTTTCTAATCGC |
| | | | TTCTCTGGCTCCAAGTCTGGCAACACGGCCTCCCTGACCATCTCTGGGCTCCAGGCTGAGGAC |
| | | | GAGGCTGAATATTACTGCAGCTCATATACAAGCAGCAGCACTCTACTTTATGTCTTCGGAACT |
| | | | GGGACCAAGGTCACCGTCCTA |
| COV2- | 799 | heavy | GAGGTGCAGCTGGTGGAGTCTGGGGGAGGCGTGGTCCAGCCTGGGAGGTCCCTGAGACTCTCC |
| 2160 | | | TGTGCAGCCTCTGGATTCACCTTCAGTAGCTATGCTATGCACTGGGTCCGCCAGGCTCCAGGC |
| | | | AAGGGGCTGGAATGGGTGGCAGTTATATCATATGATGGAAGCAATAAATACTACGCAGACTCC |
| | | | GTGAAGGGCCGATTCACCATCTCCAGAGACAATTCCAAGAACACGCTGTATCTGCAAATGAAC |
| | | | AGCCTGAGAGCTGAGGACACGGCTGTGTATTACTGTGCGAGAAGCACGAGTGGGAGCTACTAC |
| | | | TACGGTATGGACGTCTGGGGCCAAGGGACCACGGTCACCGTCTCCTCA |
| | 800 | light | CAGTCTGCCCTGACTCAGCCTGCCTCCGTGTCTGGGTCTCCTGGACAGTCGATCACCATCTCC |
| | | | TGCACTGGAACCAGCAGTGACGTTGGTGATTATAACTATGTCTCCTGGTACCAACAACACCCA |
| | | | GGCAAAGCCCCCAAACTCATGATTTATGATGTCAGTAATCGGCCCTCAGGGGTTTCTAATCGC |
| | | | TTCTCTGGCTCCAAGTCTGGCAACACGGCCTCCCTGACCATCTCTGGGCTCCAGGCTGAGGAC |
| | | | GAGGCTGAATATTACTGCAGCTCATATACAAGCAGCAGCACTCTACTTTATGTCTTCGGAACT |
| | | | GGGACCAAGGTCACCGTCCTA |
| COV2- | 801 | heavy | GAGGTGCAGCTGGTGCAGTCTGGGGCTGAGGTGAAGAAGCCTGGGTCCTCGGTGAAGGTCTCC |
| 2166 | | | TGCAAGGCTTCTGGAGGCACCTTCAGCAGCTATGCTATCATCTGGGTGCGACAGGCCCCTGGA |
| | | | CAAGGGCTTGAGTGGATGGGAGGGATCATCCCTATCTTTGGTACAACAAACTACGCACAGAAG |
| | | | TTCCAGGGCAGAGTCACGATTACCGCGGACGAATCCACGAGCACAGCCTACGTGGAACTGAGC |
| | | | AGCCTGAGATCTGAGGACACGGCCGTGTATTATTGTGCGAGAATAGGCCATTTTGATAGTAGT |
| | | | GGTTATTACTTAGACTACTGGGGCCAGGGAACCCTGGTCACCGTCTCCTCA |
| | 802 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTTCTTAGCCTGGTACCAACAGAAACCTGGCCAG |
| | | | GCTCCCAGGCTCCTCATCTATGATGCATCCAACAGGCCCACTGGCATCCCAGCCAGGTTCACT |
| | | | GGCAGTGGGTCTGGGACAGACTTCACTCTCACCATCAGCAGCCTAGAGCCTGAAGATTTTGCA |
| | | | GTTTATTACTGTCAGCACCGTACCAACTGGCCTCCCTTATTCACTTTCGGCCCTGGGACCAAA |
| | | | GTGGATATCAAA |
| COV2- | 803 | heavy | GAGGTGCAGCTGGTGCAGTCTGGGGCTGAGGTGAAGAAGCCTGGGTCCTCGGTGAAGGTCTCC |
| 2169 | | | TGCAAGGCTTCTGGAGGCACCTTCAGCAGCTATGCTATCAGCTGGGTGCGACAGGCCCCTGGA |
| | | | CAAGGGCTTGAGTGGATGGGAGGGATCATCCCTATCTTTGGTGCAGCAAACTACGCACAGAAC |
| | | | TTCCAGGGCAGAGTCACGATTACCGCGGACGAATCCACGAGCACAGGCTACATGCAACTGAGC |
| | | | AGCCTGAGATTTGAGGACACGGCCGTGTATTACTGTGCGAGAACGTCTCACTATGATAGTAGT |
| | | | GGTTCCTATTTTGAATACTGGGGCCAGGGAACCCTGGTCACCGTCTCCTCA |
| | 804 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTACTTAGCCTGGTACCAACAGAAACCTGGCCAG |
| | | | GCTCCCAGGCTCCTCATCTATGATGCATCCAACAGGGCCACTGGCATCCCAGCCAGGTTCAGT |
| | | | GGCAGTGGGTCTGGGACAGACTTCACTCTCACCATCAGCAGCCTAGACCCTGAAGATTTTGCA |
| | | | GTTTATTACTGTCACAAGCGTAGCAACTGGCCTCCTTCGCTCACTTTCGGCGGAGGGACCAAG |
| | | | GTGGAAATCAAA |
| COV2- | 805 | heavy | GAGGTGCAGCTGGTGGAGTCTGGGGGAGGCTTGGTCCAGCCTGGGGGGTCCCTGAGACTCTCC |
| 2175 | | | TGTGTAGCCTCTGGATTCACCTTTAGTTTCTATTGGATGAGCTGGGTCCGCCAGGCTCCAGGA |
| | | | AAGGGGCTCGAGTGGGTGGCCAACATAAAGCAAGATGGAGGTGAGAAATACTATGTGGACTCT |
| | | | GTGAAGGGCCGATTCACCATCTCCAGAGACAACGCCAAGAACTCACTGTATCTGCAAATGAAC |
| | | | AGCCTGAGAGCCGAGGACACGGCTGTGTATTACTGTGCGAGACTGTCTGGCAGCAGCTGGGAC |
| | | | TTTGACTACTGGGGCCAGGGAACCCTGGTCACCGTCTCCTCA |
| | 806 | light | TCCTATGAGCTGACACAGCCACCCTCAGTGTCCGTGTCCCCAGGACAGACAGCCAGCATCACC |
| | | | TGCTCTGGAGATAAATTGGGGGATAAATATGCTTGTTGGTACCAACAGAGGCCAGGCCAGTCC |
| | | | CCTGTGCTGGTCATCTATCAAGATAGTAAGCGGCCCTCAGGGATCCCTGAGCGATTCTCTGGC |
| | | | TCCAACTCTGGGAACACAGCCACTCTGACCATCAGCGGGACCCAGGCTATGGATGAGGCTGAC |
| | | | TATTACTGTCAGGCGTGGGACAGCAGCACTGGAGTCTTCGGAAGTGGGACCAAGGTCACCGTC |
| | | | CTA |
| COV2- | 807 | heavy | GAGGTGCAGCTGGTGGAGTCTGGGGGAGGTGTGGTACGGCCTGGGGGGTCCCTGAGACTCTCC |
| 2191 | | | TGTGCAGCCTCTGGATTCACCTTTGATGATTATGGCATGAGCTGGGTCCGCCAAGCTCCAGGG |
| | | | AAGGGGCTGGAGTGGGTCTCTGCTATTAATTGGAATGGTGGTAGCACAGGTTATGCAGACTCT |
| | | | GTAAAGGGCCGATTCACCATCTCCAGAGATAATGCCAAGAACTCCCTGTATCTGCAAATGAAC |
| | | | AGTCTGAGAGCCGAGGACACGGCCTTGTATCACTGTGCGAGACGGCGTAGCTCGTCCCGGTAT |
| | | | AGCAGTGGCTGGTATATGTACTACTACTACATGGACGTCTGGGGCAAAGGGACCACGGTCACC |
| | | | GTCTCCTCA |
| | 808 | light | GACATCCAGATGACCCAGTUTCCTTCCACCCTGTCTGCATCTGTAGGAGACAGAGTCACCATC |
| | | | ACTTGCCGGGCCAGTCAGAGTGTTAGTACCTGGTTGGCCTGGTATCAGCAGAAACCAGGGAAA |
| | | | GCCCCTAACCTCCTGATCTATGAGGCGTCTAGTTTAGAAAGTGGGGTCCCATCAAGGTTCAGC |
| | | | GGCAGTGGATCTGGGACAGAATTCACTCTCACCATCAGCAGCCTGCAGCCTGATGATTTTGCA |
| | | | ACTTATTACTGCCAACAGTATAATACTTATTCGGGGACGTTCGGCCAAGGGACCAAGGTGGAA |
| | | | ATCAAA |
| COV2- | 809 | heavy | GAGGTGCAGCTGGTGGAGTCTGGGGGAGGTGTGGTACGGCCTGGGGGGTCCCTGAGACTCTCC |
| 2194 | | | TGTGCAGCCTCTGGATTCACCTTTGATGATTATGGCATGAGCTGGGTCCGCCAAGCTCCAGGG |
| | | | AAGGGGCTGGAGTGGGTCTCTGCTATTAATTGGAATGGTGGTAGCACAGGTTATGCAGACTCT |
| | | | GTAAAGGGCCGATTCACCATCTCCAGAGATAATGCCAAGAACTCCCTGTATCTGCAAATGAAC |
| | | | AGTCTGAGAGCCGAGGACACGGCCTTGTATCACTGTGCGAGACGGCGTAGCTCGTCCCGGTAT |
| | | | AGCAGTGGCTGGTATATGTACTACTACTACATGGACGTCTGGGGCABAGGGACCACGGTCACC |
| | | | GTCTCCTCA |
| | 810 | light | GACATCCAGATGACCCAGTCTCCTTCCACCCTGTCTGCATCTGTAGGAGACAGAGTCACCATC |
| | | | ACTTGCCGGGCCAGTCAGAGTGTTAGTACCTGGTTGGCCTGGTATCAGCAGAAACCAGGGAAA |
| | | | GCCCCTAACCTCCTGATCTATGAGGCGTCTAGTTTAGAAAGTGGGGTCCCATCAAGGTTCAGC |
| | | | GGCAGTGGATCTGGGACAGAATTCACTCTCACCATCAGCAGCCTGCAGCCTGATGATTTTGCA |
| | | | ACTTATTACTGCCAACAGTATAATACTTATTCGGGGACGTTCGGCCAAGGGACCAAGGTGGAA |
| | | | ATCAAA |
| COV2- | 811 | heavy | GAGGTGCAGCTGGTGCAGTCTGGGGCTGAGGTGAAGAAGCCTGGGTCCTCGGTGAAGGTCTCC |
| 2198 | | | TGCAAGGCTTCTGGAGGCACCTTCAGCAGCTATGCTATCAGCTGGGTGCGACAGGCCCCTGGA |
| | | | CAAGGGCTTGAGTGGATGGGAGGGATCATCCCTATCTTTGGTGCAGCAAACTACGCACAGAAC |
| | | | TTCCAGGGCAGAGTCACGATTACCGCGGACGAATCCACGAGCACAGGCTACATGCAACTGAGC |
| | | | AGCCTGAGATTTGAGGACACGGCCGTGTATTACTGTGCGAGAACGTCTCACTATGATAGTAGT |
| | | | GGTTCCTATTTTGAATACTGGGGCCAGGGAACCCTGGTCACCGTCTCCTCA |
| | 812 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTACTTAGCCTGGTACCAACAGAAACCTGGCCAG |
| | | | GCTCCCAGGCTCCTCATCTATGATGCATCCAACAGGGCCACTGGCATCCCAGCCAGGTTCAGT |
| | | | GGCAGTGGGTCTGGGACAGACTTCACTCTCACCATCAGCAGCCTAGACCCTGAAGATTTTGCA |
| | | | GTTTATTACTGTCACAAGCGTAGCAACTGGCCTCCTTCGCTCACTTTCGGCGGAGGGACCAAG |
| | | | GTGGAGATCAAA |
| COV2- | 813 | heavy | GAGGTGCAGCTGGTGGAGTCTGGGGGAGGCGTGGTCCAGCCTGGGAGGTCCCTGAGACTCTCC |
| 2203 | | | TGTGCAGCCTCTGGATTCACCTTCAGTACCTATGCTATGCACTGGGTCCGCCAGGCTCCAGGC |
| | | | AAGGGGCTGGCGTGGGTGGCACTTATATCATATGATGGATATAATAAATACTACGCAGACTCC |
| | | | GTGAGGGGCCGATTCACCATCTCCAGAATCAATTCCAAGAACACGCTGTCTCTGCAGATGAAC |
| | | | AGCCTGAGAGCTGAGGACACGGCTGTCTATTACTGTGCGAGAGGGTCAGCTGGAAACTACTAC |
| | | | TACGGTATGGACGTCTGGGGCCAGGGGACCACGGTCACCGTCTCCTCA |
| | 814 | light | GACATCCAGATGACCCAGTCTCCATCCTCCCTGTCTGCATCTGTAGGAGACAGAGTCACCATC |
| | | | ACTTGCCGGGCAAGTCAGACCATTACCAACTATTTAAATTGGTATCAGCTGAAATCAGGGAGA |
| | | | GCCCCCAAGCTCCTGATCTATGCTGCATCCAGTTTGCAAAGTGGGGTCCCATCAAGGTTCAGT |
| | | | GGCAGTGGATCTGGGACAGATTTCACTCTCACCATCAGCAGTCTGCAACCTGAAGATTTTGCA |
| | | | ACTTACTACTGTCAACAGAGTTACAGTACGCCGTACACTTTTGGCCAGGGGACCAAGGTGGAG |
| | | | ATCAAA |
| COV2- | 815 | heavy | GAGGTGCAGCTGGTGCAGTCTGGGGCTGAGGTGAAGAAGCCTGGGTCCTCGGTGAAGGTCTCC |
| 2214 | | | TGCAAGGCTTCTGGAGGCACCTTCAGCAGCTATGCTATCATCTGGGTGCGACAGGCCCCTGGA |
| | | | CAAGGGCTTGAGTGGATGGGAGGGATCATCCCTATCTTTGGTACAACAAACTACGCACAGAAG |
| | | | TTCCAGGGCAGAGTCACGATTACCGCGGACGAATCCACGAGCACAGCCTACGTGGAACTGAGC |
| | | | AGCCTGAGATCTGAGGACACGGCCGTGTATTATTGTGCGAGAATAGGCCATTTTGATAGTAGT |
| | | | GGTTATTACTTAGACTACTGGGGCCAGGGAACCCTGGTCACCGTCTCCTCA |
| | 816 | light | GAAATTGTGTTGACACAGTCTCCAGCCACCCTGTCTTTGTCTCCAGGAGAAAGAGCCACCCTC |
| | | | TCCTGCAGGGCCAGTCAGAGTGTTAGCAGCTTCTTAGCCTGGTACCAACAGAAACCTGGCCAG |
| | | | GCTCCCAGGCTCCTCATCTATGATGCATCCAACAGGCCCACTGGCATCCCAGCCAGGTTCACT |
| | | | GGCAGTGGGTCTGGGACAGACTTCACTCTCACCATCAGCAGCCTAGAGCCTGAAGATTTTGCA |
| | | | GTTTATTACTGTCAGCACCGTACCAACTGGCCTCCCTTATTCACTTTCGGCCCTGGGACCAAA |
| | | | GTGGATATCAAA |
| COV2- | 817 | heavy | GAGGTGCAGCTGGTGCAGTCTGGGGCTGAGGTGAAGAAGCCTGGGTCCTCGGTGAAGGTCTCC |
| 2216 | | | TGCAAGGCTTCTGGAGGCACCTTCAGCAGCTATGCTATCAGCTGGGTGCGACAGGCCCCTGGA |
| | | | CAAGGGCTTGAGTGGATGGGAGGGATCATCCCTATCTTTGGTGCAGCAAACTACGCACAGAAC |
| | | | TTCCAGGGCAGAGTCACGATTACCGCGGACGAATCCACGAGCACAGGCTACATGCAACTGAGC |
| | | | AGCCTGAGATTTGAGGACACGGCCGTGTATTACTGTGCGAGAACGTCTCACTATGATAGTAGT |
| | | | GGTTCCTATTTTGAATACTGGGGCCAGGGAACCCTGGGAGGGTCTCCTCA |
| | 818 | light | GAAATTGTGTTGACACAGTCTCCAGCCACCCTGTCTTTGTCTCCAGGGGAAAGAGCCACCCTC |
| | | | TCCTGCAGGGCCAGTCAGAGTGTTAGCAGCTACTTAGCCTGGTACCAACAGAAACCTGGCCAG |
| | | | GCTCCCAGGCTCCTCATCTATGATGCATCCAACAGGGCCACTGGCATCCCAGCCAGGTTCAGT |
| | | | GGCAGTGGGTCTGGGACAGACTTCACTCTCACCATCAGCAGCCTAGACCCTGAAGATTTTGCA |
| | | | GTTTATTACTGTCACAAGCGTAGCAACTGGCCTCCTTCGCTCACTTTCGGCGGAGGGACCAAG |
| | | | GTGGAAATCAAA |
| COV2- | 819 | heavy | GAGGTGCAGCTGGTGGAGTCTGGGGGAGGCGTGGTCCAGCCTGGGAGGTCCCTGAGACTCTCC |
| 2218 | | | TGTGCAGCCTCTGGATTCACCTTCAGTAGTTATGCTCTGTTCTGGGTCCGCCAGGCTCCAGGC |
| | | | AAGGGGCTGGAGTGGGTGGCAGTTATTTCATATGATGGAAATAATAAATACTACGCAGACTCC |
| | | | GTGAGGGGCCGATTCACCATCTCCAGAGACAATTCCAAGAACACGCTGTATCTGCAAATGAAC |
| | | | AGCCTGAGACCTGAGGACACGGCTGTGTATTACTGTGCGAGACCATATACTGGGAGCTACAAG |
| | | | AGCTACATGGACGTCTGGGGCAAAGGGACCACGGTCACCGTCTCCTCA |
| | 820 | light | GACATCGTGATGACCCAGTCTCCAGACTCCCTGGCTGTGTCTCTGGGCGAGAGGGCCACCATC |
| | | | AACTGCAAGTCCAGCCAGAGTGTTTTATACAGCTCCAACAATAAGAACTCCTTAGCTTGGTAC |
| | | | CAGCAGAAACCAGGACAGCCTCCTAAGCTGCPCATTJACTGGGCATCTACCCGGGAATCCGGG |
| | | | GTCCCTGACCGATTCAGTGGCAGCGGGTCTGGGACAGATTTCACTCTCACCATCAGCAGCCTG |
| | | | CAGGCTGAAGATGTGGCAGTATATTACTGTCAGCAATATTATAGTATTTCTTGGACGTTCGGC |
| | | | CAAGGGACCAAGGTGGAAATCAAA |
| COV2- | 821 | heavy | GAGGTGCAGCTGGTGGAGTCCGGGGGAGGCGTGGTCCAGCCTGGGAGGTCCCTGAGACTCTCC |
| 2224 | | | TGTGCAGCCTCTGGATTCACCTTCAGTACCTATGGCATGCACTGGGTCCGCCAGGCTCCAGGC |
| | | | AAGGGGCTGGAGTGGGTGGCAGTTATATCATATGATGGAAGTAATAAATACTATGCAGACTCC |
| | | | GTGAAGGGCCGATTCACCATCTCCAGAGACAATTCCAAGAACACACTGTATCTGCAAATGAAC |
| | | | AGCCTGAGAGCTGAGGACACGGCTATGTATTACTGTGCGAAAGATGGGAGTATAGCAGCAGCT |
| | | | GACTACTGGGGCCAGGGAACCCTGGTCACCGTCTCCTCA |
| | 822 | light | GACATCGTGATGACCCAGTCTCCAGACTCCCTGGCTGTGTCTCTGGGCGAGAGGGCCACCATC |
| | | | AACTGCAAGTCCAGCCAGAGTGTCTTACACAGCTCCAACAACAAGGACTCCTTAGTTTGGTAC |
| | | | CAGCAGAAACCAGGACAGCCTCCTAAGCTGCTCATTTACTGGGCATCTAGCCGGGAATCCGGG |
| | | | GTCCCTGACCGATTCAGTGGCAGCGGGTCTGGGACAGATTTCACTCTCACCATCAGCAGCCTG |
| | | | CAGGCTGAAGATGTGGCAGTTTATTACTGTCAGCAATATTATAGTACTCCTTGGACGTTCGGC |
| | | | CAAGGGACCAAGGTGGAAATCAAA |
| COV2- | 823 | heavy | GAGGTGCAGCTGGTGGAGTCTGGAGGAGGCTTGATCCAGCCTGGGGGGTCCCTGAGACTCTCC |
| 2235 | | | TGTGCAGCCTCTGGGTTCATCGTCAGTAGCAACTACATGAGCTGGGTCCGCCAGGCTCCAGGG |
| | | | AAGGGGCTGGAGTGGGTCTCAGTTATTTATAGCGGTGGTAGCACATACTACGCAGACTCCGTG |
| | | | AAGGGCCGATTCACCATCTCCAGAGACAATTCCAAGAACACGCTGTATCTTCAAATGAACAGC |
| | | | CTGAGAGCCGAGGACACGGCCGTATATTACTCTGCGAGAGAGAGCACGCAATGGGGCCAGGGA |
| | | | ACCCTGGTCACCGTCTCCTCA |
| | 824 | light | GACATCCAGATGACCCAGTCTCCTTCCACCCTGTCTGCATCTGTAGGAGACAGAGTCACCATC |
| | | | ACTTGCCGGGCCAGTCACAGTATTAGTAGCTGGTTGGCCTGGTATCAGCAGAAACCAGGGAAA |
| | | | GCCCCTAAGCTCCTGATCTATAAGGCGTCTAGTTTAGAAAGTGGGGTCCCATCAAGGTTCAGC |
| | | | GGCAGTGGATCTGGGACAGAATTCACTCTCACCATCAGCAGCCTGCAGCCTGATGATTTTGCA |
| | | | ACTTATTACTGCCAACAGTATAATACTTATTCTCAAACGTTCGGCCAAGGGACCAAGGTGGAG |
| | | | ATCAAA |
| COV2- | 825 | heavy | GAGGTGCAGCTGGTGCAGTCTGGGCCTGAGGTGAAGAAGCCTGGGACCTCAGTGAAGGTCTCC |
| 2961 | | | TGCAAGGCTTCTGGATTCACCTTTATGAGCTCTGCTGTGCAGTGGGTGCGACAGGCTCGTGGA |
| | | | CAACGCCTTGAGTGGATAGGATGGATCGTCATTGGCAGTGGTAACACAAACTACGCACAGAAG |
| | | | TTCCAGGAAAGAGTCACCATTACCAGGGACATGTCCACAAGCACAGCCTACATGGAGCTGAGC |
| | | | AGCCTGAGATCCGAGGACACGGCCGTGTATTACTGTGCGGCCCCATATTGTAGTAGTATCAGC |
| | | | TGCAATGATGGTTTTGATATCTGGGGCCAAGGGACAATGGTCACCGTCTCTTCA |
| | 826 | light | GAAATTGTGTTGACGCAGTCTCCAGGCACCCTGTCTTTGTCTCCAGGGGAGAGAGCCACCCTC |
| | | | TCCTGCAGGGCCAGTCAGAGTGTTAGCAGCAGCTACTTAGCCTGGTACCAGCAGAAACCTGGC |
| | | | CAGGCTCCCAGGCTCCTCATCTATGGTGCATCCAGCAGGGCCACTGGCATCCCAGACAGGTTC |
| | | | AGTGGCAGTGGGTCTGGGACAGACTTCACTCTCACCATCAGCAGACTGGAGCCTGAAGATTTT |
| | | | GCAGTGTATTACTGTCAGCACTATGGTAGCTCACGGGGTTGGACGTTCGGCCAAGGGACCAAG |
| | | | GTGGAAATCAAA |

TABLE 2
PROTEIN SEQUENCES FOR ANTIBODY
VARIABLE REGION

| Clone | Seq ID | Chain | Variable Sequence Region |
|---|---|---|---|
| COV2- | 157 | heavy | EVQLVESGGGLVQPGGSLRLSCVASGFTFSFYWMSWVRQAPGKG |
| 2171 | | | LEWVANIKQDGGEKYYVDSVKGRFTISRDNAKNSLYLQMNSLRA |
| | | | EDTAVYYCARLSGSSWDFDYWGQGTLVTVSS |
| | 158 | light | SYELTQPPSVSVSPGQTASITCSGDKLGDKYACWYQQRPGQSPV |
| | | | LVIYQDSKRPSGIPERFSGSNSGNTATLTISGTQAMDEADYYCQ |
| | | | AWDSSTGVFGTGTKVTVL |
| COV2- | 159 | heavy | EVQLVESGGGVVRPGGSLRLSCAASGFTFDDYGMSWVRQAPGKG |
| 2173 | | | LEWVSAINWNGGSTGYADSVKGRFTISRDNAKNSLYLQMNSLRA |
| | | | EDTALYHCARRRSSSRYSSGWYMYYYYMDVWGKGTTVTV |
| | 160 | light | DIQMTQSPSTLSASVGDRVTITCRASQSVSTWLAWYQQKPGKAP |
| | | | NLLIYEASSLESGVPSRFSGSGSGTEFTLTISSLQPDDFATYYC |
| | | | QQYNTYSGTFGQGTKVEIK |
| COV2- | 161 | heavy | QITFKESGPTLVKPTETLTLTCTFSGFSVSTSGEGVGWIRQPPG |
| 2177 | | | KALEWLAVIYWDDDKRYSPSLKSRLTITRDTSKNQVVLTMTNMD |
| | | | PVDTATYYCAHRLWFRDAFDIWGQGTTVTVSS |
| | 162 | light | DIQMTQSPSSLSASVGDRVTITCRARQSISNYLNWYQQKPGKAP |
| | | | KLLIYAASSLHSGVPSRFSGSGSGTDFTLTISSLQPEDFATYYC |
| | | | QQTYSTFWTFGQGTNVEIK |
| COV2- | 163 | heavy | EVQLVESGGGLVQPGGSLRLSCAASGFTFSTYWMTWVRQAPGKG |
| 2178 | | | LEWVANIKQDGSEKYYVDSVKYRFTISRDNAKNSLYLQMNSLRA |
| | | | EDTAVYYCARVGSSSWYFDYWGQGTLVTVSS |
| | 164 | light | SYELTQPPSVSVSPGQTASITCSGDKLGDKYACWYQQKPGQSPV |
| | | | VVIYQDSKRPSGIPERFSGSNSGNTATLTISGTQAMDEADYYCQ |
| | | | AWDSSTAVFGGGTKLTVL |
| COV2- | 161 | heavy | QLQLQESGPGLVKPSETLSLTCTVSGGSISSGTYYCGWIRQPPG |
| 2179 | | | KGLEWIGSTYYGGSTLYNPSLRGRVTISVDTSKNQFSLKLSSVT |
| | | | AADTAVYYCARRGNYYDSKNWFDPWGQGTLVTVSS |
| | 166 | light | EIVMTQSPATVSVSPGERATLSCRASQSVSSNLAWYQQKPGQAP |
| | | | RLLIYGASTRATGIPARFSASGSGTEFTLTISSLQSEDFAVYYC |
| | | | QQYNNWPPMYTFGQGTKVEIK |
| COV2- | 167 | heavy | QVQLVESGGGVVQPGRSLRVSCAASGFTFSSHGMHWVRQAPGKG |
| 2181 | | | LEWVSVIWYDGSNKYYADSVKGRFTISRDNSKNTLSLQMNSLRA |
| | | | EDTAVYYCARESADISSRLDYWGRGTLVTVSS |
| | 168 | light | SYELTQPPSVSVSPGQTARITCSGDALPTKYAYWYQQKSGQAPV |
| | | | LVIYDDSKRPSGIPERFSGSSSGTMATLTISGAQVEDEADYYCY |
| | | | STDSSGNVFGTGTKVTVL |
| COV2- | 169 | heavy | QVQLVESGGGVVQPGRSLRLSCAASGFTFSSYAMHWVRQAPGKG |
| 2183 | | | LEWVAGISYDGSNKYYADSVKGRFTISRDNSKNTLYLQMNSLRA |
| | | | EDTAVYYCARADTMVRGTYFEYWGQGTLVTVSS |
| | 170 | light | QSALTQPASVSGSPGQSITISCTGTSSDVGGYNYVSWYQQHPGK |
| | | | APKLMIYDVSNRPSGVSNRFSGSKSGNTASLTISGLQAEDEADY |
| | | | CCSSYTSSRAVLFGGGTKLTVL |
| COV2- | 171 | heavy | QITLKESGPTLVKPTQTLTLTCTFSGFSLSTSGVGVAWIRQPPG |
| 2184 | | | KALEWLALIYWDDDKRYSPSLKSRLTITKDTSKNQVVLTMTNMD |
| | | | PVDTATYYCAHRLPTPQLLPSFENWFDPWGQGTLVTVSS |
| | 172 | light | QSVLTQPPSVSEAPRQRVTISCSGGSSNIGNNAVNWYQQLPGKA |
| | | | PKLLIYYDDLLPSGVSDRFSGSKSGTSASLAISGLQSEDEADYY |
| | | | CASWDDSLIGPVFGGGTKLTVL |
| COV2- | 173 | heavy | EVQLVESGGGLVQPGGSLRLSCAVSGFTFSSYWMHWVRQAPGKG |
| 2285 | | | LVWVSRINSDGSSTSYADSVKGRFTISRDNAKNTLYLQMNSLRA |
| | | | EDTAVYYCAREVEQLAHMVDYWGQGTLVTVSS |
| | 174 | light | SYELTQPPSVSVSPGQTARITCSGDALPNQYAYWYQQKPGQAPV |
| | | | LVIYKDSERPSGIPERFSGSSSGTTVTLTISGVQAEDEADYYCQ |
| | | | SADSSGTSWVFGGGTKLTVL |
| COV2- | 175 | heavy | QVQLVESGGGVVQPGRSLRLSCAASGFTFSSYAMHWVRQAPGKG |
| 2186 | | | LEWVAVISYDGINKYYADAVKGRFTISRDNSKNTLYLQMNSLRA |
| | | | EDTAVYYCARPRSGSYYAYFDYWGQGTLVTVSS |
| | 176 | light | DIQMTQSPSSLSASVGDRVTITCRASQGISNYLAWFQQKPGKAP |
| | | | KSLIYAASSLQSGVPSKFSGSGSGTDFTLTISSLQPEDVATYYC |
| | | | QQYNSHPPTFGGGTKVEIK |
| COV2- | 177 | heavy | QVQLVESGGGVVQPGRSLRLSCAASGFTFSYYPMHWLWVRQAPG |
| 2187 | | | KGLEWVAVTSYDGTNKYYADSVKGRFTISRDNSKNTLYLQMNSL |
| | | | RAEDTAVYYCARGGATNFDYWGQGTLVTVSS |
| | 178 | light | SYVLTQPPSVSVAPGKTANITCGGNNIGRKSVHWYQQKSGQAPV |
| | | | LVVYDDSDRPSGIPSRFSGSNSGNTATLTISRVEAGDEADYYCQ |
| | | | VWDSSSDHPEWVFGGGTKLTVL |
| COV2- | 179 | heavy | EAQLVESGGGLVQPGRSLRLSCAASGFTFDDSAMHWVRQAPGKG |
| 2189 | | | LEWVSGISWNSGNVGYADSVKGRFTTSRDNAKNSLYLQMNSLRA |
| | | | EDTALYYCTKASRYCSSTICYWNWFDPWGQGTLVTVSS |
| | 180 | light | DIQMTQSPSSLSASVGDRVTITCRASQSISSYLNWYQQKPGKAP |
| | | | KLLIYGASSLQTGVPSRFSGSGSGTDFTLTIRSLQPEDFASYYC |
| | | | QQSYSTPTFGGGTKVEIK |
| COV2- | 181 | heavy | EVQLVESGGGLVQPGGSLRLSCAASGFTFSSYEMNWVRQAPGKG |
| 2190 | | | LEWVSYISSSGSAIYYADSVKGRFTISRDNAKNSLYLQMNSLRV |
| | | | EDTAVYYCAREARSRYFDWLPSYYFDYWGQGTLVTVSS |
| | 182 | light | QSALTQPASVSGSPGQSITISCTGTSSDIGGYNYVSWYQQHPGK |
| | | | APKLLIYDVSNRPSGVSTRFSGSKSGNTASLTISGLQAEDEADY |
| | | | YCSSYTSSSTHVVFGGGTKLTVL |
| COV2- | 183 | heavy | QVQLVQSGSELKKPGASVKVSCKASGYTFTTYAMNWVRQAPGQG |
| 2192 | | | LEWMGWINTNTGNPTYAQGFTGRFVFSLDTSVNTAFLHIGSLKA |
| | | | EDTAVYYCARDQDSGYPTYYYYYMDVWGKGTTVTVSS |
| | 184 | light | DIVMTQTPLSLSVTPGQPASISCKSSQSLLHSDGKTYLYWYLQK |
| | | | PGQSPQLLIYEVSNRFSGVPDRFSGSGSGTDFTLKISRVEAEDV |
| | | | GVYYCMQSIQPPLTFGGGTKVEIK |
| COV2- | 185 | heavy | QVQLVESGGGVVQPGRSLRLSCAASGFTFSTYAMHWVRQAPGKG |
| 2193 | | | LAWVALISYDGYNKYYADSVRGRFTTSRINSKNTLSLQMNSLRA |
| | | | EDTAVYYCARGSAGNYYYGMDVWGQGTTVTVSS |
| | 186 | light | DIQMTQSPSSLSASVGDRVTITCRASQTITNYLNWYQLKSGRAP |
| | | | KLLIYAASSLQSGVPSRFSGSGSGTDFTLTISSLQPEDFATYYC |
| | | | QQSYSTPYTFGQGTKLEIK |
| COV2- | 187 | heavy | QVHLVESGGGVVQPGRSLRLSCAASGFTFSNYGMHWVRQAPGKG |
| 2395 | | | LEWVAVISNDEFNKFYANSVKGRFTISRDNSKNTVYLQLNSLRT |
| | | | EDTARYYCAKGGDGSGWAWDGDNPPTDYWGQGTLVIVSS |
| | 188 | light | DIVMTQSPDFLAVSLGERATISCKSSQSVLYTPKNKNYLAWYKQ |
| | | | KPGQPPKVLIYWASTRESGVPDRESGSGSGTDFTLTISSLQAED |
| | | | AAVYYCQQYYTAPLTFGGGTRVEI |
| COV2- | 191 | heavy | EVQLVQSGAEVKKPGESLKISCKGSGYSFTSYWIGWVRQMPGKG |
| 2197 | | | LEWMGIIYPGDSDTRYSPSFQGQVTISADKSISTAYLQWSSLKA |
| | | | SDTAMYYCARPDYSSGWPSYWYFDLWGRGTLVTVSS |
| | 192 | light | EIVLTQSPGTLSLSPGERATLSCRASQSVSSNFLAWYQQKPGQA |
| | | | PRLLIYGASSRATGIPDRFSGSGSGTDFTLTISRLEPEDFAVYY |
| | | | CQQYGRSPITFGQGTRLEIK |
| COV2- | 193 | heavy | QVQLVQSGAEVKKPGASVKVSCKVSGYTLTELSIHWVRQAPGKG |
| 2199 | | | LEWMGGFDPEDAETIYAQQFQGRVTMTEDTSTDTAYMELSSLKS |
| | | | EDTALYYCATGFAVFGRAAVPYWGQGTLVTVSS |
| | 194 | light | SYELTQPPSVSVSPGQTASITCFGDKLGDKYACWFQQKPGQSPV |
| | | | LIIYQGAKRPSGIPERFSGSNSGNTATLTISGTQAMDEADYYCQ |
| | | | AWDSSTVVFGGGTKLTVI |
| COV2- | 195 | heavy | QVQLVESGGGVVQPGRSLRLSCAASGFTFSSYGMHWVRQAPGKG |
| 2200 | | | LEWVAVISYDGSNKYYADSVKGRFTISRDNSKNTLYLQMNSLRA |
| | | | EDTAVYYCAKDLTIVVIPAAPNFDYWGQGTLVTVSS |
| | 196 | light | QSALTQPASVSGSPGQSITISCTGTSSDVGGYNYVSWYQQHPGK |
| | | | APKLMIYDVSNRPSGVSNRFSGSRSGNTASLTISGLQAEDEADY |
| | | | YCSSYTSSSTPVVFGGGTKLTVL |
| COV2- | 197 | heavy | EVQLVQSGAEVKKPGESLKISCKGSGYSFTSHWIGWVRQMPGKG |
| 2207 | | | LEWMGIIYPGDSDTRYSPSFQGQVTISADKSISTAYLQWSSLKA |
| | | | SDTAMYYCASALRERGVQLWSVWGQGTLVTVSS |
| | 198 | light | QSVLTQPPSVSGAPGQRVTISCTGSSSNIGAGYDVHWYQQLPGT |
| | | | APKLLIFINSNRPSGVPDRFSGSKSGTSASLAITGLQAEDEADY |
| | | | YCQSYDSSLGALFGGGTKLTVL |
| COV2- | 199 | heavy | QVQLVESGGGVVQPGRSLRLSCAASGFTFSSYAMHWVRQAPGKG |
| 2210 | | | LEWVAVISYDGSNKYYADSVKGRFTISRDNSKNTLYLQMNSLRV |
| | | | EDTAVYYCARDQEWFRELFLFDYWGQGTLVTVSS |
| | 200 | light | DIQMTQSPSSVSASVGDRVTITCRASQGISSWLAWYQQKPGKAP |
| | | | KLLIYDASSLQSGVPSRFSGSGSGTDFTLTISSLQPEDFATYYC |
| | | | QQANSFPPTFGPGTKVDIK |
| COV2- | 201 | heavy | QVQLVESGGGVVQPGRSLRLSCAASGFTFSTYGMHWVRQAPGKG |
| 2211 | | | LEWVAVISYDGSNKYYADSVKGRFTISRDNSKNTLYLQMNSLRA |
| | | | EDTAMYYCAKDGSIAAADYWGQGTLVTVSS |
| | 202 | light | DIVMTQSPDSLAVSLGERATINCKSSQSVLHSSNNKDSLVWYQQ |
| | | | KPGQPPKLLIYWASSRESGVPDRFSGSGSGTDFTLTISSLQAED |
| | | | VAVYYCQQYYSTPWTFGQGTKVEIK |
| COV2- | 203 | heavy | EVQLVESGGGLVKPGGSLRLSCAASGFTFSSYSMNWVRQAPGKG |
| 2212 | | | LEWVSSISNSNSFTYYADSMKGRFTTSRDNAKNSLYLQMNSLRA |
| | | | EDTAVYYCARVNGNSNWNFGSYYYYYMDVWGKGTTVTVSS |
| | 204 | light | EIVLTQSPAILSLSPGERATLSCRASQSVSSYLAWYQQKPGQAP |
| | | | RLLIYDTSNRATGIPARFSGSGSGTDFTLTISSLEPEDFAFYYC |
| | | | QQRGNWWTFAQGTKVEIK |
| COV2- | 205 | heavy | EVQLVESGGGLVKPGGSLRLSCAASGFTFSGYSMNWVRQAPGKG |
| 2215 | | | LEWVSSISSSSSYIYYADSVKGRFTISRDNAKNSLYLQMNSLRA |
| | | | EDTAVYYCARWLQLRSDYYYFGMDVWGQGTTVTVSS |
| | 206 | light | EIVMTQSPATLSVSPGERATLSCRASQSVSNNLAWYQHKPGQAP |
| | | | RLLIYGASTRATGIPARFSGSGSGTEFTLTISSLQSEDFAVYPC |
| | | | QQCYNWPPWTFGQGTKVEIK |
| COV2- | 207 | heavy | QVQLQESGPGLVKPSETLSLTCTVSGGSISSYYWSWTRQPPGKG |
| 2222 | | | LEWIGYIYYSGSTNYNPSLKSRVTISVDMSKNQFSLKLRSVTAA |
| | | | DTAVYYCARAPRERLQWGEYYPDYWGQGTLVTVSS |
| | 208 | light | QSALTQPASVSGSPGQSITISCTGTSSDVGSYNLVSWYQQHAGK |
| | | | APKLMIYEVIKRPSGVSNRFSGSKSGNTASLTISGLQAEDEADY |
| | | | YCCSYAVSTTYVIFGGGTKLTVL |
| COV2- | 209 | heavy | EVQLVQSGAEVKKPGESLKISCKGSGYSFTNSWIGWVRQMPGKG |
| 2226 | | | LEWMGIIYPGDSDTRYSPSFQGQVTISADKSISTAYLQWSSLKA |
| | | | SDTAIYYCATHRCSGGFCYLAYWGQGTLVTVSS |
| | 210 | light | QPVLTQPPSASASLGASVTLTCTLSSGYSNYKVDWYQQRPGKGP |
| | | | RPVMRVGTGGIVGSKGDGIPDRFSVLGSGLNRYLTIKNIQEEDE |
| | | | SDYHCGADHGSGSNFVFVVFGGGTKLTVL |
| COV2- | 211 | heavy | QVQLVESGGGVVQPGRSLRLSCAASGFTFSSYGMHWVRQAPGKG |
| 2227 | | | LEWVAVIWYDGSKKDYADSVKGRFTISRDNSKNTLYLQMNSLRA |
| | | | EDTAVYYCARDQSQGAYILTGYRGYGMDVWGQGTTVTVSS |
| | 212 | light | DIVMTQSPLSLPVTPGEPASISCRSSQSLLHSNGYNYLDWYLQK |
| | | | PGQSPQFLIYLGSNRASGVPDRFSGSGSGTDFILKISRVEAEDV |
| | | | GVYYCMQALQTPFTFGPGTKVDIK |
| COV2- | 213 | heavy | QVQLQQWGAGLLKPSETLSLTCAVYGGSFSGHYWSWTRQPPGKG |
| 2228 | | | LEWIGETNHSGSTNYNPSLKSRVTISVDTSKNQFSLKLSSVTAA |
| | | | DTAVYYCARPPQAARIHYYYYMDVWGKGTTVTVSS |
| | 214 | light | ETVMTQSPATLSVSPGERATLSCRASQSVSSNLAWYQQKPGQAP |
| | | | RLLIYGASTRATGIPARFSGSGSGTEFTLTISSLQSEDFAVYYC |
| | | | QQYNYWPPLTFGGGTKVEIK |
| COV2- | 215 | heavy | EVQLVESGGGLVQPGRSLRLSCAASGFTFDDYAMNWVRQPPGKG |
| 2231 | | | LEWVSGISWNSDSIGYADSVKGRFTISRDNAKNSLYLQMNSLRA |
| | | | EDTAMYYCAKGRGAGYTSYMDVWGKGTTVTVSS |
| | 216 | light | SYVLTQPPSVSVAPGKTARITCEGNNIGSKSVHWYQQKPGQAPV |
| | | | LVVYDDSGRPSGIPERFSGSNSGNTATLTISRVEAGDEADYFCQ |
| | | | VWDSSSDHHVVFGGGTKLTVL |
| COV2- | 217 | heavy | YTTLFRSGGHWIGWVRQMPGKGLEWMGIIYPGDSDTRYSPSFQG |
| 2233 | | | QVTISADKSISTAYLQWSSLKASDTAMYYCASALRERGVQLWSV |
| | | | WGQGTLVTVSS |
| | 218 | light | QSVLTQPPSVSGAPGQRVTISCTGSSSNIGAGYDVHWYQQLPGT |
| | | | APKLLIFINSNRPSGVPDRFSGSKSGTSASLAITGLQAEDEADY |
| | | | YCQSYDSSLGALFGGGTKLTVL |
| COV2- | 219 | heavy | EVQLVESGGGLVQPGRSLRLSCAASGFTFDDYAMHWVRQAPGKG |
| 2046 | | | LEWVSGISWNSGSIAYTDSVKGRFTISRDNAKNSLYLQMNSLRA |
| | | | EDTALYYCAKAHSTGHQYYYGMDVWGQGTTVTVSS |
| | 220 | light | DIQMTQSPSSLSASVGDRVTITCRASQSISSFLNWYQQKPGKAP |
| | | | KLLIYAAFNLQSGVPSRFSGSGSGTDFTLTISSLQSEDFATYYC |
| | | | QQSYNTPYTFGQGTKLEIK |
| COV2- | 221 | heavy | EVQLVESGGGLVQPGRSLRLSCAASGFTFDDYAMHWVRQAPGKG |
| 2047 | | | LEWVSGISWNSGSIGYADSVKGRFTISRDNAKNSLYLQMNSLRA |
| | | | EDTALYYCAKVSSITSLLGYYFDSWGQGTLVTVSS |
| | 222 | light | EIVLTQSPATLSLSPGERATLSCRASQSVSSYLAWYQQKPGQAP |
| | | | RLLIYDASNRATGIPARFSGSGSGTDFTLTISSLEPEDFAVYYC |
| | | | QHRSNWPPRLTFGGGTKVEIK |
| COV2- | 223 | heavy | QVQLVQSGAEVKKPGSSVKVSCKTSGDISSSYTVGWVRQAPGQG |
| 2048 | | | LEWMGRIIPILGIAYSAQKFQGRLTITADKSTSTSYMELSSLRS |
| | | | EDTAVYYCARGVVAATPGWFDPWGQGTLVTVSS |
| | 224 | light | ETVMTQSPATLSVSPGERVTLSCRASQSVSSNLAWYQQKPGQAP |
| | | | RLLIYGASTRATGIPARFSGGGSGTEFTLTISSLQSEDFAVYYC |
| | | | QQYNNFLTFGGGTKVEIK |
| COV2- | 225 | heavy | EVQLVESGGGLVQPGGSLRLSCAASGFTFSSYWMHWVRQVPGKG |
| 2049 | | | LVWVSRINSDGSSTSYADSVKGRFTISRDNAKNTLYLEMNSLRA |
| | | | QDTAVYYCAGSPWLRGDIDYWGQGTLVTVSS |
| | 226 | light | NFMLTQPHSVSESPGKTVTISCTGSSGSIASNYVQWYQQRPGSA |
| | | | PTTVIYEDNQRPSGVPDRFSGSIDSSSNSASLTISGLMTEDEAD |
| | | | YYCQSYDGSNHAVVPGGGTKLTVL |
| COV2- | 227 | heavy | QVQLVQSGAEVKKPGASVKVSCKASGYTFTDYYMHWVRQAPGQG |
| 2050 | | | LEWMGWINPNSRGTNYAQKFQGRVTMTRDTSISTVYMELSRLTS |
| | | | DDTAVYYCARVVVLGYGRPNNYYDGRNVWDYWGQGTLVTVSS |
| | 228 | light | QSVLTQPPSASGTPGQRVIISCSGSSSNIGSNTVKWYHQLPGTA |
| | | | PKLLICSNNQRPSGVPDRFSGSKSDTSASLAISGLQSEDEADYY |
| | | | CAAWDDSLNALVHGGGTKLTVL |
| COV2- | 229 | heavy | QVTLRESGPALVKPTQTLSLTCTPSGFSLGTSGMCVSWIRQPPG |
| 2051 | | | KALEWLARIDWDDDKYYSTSLKTRLTISKDTSKNQVVLTMTNMD |
| | | | PVDTATYYCARGVVTYDYWGQGTLVTVSS |
| | 230 | light | DIQMTQSPSSLSASVGDRVTITCRASQSIAGYLNWYQQKPGKAP |
| | | | KLLIYGTTSLQSGVPVRFSGSGSGTDFTLTISSLQPEDFATYYC |
| | | | QQSYSTPGTFGQGTRLEIK |
| COV2- | 231 | heavy | QVQLVQSGAEVKKPGSSVKVSCKASGDTFSSYTINWVRQAPGQG |
| 2054 | | | LEWMGRIIPILGIPNYAQKFQGRVTITADKSTSTAFMELSSLRS |
| | | | EDTAVYYCARGRGYSNYGASYYMDVWGKGTTVTVSS |
| | 232 | light | DIQMTQSPSSLSASVGDRVTITCQASQDINHYLNWYQQKPGKAP |
| | | | KLLIYDASNLETGVPSRFSGSGSGTDFTFTITSLQPEDVATYYC |
| | | | QQSDNLPMYTFGQGTKLEIK |
| COV2- | 233 | heavy | EVQLVESGGGLVQPGRSLRLSCAASGFTFDDYAMNWVRQPPGKG |
| 2055 | | | LEWVSGISWNSDSIGYADSVKGRFTISRDNAKNSLYLQMNSLRA |
| | | | EDTAMYYCAKGRGAGYTSYMDVWGKGTTVTVSS |
| | 234 | light | SYVLTQPPSVSVAPGKTARITCEGNNIGSKSVHWYQQKPGQAPV |
| | | | LVVYDDSGRPSGIPERFSGSNSGNTATLTISRVEAGDEADYFCQ |
| | | | VWDSSSDHHVVFGGGTKLTVL |
| COV2- | 237 | heavy | QVQLAQSGAEVKKPGASVKVSCKAAGYTFTSYDINWVRQATGQG |
| 2064 | | | LEWMGWMNSNSGNAGYAQKFQGRVTMTRDTSTSTAYMELSSLTS |
| | | | DDTAVYYCARMRTGWPTHGRPDDFWGRGTLVTVSS |
| | 238 | light | QSVLTQPPSASGTPGQRVTISCSGSNSNIGSYTVNWYQQPPGTA |
| | | | PKLLIYDNNQRTSGVPDRFSGSKSGTSASLAISGLQSEDEANYY |
| | | | CLVWDDSLNGLVFGGGTKLTVL |
| COV2- | 239 | heavy | EVQLVESGGGLVQPGGSLRLSCAASGFTVSSNYMSWVRQAPGKG |
| 2068 | | | LEWVSVIYPGGSAFYADSVKGRFTISRHNSNNTLCLQMNSLRTE |
| | | | DTAVYYCARSYDILTGYRDAFDIWGQGTMVTVSS |
| | 240 | light | QSVLTQPPSVSGAPGQRVTISCTGSSSNIGSGSDVHWYQQLPGT |
| | | | APKLLIYGNTNRPSGVPDRFSGSKSGTSASLAITGLQAEDEADY |
| | | | YCQSYDSRLSGPVVFGGGTKLTVL |
| COV2- | 241 | heavy | EVQLVESGGGLVQPGGSLRLSCAASGLTVSSNYMSWVRQAPGKG |
| 2069 | | | LECVSVIYAGGNTYYADSVKGRFTISRDNSKNTLYLQMNSLRAE |
| | | | DTAVYYCARGDGGYYSPPDYWGQGTLVTVSS |
| | 242 | light | DIQMTQSPSSLSASVGDRVTITCRASQSISSYLNWYQQKPGKAP |
| | | | KVLIYAASTMQSGVPSRFRGSGSGTDFTLTISSLQLEDFATYYC |
| | | | QQSYSTPQTFGQGTKVEIK |
| COV2- | 243 | heavy | QVQLQQWGAGLLKPSETLSLTCAVSGGSFSAYYWSWIRQPPGKG |
| 2070 | | | LEWIGEINHSGSTNYNPSLRSRVTISVDTSKNQFSLKLSSVTAA |
| | | | DTAVYYCARVGYSQGYYYYYMDVWGKGTTVTVSS |
| | 244 | light | DIQMTQSPSSLSASVGDRVTITCRASQSISNYLNWYQQKPGKAP |
| | | | KLLIYAASSLQSGVPSRFSGSGSGTDFSLTISSLQPEDFATYSC |
| | | | QQSYTTLLTFGGGTKVEIK |
| COV2- | 247 | heavy | QVQLVQSGAEVKKPGSSVKVSCKASGGTFSSYSITWVRQAPGQG |
| 2078 | | | LEWMGRIIPVLGIANYAQKFQDRVTITADKSTSTAYMELSSLRS |
| | | | EDTAVYYCARVGVSGFKSGSNWYFDLWGRGTLVTVSS |
| | 248 | light | QSVLTQPPSVSGAPGQRVTLSCTGSNSNIGAGYDVHWYQQLPGT |
| | | | APKLLIYGNSNRPSGVPDRFSGSKSGTSASLAITGLQAEDEADY |
| | | | YCQSYDSSLSDSVFGGGTKVTVL |
| COV2- | 251 | heavy | QVQLVQSGAEVKKPGASVKVSCKASGYTFTSYYMHWVRQAPGQG |
| 2081 | | | LEWMGIINPGGGSTTYAQKFQGRVTMTSDTSTSTVYMELSSLRS |
| | | | EDTAMYYCARGAIPPNSRAEIDYWGQGTLVTVSS |
| | 252 | light | ETVMTQSPATLSVSPGERVTLSCRASQSVSSNLAWCQQKPGQAP |
| | | | RLLIYGASTRATGIPARFSGSGSGTEFTLTISSLQSEDFAVYYC |
| | | | QQYYNWPLTFGGGTKVEIK |
| COV2- | 253 | heavy | EVQLVESGGGVVRPGGSLRLSCAASGFIFDDYDMTWVRQAPGKG |
| 2082 | | | LEWVSGISWNGGNTGYADSVKGRFTISRDNAKNSLYLQMNSLRA |
| | | | EDTALYHCAVIMSPIPRYSGYDWAGGAFDIWGQGTMVTVSS |
| | 254 | light | SSELTQDPAVSVALGQTVRITCQGDSLRSYYASWYQQKPGQVPI |
| | | | LVIYDKNNRPSGIPDRFSGSSSGNTASLTITGAQAEDEADYYCN |
| | | | SRDSSGNAVVFGGGTKLTVL |
| COV2- | 265 | heavy | QVQLVESGGGVVQPGRSLRLSCAASGFTFSNYGMHWVRQAPGKG |
| 2083 | | | LEWVAVMSYDGSNKYYADSVKGRFTISRDNSKNTLYLQMNSLRA |
| | | | EDTAVYYCAKNLGPYCSGGTCYSLVGDYWGQGTLVTVSS |
| | 256 | light | DIQMTQSPSSLSASVGDRVTITCQASQDISNYLNWYQQKPGKAP |
| | | | KLLIYDASNLETGVPSRFSGSGSGTDFTFTISSLQPEDIATYYC |
| | | | QQYANLPFTFGPGTKVDIK |
| COV2- | 261 | heavy | EVQLVESGGGLVQPGRSLRLSCAASGFTFDDYAMHWVRQAPGKG |
| 2097 | | | LEWVSGISWNSGTIGYADSVKGRFITSRDNAKNSLYLQMNSLRP |
| | | | EDTALYYCAKDIIRQGEDGMDVWGQGTTVTVSS |
| | 262 | light | DIQMTQSPSSLSASVGDRVTITCRASQNIASYLNWYQQKPGKAP |
| | | | KLLIYAASSLQSGVPSRFSGSGSGTDFTLTISSLQPEEFATYYC |
| | | | QQSYSTPWTFGQGTKVEIK |
| COV2- | 263 | heavy | EVQLLESGGGLIQPGGSLRLSCAASGFTFSNYAMSWVRQAPGKG |
| 2098 | | | LEWVSGIISTSGGATYNADSVRGRFTTSRDNSKNILYLQMNSLR |
| | | | GEDTAVYYCVKGLFDWFPLWGQGTMVTVSS |
| | 264 | light | DIVMTQSPATLSVSPGERAILSCRASQSVRSNLAWYQQKPGQAP |
| | | | RLLISGASTRATAIPARFSGSGSGTEFTLTITSLQSEDCAVYYC |
| | | | HQYNNWPQTFGQGTKVEIK |
| COV2- | 265 | heavy | EVQLVESGGGLVQPGGSLRLSCAASGFTFSRHWMTWVRQAPGKG |
| 2103 | | | EEWVANIKQDGSEKYYVDSVKGRLTISRDNAKNSLYLQMNSLRA |
| | | | TAVYYCARLGFYYGGADYWGQGTLVTVSS |
| | 266 | light | NFMLTQPHSVSESPGKTVTISCTGSSGSIASNYVQWYQQRPGSA |
| | | | PTTVISEDNQRPSGVPDRFSGSIDSSSNSASLTISGLKTEDEAD |
| | | | YYCQSYDGINRAWVFGGGTKLTVL |
| COV2- | 267 | heavy | NVQLVESGGGLVQPGGSLRLSCAASGFTFHHYAMHWVRQAPGKG |
| 2108 | | | LEWVSGISGSSDYRAYADSLKGRFTISRDYAKNSLWLQMNSLTS |
| | | | EDTAFYYCAKGVDYGGKLAYFDSWGQGTLVTVSS |
| | 268 | light | DIVMTQSPLSLPVTPGEPASISCRSSQSLLHSLGYNSLSWYLQK |
| | | | PGQSPHLLIYLGSNRASGVPDRFSGSGSATDPTLKISRLEAEDV |
| | | | GVYYCMQALQTPLTFGQGTRLEIK |
| COV2- | 269 | heavy | EVQLVESGGGVVQPGRSLRLSCAASGFSFSSYVMNWVRQAPGKG |
| 2110 | | | EWVAVISYDGSSKYYADSVKGRFTISRDNSKNTLYLQMNSLRA |
| | | | DTAVYYCARDIDSGYDPTPVFDYWGQGTLVTVSS |
| | 270 | light | DIQMTQSPSSLSACVGDRVTITCRASQSISSYLNWYQQKPGKGP |
| | | | KLLIYAASSLQSGVPSRFSGSGSGTDFTLTISSLQPEDFATYYC |
| | | | QQSYSSLSITFGQGTRLEIK |
| COV2- | 271 | heavy | EVQLVESGGGLVQPGGSLRLSCAASGFTFSSYDLHWVRQGTGKR |
| 2111 | | | LEWVSAIGTAGDTYYLGSVKGRFTISRENAKNSLYLQMNSLRAG |
| | | | DTAVYYCARVLYDSSGFYNWFDPWGQGTLVTVSS |
| | 272 | light | DIQMTQSPSSLSASVGDRVTITCRASQSISSYLNWYQQKPGKAP |
| | | | KLLIYAASSLESGVPSRFSGSGSGTDFTLTISSLQPEDFATYYC |
| | | | QQSYEIPPWTFGQGTKVEIK |
| COV2- | 273 | heavy | EVQLVESGGGLIQPGGSLRLSCAASEVTVSSNYMSWVRQAPGKG |
| 2113 | | | LEWVSLIYSGGTTYYADSVKGRFTISRDNSKNTLYLQMNSLRAE |
| | | | DTAVYYCARDFLRWHDLWGQGTLVTVSS |
| | 274 | light | DIQMTQSPSSLSASVGDRVTITCQASQDINNYLNWYQQKPGKAP |
| | | | KLLIYDASNLETGVPLRFSGSGSGTDFIFTISSLQPEDIATYYC |
| | | | QQYDNLPPVFGGGTKVEIK |
| COV2- | 275 | heavy | QVQLVQSGAEVKKPGSSVKVSCKASGDTFSSYTINWVRQAPGQG |
| 2114 | | | LEWMGRIIPILGIPNYAQKFQGRVTITADKSTSTAFMELSSLRS |
| | | | DTAVYYCARGRGYSNYGASYYMDVWGKGTTVTVSS |
| | 276 | light | QSVLTQPPSVSGAPGQRVTISCTGSSSNIGAGYDVHWYQQLPET |
| | | | APKLLIYANSNRPSGVPDRFSGSKSGTSASLAITGLQAEDEADY |
| | | | YCQSYDSSLSGSVFGGGTKLTVL |
| COV2- | 277 | heavy | QLQLQESGPGLVKPSETLSLTCTVSGGSISSSSYYWGWIRQPPG |
| 2128 | | | NSLEWTGSIYYSGSTYYNPSLKGRVSISVDTSKNQPSLKLSSVT |
| | | | ADTAVYYCARILVIFTLNWFDPWGQGTLVTVSS |
| | 278 | light | NFMLTQPHSVSESPGKTVTISCTGSSGSIASNYVQWYQQRPGSA |
| | | | PTTVIYEDNQRPSGVPDRFSGSIDSSSNSASLTISGLKTEDEAD |
| | | | YCQSYDSGNPIFGGGTKLTVL |
| COV2- | 281 | heavy | EVQLVESGGGLIQPGGSLRLSCAASEVTVSSNYMSWVRQAPGKG |
| 2132 | | | LEWVSLIYSGGTTYYADSVKGRFTISRDNSKNTLYLQMNSLRAE |
| | | | DTAVYYCARDFLRWHDLWGQGTLVTVSS |
| | 282 | light | DIQMTQSPSSLSASVGDRVTITCQASQDINNYLNWYQQKPGKAP |
| | | | KLLIYDASNLETGVPLRFSGSGSGTDFTFTISSLQPEDIATYYC |
| | | | QQYDNLPPVPGGGTKVEIK |
| COV2- | 283 | heavy | QVQLQESGPGLVKPSETLSLTCTVSGGSVSSGSYYWSWIRQPPG |
| 2237 | | | KGLECIGYIYYSGSSNYNPSLKSRVTISVDTSKNQFSLKMSSVT |
| | | | AADTAVYYCAGSPVPPTIVGASYWGQGTLVTVSS |
| | 284 | light | NFMLTQPHSVSESPGKTVTFSCTGSSGSIASNYVQWYQQRPGSA |
| | | | PTTVIYEDNQRPSGVPDRFSGSIDSSSNSASLTISGLKTEDEAD |
| | | | YYCQSYDGINRWLVFGGGTKLTVL |
| COV2- | 285 | heavy | EVQLVQSGAEVKKPGESLKISCKGSGYSFTSYWIDWVRQMPGKG |
| 2142 | | | LEWMGIIYPGDSDTRYSPSFQGQVTISADKSTSTAYLQWSSLKA |
| | | | SDTAMYYCARRGEAAGIWYFDLWGRGTLVTVSS |
| | 286 | light | QPVLTQPPSASASLGASVTLTCTLSSGYSNYKVDWYQQRPGKGP |
| | | | RPVMRVGTGGIVGSKGDGIPDRFSVLGSGLNRYLTIKNIQEEDE |
| | | | SDYHCGADHGSGSNFEYVVFGGGTKLTVL |
| COV2- | 287 | heavy | EVQLVESGGGLVQPGGSLRLSCAASGFTVSSNYMSWVRQAPGKG |
| 2143 | | | LEWVSVIYSAGSTYYADSVKGRFSISRDKSKNTLYLQMNSLRAE |
| | | | DTAVYYCAKEGGSGSLRYYYYGMDVWGQGTTVTVSS |
| | 288 | light | QSVVTQPPSASGTPGQRVTISCSGSSSNIGYNIVNWYQQLPGTA |
| | | | PKLLIYSNNQRPSGVPDRFSGSKSGTSASLSISGLQSEDEADYY |
| | | | CAAWDDSLNGYVFGTGTKVTVL |
| COV2- | 289 | heavy | QVQLVESGGGVVQPGRSLRLSCAASGFTFSTYAMHWVRQAPGKG |
| 2145 | | | LEWVAVISYDGSNKYYADSVKGRFTTSRDNSKNTLYLQMIGLRA |
| | | | EDTAVYYCARDWAPTYYDMPSAFDIWGQGTMVTVSS |
| | 290 | light | SYVLTQPPSVSVAPGKTARITCGGNNIGNKGVHWYQQKPGQAPV |
| | | | LVVDDDSDRPSGIPERFSGSNSGNTATLIISSVEVGDEADFYCQ |
| | | | VWDSSSDHPGVFGGGTKITVL |
| COV2- | 291 | heavy | EVQLVESGGGLVQPGGSLRLSCEASGFTFSSSEINWVRQAPGKG |
| 2146 | | | LEWVSHISSSGSIIYYADSVKGRFTISRDNAKNSLYLQMNSLRA |
| | | | EDTAVYYCARRSYRSSWYYYYGMDVWGQGTTVTVSS |
| | 292 | light | DIQLTQSPSFLSASVGDRVTITCRASQGISSYLAWYQQKPGKAP |
| | | | KLLIYAASTLQSGVPSRFSGSGSGTEFTLTISSLQPEDFATYYC |
| | | | QQLNSYPVTFGQGTKVEIK |
| COV2- | 293 | heavy | QVQLAESGGGVVQPGRSLRLSCAASGFTFSSYAMHWVRQAPGKG |
| 2147 | | | LEWVAVISYDGSNKYYADSVKGRFTISRDNSKNTLYLQMNSLRA |
| | | | EDTAVYYCARSTSGSYYYGMDVWGQGTTVTVSS |
| | 294 | light | QSALTQPASVSGSPGQSITISCTGTSSDVGDYNYVSWYQQHPGK |
| | | | APKLMIYDVSNRPSGVSNRFSGSKSGNTASLTISGLQAEDEAEY |
| | | | YCSSYTSSSTLLYVFGTGTKVTVL |
| COV2- | 295 | heavy | QVQLVQSGAEVRKPGSSVKVSCKASGGTFSSYAISWVRQAPGQG |
| 2151 | | | LEWMGGIIPVFGTANYAQKFQGRVTITADKSTSTAFMELNSLRS |
| | | | EDTAVYYCARIGSYPEYPQHWGQGTLVTVSS |
| | 296 | light | ETVLTQSPATLSLSPGERATLSCRASQSVSSFLAWYQQKPGQAP |
| | | | RLLIYDASNRATGIPARFSGSGSGTDFTLTISSLEPEDFAVYYC |
| | | | HYRSNWPPVLTFGGGTKVEI |
| COV2- | 297 | heavy | QVQLVQSGAEVKKPGSSVKVSCKASGGTFSSYAIIWVRQAPGQG |
| 2153 | | | LEWMGGIIPIFGTTNYAQKFQGRVTITADESTSTAYVELSSLRS |
| | | | EDTAVYYCARIGHFDSSGYYLDYWGQGTLVTVSS |
| | 298 | light | EIVLTQSPATLSLSPGERATLSCRASQSVSSFLAWYQQKPGQAP |
| | | | RLLIYDASNRPTGIPARFTGSGSGTDFTLTISSLEPEDFAVYYC |
| | | | QHRTNWPPLPTFGPGTKVDIK |
| COV2- | 299 | heavy | QVQLVESGGGVVQPGRSLRLSCAASGFTFSSYALFWVRQAPGKG |
| 2155 | | | LEWVAVISYDGNNKYYADSVRGRFTISRDNSKNTLYLQMNSLRP |
| | | | EDTAVYYCARPYTGSYKSYMDVWGKGTTVTVSS |
| | 300 | light | DIVMTQSPDSLAVSLGERATINCKSSQSVLYSSNNKNSLAWYQQ |
| | | | KPGQPPKLLIYWASTRESGVPDRFSGSGSGTDFTLTISSLQAED |
| | | | VAVYYCQQYYSISWTFGQGTKVEIK |
| COV2- | 301 | heavy | QVQLQESGPGLVKPSQTLSLTCTVSGGSISSGGYFWSWIRQHPG |
| 2158 | | | KGLEWIGSIYYSGSTYYNPSLRSRITISVDTSKNQFSLKLSSVT |
| | | | AADTAVYYCARGGSGSYSLFDYWGQGTLVTVSS |
| | 302 | light | DIQMTQSPSSLSASVGDRVTITCQASQDITNYLNWYQQKPGKAP |
| | | | KLLIYDASNLETGVPSRFSGSGSGTDFTFTISSLQPEDFATYYC |
| | | | QQYDNLYSVHFGQGTKLEIK |
| COV2- | 303 | heavy | QVQLVESGGGVVQPGRSLRLSCAASGFTFSRHAMHWVRQAPGKG |
| 2161 | | | LEWVAVISYDGSNKYYADSVKGRFAISRDNSKNTLYLQMNSLRP |
| | | | EDTAVYYCARDPSPLVLITSIDYWGQGTLVTVSS |
| | 304 | light | SYELTQPPSVSVSPGQTARITCSGDALPRQYTYWYQQKPGQAPV |
| | | | LVIYKDSERPSGIPERFSGSSSGTTVTLTISGVQAEDEADYYCQ |
| | | | SADTIGTYWVFGGGTKLTVL |
| COV2- | 305 | heavy | EVQLVESGGGLVQPGGSLRLSCAASGFTFSSYWMTWVRQAPGKG |
| 2162 | | | LEWVANTRQDGSERYYVDSVKGRFTTSRDNAKNSLSLQMNSLRV |
| | | | EDTAVYYCVRLGVSSWYPDYWGQGTLVTVSS |
| | | light | SYELTQPPSVSVSPGQTASITCSGDKLGDKYACWYQQKPGQSPV |
| | | | LVIYQDTKRPSGIPERFSGSNSGNTATLTISGTQAMDEADYYCQ |
| | | | AWGSSRGVFGGGTKLTVL |
| COV2- | 307 | heavy | QVQLVQSGAEVKKPGSSVKVSCKASGGTFSSYAISWVRQAPGQG |
| 2164 | | | LEWMGGIIPIFGAANYAQNFQGRVTITADESTSTGYMQLSSLRF |
| | | | EDTAVYYCARTSHYDSSGSYFEYWGQGTLVTVSS |
| | 308 | light | EIVLTQSPATLSLSPGERATLSCRASQSVSSYLAWYQQKPGQAP |
| | | | RLLIYDASNRATGIPARFSGSGSGTDFTLTISSLDPEDFAVYYC |
| | | | HKRSNWPPSLTFGGGTKVEI |
| COV2- | 827 | heavy | EVQLVESGGDLVQPGGSLRLSCAASGFTFRSYAMSWVRQAPGKG |
| 2000 | | | LEWVSAISDNAYSTYYADSVKGRFTISRDNSKNTLYLQMNSLRA |
| | | | EDTAIYYCAKNLYSGNSPFDYWGQGTLVTVSS |
| | 828 | light | XXVLTQPPSVSVSPGQTASITCSGDKLGDKYACWYQQKPGQSPV |
| | | | LVIYQDSKRPSGIPERFSGSNSGNTATLTISGTQAMDEADYYCQ |
| | | | AWDSSTAVFGTGTKVTVI |
| COV2- | 829 | heavy | EVQLVESGGGVVQPGRSLRLSCAASGFTFSSYGMHWVRQAPGKG |
| 2001 | | | LEWVAVIWHDGSKKYYADSVKGRFTISRDNSKNTLYLQMNSLRA |
| | | | EDTAVYYCARDQGGYDYVWGSYRYTFYVFDYWGQGTLVTVSS |
| | 830 | light | XXVLTQDPAVSVALGQTVRITCQGDSLRNYYASWYQQKPGQAPV |
| | | | VVMYGKNNRPSGIPDRVSGSSSGNTASLTITGAQAEDEADYYCN |
| | | | SRDSSGNHLIFGGGTKLTVL |
| COV2- | 831 | heavy | EVQLVESGGGLVQPGGSLRLSCAASGFTFSFYWMSWVRQAPGKG |
| 2002 | | | LEWVANTRQDGSERYYVDSVKGRFSTSRDNAKNSLYLQMNSLRA |
| | | | EDTAVYYCARVGSSSWYPDYWGQGTLVTVSS |
| | 832 | light | XXVLTQPPSVSVSPGQTASITCSGDKLGDKYACWYQQKPGQSPV |
| | | | LVIYQDIKRPSGIPERFSGSNSGNTATLTISGTQAMDEADYYCQ |
| | | | AWDSNTGVFGGGTKLTVL |
| COV2- | 833 | heavy | EVQLVQSGAEVKKPGASVKVSCKASGYTLTRYDIHWVRQATGQG |
| 2003 | | | LEWMGWLNPNGGNTGYAQKFQGRVTMTRNTAISTAYMELSSLRS |
| | | | EDAAVYYCARGQWELDAWYFDLWGRGTLVTVSS |
| | 834 | light | QXVLTQDPAVSVALGQTVRITCQGDSFRSYYASWYQQKPGQAPV |
| | | | LVIYGKNNRPSGIPDRFSGSSSGNTASLTITGAQAEDEADYYCN |
| | | | SRDISGNHLDVVFGGGTKLTVL |
| COV2- | 835 | heavy | EVQLVQSGAEVKKPGASLKVSCKASGYTFTRYDINWVRQATGQG |
| 2004 | | | LEWMGWMNPNSDNTGYAQKFQDRVTMTRNTSISTVYMELSSLRS |
| | | | EDTAVYYCARGQWELDVWYFDLWGRGTLVTVSS |
| | 836 | light | QXVLTQDPAVSVALGQTLRITCQGDSLRSYYASWYQQKPGQAPV |
| | | | LVIYGKNNRPSGIPDRFSGSSSGNTASLTITGAQAEDEADYYCN |
| | | | SRDSSGHHLDVVFGGGTKLTVL |
| COV2- | 837 | heavy | QVQLQESGPGLVKPSETLSLTCTVSGGSISSYYWSWIRQPPGKG |
| 2005 | | | LEWIGCIYYSGRTNYSPSLKSRVTISVDTSKNLFSLKLSSVTAA |
| | | | DTAVYYCARGGRPGAEGPYDAFDIWGQGTMVTVSS |
| | 838 | light | QXVLTQDPAVSVALGQTVRITCQGDNLRRYYASWYQQKPGQAPV |
| | | | LVIYGKNNRPSGIPDRFSGSSSGNTASLTITGAQAEDEADYYCN |
| | | | SRDSSGNHVVFGGGTTLTVL |
| COV2- | 839 | heavy | QVQLVQSGSELKKPGASVKVSCKASGYTFSDYAMNWVRQAPGQG |
| 2954 | | | LEWMGWMKSNSGNTGYAQKFQGRVTMTRNTSISTAYMELTSLRS |
| | | | EDTAVYYCARMRSGWPTHGRPDDHWGRGALVTVSS |
| | 840 | light | DIQMTQSPSSLSASVGDRVTITCRASQSIISYLNWYHQKPGKAP |
| | | | KLLIYAASSLQSGVPSRFSGSGSGTDFTFTISSLQPEDIATYYC |
| | | | QQSDNLPMYTFGQGTKLEIK |
| COV2- | 841 | heavy | QVQLVESGGGVVQPGRSLRLSCAASGFTFVTSGIHWVRQAPGKG |
| 2956 | | | LEWVAVISYDGSNKYYADSVKGRPTISRDNSKNTLYLQMNSLRA |
| | | | EDTAVYYCAKGGPNKEVLYFGELLDYGMDVWGQGTTVTVSS |
| | 842 | light | DIVMTQSPDSLAVSLGERATINCKSSQSVLYSSEIKNYLAWYQQ |
| | | | KPGQPPKLLIYWASTREFGVPDRFSGSGSGTDFTLTISSLQAED |
| | | | VAVYYCQQYYSGPLDTFGQGTKLEIK |
| COV2- | 843 | heavy | QVQLVQSGAEVKKPGASVKVSCKVSGYTFTGYVVHWVRQAPGQD |
| 2957 | | | LEWMGWINTGYGNTKYSQKFQGRVTISWDTSATTAYMELSNLKS |
| | | | EDKAVYYCASMNRMSEQTYYGMDVWGQGTTVTVS |
| | 844 | light | DIQMTQSPSSLSASVGDRVTITCQASQDISNYLNWYQQKPGKAP |
| | | | KLLIYDASNLETGVPSRFSGSGSGTDFTFTISSLQPEDIATYYC |
| | | | QQYDKLTLGGGTKVEIK |
| COV2- | 845 | heavy | QVQLQQWGAGLLKPSETLSLTCAVYGGSFSGYYWSWIRQPPGKG |
| 2958 | | | LEWTGEINHSGSTNYNPSLKSRVTTSVDTSKNHFSLKMNSVTAA |
| | | | DTAVYYCARCRQMGNFYYYYMDVWGKGTTVTVS |
| | 846 | light | QSVLTQPPSVSGVPGQRVTVSCTGSSSNIGAGFDVYWYQQFLGT |
| | | | APKLLIYGNNNRPSGVPDRFSASKSGTSASLAITGLQAEDEADY |
| | | | YCQSFDIGRGGWIFGGGTKLTVL |
| COV2- | 847 | heavy | QVHLQESGPGLVKPSETLSLTCTVSGGSINNYYWSWIRQPPGKG |
| 2959 | | | LEWIGEIHYSGSTSYSPSLKSRLSISVDRSKNQPSLKLASVTAA |
| | | | DTAVYYCVRDNYFDNSGHPVYPVPWFDPWGQGTLVTVSS |
| | 848 | light | QSVLTQPPSVSGVPGQRVTVSCTGSSSNIGAGFDVYWYQQFLGT |
| | | | APKLLIYGNNNRPSGVPDRFSASKSGTSASLAITGLQAEDEADY |
| | | | YCQSFDLGRGGWIFGGGTKLTV |
| COV2- | 849 | heavy | QVQVVQSGAEVKKPGASVKVSCKASGYTFKNYGISWVRQAPGQG |
| 2960 | | | LEWMGWISAYTGNTNYAQKFQGRMTMTTDTSTGTGYMELRSLRS |
| | | | DDTAVYYCARVQRRRLDYWGQGTLVIVSS |
| | 850 | light | DIVVTQTPLSLSVTPGQPASISCKSSETLLHSDGKTYLSWYLQK |
| | | | PGQPPQLLIYEVSNRFSGVPDRFSGSGSGTDFTLKIGRVEAEDV |
| | | | GLYYCMQSIQLAFGQGTRLEIE |
| COV2- | 851 | heavy | EVQLVESGGGVVQPGRSLRLSCAASGFTFSTYAMHWVRQAPGKGL |
| 2150 | | | EWVAVISYDGSNKYYADSVKGRFTISRDNSKNTLYLQMIGLRAED |
| | | | TAVYYCARDWAPTYYDMPSAFDIWGQGTMVTVSS |
| | 852 | light | SYELTQPPSVSVAPGKTARITCGGNNIGNKGVHWYQQKPGQAPVI |
| | | | VVDDDSDRPSGIPERFSGSNSGNTATLIISSVEVGDEADFYCQVW |
| | | | DSSSDHPGVFGGGTKLTVI |
| COV2- | 853 | heavy | EVQLVESGGGVVQPGRSLRLSCAASGFTFSSYAMHWVRQAPGKGI |
| 2159 | | | EWVAVISYDGSNKYYADSVKGRFTTSRDNSKNTLYLQMNSLRAED |
| | | | TAVYYCARSTSGSYYYGMDVWGQGTTVTVSS |
| | 854 | light | QSALTQPASVSGSPGQSITISCTGTSSDVGDYNYVSWYQQHPGKA |
| | | | PKLMIYDVSNRPSGVSNRFSGSRSGNTASLTISGLQAEDEAEYYC |
| | | | SSYTSSSTLLYVPGTGTKVTVL |
| COV2- | 855 | heavy | EVQLVESGGGVVQPGRSLRLSCAASGHTPSSYAMHWVRQAPGKGL |
| 2160 | | | EWVAVISYDGSNKYYADSVKGRFTTSRDNSKNTLYLQMNSLRAED |
| | | | TAVYYCARSTSGSYYYGMDVWGQGTTVTVSS |
| | 856 | light | QSALTQPASVSGSPGQSITISCTGTSSDVGDYNYVSWYQQHPGKA |
| | | | PKLMIYDVSNRPSGVSNRFSGSKSGNTASLTISGLQAEDEAEYYC |
| | | | SSYTSSSTLLYVFGTGTKVTVL |
| COV2- | 857 | heavy | EVQLVQSGAEVKKPGSSVKVSCKASGGTFSSYAIIWVRQAPGQGL |
| 2166 | | | EWMGGIIPIFGTTNYAQKFQGRVTITADESTSTAYVELSSLRSED |
| | | | TAVYYCARIGHFDSSGYYLDYWGQGTLVTVSS |
| | 858 | light | EIVLTQSPATLSLSPGERATLSCRASQSVSSFLAWYQQKPGQAPR |
| | | | LLIYDASNRPTGIPARFTGSGSGTDFTLTISSLEPEDFAVYYCQH |
| | | | RTNWPPLPTFGPGTKVDIK |
| COV2- | 859 | heavy | EVQLVQSGAEVKKPGSSVKVSCKASGGTFSSYAISWVRQAPGQGL |
| 2169 | | | EWMGGIIPIFGAANYAQNFQGRVTITADESTSTGYMQLSSLRPED |
| | | | TAVYYCARTSHYDSSGSYFEYWGQGTLVTVSS |
| | 860 | light | EIVLTQSPATLSLSPGERATLSCRASQSVSSYLAWYQQKPGQAPR |
| | | | LLIYDASNRATGIPARFSGSGSGTDPTLTISSLDPEDFAVYYCHK |
| | | | RSNWPPSLTPGGGTKVEIK |
| COV2- | 861 | heavy | EVQLVESGGGLVQPGGSLRLSCVASGFTFSPYWMSWVRQAPGKGL |
| 2175 | | | EWVANIKQDGGEKYYVDSVKGRPTISRDNAKNSLYLQMNSLRAED |
| | | | TAVYYCARLSGSSWDFDYWGQGTLVTVSS |
| | 862 | light | SYELTQPPSVSVSPGQTASITCSGDKLGDKYACWYQQRPGQSPVL |
| | | | VIYQDSKRPSGIPERFSGSNSGNTATLTISGTQAMDEADYYCQAW |
| | | | DSSTGVFGTGTKVTVL |
| COV2- | 863 | heavy | EVQLVESGGGVVRPGGSLRLSCAASGFTFDDYGMSWVRQAPGKGL |
| 2191 | | | EWVSAINWNGGSTGYADSVKGRFTISRDNAKNSLYLQMNSLRAED |
| | | | TALYHCARRRSSSRYSSGWYMYYYYMDVWGKGTTVTVSS |
| | 864 | light | DIQMTQSPSTLSASVGDRVTITCRASQSVSTWLAWYQQKPGKAPN |
| | | | LLIYEASSLESGVPSRFSGSGSGTEFTLTISSLQPDDFATYYCQQ |
| | | | YNTYSGTFGQGTKVEIK |
| COV2- | 865 | heavy | EVQLVESGGGVVRPGGSLRLSCAASGFTFDDYGMSWVRQAPGKGL |
| 2194 | | | EWVSAINWNGGSTGYADSVKGRFTTSRDNAKNSLYLQMNSLRAED |
| | | | TALYHCARRRSSSRYSSGWYMYYYYMDVWGKGTTVTVSS |
| | 866 | light | DIQMTQSPSTLSASVGDRVTITCRASQSVSTWLAWYQQKPGKAPN |
| | | | LLIYEASSLESGVPSRFSGSGSGTEFTLTISSLQPDDFATYYCQQ |
| | | | YNTYSGTFGQGTKVEIK |
| COV2- | 867 | heavy | EVQLVQSGAEVKKPGSSVKVSCKASGGTFSSYAISWVRQAPGQGI |
| 2198 | 868 | | EWMGGIIPIFGAANYAQNFQGRVTITADESTSTGYMQLSSLRFED |
| | | | TAVYYCARTSHYDSSGSYFEYWGQGTLVTVSS |
| | | light | EIVLTQSPATLSLSPGERATLSCRASQSVSSYLAWYQQKPGQAPR |
| | | | LLIYDASNRATGIPARFSGSGSGTDFTLTISSLDPEDFAVYYCHK |
| | | | RSNWPPSLTFGGGTKVEIK |
| COV2- | 869 | heavy | EVQLVESGGGVVQPGRSLRLSCAASGFTFSTYAMRWVRQAPGKGL |
| 2203 | | | AWVALISYDGYNKYYADSVRGRFTISRINSKNTLSLQMNSLRAED |
| | | | TAVYYCARGSAGNYYYGMDVWGQGTTVTVSS |
| | 870 | light | DIQMTQSPSSLSASVGDRVTITCRASQTITNYLNWYQLKSGRAPK |
| | | | LLIYAASSLQSGVPSRFSGSGSGTDFTLTISSLQPEDFATYYCQQ |
| | | | SYSTPYTPGQGTKVEIK |
| COV2- | 871 | heavy | EVQLVQSGAEVKKPGSSVKVSCKASGGTFSSYAIIWVRQAPGQGL |
| 2214 | | | EWMGGIIPIFGTTNYAQKFQGRVTITADESTSTAYVELSSLRSED |
| | | | TAVYYCARIGHFDSSGYYLDYWGQGTLVTVSS |
| | 872 | light | EIVLTQSPATLSLSPGERATLSCRASQSVSSFLAWYQQKPGQAPR |
| | | | LLIYDASNRPTGIPARFTGSGSGTDFTLTISSLEPEDFAVYYCQH |
| | | | RTNWPPLFTFGPGTKVDIK |
| COV2- | 873 | heavy | EVQLVQSGAEVKKPGSSVRVSCKASGGTFSSYAISWVRQAPGQGI |
| 2216 | | | EWMGGIIPIFGAANYAQNFQGRVTITADESTSTGYMQLSSLRFED |
| | | | TAVYYCARTSHYDSSGSYFEYWGQGTEVTVSS |
| | 874 | light | EIVLTQSPATLSLSPGERATLSCRASQSVSSYLAWYQQKPGQAPR |
| | | | LLIYDASNRATGIPARFSGSGSGTDFTLTISSLDPEDFAVYYCHK |
| | | | RSNWPPSLTFGGGTKVEIK |
| COV2- | 875 | heavy | EVQLVESGGGVVQPGRSLRLSCAASGFTFSSYALFWVRQAPGKGL |
| 2218 | | | EWVAVISYDGNNKYYADSVRGRFTISRDNSKNTLYLQMNSLRPED |
| | | | TAVYYCARPYTGSYKSYMDVWGKGTTVTVSS |
| | 876 | light | DIVMTQSPDSLAVSLGERATINCKSSQSVLYSSNNKNSLAWYQQK |
| | | | PGQPPKLLIYWASTRESGVPDRFSGSGSGTDFTLTISSLQAEDVA |
| | | | VYYCQQYYSISWTFGQGTKVEIK |
| COV2- | 877 | heavy | EVQLVESGGGVVQPGRSLRLSCAASGFTFSTYGMHWVRQAPGKGL |
| 2224 | | | EWVAVISYDGSNKYYADSVKGRFTISRDNSKNTLYLQMNSLRAED |
| | | | TAMYYCAKDGSIAAADYWGQGTLVTVSS |
| | 878 | light | DIVMTQSPDSLAVSLGERATINCKSSQSVLHSSNNKDSLVWYQQK |
| | | | PGQPPKLLIYWASSRESGVPDRFSGSGSGTDFTLTTSSLQAEDVA |
| | | | VYYCQQYYSTPWTFGQGTKVEIK |
| COV2- | 879 | heavy | EVQLVESGGGLIQPGGSLRLSCAASGFIVSSNYMSWVRQAPGKGL |
| 2235 | | | EWVSVIYSGGSTYYADSVKGRFTISRDNSKNTLYLQMNSLRAEDT |
| | | | AVYYCARESTQWGQGTLVTVSS |
| | 880 | light | DIQMTQSPSTLSASVGDRVTITCRASHSISSWLAWYQQKPGKAPK |
| | | | LLIYKASSLESGVPSRFSGSGSGTEFTLTISSLQPDEFATYYCQQ |
| | | | YNTYSQTFGQGTKVEIK |
| COV2- | 881 | heavy | EVQLVQSGPEVKKPGTSVKVSCKASGFTFMSSAVQWVRQARGQRL |
| 2961 | | | EWIGWIVIGSGNINYAQKFQERVTITRDMSTSTAYMELSSLRSED |
| | | | TAVYYCAAPYCSSISCNDGPDIWGQGTMVTVSS |
| | 882 | light | EIVLTQSPGTLSLSPGERATLSCRASQSVSSSYLAWYQQKPGQAP |
| | | | RLLIYGASSRATGIPDRFSGSGSGTDFTLTISRLEPEDFAVYYCQ |
| | | | HYGSSRGWTFGQGTKVEIK |

TABLE 3
HEAVY CHAIN SEQUENCES

| | CDRH1 | CDRH2 | CDRH3 |
|---|---|---|---|
| Clone | SEQ ID NO: | SEQ ID NO: | SEQ ID NO: |
| COV2-2171 | GFTFSFYW | IKQDGGSK | ARLSGSSWDFDY |
| | 312 | 313 | 314 |
| COV2-2173 | GFTFDDYG | INWNGGST | ARRRSSSRYSSGWYMYYYYMDV |
| | 315 | 316 | 317 |
| COV2-2177 | GFSVSTSGEG | IYWDDDK | AHRLWFRDAFDI |
| | 318 | 319 | 320 |
| COV2-2178 | GFTFSTYW | IKQDGSEK | ARVGSSSWYFDY |
| | 321 | 322 | 323 |
| COV2-2179 | GGSISSGTYY | TYYGGST | ARRGNYYDSKNWFDR |
| | 324 | 323 | 326 |
| COV2-2181 | GFTFSSHG | IWYDGSNK | ARESADISSRLDY |
| | 327 | 328 | 329 |
| COV2-2183 | GFTFSSYA | ISYDGSNK | ARADTMVRGTYFEY |
| | 330 | 331 | 332 |
| COV2-2184 | GFSLSTSGVG | IYWDDDK | AHRLPTPQLLPSPENWFDP |
| | 333 | 334 | 335 |
| COV2-2185 | GFTFSSYW | INSDGSST | AREVEQLAHMVDY |
| | 336 | 337 | 338 |
| COV2-2186 | GFTFSSYA | ISYDGINK | ARPRSGSYYAYFDY |
| | 337 | 340 | 341 |
| COV2-2187 | GFTFSYYP | TSYDGTNK | ARGGATNFDY |
| | 342 | 343 | 344 |
| COV2-2189 | GFTFDDSA | ISWNSGNV | TKASRYCSSTICYWNWFDP |
| | 345 | 346 | 347 |
| COV2-2190 | GFTFSSYE | ISSSGSAI | AREARSRYPDWLPSYYFDY |
| | 348 | 349 | 350 |
| COV2-2192 | GYTFTTYA | INTNTGNP | ARDQDSGYPTYYYYYMDV |
| | 351 | 352 | 353 |
| COV2-2193 | GFTFSTYA | ISYDGYNK | ARGSAGNYYYGMDV |
| | 354 | 355 | 356 |
| COV2-2195 | GFTFSNYG | ISNDEFNK | AKGGDGSGWAWDGDNPPTDY |
| | 357 | 358 | 359 |
| COV2-2197 | GYSFTSYW | IYPGDSDT | ARPDYSSGWFSYWYPDL |
| | 363 | 364 | 365 |
| COV2-2199 | GYTLTELS | FDPEDAET | ATGFAVFGRAAVPY |
| | 366 | 367 | 368 |
| COV2-2200 | GFTFSSYG | ISYDGSNK | AKDLTIVVIPAAPNFDY |
| | 369 | 370 | 371 |
| COV2-2207 | GYSFTSHW | IYPGDSDT | ASALRERGVQLWSV |
| | 372 | 373 | 374 |
| COV2-2210 | GFTFSSYA | ISYDGSNK | ARDQEWFRELFLFDY |
| | 375 | 376 | 377 |
| COV2-2211 | GFTFSTYG | ISYDGSNK | AKDGSIAAADY |
| | 378 | 379 | 380 |
| COV2-2212 | GFTFSSYS | ISNSNSFI | ARVNGNSNWNFGSYYYYYMDV |
| | 381 | 382 | 383 |
| COV2-2215 | GFTFSGYS | ISSSSSYI | ARWLQLRSDYYYFGMDV |
| | 384 | 385 | 386 |
| COV2-2222 | GGSISSYY | IYYSGST | ARAPRERLQWGEYYFDY |
| | 387 | 388 | 389 |
| COV2-2226 | GYSFTNSW | IYPGDSDT | ATHRCSGGFCYLAY |
| | 390 | 391 | 392 |
| COV2-2227 | GFTFSSYG | IWYDGSKK | ARDQSQGAYILTGYRGYGMDV |
| | 393 | 394 | 395 |
| COV2-2228 | GGSFSGHY | INHSGST | ARPPQAARIHYYYYMDV |
| | 396 | 397 | 398 |
| COV2-2231 | GFTFDDYA | ISWNSDSI | ARGKGAGYTSYMDV |
| | 399 | 400 | 401 |
| COV2-2233 | LFRSGGHW | IYPGDSDT | ASALRERGVQLWSV |
| | 402 | 403 | 404 |
| COV2-2046 | GFTFDDYA | ISWNSGSI | AKAHSTGHQYYYGMDV |
| | 405 | 406 | 407 |
| COV2-2047 | GFTFDDYA | ISWNSGSI | AKVSSITSLLGYYFDS |
| | 408 | 409 | 410 |
| COV2-2048 | GDTSSSYT | IIPILGIA | ARGVVAATPGWFDP |
| | 411 | 412 | 413 |
| COV2-2049 | GFTFSSYW | INSDGSST | AGSPWLRGDIDY |
| | 414 | 415 | 416 |
| COV2-2050 | GYTFTDYY | INPNSRGT | ARVVVIGYGRPNNYYDGRNVWDY |
| | 417 | 418 | 419 |
| COV2-2051 | GFSLGTSGMC | IDWDDDK | ARGVVTYDY |
| | 420 | 421 | 422 |
| COV2-2054 | GDTFSSYT | IIPILGIP | ARGRGYSNYGASYYMDV |
| | 423 | 424 | 425 |
| COV2-2055 | GFTFDDYA | ISWNSDSI | AKGRGAGYTSYMDV |
| | 426 | 427 | 428 |
| COV2-2064 | GYTFTSYD | MNSNSGNA | ARMRTGWPTHGRPDDE |
| | 432 | 433 | 434 |
| COV2-2068 | GFTVSSNY | IYPGGSA | ARSYDILTGYRDAFDT |
| | 435 | 436 | 437 |
| COV2-2069 | GLTVSSNY | IYAGGNT | ARGDGGYYSPFDY |
| | 438 | 439 | 440 |
| COV2-2070 | GGSFSAYY | INHSGST | ARVGYSQGYYYYYMDV |
| | 441 | 442 | 443 |
| COV2-2078 | GGTFSSYS | IIPVLGIA | ARVGVSGFKSGSNWYFDL |
| | 447 | 448 | 449 |
| COV2-2081 | GYTFTSYY | INPGGGST | ARGAIPPNSRAEIDY |
| | 453 | 454 | 455 |
| COV2-2082 | GFIFDDYD | ISWNGGNT | AVIMSPIPRYSGYDWAGGAFDI |
| | 456 | 457 | 458 |
| COV2-2083 | GFTFSNYG | MSYDGSNK | AKNLGPYCSGGTCYSLVGDY |
| | 459 | 460 | 461 |
| COV2-2097 | GFTFDDYA | ISWNSGTI | AKDIIRQGEDGMDV |
| | 468 | 469 | 470 |
| COV2-2098 | GFTFSNYA | IISTSGGAT | VKGLFDWFPL |
| | 471 | 472 | 473 |
| COV2-2103 | GFTFSRHW | IKQDGSEK | ARLGFYYGGADY |
| | 474 | 475 | 476 |
| COV2-2108 | GFTFHHYA | ISGSSDYR | AKGVDYGGKLAYFDS |
| | 477 | 478 | 479 |
| COV2-2110 | GFSFSSYV | ISYDGSSK | ARDIDSGYDPIPVFDY |
| | 480 | 481 | 482 |
| COV2-2111 | GFTFSSYD | IGTAGDT | ARVLYDSSGPYNWFDP |
| | 483 | 484 | 485 |
| COV2-2113 | EVTVSSNY | IYSGGTT | ARDFLRWHDL |
| | 486 | 487 | 488 |
| COV2-2114 | GDTFSSYT | IIPILGIP | ARGRGYSNYGASYYMDV |
| | 489 | 490 | 491 |
| COV2-2128 | GGSISSSSYY | IYYSGST | ARILVIFTLNWFDP |
| | 492 | 493 | 494 |
| COV2-2132 | EVTVSSNY | IYSGGTT | ARDFLRWHDL |
| | 498 | 499 | 500 |
| COV2-2137 | GGSVSSGSYY | IYYSGSS | AGSPVPPTIVGASY |
| | 501 | 502 | 503 |
| COV2-2142 | GYSFTSYW | IYPGDSDT | ARRGEAAGIWYFDL |
| | 504 | 505 | 506 |
| COV2-2143 | GFTVSSNY | IYSAGST | AKEGGSGSLRYYYYGMDV |
| | 507 | 508 | 509 |
| COV2-2145 | GFTFSTYA | ISYDGSNK | ARDWAPTYYDMPSAFDI |
| | 510 | 511 | 512 |
| COV2-2146 | GFTFSSSE | ISSSGSII | ARRSYRSSWYYYYGMDV |
| | 513 | 514 | 515 |
| COV2-2147 | GFTFSSYA | ISYDGSNK | ARSTSGSYYYGMDV |
| | 516 | 517 | 518 |
| COV2-2151 | GGTFSSYA | IIPVFGTA | ARIGSYPEYFQH |
| | 519 | 520 | 521 |
| COV2-2153 | GGTFSSYA | IIPIFGTT | ARIGHFDSSGYYLDY |
| | 522 | 523 | 524 |
| COV2-2155 | GFTFSSYA | ISYDGNNK | ARPYTGSYRSYMDV |
| | 525 | 526 | 527 |
| COV2-2158 | GGSISSGGYF | IYYSGST | ARGGSGSYSLFDY |
| | 528 | 529 | 530 |
| COV2-2161 | GFTFSRHA | ISYDGSNK | ARDPSPLVLITSIDY |
| | 531 | 532 | 533 |
| COV2-2162 | GFTFSSYW | IRQDGSEK | VRLGVSSWYFDY |
| | 534 | 535 | 536 |
| COV2-2164 | GGTFSSYA | IIPIFGAA | ARTSHYDSSGSYFEY |
| | 537 | 538 | 539 |
| COV2-2000 | GFTFRSYA | ISDNAYST | AKNLYSGNSPFDY |
| | 833 | 884 | 885 |
| COV2-2001 | GFTPSSYG | IWHDGSKK | ARDQGGYDYVWGSYRYTFYVFDY |
| | 886 | 887 | 888 |
| COV2-2002 | GFTFSFYW | IKQDGSEK | ARVGSSSWYFDY |
| | 889 | 890 | 891 |
| COV2-2003 | GYTLTRYD | LNPNGGNT | ARGQWELDAWYFDI |
| | 892 | 893 | 894 |
| COV2-2004 | GYTFTRYD | MNPNSDNT | ARGQWELDVWYFDI |
| | 895 | 896 | 897 |
| COV2-2005 | GGSISSYY | IYYSGRT | ARGGRPGAEGPYDAFDI |
| | 898 | 899 | 900 |
| COV2-2954 | GYTFSDYA | MKSNSGNT | ARMRSGWPTHGRPDDH |
| | 901 | 902 | 903 |
| COV2-2956 | GFTFVTSG | ISYDGSNK | AKGGPNKEVLYFGELLDYGMDV |
| | 904 | 905 | 906 |
| COV2-2957 | GYTFTGYV | INTGYGNT | ASMNRMSE0TYYGMDV |
| | 907 | 908 | 909 |
| COV2-2958 | GGSFSGYY | INHSGST | ARCRQMGNEYYYYMDV |
| | 910 | 911 | 912 |
| COV2-2959 | GGSINNYY | IHYSGST | VRDNYFDNSGHPVYPVPWFDP |
| | 913 | 914 | 915 |
| COV2-2960 | GYTFKNYG | ISAYTGNT | ARVQRRRLDY |
| | 916 | 917 | 918 |
| COV2-2150 | GFTFSTYA | ISYDGSNK | ARDWAPTYYDMPSAFDI |
| | 919 | 920 | 921 |
| COV2-2159 | GFTFSSYA | ISYDGSNK | ARSTSGSYYYGMDV |
| | 922 | 923 | 924 |
| COV2-2160 | GFTFSSYA | ISYDGSNK | ARSTSGSYYYGMDV |
| | 925 | 926 | 927 |
| COV2-2166 | GGTFSSYA | IIPIFGTT | ARIGHFDSSGYYLDY |
| | 928 | 929 | 930 |
| COV2-2169 | GGTFSSYA | IIPIFGAA | ARTSHYDSSGSYFEY |
| | 931 | 932 | 933 |
| COV2-2175 | GFTFSFYW | IKQDGGEK | ARLSGSSWDFDY |
| | 934 | 935 | 936 |
| COV2-2191 | GFTFDDYG | INWNGGST | ARRRSSSRYSSGWYMYYYYMDV |
| | 937 | 938 | 939 |
| COV2-2194 | GFTFDDYG | INWNGGST | ARRRSSSRYSSGWYMYYYYMDV |
| | 940 | 941 | 942 |
| COV2-2198 | GGTFSSYA | IIPIFGAA | ARTSHYDSSGSYFEY |
| | 943 | 944 | 945 |
| COV2-2203 | GPTFSTYA | ISYDGYNK | ARGSAGNYYYGMDV |
| | 946 | 947 | 948 |
| COV2-2214 | GGTFSSYA | IIPIPGTT | ARIGHFDSSGYYLDY |
| | 949 | 950 | 951 |
| COV2-2216 | GGTFSSYA | IIPIPGAA | ARTSHYDSSGSYFEY |
| | 952 | 953 | 954 |
| COV2-2218 | GFTFSSYA | ISYDGNNK | ARPYTGSYKSYMDV |
| | 955 | 956 | 957 |
| COV2-2224 | GFTFSTYG | ISYDGSNK | AKDGSIAAADY |
| | 958 | 959 | 960 |
| COV2-2235 | GFIVSSNY | IYSGGST | ARESTQ |
| | 961 | 962 | 963 |
| COV2-2961 | GFTFMSSA | IVIGSGNT | AAPYCSSISCNDGFDI |
| | 964 | 965 | 966 |

TABLE 4
LIGHT CHAIN SEQUENCES

| | CDRL1 | CDRL2 | CDRL3 |
|---|---|---|---|
| Clone | SEQ ID NO: | SEQ ID NO: | SEQ ID NO: |
| COV2-2171 | KLGDKY | QDS | QAWDSSTGV |
| | 543 | 544 | 545 |
| COV2-2173 | QSVSTW | EAS | QQYNTYSGT |
| | 546 | 547 | 548 |
| COV2-2177 | QSISNY | AAS | QQTYSTFWT |
| | 549 | 550 | 551 |
| COV2-2178 | KLGDKY | QDS | QAWDSSTAV |
| | 5b2 | 553 | 554 |
| COV2-2179 | QSVSSN | GAS | QQYNNWPPMYT |
| | 555 | 556 | 557 |
| COV2-2181 | ALPTKY | DDS | YSTDSSGNV |
| | 558 | 559 | 560 |
| COV2-2183 | SSDVGGYNY | DVS | SSYTSSRAVL |
| | 561 | 562 | 563 |
| COV2-2184 | SSNIGNNA | YDD | ASWDDSLIGPV |
| | 564 | 565 | 566 |
| COV2-2185 | ALPNQY | KDS | QSADSSGTSWV |
| | 567 | 568 | 569 |
| COV2-2186 | QGISNY | AAS | QQYNSHPPT |
| | 570 | 571 | 572 |
| COV2-2187 | NIGRKS | DDS | QVWDSSSDHPEWV |
| | 573 | 574 | 575 |
| COV2-2189 | QSISSY | GAS | QQSYSTPT |
| | 576 | 577 | 578 |
| COV2-2190 | SSDIGGYNY | DVS | SSYTSSSTHVV |
| | 579 | 580 | 581 |
| COV2-2192 | QSLLHSDGKTY | EVS | MQSIQPPLT |
| | 582 | 583 | 584 |
| COV2-2193 | QTITNY | AAS | QQSYSTPYT |
| | 585 | 586 | 587 |
| COV2-2195 | QSVLYTPRNKNY | WAS | QQYYTAPLT |
| | 588 | 589 | 590 |
| COV2-2191 | QSVSSNF | GAS | QQYGRSPIT |
| | 594 | 595 | 596 |
| COV2-2199 | KLGDKY | QGA | QAWDSSTVV |
| | 597 | 598 | 599 |
| COV2-2200 | SSDVGGYNY | DVS | SSYTSSSTPVV |
| | 600 | 601 | 602 |
| COV2-2207 | SSNIGAGYD | INS | QSYDSSLGAL |
| | 603 | 604 | 605 |
| COV2-2210 | QGISSW | DAS | QQANSFPPY |
| | 606 | 607 | 608 |
| COV2-2211 | QSVLHSSNNKDS | WAS | QQYYSTPWT |
| | 609 | 610 | 611 |
| COV2-2212 | QSVSSY | DTS | QQRGNWWT |
| | 612 | 613 | 614 |
| COV2-2215 | QSVSNN | GAS | QQCYNWPPWT |
| | 615 | 616 | 617 |
| COV2-2222 | SSDVGSYNL | EVI | CSYAVSTTYVI |
| | 618 | 619 | 620 |
| COV2-2226 | SGYSNYK | VGTGGIVG | GADHGSGSNFVEW |
| | 621 | 622 | 623 |
| COV2-2227 | QSLLHSNGYNY | LGS | MQALQTPPT |
| | 624 | 625 | 626 |
| COV2-2228 | QSVSSN | GAS | QQYNYWPPLT |
| | 627 | 628 | 629 |
| COV2-2231 | NIGSKS | DDS | QVWDSSSDHHVV |
| | 630 | 631 | 632 |
| COV2-2233 | SSNIGAGYD | INS | QSYDSSLGAL |
| | 633 | 634 | 635 |
| COV2-2046 | QSISSF | AAF | QQSYNTPYT |
| | 636 | 637 | 638 |
| COV2-2047 | QSVSSY | DAS | QHRSNWPPRLT |
| | 639 | 640 | 641 |
| COV2-2048 | QSVSSN | GAS | QQYNNFLT |
| | 642 | 643 | 644 |
| COV2-2049 | SGSIASNY | SDN | QSYDGSNHAVV |
| | 645 | 646 | 644 |
| COV2-2050 | SSNIGSNT | SNN | AAWDDSLNALV |
| | 648 | 649 | 650 |
| COV2-2051 | QSIAGY | GTT | QQSYSTDGT |
| | 651 | 652 | 653 |
| COV2-2054 | QDINHY | DAS | QQSDNLPMYT |
| | 654 | 655 | 656 |
| COV2-2055 | NIGSKS | DDS | QVWDSSSDHHVV |
| | 657 | 658 | 659 |
| COV2-2064 | NSNTGSYT | DNN | LVWDDSLNGLV |
| | 663 | 664 | 665 |
| COV2-2068 | SSNIGSGSD | GNT | QSYDSRLSGFVV |
| | 666 | 667 | 668 |
| COV2-2069 | QSISSY | AAS | QQSYSTPQT |
| | 669 | 670 | 671 |
| COV2-2070 | QSISNY | AAS | QQSYTTLLT |
| | 672 | 673 | 674 |
| COV2-2078 | NSNIGAGYD | GNS | QSYDSSLSDSV |
| | 678 | 679 | 680 |
| COV2-2081 | QSVSSN | GAS | QQYYNWPLT |
| | 684 | 685 | 686 |
| COV2-2082 | SLRSYY | DKN | NSRDSSGNAVV |
| | 687 | 688 | 689 |
| COV2-2083 | QDISNY | DAS | QQYANLPFT |
| | 690 | 691 | 692 |
| COV2-2097 | QNIASY | AAS | QQSYSTPWT |
| | 699 | 700 | 701 |
| COV2-2098 | QSVRSN | GAS | HQYNNWPQT |
| | 702 | 703 | 704 |
| COV2-2103 | SGSIASNY | EDN | QSYDGINRAWV |
| | 705 | 706 | 707 |
| COV2-2108 | QSLLHSLGYNS | LGS | MQALQTPLT |
| | 108 | 709 | 710 |
| COV2-2110 | QSISSY | AAS | QQSYSSLSIT |
| | 711 | 712 | 713 |
| COV2-2111 | QSISSY | AAS | QQSYEIPPWT |
| | 714 | 715 | 716 |
| COV2-2113 | QDINNY | DAS | QQYDNLPPV |
| | 717 | 718 | 719 |
| COV2-2114 | SSNIGAGYD | ANS | QSYDSSLSGSV |
| | 720 | 721 | 722 |
| COV2-2128 | SGSIASNY | EDN | QSYDSGNPI |
| | 723 | 724 | 725 |
| COV2-2132 | QDINNY | DAS | QQYDNLPPV |
| | 729 | 730 | 731 |
| COV2-2137 | SGSIASNY | EDN | QSYDGINRWLV |
| | 732 | 733 | 734 |
| COV2-2142 | SGYSNYK | VGTGGIVG | GADHGSGSNFEYVV |
| | 735 | 736 | 737 |
| COV2-2143 | SSNIGYNI | SNN | AAWDDSLNGYV |
| | 738 | 730 | 740 |
| COV2-2195 | NIGNKG | DDS | QVWDSSSDHPGV |
| | 741 | 742 | 743 |
| COV2-2146 | QGISSY | AAS | QQLNSYPVI |
| | 744 | 745 | 746 |
| COV2-2147 | SSDVGDYNY | DVS | SSYTSSSTLLYV |
| | 747 | 748 | 749 |
| COV2-2151 | QSVSSF | DAS | HYRSNWPPVIT |
| | 750 | 751 | 752 |
| COV2-2153 | QSVSSF | DAS | QHRTNWPPLFT |
| | 753 | 754 | 755 |
| COV2-2155 | QSVLYSSNNKNS | WAS | QQYYSISWT |
| | 756 | 757 | 758 |
| COV2-2158 | QDITNY | DAS | QQYDNLYSVH |
| | 759 | 760 | 761 |
| COV2-2161 | ALPRQY | Kns | QSADTIGTYWV |
| | 762 | 763 | 764 |
| COV2-2162 | KLGDKY | QDT | QAWGSSRGV |
| | 765 | 766 | 767 |
| COV2-2164 | QSVSSY | DAS | HKRSNWPPSLT |
| | 768 | 769 | 770 |
| COV2-2000 | KLGDKY | QDS | QAWLSSIAV |
| | 967 | 968 | 969 |
| COV2-2001 | SLRNYY | GKN | NSRDSSGNHLI |
| | 970 | 971 | 972 |
| COV2-2002 | KLGDKY | QDI | QAWDSNTGV |
| | 973 | 974 | 975 |
| COV2-2003 | SFRSYY | GKN | NSRDTSGNHLDVV |
| | 976 | 977 | 978 |
| COV2-2004 | SLRSYY | GKN | NSRDSSGHHLDVV |
| | 979 | 980 | 981 |
| COV2-2005 | NLRRYY | GKN | NSRDSSGNHVV |
| | 982 | 983 | 984 |
| COV2-2954 | QSIISY | AAS | QQSDNLPMYT |
| | 985 | 986 | 987 |
| COV2-2306 | QSVLYSSEIKNY | WAS | QQYYSGPLDT |
| | 988 | 989 | 990 |
| COV2-2957 | QDISNY | DAS | QQYDKLT |
| | 991 | 992 | 993 |
| COV2-2958 | SSNIGAGFD | GNN | QSFDIGRGGWI |
| | 994 | 995 | 996 |
| COV2-2959 | SSNIGAGFD | GNN | QSFDIGRGGWI |
| | 997 | 998 | 999 |
| COV2-2960 | ETLLHSDGKTY | EVS | MQSIQLA |
| | 1000 | 1001 | 1002 |
| COV2-2150 | NIGNKG | DDS | QVWDSSSDHPGV |
| | 1003 | 1004 | 1005 |
| COV2-2159 | SSDVGDYNY | DVS | SSYTSSSTLLYV |
| | 1006 | 1007 | 1008 |
| COV2-2160 | SSDVGDYNY | DVS | SSYTSSSTLLYV |
| | 1009 | 1010 | 1011 |
| COV2-2166 | QSVSSF | DAS | QHRTNWPPLFT |
| | 1012 | 1013 | 1014 |
| COV2-2169 | QSVSSY | DAS | HKRSNWPPSLT |
| | 1015 | 1016 | 1017 |
| COV2-2175 | KLGDKY | QDS | QAWDSSTGV |
| | 1018 | 1019 | 1020 |
| COV2-2191 | QSVSTW | EAS | QQYNTYSGI |
| | 1021 | 1022 | 1023 |
| COV2-2194 | QSVSTW | EAS | QQYNTYSGI |
| | 1024 | 1025 | 1026 |
| COV2-2198 | QSVSSY | DAS | HKRSNWPPSLT |
| | 540 | 1027 | 1028 |
| COV2-2203 | QTITNY | AAS | QQSYSTPYT |
| | 1029 | 1030 | 1031 |
| COV2-2214 | QSVSSF | DAS | QHRTNWPPLFT |
| | 1032 | 1033 | 1034 |
| COV2-2216 | QSVSSY | DAS | HKRSNWPPSLT |
| | 1035 | 1036 | 1037 |
| COV2-2218 | QSVLYSSNNKNS | WAS | QQYYSISWT |
| | 1038 | 1039 | 1040 |
| COV2-2224 | QSVLHSSNNKDS | WAS | QQYYSTPWT |
| | 1041 | 1042 | 1043 |
| COV2-2235 | HSISSW | KAS | QQYNTYSQT |
| | 1044 | 1045 | 1046 |
| COV2-2961 | QSVSSSY | GAS | QHYGSSRGWT |
| | 1047 | 1048 | 1049 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. References

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Allred et al., Arch. Surg., 125(1), 107-113, 1990.
Atherton et al., Biol. of Reproduction, 32, 155-171, 1985.
Barzon et al., Euro Surveill. 2016 Aug. 11; 21(32).
Beltramello et al., Cell Host Microbe 8, 271-283, 2010.
Brown et al., J. Immunol. Meth., 12:130(1), :111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
De Jager et al., Semin. Nucl. Med. 23(2), 165-179, 1993.
Dholakia et al., J. Biol. Chem., 264, 20638-20642, 1989.
Diamond et al., J Virol 77, 2578-2586, 2003.
Doolittle and Ben-Zeev, Methods Mol. Biol., 109, :215-237, 1999.
Duffy et al., N. Engl. J. Med. 360, 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet., 3:231-236, 1977.
Gornet et al., Semin Reprod Med. 2016 September: 34(5):285-292. Epub 2016 September 14.
Gulbis and Galand, Hum. Pathol. 24(12), 1271-1285, 1993.
Halfon et al., PLoS ONE 2010; 5 (5) e10569
Hessell et al., Nature 449, 101-4, 2007.
Khatoon et al., Ann. of Neurology, 26, 210-219, 1989.
King et al., J. Biol. Chem., 269, 10210-10218, 1989.
Kohler and Milstein, Eur. J. Immunol., 6, 511-519, 1976.
Kohler and Milstein, Nature, 256, 495-497, 1975.
Kyte and Doolittle, J. Mol. Biol., 157(1):105-132, 1982.
Mansuy et al., Lancet Infect Dis. 2016 October; 16(10):1106-7.
Nakamura et al., In: Enzyme Immunoassays: Heterogeneous and Homogeneous Systems, Chapter 27, 1987.
O'Shannessy et al., J. Immun. Meth., 99, 153-161, 1987.
Persic et al., Gene 187:1, 1997
Potter and Haley, Meth. Enzymol., 91, 613-633, 1983.
Purpura et al., Lancet Infect Dis. 2016 October; 16(10):1107-8. Epub 2016 September 19.
Remington's Pharmaceutical Sciences, 15th Ed., 3:624-652, 1990.
Tang et al., J. Biol. Chem., 271:28324-28330, 1996.
Wawrzynczak & Thorpe, In: Immunoconjugates, Antibody Conjugates In Radioimaging And Therapy Of Cancer, Vogel (Ed.), NY, Oxford University Press, 28, 1987.
Yu et al., J Immunol Methods 336, 142-151, doi:10.1016/j.jim.2008.04.008, 2008.

1. A method of detecting COVID-19 infection with SARS-CoV-2 in a subject comprising:
(a) contacting a sample from said subject with an antibody or antibody fragment having clone-paired heavy and light chain CDR sequences from Tables 3 and 4, respectively; and
(b) detecting SARS-CoV-2 in said sample by binding of said antibody or antibody fragment to a SARS-CoV-2 antigen in said sample.

2. The method of claim 1, wherein said sample is a body fluid.

3. The method of claim 1, wherein said sample is blood, sputum, tears, saliva, mucous or serum, semen, cervical or vaginal secretions, amniotic fluid, placental tissues, urine, exudate, transudate, tissue scrapings or feces.

4. The method of claim 1, wherein detection comprises ELISA, RIA, lateral flow assay or western blot.

5. The method of claim 1, further comprising performing steps (a) and (b) a second time and determining a change in SARS-CoV-2 antigen levels as compared to the first assay.

6. The method of claim 1, wherein the antibody or antibody fragment is encoded by clone-paired variable sequences as set forth in Table 1.

7. The method of claim 1, wherein said antibody or antibody fragment is encoded by light and heavy chain variable sequences having at least 70%, 80%, 90% or 95% identity to clone-paired variable sequences as set forth in Table 1.

8. The method of claim 1, wherein said antibody or antibody fragment is encoded by light and heavy chain variable sequences having 100% identity to clone-paired sequences as set forth in Table 1.

9. The method of claim 1, wherein said antibody or antibody fragment comprises light and heavy chain variable sequences according to clone-paired sequences from Table 2.

10. The method of claim 1, wherein said antibody or antibody fragment comprises light and heavy chain variable sequences having at least 70%, 80%, 90% or 95% identity to clone-paired sequences from Table 2.

11. The method of claim 1, wherein said antibody or antibody fragment binds to a SARS-CoV-2 surface spike protein.

12. The method of claim 1, wherein the antibody fragment is a recombinant scFv (single chain fragment variable) antibody, Fab fragment, F(ab′)₂ fragment, or Fv fragment.

13. A method of treating a subject infected with SARS-CoV-2 or reducing the likelihood of infection of a subject at risk of contracting SARS-CoV-2, comprising delivering to said subject an antibody or antibody fragment having clone-paired heavy and light chain CDR sequences from Tables 3 and 4, respectively.

14. The method of claim 13, the antibody or antibody fragment is encoded by clone-paired light and heavy chain variable sequences as set forth in Table 1.

15. The method of claim 13, wherein said antibody or antibody fragment is encoded by light and heavy chain variable sequences having at least 70%, 80%, 90% or 95% identity to clone-paired sequences from Table 1.

16. The method of claim 13, wherein said antibody or antibody fragment comprises light and heavy chain variable sequences according to clone-paired sequences from Table 2.

17. The method of claim 13, wherein said antibody or antibody fragment comprises light and heavy chain variable sequences having at least 70%, 80% or 90% identity to clone-paired sequences from Table 2.

18. The method of claim 13, wherein said antibody or antibody fragment comprises light and heavy chain variable sequences having at least 95% identity to clone-paired sequences from Table 2.

19. The method of claim 13, wherein the antibody fragment is a recombinant scFv (single chain fragment variable) antibody, Fab fragment, F(ab′)₂ fragment, or Fv fragment.

20. The method of claim 13, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

21. The method of claim 13, wherein said antibody is a chimeric antibody or a bispecific antibody.

22. The method of claim 13, wherein said antibody or antibody fragment binds to a SARS-CoV-2 surface spike protein.

23. The method of claim 13, wherein said antibody or antibody fragment is administered prior to infection or after infection.

24. The method of claim 13, wherein said subject is of age 60 or older, is immunocompromised, or suffers from a respiratory and/or cardiovascular disorder.

25. The method of claim 13, wherein delivering comprises antibody or antibody fragment administration, or genetic delivery with an RNA or DNA sequence or vector encoding the antibody or antibody fragment.

26. A monoclonal antibody, wherein the antibody or antibody fragment is characterized by clone-paired heavy and light chain CDR sequences from Tables 3 and 4, respectively.

27. The monoclonal antibody of claim 26, wherein said antibody or antibody fragment is encoded by light and heavy chain variable sequences according to clone-paired sequences from Table 1.

28. The monoclonal antibody of claim 26, wherein said antibody or antibody fragment is encoded by light and heavy chain variable sequences having at least 70%, 80%, 90%, or 95% identity to clone-paired sequences from Table 1.

29. The monoclonal antibody of claim 26, wherein said antibody or antibody fragment comprises light and heavy chain variable sequences according to clone-paired sequences from Table 2.

30. The monoclonal antibody of claim 26, wherein said antibody or antibody fragment comprises light and heavy chain variable sequences having at least 70%, 80%, 90%, or 95% identity to clone-paired sequences from Table 2.

31. The monoclonal antibody of claim 26, wherein the antibody fragment is a recombinant scFv (single chain fragment variable) antibody, Fab fragment, F(ab′)₂ fragment, or Fv fragment.

32. The monoclonal antibody of claim 26, wherein said antibody is a chimeric antibody, or is a bispecific antibody.

33. The monoclonal antibody of claim 26, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

34. The monoclonal antibody of claim 26, wherein said antibody or antibody fragment binds to a SARS-CoV-2 antigen such as a surface spike protein.

35. The monoclonal antibody of claim 26, wherein said antibody is an intrabody.

36. A hybridoma or engineered cell encoding an antibody or antibody fragment wherein the antibody or antibody fragment is characterized by clone-paired heavy and light chain CDR sequences from Tables 3 and 4, respectively.

37. The hybridoma or engineered cell of claim 36, wherein said antibody or antibody fragment is encoded by light and heavy chain variable sequences according to clone-paired sequences from Table 1.

38. The hybridoma or engineered cell of claim 36, wherein said antibody or antibody fragment is encoded by light and heavy chain variable sequences having at least 70%, 80%, or 90% identity to clone-paired variable sequences from Table 1.

39. The hybridoma or engineered cell of claim 36, wherein said antibody or antibody fragment is encoded by light and heavy chain variable sequences having at least 95% identity to clone-paired variable sequences from Table 1.

40. The hybridoma or engineered cell of claim 36, wherein said antibody or antibody fragment comprises light and heavy chain variable sequences according to clone-paired sequences from Table 2.

41. The hybridoma or engineered cell of claim 36, wherein said antibody or antibody fragment is encoded by light and heavy chain variable sequences having at least 70%, 80%, or 90% identity to clone-paired variable sequences from Table 2.

42. The hybridoma or engineered cell of claim 36, wherein said antibody or antibody fragment comprises light and heavy chain variable sequences having at least 95% identity to clone-paired sequences from Table 2.

43. The hybridoma or engineered cell of claim 36, wherein the antibody fragment is a recombinant scFv (single chain fragment variable) antibody, Fab fragment, F(ab′)₂ fragment, or Fv fragment.

44. The hybridoma or engineered cell of claim 36, wherein said antibody is a chimeric antibody, a bispecific antibody, or an intrabody.

45. The hybridoma or engineered cell of claim 36, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

46. The hybridoma or engineered cell of claim 36, wherein said antibody or antibody fragment binds to a SARS-CoV-2 surface spike protein.

47. A vaccine formulation comprising one or more antibodies or antibody fragments characterized by clone-paired heavy and light chain CDR sequences from Tables 3 and 4, respectively.

48. The vaccine formulation of claim 47, wherein at least one of said antibodies or antibody fragments is encoded by light and heavy chain variable sequences according to clone-paired sequences from Table 1.

49. The vaccine formulation of claim 47, wherein at least one of said antibodies or antibody fragments is encoded by light and heavy chain variable sequences having at least 70%, 80%, or 90% identity to clone-paired sequences from Table 1.

50. The vaccine formulation of claim 47, wherein at least one of said antibodies or antibody fragments is encoded by light and heavy chain variable sequences having at least 95% identity to clone-paired sequences from Table 1.

51. The vaccine formulation of claim 47, wherein at least one of said antibodies or antibody fragments comprises light and heavy chain variable sequences according to clone-paired sequences from Table 2.

52. The vaccine formulation of claim 47, wherein at least one of said antibodies or antibody fragments comprises light and heavy chain variable sequences having at least 70%, 80%, 90% or 95% identity to clone-paired sequences from Table 2.

53. The vaccine formulation of claim 47, wherein at least one of said antibody fragments is a recombinant scFv (single chain fragment variable) antibody, Fab fragment, F(ab′)₂ fragment, or Fv fragment.

54. The vaccine formulation of claim 47, wherein at least one of said antibodies is a chimeric antibody, is bispecific antibody or an intrabody.

55. The vaccine formulation of claim 47, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS YTE or LS mutation or glycan modified to alter (eliminate or enhance) FcR interactions such as enzymatic or chemical addition or removal of glycans or expression in a cell line engineered with a defined glycosylating pattern.

56. The vaccine formulation of claim 47, wherein said antibody or antibody fragment binds to a SARS-CoV-2 surface spike protein.

57. A vaccine formulation comprising one or more expression vectors encoding a first antibody or antibody fragment according to claim 26.

58. The vaccine formulation of claim 57, wherein said expression vector(s) is/are Sindbis virus or VEE vector(s).

59. The vaccine formulation of claim 57, formulated for delivery by needle injection, jet injection, or electroporation.

60. The vaccine formulation of claim 57, further comprising one or more expression vectors encoding for a second antibody or antibody fragment.

61. A method of protecting the health of a subject of age 60 or older, an immunocompromised, subject or a subject suffering from a respiratory and/or cardiovascular disorder that is infected with or at risk of infection with SARS-CoV-2 comprising delivering to said subject an antibody or antibody fragment having clone-paired heavy and light chain CDR sequences from Tables 3 and 4, respectively.

62. The method of claim 61, the antibody or antibody fragment is encoded by clone-paired light and heavy chain variable sequences as set forth in Table 1.

63. The method of claim 61, the antibody or antibody fragment is encoded by clone-paired light and heavy chain variable sequences having at least 95% identity to as set forth in Table 1.

64. The method of claim 61, wherein said antibody or antibody fragment is encoded by light and heavy chain variable sequences having at least 70%, 80%, or 90% identity to clone-paired sequences from Table 1.

65. The method of claim 61, wherein said antibody or antibody fragment comprises light and heavy chain variable sequences according to clone-paired sequences from Table 2.

66. The method of claim 61, wherein said antibody or antibody fragment comprises light and heavy chain variable sequences having at least 70%, 80% or 90% identity to clone-paired sequences from Table 2.

67. The method of claim 61, wherein said antibody or antibody fragment comprises light and heavy chain variable sequences having at least 95% identity to clone-paired sequences from Table 2

68. The method of claim 61, wherein the antibody fragment is a recombinant scFv (single chain fragment variable) antibody, Fab fragment, F(ab′)₂ fragment, or Fv fragment.

69. The method of claim 61, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

70. The method of claim 61, wherein said antibody is a chimeric antibody or a bispecific antibody.

71. The method of claim 61, wherein said antibody or antibody fragment is administered prior to infection or after infection.

72. The method of claim 61, wherein said antibody or antibody fragment binds to a SARS-CoV-2 surface spike protein.

73. The method of claim 61, wherein delivering comprises antibody or antibody fragment administration, or genetic delivery with an RNA or DNA sequence or vector encoding the antibody or antibody fragment.

74. The method of claim 61, wherein the antibody or antibody fragment improves the subject's respiration as compared to an untreated control.

75. The method of claim 61, wherein the antibody or antibody fragment reduces viral load as compared to an untreated control.

76. A method of determining the antigenic integrity, correct conformation and/or correct sequence of a SARS-CoV-2 surface spike protein comprising:
(a) contacting a sample comprising said antigen with a first antibody or antibody fragment having clone-paired heavy and light chain CDR sequences from Tables 3 and 4, respectively; and
(b) determining antigenic integrity, correct conformation and/or correct sequence of said antigen by detectable binding of said first antibody or antibody fragment to said antigen.

77. The method of claim 76, wherein said sample comprises recombinantly produced antigen.

78. The method of claim 76, wherein said sample comprises a vaccine formulation or vaccine production batch.

79. The method of claim 76, wherein detection comprises ELISA, RIA, western blot, a biosensor using surface plasmon resonance or biolayer interferometry, or flow cytometric staining.

80. The method of claim 76, wherein the first antibody or antibody fragment is encoded by clone-paired variable sequences as set forth in Table 1.

81. The method of claim 76, wherein said first antibody or antibody fragment is encoded by light and heavy chain variable sequences having at least 70%, 80%, or 90% identity to clone-paired variable sequences as set forth in Table 1.

82. The method of claim 76, wherein said first antibody or antibody fragment is encoded by light and heavy chain variable sequences having at least 95% identity to clone-paired sequences as set forth in Table 1.

83. The method of claim 76, wherein said first antibody or antibody fragment comprises light and heavy chain variable sequences according to clone-paired sequences from Table 2.

84. The method of claim 76, wherein said first antibody or antibody fragment comprises light and heavy chain variable sequences having at least 70%, 80% or 90% identity to clone-paired sequences from Table 2.

85. The method of claim 76, wherein said first antibody or antibody fragment comprises light and heavy chain variable sequences having at least 95% identity to clone-paired sequences from Table 2.

86. The method of claim 76, wherein the first antibody fragment is a recombinant scFv (single chain fragment variable) antibody, Fab fragment, F(ab′)₂ fragment, or Fv fragment.

87. The method of claim 76, further comprising performing steps (a) and (b) a second time to determine the antigenic stability of the antigen over time.

88. The method of claim 76, further comprising:
(c) contacting a sample comprising said antigen with a second antibody or antibody fragment having clone-paired heavy and light chain CDR sequences from Tables 3 and 4, respectively; and
(d) determining antigenic integrity of said antigen by detectable binding of said second antibody or antibody fragment to said antigen.

89. The method of claim 88, wherein the second antibody or antibody fragment is encoded by clone-paired variable sequences as set forth in Table 1.

90. The method of claim 89, wherein said second antibody or antibody fragment is encoded by light and heavy chain variable sequences having at least 70%, 80%, or 90% identity to clone-paired variable sequences as set forth in Table 1.

91. The method of claim 89, wherein said second antibody or antibody fragment is encoded by light and heavy chain variable sequences having at least 95% identity to clone-paired sequences as set forth in Table 1.

92. The method of claim 89, wherein said second antibody or antibody fragment comprises light and heavy chain variable sequences according to clone-paired sequences from Table 2.

93. The method of claim 89, wherein said second antibody or antibody fragment comprises light and heavy chain variable sequences having at least 70%, 80% or 90% identity to clone-paired sequences from Table 2.

94. The method of claim 89, wherein said second antibody or antibody fragment comprises light and heavy chain variable sequences having at least 95% identity to clone-paired sequences from Table 2.

95. The method of claim 89, wherein the second antibody fragment is a recombinant scFv (single chain fragment variable) antibody, Fab fragment, F(ab′)₂ fragment, or Fv fragment.

96. The method of claim 89, further comprising performing steps (c) and (d) a second time to determine the antigenic stability of the antigen over time.

97. A human monoclonal antibody or antibody fragment, or hybridoma or engineered cell producing the same, wherein said antibody binds to a SARS-CoV-2 surface spike protein.


SEQUENCE LISTING (1073 sequences)

SEQ ID NO: 1 - DNA, 20 bp, Artificial Sequence
  Synthetic primer
atgctgcaatcgtgctacaa

SEQ ID NO: 2 - DNA, 17 bp, Artificial Sequence
  Synthetic primer
gactgccgcctctgctc

SEQ ID NO: 3 - DNA, 357 bp, Artificial Sequence
  Description of Artificial Sequence: Synthetic antibody
gaggtgcagctggtggagtctgggggaggcttggtccagcctggggggtccctgagactctcctgtgtagcctctggattcacctttagtttctattggatgagctgggtccgccaggctccaggaaaggggctcgagtgggtggccaacataaagcaagatggaggtgagaaatactatgtggactctgtgaagggccgattcaccatctccagagacaacgccaagaactcactgtatctgcaaatgaacagcctgagagccgaggacacggctgtgtattactgtgcgagactgtctggcagcagctgggactttgactactggggccagggaaccctggtcaccgtctcctca

SEQ ID NO: 4 - DNA, 318 bp, Artificial Sequence
  Description of Artificial Sequence: Synthetic antibody
tcctatgagctgactcagccaccctcagtgtccgtgtccccaggacagacagccagcatcacctgctctggagataaattgggggataaatatgcttgttggtaccaacagaggccaggccagtcccctgtgctggtcatctatcaagatagtaagcggccctcagggatccctgagcgattctctggctccaactctgggaacacagccactctgaccatcagcgggacccaggctatggatgaggctgactattactgtcaggcgtgggacagcagcactggagtcttcggaactgggaccaaggtcaccgtccta

SEQ ID NO: 5 - DNA, 387 bp, Artificial Sequence
  Description of Artificial Sequence: Synthetic antibody
gaggtgcagctggtggagtctgggggaggtgtggtacggcctggggggtccctgagactctcctgtgcagcctctggattcacctttgatgattatggcatgagctgggtccgccaagctccagggaaggggctggagtgggtctctgctattaattggaatggtggtagcacaggttatgcagactctgtaaagggccgattcaccatctccagagataatgccaagaactccctgtatctgcaaatgaacagtctgagagccgaggacacggccttgtatcactgtgcgagacggcgtagctcgtcccggtatagcagtggctggtatatgtactactactacatggacgtctggggcaaagggaccacggtcaccgtctcctca

SEQ ID NO: 6 - DNA, 321 bp, Artificial Sequence
  Description of Artificial Sequence: Synthetic antibody
gacatccagatgacccagtctccttccaccctgtctgcatctgtaggagacagagtcaccatcacttgccgggccagtcagagtgttagtacctggttggcctggtatcagcagaaaccagggaaagcccctaacctcctgatctatgaggcgtctagtttagaaagtggggtcccatcaaggttcagcggcagtggatctgggacagaattcactctcaccatcagcagcctgcagcctgatgattttgcaacttattactgccaacagtataatacttattcggggacgttcggccaagggaccaaggtggaaatcaaa

SEQ ID NO: 7 - DNA, 360 bp, Artificial Sequence
  Description of Artificial Sequence: Synthetic antibody
cagatcaccttcaaggagtctggtcctacgctggtgaaacccacagagaccctcacgctgacctgcaccttctctgggttctcagtcagcactagtggagagggtgtgggctggatccgtcagcccccaggaaaggccctggagtggcttgcagtcatttattgggatgatgataagcgctacagcccatctctgaagagcaggctcaccatcaccagggacacctccaaaaaccaggtggtccttacaatgaccaacatggaccctgtggacacagccacatattactgtgcacacaggctttggttcagggatgcttttgatatttggggccaagggacaacggtcaccgtctcctca

SEQ ID NO: 8 - DNA, 321 bp, Artificial Sequence
  Description of Artificial Sequence: Synthetic antibody
gacatccagatgacccagtctccatcgtccctgtctgcatctgtaggagacagagtcacaatcacttgccgggcacgtcagagcattagcaactatttaaattggtatcagcagaaaccagggaaagcccctaagctcctgatctatgctgcatccagtttgcacagtggggtcccatcaaggttcagtggcagtggatctgggacagatttcactctcaccatcagcagtctgcaacctgaagattttgcaacttactactgtcaacagacttacagtaccttctggacgttcggccaagggaccaacgtggaaatcaaa

SEQ ID NO: 9 - DNA, 357 bp, Artificial Sequence
  Description of Artificial Sequence: Synthetic antibody
gaggtgcagctggtggagtctgggggaggcttggtccagcctggggggtccctaagactctcctgtgcagcctctggattcacctttagtacctattggatgacctgggtccgccaggctccagggaaggggctggagtgggtggccaacataaagcaagatggaagtgagaaatactatgtggactctgttaagtaccgattcaccatctccagagacaacgccaagaactcactgtatctgcaaatgaacagcctgagagccgaggacacggctgtgtattactgtgcgagagtcgggagcagcagctggtactttgactactggggccagggaaccctggtcaccgtctcctca

SEQ ID NO: 10 - DNA, 318 bp, Artificial Sequence
  Description of Artificial Sequence: Synthetic antibody
tcctatgaactgactcagccaccctcagtgtccgtgtccccaggacagacagccagcatcacctgctctggagataaattgggggataaatatgcttgctggtatcagcagaagccaggccagtcccctgtggtggtcatctatcaagatagcaagcggccctcagggatccctgagcgattctctggctccaactctgggaacacagccactctgaccatcagcgggacccaggctatggatgaggctgactattactgtcaggcgtgggacagcagcactgcggtattcggcggagggaccaagctgaccgtccta

SEQ ID NO: 11 - DNA, 369 bp, Artificial Sequence
  Description of Artificial Sequence: Synthetic antibody
cagctacagctgcaggagtcgggcccaggactggtgaagccttcggagaccctgtccctcacctgcactgtctctggtggctccatcagcagtggaacttactactgcggctggatccgccagcccccagggaaggggctggagtggattgggagtacatattatggtgggagcaccctctacaacccgtccctcaggggtcgagtcaccatatccgtagacacgtccaagaaccagttttccctgaagctgagctctgtgaccgccgcagacacggctgtgtattactgtgcgagacggggtaattactatgatagtaagaactggttcgacccctggggccagggaaccctggtcaccgtctcctca

SEQ ID NO: 12 - DNA, 327 bp, Artificial Sequence
  Description of Artificial Sequence: Synthetic antibody
gaaatagtgatgacgcagtctccagccaccgtgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtgccagtgggtctgggacagagttcactctcaccatcagcagcctgcagtctgaagattttgcagtttattactgtcagcagtataataactggccacctatgtacacttttggccaggggaccaaggtggagatcaaa

SEQ ID NO: 13 - DNA, 360 bp, Artificial Sequence
  Description of Artificial Sequence: Synthetic antibody
caggtgcagctggtggagtctgggggaggcgtggtccagcctgggaggtccctgagagtctcctgtgcagcgtctggattcaccttcagtagccatggcatgcactgggtccgccaggctccaggcaaggggctggagtgggtgtcagttatatggtatgatggaagtaataaatactatgcagactccgtgaagggccgattcaccatctccagagacaattccaagaacacgctgtctctgcaaatgaacagcctgagagccgaagacacggctgtgtattactgtgcgagagagagcgcggacatatcatctcgtcttgactactggggccggggaaccctggtcaccgtctcctca

SEQ ID NO: 14 - DNA, 318 bp, Artificial Sequence
  Description of Artificial Sequence: Synthetic antibody
tcctatgagctgacacagccaccctcggtgtcagtgtccccaggacaaacggccaggatcacctgctctggagatgcattgccaacaaaatatgcttattggtaccagcagaagtcaggccaggcccctgtgctggtcatctatgacgacagcaaacgaccctccgggatccctgagagattctctggctccagctcagggacaatggccaccttgactatcagtggggcccaggtggaggatgaagctgactactactgttactcaacagacagcagtggtaatgtcttcggaactgggaccaaggtcaccgtccta

SEQ ID NO: 15 - DNA, 363 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcaggtatatcatatgatggaagcaataaatactacgcagactccgtgaagggccgattcaccatctccagagacaattccaagaacacgctgtatctgcaaatgaacagcctgagagctgaggacacggctgtgtattactgtgcgagagcggacactatggttcggggaacttattttgagtactggggccagggaaccctggtcaccgtctcctca

SEQ ID NO: 16 - DNA, 330 bp, Artificial Sequence
  Description of Artificial Sequence: Synthetic antibody
cagtctgccctgactcagcctgcctccgtgtctgggtctcctggacagtcgatcaccatctcctgcactggaaccagcagtgacgttggtggttataactatgtctcttggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattgctgcagctcatatacaagcagcagagctgtgctattcggcggagggaccaagctgaccgtccta

SEQ ID NO: 17 - DNA, 381 bp, Artificial Sequence
  Description of Artificial Sequence: Synthetic antibody
cagatcaccttgaaggagtctggtcctacgctggtaaaacccacacagaccctcacgctgacctgcaccttctctgggttctcactcagcactagtggagtgggtgtggcctggatccgtcagcccccaggaaaggccctggagtggcttgctctcatttattgggatgatgataagcgctacagcccatctctgaagagcaggctcaccatcaccaaggacacctccaaaaaccaggtggtccttacaatgaccaacatggaccctgtggacacagccacatattactgtgcacacagacttccaacgccccaactgctaccatcttttgaaaactggttcgacccctggggccagggaaccctggtcaccgtctcctca

SEQ ID NO: 18 - DNA, 330 bp, Artificial Sequence
  Description of Artificial Sequence: Synthetic antibody
cagtctgtgctgactcagccaccctcggtgtctgaagcccccaggcagagggtcaccatctcctgttctggaggcagctccaacatcggaaataatgctgtaaactggtaccagcagctcccaggaaaggctcccaaactcctcatctattatgatgatctgctgccctcaggggtctctgaccgattctctggctccaagtctggcacctcagcctccctggccatcagtgggctccagtctgaagatgaggctgattattactgtgcatcatgggatgacagcctgattggtccggtattcggcggagggaccaagctgaccgtccta

SEQ ID NO: 19 - DNA, 360 bp, Artificial Sequence
  Description of Artificial Sequence: Synthetic antibody
gaggtgcagctggtggagtccgggggaggcttagttcagcctggggggtccctgagactctcctgtgcagtctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagaagtggagcagctggctcatatggttgactactggggccagggaaccctggtcaccgtctcctca

SEQ ID NO: 20 - DNA, 324 bp, Artificial Sequence
  Description of Artificial Sequence: Synthetic antibody
tcctatgagctgacacagccaccctcggtgtcagtgtccccaggacagacggccaggatcacctgctctggagatgcattgccaaaccaatatgcttattggtaccagcagaagccaggccaggcccctgtgctggtgatatataaagacagtgagaggccctcagggatccctgagcgattctctggctccagctcagggacaacagtcacgttgaccatcagtggagtccaggcagaagacgaggctgactattactgtcaatcagcagacagcagtggtacatcttgggtgttcggcggagggaccaagctgaccgtccta

SEQ ID NO: 21 - DNA, 363 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaatcaataaatactacgcagacgccgtgaagggccgattcaccatctccagagacaattccaagaacacgctgtatctgcaaatgaacagcctgagagctgaggacacggctgtgtattactgtgcgaggccacgaagtgggagctactacgcctactttgactactggggccagggaaccctggtcaccgtctcctca

SEQ ID NO: 22 - DNA, 321 bp, Artificial Sequence
  Description of Artificial Sequence: Synthetic antibody
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttacaaagtggggtcccatcaaagttcagcggcagtggatctgggacagatttcactctcaccatcagcagcctgcagcctgaagatgttgcaacttattactgccaacagtataatagtcaccctcccactttcggcggagggaccaaggtggagatcaaa

SEQ ID NO: 23 - DNA, 357 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagttactatcctatgcactggctctgggtccgccaggctccaggcaaggggctggagtgggtggcagttacatcatatgatggaaccaataaatactacgcagactccgtgaagggccgattcaccatctccagagacaattccaagaacactctgtatctgcaaatgaacagcctgagagctgaggacacggctgtgtattactgtgcgagagggggagctactaactttgactactggggccagggaaccctggtcaccgtctcctca

SEQ ID NO: 24 - DNA, 330 bp, Artificial Sequence
  Description of Artificial Sequence: Synthetic antibody
tcctatgtgctgactcagccaccctcggtgtcagtggccccaggaaagacggccaacattacctgtgggggaaacaacattggaagaaaaagtgtgcactggtaccagcagaagtcaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatccggagtgggtgttcggcggagggaccaagctgaccgtccta

SEQ ID NO: 25 - DNA, 378 bp, Artificial Sequence
  Description of Artificial Sequence: Synthetic antibody
gaagcgcagctggtggagtctgggggaggcttggtacagcctggcaggtccctgagactctcctgtgcagcctctggattcacctttgatgattctgccatgcactgggtccggcaagctccagggaagggcctggagtgggtctcaggtattagttggaatagtggtaacgtaggctatgcggactctgtgaagggccgattcaccatctccagagacaacgccaagaactccctgtatctgcaaatgaacagtctgagagctgaggacacggccttgtattactgtacaaaagcctctcgatattgtagtagtaccatctgctattggaactggttcgacccctggggccagggaaccctggtcaccgtctcctca

SEQ ID NO: 26 - DNA, 318 bp, Artificial Sequence
  Description of Artificial Sequence: Synthetic antibody
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaactggggtcccatcaaggttcagtggcagtggatctgggacagatttcactctcaccatcagaagtctgcaacctgaagattttgcaagttactactgtcaacagagttacagtacccccactttcggcggagggaccaaggtggagatcaaa

SEQ ID NO: 27 - DNA, 378 bp, Artificial Sequence
  Description of Artificial Sequence: Synthetic antibody
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtgccatatactacgcagactctgtgaagggccgattcaccatctccagagacaacgccaagaactcactgtatctgcaaatgaacagcctgagagtcgaggacacggctgtttattactgtgcgagagaagcgcggtcacgatattttgactggttaccctcgtactactttgactactggggccagggaaccctggtcaccgtctcctca

SEQ ID NO: 28 - DNA, 333 bp, Artificial Sequence
  Description of Artificial Sequence: Synthetic antibody
cagtctgccctgactcagcctgcctccgtgtctgggtctcctggacagtcgatcaccatctcctgcactggaaccagcagtgacattggtggttataactatgtctcctggtaccaacaacacccaggcaaagcccccaaactcctgatttatgatgtcagtaatcggccctcaggggtttctactcgcttctctggctccaagtctggcaacacggcctccctgaccatctctgggctccaggctgaggacgaggctgattattactgcagctcatatacaagcagcagcacccatgtggtgttcggcggagggaccaagctgaccgtccta

SEQ ID NO: 29 - DNA, 374 bp, Artificial Sequence
  Description of Artificial Sequence: Synthetic antibody
caggtgcagctggtgcaatctgggtctgagttgaagaagcctggggcctcagtgaaggtttcctgcaaggcttctggatacaccttcactacctatgctatgaattgggtgcgacaggcccctggacaaggacttgagtggatgggatggatcaacaccaacactgggaacccaacgtatgcccagggcttcacaggacggtttgtcttctccttggacacctctgtcaacacggcatttctgcatatcggcagcctaaaggctgaggacactgccgtgtattactgtgcgagagatcaggacagtggctacccaacttactactactactacatggacgtctggggcaaagggaccacggtcaccgtctcctc

SEQ ID NO: 30 - DNA, 336 bp, Artificial Sequence
  Description of Artificial Sequence: Synthetic antibody
gatattgtgatgacccagactccactctctctgtccgtcacccctggacagccggcctccatctcctgcaagtctagtcagagcctcctgcatagtgatggaaagacctatttgtattggtacctgcagaagccaggccagtctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacagcctcctctcactttcggcggagggaccaaggtggagatcaaa

SEQ ID NO: 31 - DNA, 363 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagtacctatgctatgcactgggtccgccaggctccaggcaaggggctggcgtgggtggcacttatatcatatgatggatataataaatactacgcagactccgtgaggggccgattcaccatctccagaatcaattccaagaacacgctgtctctgcagatgaacagcctgagagctgaggacacggctgtctattactgtgcgagagggtcagctggaaactactactacggtatggacgtctggggccaggggaccacggtcaccgtctcctca

SEQ ID NO: 32 - DNA, 321 bp, Artificial Sequence
  Description of Artificial Sequence: Synthetic antibody
gacatccagatgacccagtctccatcctccctgtctgcatctgtaggagacagagtcaccatcacttgccgggcaagtcagaccattaccaactatttaaattggtatcagctgaaatcagggagagcccccaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacgccgtacacttttggccaggggaccaagctggagatcaaa

SEQ ID NO: 33 - DNA, 381 bp, Artificial Sequence
  Description of Artificial Sequence: Synthetic antibody
caggtgcatctggtggagtctgggggaggcgtggtccagcctgggaggtccctgagactctcctgtgcagcctctggattcaccttcagtaactatggcatgcactgggtccgccaggctccaggcaaggggctggagtgggtggcagttatctcaaatgatgaatttaataaattctatgcaaactccgtgaagggccgattcaccatctccagagacaattcgaagaacacggtgtatttgcaattgaacagtctgagaactgaggacacggctcgatattactgtgcgaaagggggggatggcagtggctgggcctgggacggtgataaccccccaacggactactggggccagggaaccctggtcatcgtctcttca

SEQ ID NO: 34 - DNA, 338 bp, Artificial Sequence
  Description of Artificial Sequence: Synthetic antibody
gacatcgtgatgacccagtctccggacttcctggctgtgtctctgggcgagagggccaccatcagctgcaagtccagtcagagtgttttatacacccccaagaataagaactacttagcttggtacaagcagaaaccaggacagcctcctaaggtgctcatttactgggcatctacccgggaatccggggtccctgaccgattcagtggcagcgggtctgggacagatttcactctcatcatcagcagcctgcaggctgaggatgcggcagtttattactgtcagcaatattatactgctcctctcactttcggcggtgggaccagggtggagatcaa

SEQ ID NO: 35-36 - skipped (2 entries)

SEQ ID NO: 37 - DNA, 372 bp, Artificial Sequence
  Description of Artificial Sequence: Synthetic antibody
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacccgactatagcagtggctggtttagctactggtacttcgatctctggggccgtggcaccctggtcactgtctcctca

SEQ ID NO: 38 - DNA, 324 bp, Artificial Sequence
  Description of Artificial Sequence: Synthetic antibody
gaaattgtgttgacgcagtctccaggcaccctgtctttgtctccaggggaaagagccaccctctcctgcagggccagtcagagtgttagcagcaacttcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgccagcagtatggtcgctcaccgatcaccttcggccaagggacacgactggagattaaa

SEQ ID NO: 39 - DNA, 363 bp, Artificial Sequence
  Description of Artificial Sequence: Synthetic antibody
caggtccagctggtacagtctggggctgaggtgaagaagcctggggcctcagtgaaggtctcctgcaaggtttccggatacaccctcactgaactatccatacactgggtgcgacaggctcctggaaaagggcttgagtggatgggaggttttgatcctgaagatgctgaaacaatctacgcacagcagttccagggcagagtcaccatgaccgaggacacatctacagacacagcctacatggagctgagcagcctgaaatctgaggacacggccctgtattactgtgcaacagggttcgcggtgtttgggagggcagcagttccctactggggccagggaaccctggtcaccgtctcctca

SEQ ID NO: 40 - DNA, 318 bp, Artificial Sequence
  Description of Artificial Sequence: Synthetic antibody
tcctatgagctgactcagccaccctcagtgtccgtgtccccaggacagacagccagcatcacctgctttggagataaattgggggataaatatgcttgctggtttcagcagaagccaggccagtcccctgtgctgatcatctatcaaggtgccaagcggccctcagggatccctgagcgattctctggctccaactctgggaacacagccactctgaccatcagcgggacccaggctatggatgaggctgactattactgtcaggcgtgggacagcagcactgtggttttcggcggagggaccaagctgaccgtccta

SEQ ID NO: 41 - DNA, 372 bp, Artificial Sequence
  Description of Artificial Sequence: Synthetic antibody
caggtgcagctggtggagtctgggggaggcgtggtccagcctgggaggtccctgagactctcctgtgcagcctctggattcacgttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatctcacaattgtagtaataccagctgccccaaattttgactactggggccagggaaccctggtcaccgtctcctca

SEQ ID NO: 42 - DNA, 333 bp, Artificial Sequence
  Description of Artificial Sequence: Synthetic antibody
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaggtctggcaacacggcctccctgaccatctctgggctccaggctgaggacgaggctgattattactgcagctcatatacaagcagcagcactcctgtggtgttcggcggagggaccaagctgaccgtccta

SEQ ID NO: 43 - DNA, 363 bp, Artificial Sequence
  Description of Artificial Sequence: Synthetic antibody
gaggtgcagctggtgcagtctggagcagaggtgaaaaagcccggggagtctctgaagatctcctgtaagggttctggatacagctttaccagtcactggatcggctgggtgcgccagatgcccgggaaaggcctggagtggatggggatcatctatcctggtgactctgataccagatatagcccgtccttccaagggcaggtcaccatctcagccgacaagtccatcagcaccgcctacctgcagtggagcagcctgaaggcctcggacaccgccatgtattactgtgcgagtgccctcagagagcgaggcgtacagctgtggtcagtttggggccagggaaccctggtcaccgtctcctca

SEQ ID NO: 44 - DNA, 330 bp, Artificial Sequence
  Description of Artificial Sequence: Synthetic antibody
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctttattaactccaatcggccctcaggggtccctgaccgcttctctggctccaagtctggcacctcagcctccctggccatcactgggctccaggctgaggatgaggctgattattactgccagtcctatgacagcagcctgggtgccttgttcggcggagggaccaagctgaccgtccta

SEQ ID NO: 45 - DNA, 366 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacactctttatctgcaaatgaacagcctgagagttgaggacacggctgtgtattactgtgcgagagatcaagaatggttcagggagttattcctttttgactactggggccagggaaccctggtcaccgtctcctca

SEQ ID NO: 46 - DNA, 321 bp, Artificial Sequence
  Description of Artificial Sequence: Synthetic antibody
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgatgcatccagtttgcaaagtggggtcccatcaaggttcagcggcagtggatctgggacagatttcactctcaccatcagcagcctgcagcctgaagattttgcaacttactattgtcaacaggctaacagtttccctccgactttcggccctgggaccaaagtggatatcaaa

SEQ ID NO: 47 - DNA, 353 bp, Artificial Sequence
  Description of Artificial Sequence: Synthetic antibody
caggtgcagctggtggagtccgggggaggcgtggtccagcctgggaggtccctgagactctcctgtgcagcctctggattcaccttcagtacctatggcatgcactgggtccgccaggctccaggcaaggggctggagtgggtggcagttatatcatatgatggaagtaataaatactatgcagactccgtgaagggccgattcaccatctccagagacaattccaagaacacactgtatctgcaaatgaacagcctgagagctgaggacacggctatgtattactgtgcgaaagatgggagtatagcagcagctgactactggggccagggaaccctggtcaccgtctcctc

SEQ ID NO: 48 - DNA, 339 bp, Artificial Sequence
  Description of Artificial Sequence: Synthetic antibody
gacatcgtgatgacccagtctccagactccctggctgtgtctctgggcgagagggccaccatcaactgcaagtccagccagagtgtcttacacagctccaacaacaaggactccttagtttggtaccagcagaaaccaggacagcctcctaagctgctcatttactgggcatctagccgggaatccggggtccctgaccgattcagtggcagcgggtctgggacagatttcactctcaccatcagcagcctgcaggctgaagatgtggcagtttattactgtcagcaatattatagtactccttggacgttcggccaagggaccaaggtggaaatcaaa

SEQ ID NO: 49 - DNA, 384 bp, Artificial Sequence
  Description of Artificial Sequence: Synthetic antibody
gaggtgcagctggtggagtctgggggaggcctggtcaagcctggggggtccctgagactctcctgtgcagcctctggattcaccttcagtagttatagcatgaactgggtccgccaggctccagggaaggggctggagtgggtctcatccataagtaatagtaatagtttcatatactacgcagactcaatgaagggccgattcaccatctccagagacaacgccaagaactcactgtatctgcaaatgaacagcctgagagccgaggacacggctgtgtattactgtgcgcgagttaacggcaactcaaactggaactttgggtcttactactactactacatggacgtctggggcaaagggaccacggtcaccgtctcctca

SEQ ID NO: 50 - DNA, 318 bp, Artificial Sequence
  Description of Artificial Sequence: Synthetic antibody
gaaattgtgttgacacagtctccagccattttgtctttgtctccaggggaaagagccaccctctcctgcagggccagtcagagtgttagtagctacttagcctggtaccaacagaagcctggccaggctcccaggctcctcatctatgatacatccaacagggccactggcatcccagccaggttcagtggcagtgggtctgggacagacttcactctcactatcagcagcctagagcctgaagattttgcattttattactgtcagcagcgtggcaactggtggacgttcgcccaagggaccaaggtggaaatcaaa

SEQ ID NO: 51 - DNA, 372 bp, Artificial Sequence
  Description of Artificial Sequence: Synthetic antibody
gaggtgcagctggtggagtctgggggaggcctggtcaagcctggggggtccctgagactctcctgtgcagcctctggattcactttcagtggctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagggccgaggacacggctgtgtattactgtgcgagatggctacagttaagatcagactactattacttcggtatggacgtctggggccaagggaccacggtcaccgtctcctca

SEQ ID NO: 52 - DNA, 324 bp, Artificial Sequence
  Description of Artificial Sequence: Synthetic antibody
gaaatagtgatgacgcagtctccagccaccctgtctgtgtctccaggggaaagagccaccctctcctgcagggccagtcagagtgttagcaacaacttagcctggtaccagcacaaacctggccaggctcccaggctcctcatctatggtgcatccaccagggccactggtatcccagccaggttcagtggcagtgggtctgggacagagttcactctcaccatcagcagcctacagtctgaagattttgcagtttatttctgtcagcagtgttataactggcctccgtggacgttcggccaagggaccaaggtggaaatcaaa

SEQ ID NO: 53 - DNA, 369 bp, Artificial Sequence
  Description of Artificial Sequence: Synthetic antibody
caggtgcagctgcaggagtcgggcccaggactggtgaagccttcggagaccctgtccctcacctgcactgtctctggtggctccatcagtagttactactggagctggatccggcagcccccagggaagggactggagtggattggttatatctattacagtgggagcaccaactacaacccctccctcaagagtcgagtcaccatatcagtagacatgtccaagaaccagttctccctgaagctgaggtctgtgaccgctgcggacacggccgtgtattactgtgcgagagccccgagggaaaggctccaatggggggagtactacttcgactactggggccagggaaccctggtcaccgtctcctca

SEQ ID NO: 54 - DNA, 333 bp, Artificial Sequence
  Description of Artificial Sequence: Synthetic antibody
cagtctgccctgactcagcctgcctccgtgtctgggtctcctggacagtcgatcaccatctcctgcactggaaccagcagtgatgttgggagttataaccttgtctcctggtaccaacagcatgcaggcaaagcccccaaactcatgatttatgaggtcattaagcggccctcaggggtttctaatcgcttctctggctccaagtctggcaacacggcctccctgacaatctctgggctccaggctgaggacgaggctgattattactgctgctcatatgcagttagtaccacttatgttatattcggcggagggaccaagctgaccgtccta

SEQ ID NO: 55 - DNA, 363 bp, Artificial Sequence
  Description of Artificial Sequence: Synthetic antibody
gaggtgcagctggtgcagtctggagcagaggtgaaaaagcccggggagtctctgaagatctcctgtaagggttctggatacagctttaccaactcctggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatctattactgtgcgacacatcgttgtagtggtggtttctgctacttagcctactggggccagggaaccctggtcaccgtctcctca

SEQ ID NO: 56 - DNA, 351 bp, Artificial Sequence
  Description of Artificial Sequence: Synthetic antibody
cagcctgtgctgactcagccaccttctgcatcagcctccctgggagcctcggtcacactcacctgcaccctgagcagcggctacagtaattataaagtggactggtaccagcagagaccagggaagggcccccggtttgtgatgcgagtgggcactggtgggattgtgggatccaagggggatggcatccctgatcgcttctcagtcttgggctcaggcctgaatcggtacctgaccatcaagaacatccaggaagaggatgagagtgactaccactgtggggcagaccatggcagtgggagcaacttcgtttttgtggttttcggcggagggaccaagctgaccgtccta

SEQ ID NO: 57 - DNA, 384 bp, Artificial Sequence
  Description of Artificial Sequence: Synthetic antibody
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaaaaaagactatgcagactccgtgaagggccgattcaccatctccagagacaattccaagaacacgctgtatctgcaaatgaacagcctgagagccgaggacacggctgtgtattactgtgcgagagatcaatctcagggcgcttatattttgactggttataggggctacggtatggacgtctggggccaggggaccacggtcaccgtctcctca

SEQ ID NO: 58 - DNA, 336 bp, Artificial Sequence
  Description of Artificial Sequence: Synthetic antibody
gatattgtgatgactcagtctccactctccctgcccgtcacccctggagagccggcctccatctcctgcagatctagtcagagcctcctgcatagtaatggatacaactatttggattggtacttgcagaagccagggcagtctccacagttcctgatctatttgggttctaatcgggcctccggggtccctgacaggttcagtggcagtggatcaggcacagattttatactgaaaatcagcagagtggaggctgaggatgttggggtttattactgcatgcaagctcttcaaactccattcactttcggccctgggaccaaagtggatatcaaa

SEQ ID NO: 59 - DNA, 369 bp, Artificial Sequence
  Description of Artificial Sequence: Synthetic antibody
caggtgcagctacagcagtggggcgcaggactgttgaagccttcggagaccctgtccctcacctgcgctgtctatggtgggtccttcagtggtcactactggagctggatccgccagcccccagggaagggactggagtggattggggaaatcaatcacagtggaagcaccaactacaacccgtccctcaagagtcgagtcaccatatcagtagacacgtccaagaaccagttctccctgaagctgagctctgtgaccgccgcggacacggctgtgtattactgtgcgagacccccccaagcagctcgtattcattactactactacatggacgtctggggcaaagggaccacggtcaccgtctcctca

SEQ ID NO: 60 - DNA, 324 bp, Artificial Sequence
  Description of Artificial Sequence: Synthetic antibody
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataattactggcctcccctcactttcggcggagggaccaaggtggagatcaaa

SEQ ID NO: 61 - DNA, 363 bp, Artificial Sequence
  Description of Artificial Sequence: Synthetic antibody
gaagtgcagctggtggagtctgggggaggcttggtacagcctggcaggtccctgagactctcctgtgcagcctctggattcacctttgatgattatgccatgaactgggtccggcaacctccagggaagggcctggagtgggtctcaggtattagttggaatagtgatagcataggctatgcggactccgtgaagggccgattcaccatctccagagacaacgccaagaactccctgtatctgcaaatgaacagtctgagagctgaggacacggccatgtattactgtgcaaaaggaaggggtgctggttatacttcctacatggacgtctggggcaaagggaccacggtcaccgtctcctca

SEQ ID NO: 62 - DNA, 326 bp, Artificial Sequence
  Description of Artificial Sequence: Synthetic antibody
tcctatgtgctgactcagccaccctcggtgtcagtggccccaggaaagacggccaggattacctgtgagggaaacaacattggaagtaaaagtgtgcactggtaccagcagaagccaggccaggcccctgtgctggtcgtctatgatgatagcggccggccctcagggatccctgaacgattctctggttccaactctgggaacacggccaccctgaccatcagcagggtcgaagccggggatgaggccgactatttctgtcaggtgtgggatagtagtagtgatcatcatgtggttttcggcggagggaccaagctgaccgtcct

SEQ ID NO: 63 - DNA, 300 bp, Artificial Sequence
  Description of Artificial Sequence: Synthetic antibody
cctacacgacgctcttccgatctgggggtcactggatcggctgggtgcgccagatgcccgggaaaggcctggagtggatggggatcatctatcctggtgactctgataccagatatagcccgtccttccaagggcaggtcaccatctcagccgacaagtccatcagcaccgcctacctgcagtggagcagcctgaaggcctcggacaccgccatgtattactgtgcgagtgccctcagagagcgaggcgtacagctgtggtcagtttggggccagggaaccctggtcaccgtctcctcag

SEQ ID NO: 64 - DNA, 330 bp, Artificial Sequence; identical to SEQ ID NO: 44
  Description of Artificial Sequence: Synthetic antibody

SEQ ID NO: 65 - DNA, 369 bp, Artificial Sequence
  Description of Artificial Sequence: Synthetic antibody
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcatagcctatacggactctgtgaagggccgattcaccatctccagagataacgccaagaactccctgtatctgcaaatgaacagtctgagagctgaggacacggccttgtattactgtgcaaaagcacactcgactggacaccaatactactacggtatggacgtctggggccaagggaccacggtcactgtctcctca

SEQ ID NO: 66 - DNA, 321 bp, Artificial Sequence
  Description of Artificial Sequence: Synthetic antibody
gacatccagatgacccagtctccatcctccttgtctgcatctgtaggagacagagtcaccatcacttgccgggcaagtcagagcattagcagctttttaaattggtatcagcagaaaccagggaaagcccctaagctcctgatctatgctgcattcaatttgcaaagtggggtcccatcaaggttcagtggcagtggatctgggacagatttcactctcaccatcagcagtctgcaatctgaagattttgcaacttactactgtcaacagagttacaataccccttacacttttggccaggggaccaagctggagatcaaa

SEQ ID NO: 67 - DNA, 369 bp, Artificial Sequence
  Description of Artificial Sequence: Synthetic antibody
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctatatctgcaaatgaacagtctgagagctgaggacacggccttgtattactgtgcaaaagtgtcgtccattactagcctattgggatactactttgactcctggggccagggaaccctggtcaccgtctcctca

SEQ ID NO: 68 - DNA, 327 bp, Artificial Sequence
  Description of Artificial Sequence: Synthetic antibody
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaccgtagcaactggcctccgaggctcactttcggcggagggaccaaggtggagatcaaa

SEQ ID NO: 69 - DNA, 363 bp, Artificial Sequence
  Description of Artificial Sequence: Synthetic antibody
caggtccagctggttcaatctggggctgaggtgaagaagcctgggtcctcggtgaaggtctcctgcaagacttctggagacacctccagcagttatactgtcggctgggtgcgacaggcccctggacaagggcttgagtggatgggaaggatcatccctatccttggtatagcatactccgcacagaagttccagggcagactcacgattaccgcggacaaatccacgagcacatcctacatggagctgagcagcctgagatctgaggacacggccgtttattactgtgcgagaggggtggtagctgctactccgggttggttcgacccctggggccagggaaccctggtcaccgtctcctca

SEQ ID NO: 70 - DNA, 318 bp, Artificial Sequence
  Description of Artificial Sequence: Synthetic antibody
gaaatagtgatgacgcagtctccagccaccctgtctgtgtctccaggggaaagagtcaccctctcctgcagggccagtcagagtgttagcagcaacttagcctggtaccagcagaaacctggccaggctcccaggctcctcatctatggtgcatccaccagggccactggtatcccagccaggttcagtggcggtgggtctgggacagagttcactctcaccatcagcagcctgcagtctgaagattttgcagtttattactgtcagcagtataataacttcctcactttcggcggagggaccaaggtagagatcaaa

SEQ ID NO: 71 - DNA, 357 bp, Artificial Sequence
  Description of Artificial Sequence: Synthetic antibody
gaggtgcagctggtggagtccgggggaggcttagttcagcctggggggtccctgagactctcctgtgcagcctctggattcaccttcagtagctactggatgcactgggtccgccaagttccagggaaggggctggtgtgggtctcacgtattaatagtgatgggagtagcacaagctacgcggactccgtgaagggccgattcaccatctccagagacaacgccaagaacacgctgtatctggaaatgaacagtctgagagcccaggacacggctgtttattactgtgcaggttccccgtggctacgaggcgacattgactactggggccagggaaccctggtcaccgtctcctca

SEQ ID NO: 72 - DNA, 336 bp, Artificial Sequence
  Description of Artificial Sequence: Synthetic antibody
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaaggccctctggggtccctgatcggttctctggctccatcgacagctcctccaactctgcctccctcaccatctctgggctgatgactgaggacgaggctgactactactgtcagtcttatgatggcagcaatcatgctgtggtattcggcggagggaccaagctgaccgtccta

SEQ ID NO: 73 - DNA, 390 bp, Artificial Sequence
  Description of Artificial Sequence: Synthetic antibody
caggtgcagctggtgcagtctggggctgaggtgaagaagcctggggcctcagtgaaggtctcctgcaaggcttctggatacaccttcaccgactactatatgcactgggtgcgacaggcccctggacaagggcttgagtggatgggatggatcaaccctaacagtcgtggcacaaactatgcacagaagtttcagggcagggtcactatgaccagggacacgtccatcagcacagtctacatggagctgagcaggctgacatctgacgacacggccgtctattactgtgcgagagtggtggtcctcggctatggccgcccaaacaattactatgatggtaggaatgtgtgggactactggggccagggaaccctggtcaccgtctcctca

SEQ ID NO: 74 - DNA, 330 bp, Artificial Sequence
  Description of Artificial Sequence: Synthetic antibody
cagtctgtgctgactcagccaccctcagcgtctgggacccccgggcagagggtcatcatctcttgttctggaagcagctccaacatcggaagtaatactgtaaagtggtatcatcagctcccaggaacggcccccaaactcctcatctgtagtaataatcagcggccctcaggggtccctgaccgattctctggctccaagtctgacacctcagcctccctggccatcagtgggctccagtctgaggatgaggctgattattactgtgcagcatgggatgacagcctgaatgctttggtattcggcggagggaccaagctgaccgtccta

SEQ ID NO: 75 - DNA, 351 bp, Artificial Sequence
  Description of Artificial Sequence: Synthetic antibody
caggtcaccttgagggagtctggtcctgcgctggtgaaacccacacagaccctctcactgacctgcaccttctctgggttctcactcggcactagtggaatgtgtgtgagctggatccgtcagcccccagggaaggccctggagtggcttgcacgcattgattgggatgatgataaatactacagcacatctctgaagaccaggctcaccatctccaaggacacctccaaaaaccaggtggtccttacaatgaccaacatggaccctgtggacacagccacgtattactgtgcacggggggtggttacttatgactattggggccagggaaccctggtcaccgtctcctca

SEQ ID NO: 76 - DNA, 321 bp, Artificial Sequence
  Description of Artificial Sequence: Synthetic antibody
gacatccagatgacccagtctccatcctccctgtctgcatctgtaggagacagagtgaccatcacttgccgggcaagtcagagcattgccggctatttaaactggtatcagcagaaaccagggaaagcccctaagctcctgatctatggtacaaccagtttgcaaagtggggtcccagtaaggttcagtggcagtggatctgggacagatttcactctcaccatcagcagtctgcaacctgaagattttgcaacttactactgtcaacagagttacagtacccctggcaccttcggccaagggacacgactggagattaaa

SEQ ID NO: 77 - DNA, 372 bp, Artificial Sequence
  Description of Artificial Sequence: Synthetic antibody
caggtccagctggtgcaatcaggggctgaggtgaagaagcctgggtcctcggtgaaggtctcctgcaaggcttctggagacaccttcagcagctatactatcaactgggtgcgacaggcccctggacaagggcttgagtggatgggaaggatcatccctatccttggtataccaaactacgcacagaaattccagggcagagtcaccattaccgcggacaaatccacgagcacagccttcatggagctgagcagcctgagatctgaggacacggccgtctattactgtgcgagagggaggggctacagtaactacggggcctcctactacatggacgtctggggcaaagggaccacggtcaccgtctcctca

SEQ ID NO: 78 - DNA, 324 bp, Artificial Sequence
  Description of Artificial Sequence: Synthetic antibody
gacatccagatgacccagtctccatcctccctgtctgcatctgtaggagacagagtcaccatcacttgccaggcgagtcaggacattaaccactatttaaattggtatcagcagaaaccagggaaagcccctaagctcctgatctacgatgcatccaatttggaaaccggggtcccatcaaggttcagtggaagtggatctgggacagattttactttcaccatcaccagcctgcagcctgaagatgttgcaacatattactgtcaacagtctgataatctccccatgtacacttttggccaggggaccaagctggagatcaaa

SEQ ID NO: 79 - DNA, 363 bp, Artificial Sequence; identical to SEQ ID NO: 61
  Description of Artificial Sequence: Synthetic antibody

SEQ ID NO: 80 - DNA, 326 bp, Artificial Sequence; identical to SEQ ID NO: 62
  Description of Artificial Sequence: Synthetic antibody

SEQ ID NO: 81-82 - skipped (2 entries)

SEQ ID NO: 83 - DNA, 369 bp, Artificial Sequence
  Description of Artificial Sequence: Synthetic antibody
caggtgcagctggcgcagtctggggctgaggtgaagaagcctggggcctcagtgaaggtctcctgcaaggctgctggatacaccttcaccagttatgatatcaactgggtgcgacaggccactggacaagggcttgagtggatggggtggatgaactctaacagtggtaacgcaggctatgcacagaagttccagggcagagtcactatgaccagggacacctccacaagtacagcctacatggagttgagcagcctgacatctgatgacacggccgtgtattattgtgcgagaatgcgcaccggctggcccacacatggccgcccggatgacttctgggggcggggaaccctggtcaccgtctcctca

SEQ ID NO: 84 - DNA, 330 bp, Artificial Sequence
  Description of Artificial Sequence: Synthetic antibody
cagtctgtgctgactcagccaccctcagcgtctgggacccccgggcagagggtcaccatctcttgttctggaagcaactccaacatcggaagttatactgtaaactggtaccagcagttcccaggaacggcccccaaactcctcatttatgataataatcagcggacctcaggggtccctgaccgattctctggctccaagtctggcacctcagcctccctggccatcagtgggctccagtctgaggatgaggctaattattactgtttagtatgggatgacagcctgaatggcctggtattcggcggagggaccaagctgaccgtccta

SEQ ID NO: 85 - DNA, 365 bp, Artificial Sequence
  Description of Artificial Sequence: Synthetic antibody
gaagtgcagctggtggagtctggaggaggcttggtccagccgggggggtccctgagactctcctgtgcagcctctgggttcaccgtcagtagcaactacatgagctgggtccgccaggctccagggaaggggctggagtgggtctcagttatttatcccggtggtagcgcattctacgcagactccgtgaagggccgattcaccatctccagacacaattccaacaacacactgtgtcttcaaatgaacagcctgcgaactgaggacacggccgtgtattattgtgcgagatcttacgatattttgactggttatagagatgcttttgatatctggggccaagggacaatggtcaccgtctcttc

SEQ ID NO: 86 - DNA, 336 bp, Artificial Sequence
  Description of Artificial Sequence: Synthetic antibody
cagtctgtgctgacgcagccgccctcagtgtctggggccccagggcagagggtcaccatctcctgcactgggagcagctccaacatcgggtcaggttctgatgtacactggtaccagcagcttccaggaacagcccccaaactcctcatctatggcaacaccaatcggccctcaggggtccctgaccgattctctggctccaagtctggcacctcagcctccctggccatcactgggctccaggctgaggatgaggctgattattactgccagtcctatgacagcaggctgagtggttttgtggtattcggcggagggaccaagctgaccgtccta

SEQ ID NO: 87 - DNA, 357 bp, Artificial Sequence
  Description of Artificial Sequence: Synthetic antibody
gaggtgcagctggtggagtctgggggaggcttggtccagcctggggggtccctgagactctcctgtgcagcctctggactcaccgtcagtagcaactacatgagttgggtccgccaggctccagggaaggggctggagtgcgtctcagttatttatgccggtggtaatacatactacgcagactccgtgaagggcagattcaccatctccagagacaattccaagaacacgctgtatcttcaaatgaacagcctgagagccgaggacacggctgtgtattactgtgcgagaggcgatggtggttattactcaccctttgactactggggccagggaaccctggtcaccgtctcctca

SEQ ID NO: 88 - DNA, 321 bp, Artificial Sequence
  Description of Artificial Sequence: Synthetic antibody
gacatccagatgacccagtctccatcctccctgtctgcatctgtaggagacagagtcaccatcacttgccgggcaagtcagagcattagcagctatttaaattggtatcagcagaaaccagggaaagcccctaaggtcctgatctatgctgcatccactatgcaaagtggggtcccatcaaggttcaggggcagtggctctgggacagatttcactctcaccatcagcagtctgcaacttgaggattttgcaacttactactgtcaacagagttacagtacccctcagacgtttggccaagggaccaaggtggaaatcaaa

SEQ ID NO: 89 - DNA, 366 bp, Artificial Sequence
  Description of Artificial Sequence: Synthetic antibody
caggtgcagttacagcagtggggcgcagggctgttgaagccttcggagaccctgtccctcacctgcgctgtctctggtgggtccttcagtgcttactactggagctggatccgccagcccccagggaaggggctggagtggattggggaaatcaatcatagtggaagcaccaactacaacccgtccctcaggagtcgagtcaccatatcagtagacacgtccaagaaccaattctccctgaagctgagctctgtgaccgccgcggacacggctgtgtattactgtgcgagagtgggttattcccaagggtactactactactacatggacgtctggggcaaagggaccacggtcaccgtctcctca

SEQ ID NO: 90 - DNA, 321 bp, Artificial Sequence
  Description of Artificial Sequence: Synthetic antibody
gacatccagatgacccagtctccatcctccctgtctgcatctgtaggagacagagtcaccatcacttgccgggcaagtcagagcattagcaactatttaaattggtatcagcagaaaccggggaaagcccctaagctcctgatctatgctgcatccagtttgcaaagtggggtcccctcaaggttcagtggcagtggatctgggacagatttctctctcaccatcagcagtctgcaacctgaagattttgcaacttactcctgtcaacagagttacactaccctcctcactttcggcggagggaccaaggtggagatcaaa

SEQ ID NO: 91-92 - skipped (2 entries)

SEQ ID NO: 93 - DNA, 375 bp, Artificial Sequence
  Description of Artificial Sequence: Synthetic antibody
caggtccagctggtgcagtctggggctgaggtgaagaagcctgggtcctcggtgaaggtctcctgcaaggcttctggaggcaccttcagcagctatagtatcacctgggtgcgacaggcccctggacaagggcttgagtggatgggaaggatcatccctgtccttggtatagcaaactacgcacagaagttccaggacagagtcacgattacggcggacaaatccacgagcacagcctacatggagctgagcagcctgagatctgaggacacggccgtctattactgtgcgagagtgggcgtgagtggttttaaaagtggctcgaactggtacttcgatctctggggccgtggcaccctggtcactgtctcctca

SEQ ID NO: 94 - DNA, 333 bp, Artificial Sequence
  Description of Artificial Sequence: Synthetic antibody
cagtctgtgctgacgcagccgccctcagtgtctggggccccagggcagagggtcaccctctcctgcactgggagcaactccaacatcggggcaggttatgatgtacactggtaccagcagcttccaggaacagcccccaaactcctcatctatggtaatagcaatcggccctccggggtccctgaccgattctctggctccaagtctggcacctcagcctccctggccatcactgggctccaggctgaggatgaggctgattattactgccagtcctatgacagcagcctgagtgattcggtgttcggcggagggaccaaggtgaccgtccta

SEQ ID NO: 95-96 - skipped (2 entries)

SEQ ID NO: 97 - DNA, 366 bp, Artificial Sequence
  Description of Artificial Sequence: Synthetic antibody
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctggtggtggtagtacaacctacgcacagaagttccagggcagagtcaccatgaccagtgacacgtccacgagcacagtctacatggagctgagcagcctgagatctgaggacacggccatgtattactgtgcgagaggggcaattcccccaaatagcagagccgaaattgactactggggccagggaaccctggtcaccgtctcctca

SEQ ID NO: 98 - DNA, 321 bp, Artificial Sequence
  Description of Artificial Sequence: Synthetic antibody
gaaatagtgatgacgcagtctccagccaccctgtctgtgtctccaggggaaagagtcaccctctcctgcagggccagtcagagtgttagcagcaacttagcctggtgccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcaatattataactggccgctcactttcggcggagggaccaaggtggagatcaaa

SEQ ID NO: 99 - DNA, 387 bp, Artificial Sequence
  Description of Artificial Sequence: Synthetic antibody
gaggtgcagctggtggagtctgggggaggtgtggtacggcctggggggtccctgagactctcctgtgcagcctctggattcatctttgatgattatgacatgacctgggtccgccaagctccagggaaggggctggagtgggtctctggtattagttggaatggtggtaacacaggttatgcagactctgtgaagggccgattcaccatctccagagacaacgccaagaactccctgtatctgcaaatgaacagtctgagagccgaggacacggccttgtatcactgtgcagtgattatgtctccaatcccccgttatagtggctacgattgggcgggtggtgcttttgatatctggggccaagggacaatggtcaccgtctcttca

SEQ ID NO: 100 - DNA, 324 bp, Artificial Sequence
  Description of Artificial Sequence: Synthetic antibody
tcttctgagctgactcaggaccctgctgtgtctgtggccttgggacagacagtcaggatcacatgccaaggagacagcctcagaagctattatgcaagctggtaccagcagaagccaggacaggtccctatacttgtcatctatgataaaaacaaccggccctcagggatcccagaccgattctctggctccagctcaggaaacacagcttccttgaccatcactggggctcaggcggaagatgaggctgactattactgtaactcccgggacagcagtggtaacgccgtggtgttcggcggagggaccaagctgaccgtccta

SEQ ID NO: 101 - DNA, 381 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagtaactatggcatgcactgggtccgccaggctccaggcaaggggctggagtgggtggcagttatgtcatatgatggaagtaataaatactatgcagactccgtgaagggccgattcaccatctccagagacaattccaagaacacgctgtatctgcaaatgaacagcctgagagctgaggacacggctgtatattactgtgcgaaaaatttaggaccctattgtagtggtggtacctgctattccttagttggtgactactggggccagggaaccctggtcaccgtctcctca

SEQ ID NO: 102 - DNA, 321 bp, Artificial Sequence
  Description of Artificial Sequence: Synthetic antibody
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgctaatctcccattcactttcggccctgggaccaaagtggatatcaaa

SEQ ID NO: 103-106 - skipped (4 entries)

SEQ ID NO: 107 - DNA, 363 bp, Artificial Sequence
  Description of Artificial Sequence: Synthetic antibody
gaagtgcaactggtggagtctgggggaggcttggtacagcctggcaggtccctgagactctcctgtgcagcctctggattcacctttgatgattatgccatgcattgggtccggcaagctccagggaagggcctagagtgggtctcaggtatcagttggaacagtggtaccataggctatgcggactctgtgaagggccgattcatcatctccagagacaacgccaagaactccttgtatctgcaaatgaacagtctgagacctgaggacacggccttgtattactgtgcaaaagatataatacgtcagggcgaagacggtatggacgtctggggccaagggaccacggtcaccgtctcctca

SEQ ID NO: 108 - DNA, 321 bp, Artificial Sequence
  Description of Artificial Sequence: Synthetic antibody
gacatccagatgacccagtctccatcctccctgtctgcatctgtaggagacagagtcaccatcacttgccgggcaagtcagaacattgccagctatttgaattggtatcagcagaaaccagggaaagcccctaagctcctgatctatgctgcatccagtttgcaaagtggggtcccatcaaggttcagtggcagtggatcggggacagatttcactctcaccatcagcagtctgcaacctgaagagtttgcaacttactactgtcaacagagttacagtaccccgtggacgttcggccaagggaccaaggtggaaatcaaa

SEQ ID NO: 109 - DNA, 354 bp, Artificial Sequence
  Description of Artificial Sequence: Synthetic antibody
gaggtgcaactgttggagtctgggggaggcttgatacagcctggggggtccctgagactctcctgtgcagcctctggattcacctttagcaactatgccatgtcctgggtccgccaggccccagggaaggggctggagtgggtctcaggtattattagtaccagtggtggtgccacatacaacgcagactccgtgaggggccggttcaccacctccagggacaattccaagaacatactgtatctgcaaatgaacagcctcagaggcgaggacacggccgtttattactgtgtgaaaggtctctttgactggttcccgctctggggccaagggacaatggtcaccgtctcttca

SEQ ID NO: 110 - DNA, 321 bp, Artificial Sequence
  Description of Artificial Sequence: Synthetic antibody
gacatagtgatgacgcagtctccagccaccctgtctgtgtctccaggggaaagagccatcctctcctgcagggccagtcagagtgttagaagcaatttagcctggtaccagcagaaacctggccaggctcccaggctcctcatctctggtgcatccaccagggccactgctatcccagccaggttcagtggcagtgggtctgggacagagttcactctcaccatcaccagcctgcagtctgaagattgtgcagtttattactgtcaccagtataataactggcctcagacgttcggccaagggaccaaggtggaaatcaaa

SEQ ID NO: 111 - DNA, 357 bp, Artificial Sequence
  Description of Artificial Sequence: Synthetic antibody
gaggtgcagctggtggagtctgggggaggcttggtccagcctggggggtccctgagactctcctgtgcagcctctggattcacctttagtaggcattggatgacctgggtccgccaggctccagggaaggggctggagtgggtggccaacataaagcaagatggaagtgagaaatactatgtggactctgtgaagggccgactcaccatctccagagacaacgccaagaactcactgtatctgcaaatgaacagcctgagagccgaggacacggctgtgtattactgtgcgagactggggttctattacggtggagccgactactggggccagggaaccctggtcaccgtctcctca

SEQ ID NO: 112 - DNA, 336 bp, Artificial Sequence
  Description of Artificial Sequence: Synthetic antibody
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctctgaggataaccaaagaccctctggggtccctgatcggttctctggctccatcgacagctcctccaactctgcctccctcaccatctctggactgaagactgaggacgaggctgactactactgtcagtcttatgatggcatcaatcgggcatgggtgttcggcggagggaccaagctgaccgtccta

SEQ ID NO: 113 - DNA, 366 bp, Artificial Sequence
  Description of Artificial Sequence: Synthetic antibody
aacgtgcaattagtggagtctgggggaggcttggttcagcctggcgggtccctgagactctcctgtgcagcctctggattcacctttcatcattatgccatgcactgggtccgacaagctccagggaagggcctggagtgggtctcaggtattagtgggagtagtgattacagagcctatgcggactctctgaagggccgattcaccatctccagagactacgccaagaactccctgtggctgcaaatgaacagtctgacatctgaggacacggccttctattactgtgcaaagggcgttgactatggcggcaaacttgcctactttgactcctggggccagggaaccctggtcaccgtctcctca

SEQ ID NO: 114 - DNA, 336 bp, Artificial Sequence
  Description of Artificial Sequence: Synthetic antibody
gatattgtgatgactcagtctccactctccctgcccgtcacccctggagagccggcctccatctcctgcaggtctagtcagagcctcctgcatagtcttggatacaactctttgagttggtacctgcagaagccagggcagtctccacacctcctgatctatttgggctctaatcgggcctccggggtccctgacaggttcagtggcagtggatcagccacagactttacactgaaaatcagcagattggaggctgaggatgttggcgtttattactgcatgcaagctctacaaactcccctcaccttcggccaagggacacgactggagattaaa

SEQ ID NO: 115 - DNA, 369 bp, Artificial Sequence
  Description of Artificial Sequence: Synthetic antibody
caggtgcagctggtggagtctgggggaggcgtggtccagcctgggaggtccctgagactctcctgtgcagcctctggattcagcttcagtagctatgttatgaactgggtccgccaggctccaggcaaggggctggagtgggtggcagttatatcatatgatggaagcagtaaatactacgcagactccgtgaagggccgattcaccatctccagagacaattccaagaatacactgtatctgcaaatgaacagcctgagagctgaggacacggctgtgtattactgtgcgagagacattgatagtggctacgatcctacccccgtttttgactactggggccagggaaccctggtcaccgtctcctca

SEQ ID NO: 116 - DNA, 324 bp, Artificial Sequence
  Description of Artificial Sequence: Synthetic antibody
gacatccagatgacccagtctccatcctccctgtctgcatgtgtaggagacagagtcaccatcacttgccgggcaagtcagagcattagcagctatttaaattggtatcagcagaaaccagggaaaggccctaagctcctgatctatgctgcatccagtttgcaaagtggggtcccatcaaggttcagtggcagtggatctgggacagacttcactctcaccatcagcagtctgcaacctgaagattttgcaacttactactgtcaacagagttacagttccctttcgatcaccttcggccaagggacacgactggagattaaa

SEQ ID NO: 117 - DNA, 366 bp, Artificial Sequence
  Description of Artificial Sequence: Synthetic antibody
gaggtgcagctggtggagtctgggggaggcttggtacagcctggggggtccctgagactctcctgtgcagcctctggattcaccttcagtagctacgacctgcactgggtccgccaaggtacaggaaaacgtctggagtgggtctcagctattggtactgctggtgacacatactatctaggctccgtgaagggccgattcaccatctccagagaaaatgccaagaactccttgtatcttcaaatgaacagcctgagagccggggacacggctgtgtattactgtgcaagagtcctctatgatagtagtggtttttacaactggttcgacccctggggccagggaaccctggtcaccgtctcctca

SEQ ID NO: 118 - DNA, 324 bp, Artificial Sequence
  Description of Artificial Sequence: Synthetic antibody
gacatccagatgacccagtctccatcctccctgtctgcatctgttggagacagagtcaccatcacttgccgggcaagtcagagcattagcagctatttaaattggtatcagcagaaaccagggaaagcccctaagctcctgatctatgctgcatccagtttggaaagtggggtcccatcaaggttcagtggcagtggatctgggacagatttcactctcaccatcagcagtctgcaacctgaagattttgcaacttactactgtcaacagagttacgaaatccctccgtggacgttcggccaagggaccaaggtggaaatcaaa

SEQ ID NO: 119 - DNA, 348 bp, Artificial Sequence
  Description of Artificial Sequence: Synthetic antibody
gaggtgcagctggtggagtctggaggaggcttgatccagcctggggggtccctgagactctcctgtgcagcctctgaggtcaccgtcagtagcaactacatgagctgggtccgccaggctccagggaaggggctggagtgggtctcacttatttatagcggtggtactacatactacgcagactccgtgaagggccgattcaccatctccagagacaattccaagaacaccctgtatcttcaaatgaacagcctgagagccgaggacacggccgtgtattactgtgcgagagactttctacggtggcacgacctctggggccagggaaccctggtcaccgtctcctca

SEQ ID NO: 120 - DNA, 321 bp, Artificial Sequence
  Description of Artificial Sequence: Synthetic antibody
gacatccagatgacccagtctccatcctccctgtctgcatctgtaggagacagagtcaccatcacttgccaggcgagtcaggacattaacaactatttaaattggtatcagcagaaaccagggaaagcccctaagctcctgatctacgatgcatccaatttggaaacaggggtcccattaaggttcagtggaagtggatctgggacagattttattttcaccatcagcagcctgcagcctgaagatattgcaacatattactgtcaacagtatgataatctccctccagttttcggcggagggaccaaggtggagatcaaa

SEQ ID NO: 121 - DNA, 372 bp, Artificial Sequence; identical to SEQ ID NO: 77
  Description of Artificial Sequence: Synthetic antibody

SEQ ID NO: 122 - DNA, 333 bp, Artificial Sequence
  Description of Artificial Sequence: Synthetic antibody
cagtctgtgctgacgcagccgccctcagtgtctggggccccagggcagagggtcaccatctcctgcactgggagcagctccaacatcggggcaggttatgatgtacactggtaccagcagcttccagaaacagcccccaaactcctcatctatgctaacagcaatcggccctcaggggtccctgaccgattctctggctccaagtctggcacctcagcctccctggccatcactgggctccaggctgaggatgaggctgattattactgccagtcctatgacagcagcctgagtggttcggtattcggcggagggaccaagctgaccgtccta

SEQ ID NO: 123 - DNA, 366 bp, Artificial Sequence
  Description of Artificial Sequence: Synthetic antibody
cagctgcagctacaggagtcgggcccaggactggtgaagccttcggagaccctatccctcacctgcactgtctctggtggctccatcagcagtagtagttactactggggctggatccgccagcccccagggaacgggctggagtggattgggagtatctattatagtgggagcacctactacaacccgtccctcaagggtcgagtctccatatccgtagacacgtccaagaaccagttctccctgaagctgagctctgtgaccgccgcagacacggctgtctattactgtgcgagaatcttagtaatttttactttaaactggttcgacccctggggccagggaaccctggtcaccgtctcctca

SEQ ID NO: 124 - DNA, 330 bp, Artificial Sequence
  Description of Artificial Sequence: Synthetic antibody
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcggcaatcccatattcggcggagggaccaagctgaccgtccta

SEQ ID NO: 125-126 - skipped (2 entries)

SEQ ID NO: 127 - DNA, 348 bp, Artificial Sequence; identical to SEQ ID NO: 119
  Description of Artificial Sequence: Synthetic antibody

SEQ ID NO: 128 - DNA, 321 bp, Artificial Sequence
  Description of Artificial Sequence: Synthetic antibody
gacatccagatgacccagtctccatcctccctgtctgcatctgtaggagacagagtcaccatcacttgccaggcgagtcaggacattaacaactatttaaattggtatcagcagaaaccagggaaagcccctaagctcctgatctacgatgcatccaatttggaaacaggggtcccattaaggttcagtggaagtggatctgggacagattttactttcaccatcagcagcctgcagcctgaagatattgcaacatattactgtcaacagtatgataatctccctccagttttcggcggagggaccaaggtggagatcaaa

SEQ ID NO: 129 - DNA, 366 bp, Artificial Sequence
  Description of Artificial Sequence: Synthetic antibody
caggtgcagctgcaggagtcgggcccaggactggtgaagccttcggagaccctgtccctcacctgcactgtctctggtggctccgtcagcagtggtagttactactggagctggatccggcagcccccagggaagggactggagtgcattgggtatatctattacagtgggagctccaactacaacccctccctcaagagtcgagtcaccatatcagtagacacgtccaagaaccagttctccctgaagatgagctctgtgaccgctgcggacacggccgtatattactgtgcggggagccctgtccctcccacgattgtgggagcttcgtactggggccagggaaccctggtcaccgtctcctca

SEQ ID NO: 130 - DNA, 336 bp, Artificial Sequence
  Description of Artificial Sequence: Synthetic antibody
aattttatgctgactcagccccactctgtgtcggagtctccggggaagacggtaaccttctcctgcaccggcagcagtggcagcattgccagcaactatgtgcagtggtaccagcagcgcccgggcagtgcccccaccactgtgatctatgaggataaccaaagaccctctggggtccctgatcgattctctggctccatcgacagctcctccaactctgcctccctcaccatctctggactgaagactgaggacgaggctgactactactgtcagtcttatgatggcatcaatcggtggctggtattcggcggagggaccaagctgaccgtccta

SEQ ID NO: 131 - DNA, 363 bp, Artificial Sequence
  Description of Artificial Sequence: Synthetic antibody
gaggtgcagctggtgcagtctggagcagaggtgaaaaagcccggggagtctctgaagatctcctgtaagggttctggatacagctttaccagctactggatcgactgggtgcgccagatgcccgggaaaggcctggagtggatggggatcatttatcctggtgactctgataccagatatagcccgtccttccaaggccaggtcaccatctcagccgacaagtccaccagcaccgcctacctgcagtggagcagcttgaaggcctcggacaccgccatgtattactgtgcgagacgtggagaagcagctggtatttggtacttcgatctctggggccgtggcaccctggtcactgtctcctca

SEQ ID NO: 132 - DNA, 351 bp, Artificial Sequence
  Description of Artificial Sequence: Synthetic antibody
cagcctgtgctgactcagccaccttctgcatcagcctccctgggagcctcggtcacactcacctgcaccctgagcagcggctacagtaattataaagtggactggtaccagcagagaccagggaagggcccccggtttgtgatgcgagtgggcactggtgggattgtgggatccaagggggatggcatccctgatcgcttctcagtcttgggctcaggcctgaatcggtatctgacaatcaagaacatccaagaagaggatgagagtgactaccactgtggggcagaccatggcagtgggagcaacttcgagtatgtggtattcggcggagggaccaagctgaccgtccta

SEQ ID NO: 133 - DNA, 372 bp, Artificial Sequence
  Description of Artificial Sequence: Synthetic antibody
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcgctggtagcacatactacgcagactccgtgaagggcagattcagcatctccagagacaagtccaagaacacgctgtatcttcaaatgaacagcctgagagccgaggacacggctgtatattactgtgcgaaagaaggtggatcggggagcctccgctactactactacggtatggacgtctggggccaagggaccacggtcaccgtctcctca

SEQ ID NO: 134 - DNA, 330 bp, Artificial Sequence
  Description of Artificial Sequence: Synthetic antibody
cagtctgtggtgactcagccaccctcagcgtctgggacccccgggcagagggtcaccatctcttgttctggaagcagctccaacatcggatataatattgtaaactggtaccagcagctcccaggaacggcccccaaactcctcatctatagtaataatcagaggccctcaggggtccctgaccgattctctggctccaagtctggcacctcagcctccctgtccatcagtgggctccagtctgaggatgaggctgattattactgtgcagcatgggatgacagcctgaatggttatgtcttcggaactgggaccaaggtcaccgtccta

SEQ ID NO: 135 - DNA, 372 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagtacgtatgctatgcactgggtccgccaggctccaggcaaggggctggagtgggtggcagttatatcatatgatggaagcaataaatactacgcagactccgtgaagggccgattcaccatctccagagacaattccaagaacacactatatctgcaaatgatcggcctgagagctgaggacacggctgtgtattactgtgcgagagattgggcacctacgtactacgatatgccgagtgcttttgatatctggggccaagggacaatggtcaccgtctcttca

SEQ ID NO: 136 - DNA, 327 bp, Artificial Sequence
  Description of Artificial Sequence: Synthetic antibody
tcctatgtgctgactcagccaccctcggtgtcagtggccccaggaaagacggccaggattacctgtgggggaaacaacattggaaataaaggtgtgcattggtaccagcagaagccaggccaggcccctgtgctggtcgtcgatgatgatagcgaccggccctcagggatccctgagcgattctctggctccaactctgggaacacggccaccctgatcatcagcagtgtcgaagtcggggatgaggccgacttttactgtcaggtgtgggatagtagtagtgatcatccgggggtgttcggcggagggaccaagctgaccgtccta

SEQ ID NO: 137 - DNA, 372 bp, Artificial Sequence
  Description of Artificial Sequence: Synthetic antibody
gaggtgcagctggtggagtctgggggaggcttggtacagcctggagggtccctgagactctcctgtgaagcctctggattcaccttcagtagttctgaaatcaactgggtccgccaggctccagggaaggggctggagtgggtttcacacattagtagtagtggtagtatcatatactacgcagactctgtgaagggccgattcaccatctccagagacaacgccaagaactcactgtatctgcaaatgaacagcctgagagccgaggacacggctgtttattactgtgcgaggagatcttatagaagcagctggtactactactacggtatggacgtctggggccaagggaccacggtcaccgtctcctca

SEQ ID NO: 138 - DNA, 321 bp, Artificial Sequence
  Description of Artificial Sequence: Synthetic antibody
gacatccagttgacccagtctccatccttcctgtctgcttctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccccgtgacgttcggccaagggaccaaggtggaaatcaaa

SEQ ID NO: 139 - DNA, 363 bp, Artificial Sequence
  Description of Artificial Sequence: Synthetic antibody
caggtgcagctggcggagtctgggggaggcgtggtccagcctgggaggtccctgagactctcctgtgcagcctctggattcaccttcagtagctatgctatgcactgggtccgccaggctccaggcaaggggctggaatgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagaagcacgagtgggagctactactacggtatggacgtctggggccaagggaccacggtcaccgtctcctca

SEQ ID NO: 140 - DNA, 336 bp, Artificial Sequence
  Description of Artificial Sequence: Synthetic antibody
cagtctgccctgactcagcctgcctccgtgtctgggtctcctggacagtcgatcaccatctcctgcactggaaccagcagtgacgttggtgattataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgaatattactgcagctcatatacaagcagcagcactctactttatgtcttcggaactgggaccaaggtcaccgtccta

SEQ ID NO: 141 - DNA, 357 bp, Artificial Sequence
  Description of Artificial Sequence: Synthetic antibody
caggtgcagctggtgcagtctggggctgaggtgaggaagcctgggtcctcggtgaaggtctcctgcaaggcttctggaggcaccttcagcagctatgctatcagctgggtgcggcaggcccctggacaagggcttgagtggatgggagggatcatccctgtctttggtacagcaaactacgcacagaagttccagggccgagtcacgattaccgcggacaaatccacgagcacagccttcatggagctgaacagcctgagatctgaggacacggccgtctattactgtgcgagaattgggagctaccctgaatacttccagcactggggccagggcaccctggtcaccgtctcctca

SEQ ID NO: 142 - DNA, 324 bp, Artificial Sequence
  Description of Artificial Sequence: Synthetic antibody
gaaattgtgttgacacagtctccagccaccctgtctttgtctccaggggaaagagccaccctctcctgcagggccagtcagagtgttagcagcttcttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcactaccgtagcaactggcctccggttctcactttcggcggagggaccaaggtggagatc

SEQ ID NO: 143 - DNA, 366 bp, Artificial Sequence
  Description of Artificial Sequence: Synthetic antibody
caggtgcagctggtgcagtctggggctgaggtgaagaagcctgggtcctcggtgaaggtctcctgcaaggcttctggaggcaccttcagcagctatgctatcatctgggtgcgacaggcccctggacaagggcttgagtggatgggagggatcatccctatctttggtacaacaaactacgcacagaagttccagggcagagtcacgattaccgcggacgaatccacgagcacagcctacgtggaactgagcagcctgagatctgaggacacggccgtgtattattgtgcgagaataggccattttgatagtagtggttattacttagactactggggccagggaaccctggtcaccgtctcctca

SEQ ID NO: 144 - DNA, 327 bp, Artificial Sequence
  Description of Artificial Sequence: Synthetic antibody
gaaattgtgttgacacagtctccagccaccctgtctttgtctccaggggaaagagccaccctctcctgcagggccagtcagagtgttagcagcttcttagcctggtaccaacagaaacctggccaggctcccaggctcctcatctatgatgcatccaacaggcccactggcatcccagccaggttcactggcagtgggtctgggacagacttcactctcaccatcagcagcctagagcctgaagattttgcagtttattactgtcagcaccgtaccaactggcctcccttattcactttcggccctgggaccaaagtggatatcaaa

SEQ ID NO: 145 - DNA, 363 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcagtagttatgctctgttctgggtccgccaggctccaggcaaggggctggagtgggtggcagttatttcatatgatggaaataataaatactacgcagactccgtgaggggccgattcaccatctccagagacaattccaagaacacgctgtatctgcaaatgaacagcctgagacctgaggacacggctgtgtattactgtgcgagaccatatactgggagctacaagagctacatggacgtctggggcaaagggaccacggtcaccgtctcctca

SEQ ID NO: 146 - DNA, 339 bp, Artificial Sequence
  Description of Artificial Sequence: Synthetic antibody
gacatcgtgatgacccagtctccagactccctggctgtgtctctgggcgagagggccaccatcaactgcaagtccagccagagtgttttatacagctccaacaataagaactccttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtatattactgtcagcaatattatagtatttcttggacgttcggccaagggaccaaggtggaaatcaaa

SEQ ID NO: 147 - DNA, 363 bp, Artificial Sequence
  Description of Artificial Sequence: Synthetic antibody
caggtgcagctgcaggagtcgggcccaggactggtgaagccttcacagaccctgtccctcacctgcactgtctctggtggctccatcagcagtggtggttacttctggagctggatccgccagcacccagggaagggcctggagtggattgggtccatctattacagtgggagcacctactacaacccgtccctcaggagtcgaattaccatatcagtagacacgtctaagaaccagttctccctgaagctgagctctgtgactgccgcggacacggccgtgtattactgtgcgagggggggttcggggagttattctctttttgactactggggccagggaaccctggtcaccgtctcctca

SEQ ID NO: 148 - DNA, 324 bp, Artificial Sequence
  Description of Artificial Sequence: Synthetic antibody
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagattttgcaacatattactgtcaacagtatgataatctctactctgtacactttggccaggggaccaagctggagatcaaa

SEQ ID NO: 149 - DNA, 366 bp, Artificial Sequence
  Description of Artificial Sequence: Synthetic antibody
caggtgcagctggtggagtccgggggaggcgtggtccagcctgggaggtccctgagactctcctgtgcagcctctggattcaccttcagtaggcatgctatgcactgggtccgccaggctccaggcaaggggctggagtgggtggcagttatatcatatgatggaagcaataaatactatgcagactccgtgaagggccgattcgccatctccagagacaattccaagaacacgctgtatctgcaaatgaacagcctgagacctgaggacacggctgtatattactgtgcgagagatccgagcccgttagtgcttattacttcaattgactactggggccagggaaccctggtcaccgtctcctca

SEQ ID NO: 150 - DNA, 323 bp, Artificial Sequence
  Description of Artificial Sequence: Synthetic antibody
tcctatgagctgacacagccaccctcggtgtcagtgtccccaggacagacggccaggatcacctgctctggagatgcattgccaaggcaatatacttattggtaccagcagaagccaggccaggcccctgtgttggtgatatataaagacagtgagaggccctcagggatccctgagcgattctctggctccagctcagggacaacagtcacgttgaccatcagtggagtccaggcagaagacgaggctgactattactgtcaatcagcagacaccattggtacttattgggtattcggcggagggaccaagctgaccgtcct

SEQ ID NO: 151 - DNA, 357 bp, Artificial Sequence
  Description of Artificial Sequence: Synthetic antibody
gaggtgcagctggtggagtctgggggaggcttggtccagcctggggggtccctgagactctcctgtgcagcctctggattcacctttagtagctattggatgacctgggtccgccaggctccagggaaggggctggagtgggtggccaacataaagcaagatggaagtgagaaatactatgtggactctgtgaagggccgattcaccatctccagagacaacgccaagaactcactgtctctgcaaatgaacagcctgagagtcgaggacacggctgtgtattattgtgtgagactgggggtcagcagctggtactttgactactggggccagggaaccctggtcaccgtgtcctca

SEQ ID NO: 152 - DNA, 318 bp, Artificial Sequence
  Description of Artificial Sequence: Synthetic antibody
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagataccaagcggccctcagggatccctgagcgattctctggctccaactctgggaacacagccactctgaccatcagcgggacccaggctatggatgaggctgactattactgtcaggcgtggggcagtagcaggggagtgttcggcggagggaccaagctgaccgtccta

SEQ ID NO: 153 - DNA, 366 bp, Artificial Sequence
  Description of Artificial Sequence: Synthetic antibody
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtgcagcaaactacgcacagaacttccagggcagagtcacgattaccgcggacgaatccacgagcacaggctacatgcaactgagcagcctgagatttgaggacacggccgtgtattactgtgcgagaacgtctcactatgatagtagtggttcctattttgaatactggggccagggaaccctggtcaccgtctcctca

SEQ ID NO: 154 - DNA, 326 bp, Artificial Sequence
  Description of Artificial Sequence: Synthetic antibody
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagaccctgaagattttgcagtttattactgtcacaagcgtagcaactggcctccttcgctcactttcggcggagggaccaaggtggagatcaa

SEQ ID NO: 155-156 - skipped (2 entries)

SEQ ID NO: 157 - protein, 119 aa, Artificial Sequence
  Description of Artificial Sequence: Synthetic antibody
EVQLVESGGGLVQPGGSLRLSCVASGFTFSFYWMSWVRQAPGKGLEWVANIKQDGGEKYYVDSVKGRFTISRDNAKNSLYLQMNSLRAEDTAVYYCARLSGSSWDFDYWGQGTLVTVSS

SEQ ID NO: 158 - protein, 106 aa, Artificial Sequence
  Description of Artificial Sequence: Synthetic antibody
SYELTQPPSVSVSPGQTASITCSGDKLGDKYACWYQQRPGQSPVLVIYQDSKRPSGIPERFSGSNSGNTATLTISGTQAMDEADYYCQAWDSSTGVFGTGTKVTVL

SEQ ID NO: 159 - protein, 129 aa, Artificial Sequence
  Description of Artificial Sequence: Synthetic antibody
EVQLVESGGGVVRPGGSLRLSCAASGFTFDDYGMSWVRQAPGKGLEWVSAINWNGGSTGYADSVKGRFTISRDNAKNSLYLQMNSLRAEDTALYHCARRRSSSRYSSGWYMYYYYMDVWGKGTTVTVSS

SEQ ID NO: 160 - protein, 107 aa, Artificial Sequence
  Description of Artificial Sequence: Synthetic antibody
DIQMTQSPSTLSASVGDRVTITCRASQSVSTWLAWYQQKPGKAPNLLIYEASSLESGVPSRFSGSGSGTEFTLTISSLQPDDFATYYCQQYNTYSGTFGQGTKVEIK

SEQ ID NO: 161 - protein, 120 aa, Artificial Sequence
  Description of Artificial Sequence: Synthetic antibody
QITFKESGPTLVKPTETLTLTCTFSGFSVSTSGEGVGWIRQPPGKALEWLAVIYWDDDKRYSPSLKSRLTITRDTSKNQVVLTMTNMDPVDTATYYCAHRLWFRDAFDIWGQGTTVTVSS

SEQ ID NO: 162 - protein, 107 aa, Artificial Sequence
  Description of Artificial Sequence: Synthetic antibody
DIQMTQSPSSLSASVGDRVTITCRARQSISNYLNWYQQKPGKAPKLLIYAASSLHSGVPSRFSGSGSGTDFTLTISSLQPEDFATYYCQQTYSTFWTFGQGTNVEIK

SEQ ID NO: 163 - protein, 119 aa, Artificial Sequence
  Description of Artificial Sequence: Synthetic antibody
EVQLVESGGGLVQPGGSLRLSCAASGFTFSTYWMTWVRQAPGKGLEWVANIKQDGSEKYYVDSVKYRFTISRDNAKNSLYLQMNSLRAEDTAVYYCARVGSSSWYFDYWGQGTLVTVSS

SEQ ID NO: 164 - protein, 106 aa, Artificial Sequence
  Description of Artificial Sequence: Synthetic antibody
SYELTQPPSVSVSPGQTASITCSGDKLGDKYACWYQQKPGQSPVVVIYQDSKRPSGIPERFSGSNSGNTATLTISGTQAMDEADYYCQAWDSSTAVFGGGTKLTVL

SEQ ID NO: 165 - protein, 123 aa, Artificial Sequence
  Description of Artificial Sequence: Synthetic antibody
QLQLQESGPGLVKPSETLSLTCTVSGGSISSGTYYCGWIRQPPGKGLEWIGSTYYGGSTLYNPSLRGRVTISVDTSKNQFSLKLSSVTAADTAVYYCARRGNYYDSKNWFDPWGQGTLVTVSS

SEQ ID NO: 166 - protein, 109 aa, Artificial Sequence
  Description of Artificial Sequence: Synthetic antibody
EIVMTQSPATVSVSPGERATLSCRASQSVSSNLAWYQQKPGQAPRLLIYGASTRATGIPARFSASGSGTEFTLTISSLQSEDFAVYYCQQYNNWPPMYTFGQGTKVEIK

SEQ ID NO: 167 - protein, 120 aa, Artificial Sequence
  Description of Artificial Sequence: Synthetic antibody
QVQLVESGGGVVQPGRSLRVSCAASGFTFSSHGMHWVRQAPGKGLEWVSVIWYDGSNKYYADSVKGRFTISRDNSKNTLSLQMNSLRAEDTAVYYCARESADISSRLDYWGRGTLVTVSS

SEQ ID NO: 168 - protein, 106 aa, Artificial Sequence
  Description of Artificial Sequence: Synthetic antibody
SYELTQPPSVSVSPGQTARITCSGDALPTKYAYWYQQKSGQAPVLVIYDDSKRPSGIPERFSGSSSGTMATLTISGAQVEDEADYYCYSTDSSGNVFGTGTKVTVL

SEQ ID NO: 169 - protein, 121 aa, Artificial Sequence
  Description of Artificial Sequence: Synthetic antibody
QVQLVESGGGVVQPGRSLRLSCAASGFTFSSYAMHWVRQAPGKGLEWVAGISYDGSNKYYADSVKGRFTISRDNSKNTLYLQMNSLRAEDTAVYYCARADTMVRGTYFEYWGQGTLVTVSS

SEQ ID NO: 170 - protein, 110 aa, Artificial Sequence
  Description of Artificial Sequence: Synthetic antibody
QSALTQPASVSGSPGQSITISCTGTSSDVGGYNYVSWYQQHPGKAPKLMIYDVSNRPSGVSNRFSGSKSGNTASLTISGLQAEDEADYCCSSYTSSRAVLFGGGTKLTVL

SEQ ID NO: 171 - protein, 127 aa, Artificial Sequence
  Description of Artificial Sequence: Synthetic antibody
QITLKESGPTLVKPTQTLTLTCTFSGFSLSTSGVGVAWIRQPPGKALEWLALIYWDDDKRYSPSLKSRLTITKDTSKNQVVLTMTNMDPVDTATYYCAHRLPTPQLLPSFENWFDPWGQGTLVTVSS

SEQ ID NO: 172 - protein, 110 aa, Artificial Sequence
  Description of Artificial Sequence: Synthetic antibody
QSVLTQPPSVSEAPRQRVTISCSGGSSNIGNNAVNWYQQLPGKAPKLLIYYDDLLPSGVSDRFSGSKSGTSASLAISGLQSEDEADYYCASWDDSLIGPVFGGGTKLTVL

SEQ ID NO: 173 - protein, 120 aa, Artificial Sequence
  Description of Artificial Sequence: Synthetic antibody
EVQLVESGGGLVQPGGSLRLSCAVSGFTFSSYWMHWVRQAPGKGLVWVSRINSDGSSTSYADSVKGRFTISRDNAKNTLYLQMNSLRAEDTAVYYCAREVEQLAHMVDYWGQGTLVTVSS

SEQ ID NO: 174 - protein, 108 aa, Artificial Sequence
  Description of Artificial Sequence: Synthetic antibody
SYELTQPPSVSVSPGQTARITCSGDALPNQYAYWYQQKPGQAPVLVIYKDSERPSGIPERFSGSSSGTTVTLTISGVQAEDEADYYCQSADSSGTSWVFGGGTKLTVL

SEQ ID NO: 175 - protein, 121 aa, Artificial Sequence
  Description of Artificial Sequence: Synthetic antibody
QVQLVESGGGVVQPGRSLRLSCAASGFTFSSYAMHWVRQAPGKGLEWVAVISYDGINKYYADAVKGRFTISRDNSKNTLYLQMNSLRAEDTAVYYCARPRSGSYYAYFDYWGQGTLVTVSS

SEQ ID NO: 176 - protein, 107 aa, Artificial Sequence
  Description of Artificial Sequence: Synthetic antibody
DIQMTQSPSSLSASVGDRVTITCRASQGISNYLAWFQQKPGKAPKSLIYAASSLQSGVPSKFSGSGSGTDFTLTISSLQPEDVATYYCQQYNSHPPTFGGGTKVEIK

SEQ ID NO: 177 - protein, 119 aa, Artificial Sequence
  Description of Artificial Sequence: Synthetic antibody
QVQLVESGGGVVQPGRSLRLSCAASGFTFSYYPMHWLWVRQAPGKGLEWVAVTSYDGTNKYYADSVKGRFTISRDNSKNTLYLQMNSLRAEDTAVYYCARGGATNFDYWGQGTLVTVSS

SEQ ID NO: 178 - protein, 110 aa, Artificial Sequence
  Description of Artificial Sequence: Synthetic antibody
SYVLTQPPSVSVAPGKTANITCGGNNIGRKSVHWYQQKSGQAPVLVVYDDSDRPSGIPERFSGSNSGNTATLTISRVEAGDEADYYCQVWDSSSDHPEWVFGGGTKLTVL

SEQ ID NO: 179 - protein, 126 aa, Artificial Sequence
  Description of Artificial Sequence: Synthetic antibody
EAQLVESGGGLVQPGRSLRLSCAASGFTFDDSAMHWVRQAPGKGLEWVSGISWNSGNVGYADSVKGRFTISRDNAKNSLYLQMNSLRAEDTALYYCTKASRYCSSTICYWNWFDPWGQGTLVTVSS

SEQ ID NO: 180 - protein, 106 aa, Artificial Sequence
  Description of Artificial Sequence: Synthetic antibody
DIQMTQSPSSLSASVGDRVTITCRASQSISSYLNWYQQKPGKAPKLLIYGASSLQTGVPSRFSGSGSGTDFTLTIRSLQPEDFASYYCQQSYSTPTFGGGTKVEIK

SEQ ID NO: 181 - protein, 126 aa, Artificial Sequence
  Description of Artificial Sequence: Synthetic antibody
EVQLVESGGGLVQPGGSLRLSCAASGFTFSSYEMNWVRQAPGKGLEWVSYISSSGSAIYYADSVKGRFTISRDNAKNSLYLQMNSLRVEDTAVYYCAREARSRYFDWLPSYYFDYWGQGTLVTVSS

SEQ ID NO: 182 - protein, 111 aa, Artificial Sequence
  Description of Artificial Sequence: Synthetic antibody
QSALTQPASVSGSPGQSITISCTGTSSDIGGYNYVSWYQQHPGKAPKLLIYDVSNRPSGVSTRFSGSKSGNTASLTISGLQAEDEADYYCSSYTSSSTHVVFGGGTKLTVL

SEQ ID NO: 183 - protein, 125 aa, Artificial Sequence
  Description of Artificial Sequence: Synthetic antibody
QVQLVQSGSELKKPGASVKVSCKASGYTFTTYAMNWVRQAPGQGLEWMGWINTNTGNPTYAQGFTGRFVFSLDTSVNTAFLHIGSLKAEDTAVYYCARDQDSGYPTYYYYYMDVWGKGTTVTVSS

SEQ ID NO: 184 - protein, 112 aa, Artificial Sequence
  Description of Artificial Sequence: Synthetic antibody
DIVMTQTPLSLSVTPGQPASISCKSSQSLLHSDGKTYLYWYLQKPGQSPQLLIYEVSNRFSGVPDRFSGSGSGTDFTLKISRVEAEDVGVYYCMQSIQPPLTFGGGTKVEIK

SEQ ID NO: 185 - protein, 121 aa, Artificial Sequence
  Description of Artificial Sequence: Synthetic antibody
QVQLVESGGGVVQPGRSLRLSCAASGFTFSTYAMHWVRQAPGKGLAWVALISYDGYNKYYADSVRGRFTISRINSKNTLSLQMNSLRAEDTAVYYCARGSAGNYYYGMDVWGQGTTVTVSS

SEQ ID NO: 186 - protein, 107 aa, Artificial Sequence
  Description of Artificial Sequence: Synthetic antibody
DIQMTQSPSSLSASVGDRVTITCRASQTITNYLNWYQLKSGRAPKLLIYAASSLQSGVPSRFSGSGSGTDFTLTISSLQPEDFATYYCQQSYSTPYTFGQGTKLEIK

SEQ ID NO: 187 - protein, 127 aa, Artificial Sequence
  Description of Artificial Sequence: Synthetic antibody
QVHLVESGGGVVQPGRSLRLSCAASGFTFSNYGMHWVRQAPGKGLEWVAVISNDEFNKFYANSVKGRFTISRDNSKNTVYLQLNSLRTEDTARYYCAKGGDGSGWAWDGDNPPTDYWGQGTLVIVSS

SEQ ID NO: 188 - protein, 112 aa, Artificial Sequence
  Description of Artificial Sequence: Synthetic antibody
DIVMTQSPDFLAVSLGERATISCKSSQSVLYTPKNKNYLAWYKQKPGQPPKVLIYWASTRESGVPDRFSGSGSGTDFTLIISSLQAEDAAVYYCQQYYTAPLTFGGGTRVEI

SEQ ID NO: 189-190 - skipped (2 entries)

SEQ ID NO: 191 - protein, 124 aa, Artificial Sequence
  Description of Artificial Sequence: Synthetic antibody
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARPDYSSGWFSYWYFDLWGRGTLVTVSS

SEQ ID NO: 192 - protein, 108 aa, Artificial Sequence
  Description of Artificial Sequence: Synthetic antibody
EIVLTQSPGTLSLSPGERATLSCRASQSVSSNFLAWYQQKPGQAPRLLIYGASSRATGIPDRFSGSGSGTDFTLTISRLEPEDFAVYYCQQYGRSPITFGQGTRLEIK

SEQ ID NO: 193 - protein, 121 aa, Artificial Sequence
  Description of Artificial Sequence: Synthetic antibody
QVQLVQSGAEVKKPGASVKVSCKVSGYTLTELSIHWVRQAPGKGLEWMGGFDPEDAETIYAQQFQGRVTMTEDTSTDTAYMELSSLKSEDTALYYCATGFAVFGRAAVPYWGQGTLVTVSS

SEQ ID NO: 194 - protein, 106 aa, Artificial Sequence
  Description of Artificial Sequence: Synthetic antibody
SYELTQPPSVSVSPGQTASITCFGDKLGDKYACWFQQKPGQSPVLIIYQGAKRPSGIPERFSGSNSGNTATLTISGTQAMDEADYYCQAWDSSTVVFGGGTKLTVL

SEQ ID NO: 195 - protein, 124 aa, Artificial Sequence
  Description of Artificial Sequence: Synthetic antibody
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLTIVVIPAAPNFDYWGQGTLVTVSS

SEQ ID NO: 196 - protein, 111 aa, Artificial Sequence
  Description of Artificial Sequence: Synthetic antibody
QSALTQPASVSGSPGQSITISCTGTSSDVGGYNYVSWYQQHPGKAPKLMIYDVSNRPSGVSNRFSGSRSGNTASLTISGLQAEDEADYYCSSYTSSSTPVVFGGGTKLTVL

SEQ ID NO: 197 - protein, 121 aa, Artificial Sequence
  Description of Artificial Sequence: Synthetic antibody
EVQLVQSGAEVKKPGESLKISCKGSGYSFTSHWIGWVRQMPGKGLEWMGIIYPGDSDTRYSPSFQGQVTISADKSISTAYLQWSSLKASDTAMYYCASALRERGVQLWSVWGQGTLVTVSS

SEQ ID NO: 198 - protein, 110 aa, Artificial Sequence
  Description of Artificial Sequence: Synthetic antibody
QSVLTQPPSVSGAPGQRVTISCTGSSSNIGAGYDVHWYQQLPGTAPKLLIFINSNRPSGVPDRFSGSKSGTSASLAITGLQAEDEADYYCQSYDSSLGALFGGGTKLTVL

SEQ ID NO: 199 - protein, 122 aa, Artificial Sequence
  Description of Artificial Sequence: Synthetic antibody
QVQLVESGGGVVQPGRSLRLSCAASGFTFSSYAMHWVRQAPGKGLEWVAVISYDGSNKYYADSVKGRFTISRDNSKNTLYLQMNSLRVEDTAVYYCARDQEWFRELFLFDYWGQGTLVTVSS

SEQ ID NO: 200 - protein, 107 aa, Artificial Sequence
  Description of Artificial Sequence: Synthetic antibody
DIQMTQSPSSVSASVGDRVTITCRASQGISSWLAWYQQKPGKAPKLLIYDASSLQSGVPSRFSGSGSGTDFTLTISSLQPEDFATYYCQQANSFPPTFGPGTKVDIK

SEQ ID NO: 201 - protein, 118 aa, Artificial Sequence
  Description of Artificial Sequence: Synthetic antibody
QVQLVESGGGVVQPGRSLRLSCAASGFTFSTYGMHWVRQAPGKGLEWVAVISYDGSNKYYADSVKGRFTISRDNSKNTLYLQMNSLRAEDTAMYYCAKDGSIAAADYWGQGTLVTVSS

SEQ ID NO: 202 - protein, 113 aa, Artificial Sequence
  Description of Artificial Sequence: Synthetic antibody
DIVMTQSPDSLAVSLGERATINCKSSQSVLHSSNNKDSLVWYQQKPGQPPKLLIYWASSRESGVPDRFSGSGSGTDFTLTISSLQAEDVAVYYCQQYYSTPWTFGQGTKVEIK

SEQ ID NO: 203 - protein, 128 aa, Artificial Sequence
  Description of Artificial Sequence: Synthetic antibody
EVQLVESGGGLVKPGGSLRLSCAASGFTFSSYSMNWVRQAPGKGLEWVSSISNSNSFIYYADSMKGRFTISRDNAKNSLYLQMNSLRAEDTAVYYCARVNGNSNWNFGSYYYYYMDVWGKGTTVTVSS

SEQ ID NO: 204 - protein, 106 aa, Artificial Sequence
  Description of Artificial Sequence: Synthetic antibody
EIVLTQSPAILSLSPGERATLSCRASQSVSSYLAWYQQKPGQAPRLLIYDTSNRATGIPARFSGSGSGTDFTLTISSLEPEDFAFYYCQQRGNWWTFAQGTKVEIK

SEQ ID NO: 205 - protein, 124 aa, Artificial Sequence
  Description of Artificial Sequence: Synthetic antibody
EVQLVESGGGLVKPGGSLRLSCAASGFTFSGYSMNWVRQAPGKGLEWVSSISSSSSYIYYADSVKGRFTISRDNAKNSLYLQMNSLRAEDTAVYYCARWLQLRSDYYYFGMDVWGQGTTVTVSS

SEQ ID NO: 206 - protein, 108 aa, Artificial Sequence
  Description of Artificial Sequence: Synthetic antibody
EIVMTQSPATLSVSPGERATLSCRASQSVSNNLAWYQHKPGQAPRLLIYGASTRATGIPARFSGSGSGTEFTLTISSLQSEDFAVYFCQQCYNWPPWTFGQGTKVEIK

SEQ ID NO: 207 - protein, 123 aa, Artificial Sequence
  Description of Artificial Sequence: Synthetic antibody
QVQLQESGPGLVKPSETLSLTCTVSGGSISSYYWSWIRQPPGKGLEWIGYIYYSGSTNYNPSLKSRVTISVDMSKNQFSLKLRSVTAADTAVYYCARAPRERLQWGEYYFDYWGQGTLVTVSS

SEQ ID NO: 208 - protein, 111 aa, Artificial Sequence
  Description of Artificial Sequence: Synthetic antibody
QSALTQPASVSGSPGQSITISCTGTSSDVGSYNLVSWYQQHAGKAPKLMIYEVIKRPSGVSNRFSGSKSGNTASLTISGLQAEDEADYYCCSYAVSTTYVIFGGGTKLTVL

SEQ ID NO: 209 - protein, 121 aa, Artificial Sequence
  Description of Artificial Sequence: Synthetic antibody
EVQLVQSGAEVKKPGESLKISCKGSGYSFTNSWIGWVRQMPGKGLEWMGIIYPGDSDTRYSPSFQGQVTISADKSISTAYLQWSSLKASDTAIYYCATHRCSGGFCYLAYWGQGTLVTVSS

SEQ ID NO: 210 - protein, 117 aa, Artificial Sequence
  Description of Artificial Sequence: Synthetic antibody
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVFVVFGGGTKLTVL

SEQ ID NO: 211 - protein, 128 aa, Artificial Sequence
  Description of Artificial Sequence: Synthetic antibody
QVQLVESGGGVVQPGRSLRLSCAASGFTFSSYGMHWVRQAPGKGLEWVAVIWYDGSKKDYADSVKGRFTISRDNSKNTLYLQMNSLRAEDTAVYYCARDQSQGAYILTGYRGYGMDVWGQGTTVTVSS

SEQ ID NO: 212 - protein, 112 aa, Artificial Sequence
  Description of Artificial Sequence: Synthetic antibody
DIVMTQSPLSLPVTPGEPASISCRSSQSLLHSNGYNYLDWYLQKPGQSPQFLIYLGSNRASGVPDRFSGSGSGTDFILKISRVEAEDVGVYYCMQALQTPFTFGPGTKVDIK

SEQ ID NO: 213 - protein, 123 aa, Artificial Sequence
  Description of Artificial Sequence: Synthetic antibody
QVQLQQWGAGLLKPSETLSLTCAVYGGSFSGHYWSWIRQPPGKGLEWIGEINHSGSTNYNPSLKSRVTISVDTSKNQFSLKLSSVTAADTAVYYCARPPQAARIHYYYYMDVWGKGTTVTVSS

SEQ ID NO: 214 - protein, 108 aa, Artificial Sequence
  Description of Artificial Sequence: Synthetic antibody
EIVMTQSPATLSVSPGERATLSCRASQSVSSNLAWYQQKPGQAPRLLIYGASTRATGIPARFSGSGSGTEFTLTISSLQSEDFAVYYCQQYNYWPPLTFGGGTKVEIK

SEQ ID NO: 215 - protein, 121 aa, Artificial Sequence
  Description of Artificial Sequence: Synthetic antibody
EVQLVESGGGLVQPGRSLRLSCAASGFTFDDYAMNWVRQPPGKGLEWVSGISWNSDSIGYADSVKGRFTISRDNAKNSLYLQMNSLRAEDTAMYYCAKGRGAGYTSYMDVWGKGTTVTVSS

SEQ ID NO: 216 - protein, 109 aa, Artificial Sequence
  Description of Artificial Sequence: Synthetic antibody
SYVLTQPPSVSVAPGKTARITCEGNNIGSKSVHWYQQKPGQAPVLVVYDDSGRPSGIPERFSGSNSGNTATLTISRVEAGDEADYFCQVWDSSSDHHVVFGGGTKLTVL

SEQ ID NO: 217 - protein, 99 aa, Artificial Sequence
  Description of Artificial Sequence: Synthetic antibody
YTTLFRSGGHWIGWVRQMPGKGLEWMGIIYPGDSDTRYSPSFQGQVTISADKSISTAYLQWSSLKASDTAMYYCASALRERGVQLWSVWGQGTLVTVSS

SEQ ID NO: 218 - protein, 110 aa, Artificial Sequence; identical to SEQ ID NO: 198
  Description of Artificial Sequence: Synthetic antibody

SEQ ID NO: 219 - protein, 123 aa, Artificial Sequence
  Description of Artificial Sequence: Synthetic antibody
EVQLVESGGGLVQPGRSLRLSCAASGFTFDDYAMHWVRQAPGKGLEWVSGISWNSGSIAYTDSVKGRFTISRDNAKNSLYLQMNSLRAEDTALYYCAKAHSTGHQYYYGMDVWGQGTTVTVSS

SEQ ID NO: 220 - protein, 107 aa, Artificial Sequence
  Description of Artificial Sequence: Synthetic antibody
DIQMTQSPSSLSASVGDRVTITCRASQSISSFLNWYQQKPGKAPKLLIYAAFNLQSGVPSRFSGSGSGTDFTLTISSLQSEDFATYYCQQSYNTPYTFGQGTKLEIK

SEQ ID NO: 221 - protein, 123 aa, Artificial Sequence
  Description of Artificial Sequence: Synthetic antibody
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVSSITSLLGYYFDSWGQGTLVTVSS

SEQ ID NO: 222 - protein, 109 aa, Artificial Sequence
  Description of Artificial Sequence: Synthetic antibody
EIVLTQSPATLSLSPGERATLSCRASQSVSSYLAWYQQKPGQAPRLLIYDASNRATGIPARFSGSGSGTDFTLTISSLEPEDFAVYYCQHRSNWPPRLTFGGGTKVEIK

SEQ ID NO: 223 - protein, 121 aa, Artificial Sequence
  Description of Artificial Sequence: Synthetic antibody
QVQLVQSGAEVKKPGSSVKVSCKTSGDTSSSYTVGWVRQAPGQGLEWMGRIIPILGIAYSAQKFQGRLTITADKSTSTSYMELSSLRSEDTAVYYCARGVVAATPGWFDPWGQGTLVTVSS

SEQ ID NO: 224 - protein, 106 aa, Artificial Sequence
  Description of Artificial Sequence: Synthetic antibody
EIVMTQSPATLSVSPGERVTLSCRASQSVSSNLAWYQQKPGQAPRLLIYGASTRATGIPARFSGGGSGTEFTLTISSLQSEDFAVYYCQQYNNFLTFGGGTKVEIK

SEQ ID NO: 225 - protein, 119 aa, Artificial Sequence
  Description of Artificial Sequence: Synthetic antibody
EVQLVESGGGLVQPGGSLRLSCAASGFTFSSYWMHWVRQVPGKGLVWVSRINSDGSSTSYADSVKGRFTISRDNAKNTLYLEMNSLRAQDTAVYYCAGSPWLRGDIDYWGQGTLVTVSS

SEQ ID NO: 226 - protein, 112 aa, Artificial Sequence
  Description of Artificial Sequence: Synthetic antibody
NFMLTQPHSVSESPGKTVTISCTGSSGSIASNYVQWYQQRPGSAPTTVIYEDNQRPSGVPDRFSGSIDSSSNSASLTISGLMTEDEADYYCQSYDGSNHAVVFGGGTKLTVL

SEQ ID NO: 227 - protein, 130 aa, Artificial Sequence
  Description of Artificial Sequence: Synthetic antibody
QVQLVQSGAEVKKPGASVKVSCKASGYTFTDYYMHWVRQAPGQGLEWMGWINPNSRGTNYAQKFQGRVTMTRDTSISTVYMELSRLTSDDTAVYYCARVVVLGYGRPNNYYDGRNVWDYWGQGTLVTVSS

SEQ ID NO: 228 - protein, 110 aa, Artificial Sequence
  Description of Artificial Sequence: Synthetic antibody
QSVLTQPPSASGTPGQRVIISCSGSSSNIGSNTVKWYHQLPGTAPKLLICSNNQRPSGVPDRFSGSKSDTSASLAISGLQSEDEADYYCAAWDDSLNALVFGGGTKLTVL

SEQ ID NO: 229 - protein, 117 aa, Artificial Sequence
  Description of Artificial Sequence: Synthetic antibody
QVTLRESGPALVKPTQTLSLTCTFSGFSLGTSGMCVSWIRQPPGKALEWLARIDWDDDKYYSTSLKTRLTISKDTSKNQVVLTMTNMDPVDTATYYCARGVVTYDYWGQGTLVTVSS

SEQ ID NO: 230 - protein, 107 aa, Artificial Sequence
  Description of Artificial Sequence: Synthetic antibody
DIQMTQSPSSLSASVGDRVTITCRASQSIAGYLNWYQQKPGKAPKLLIYGTTSLQSGVPVRFSGSGSGTDFTLTISSLQPEDFATYYCQQSYSTPGTFGQGTRLEIK

SEQ ID NO: 231 - protein, 124 aa, Artificial Sequence
  Description of Artificial Sequence: Synthetic antibody
QVQLVQSGAEVKKPGSSVKVSCKASGDTFSSYTINWVRQAPGQGLEWMGRIIPILGIPNYAQKFQGRVTITADKSTSTAFMELSSLRSEDTAVYYCARGRGYSNYGASYYMDVWGKGTTVTVSS

SEQ ID NO: 232 - protein, 108 aa, Artificial Sequence
  Description of Artificial Sequence: Synthetic antibody
DIQMTQSPSSLSASVGDRVTITCQASQDINHYLNWYQQKPGKAPKLLIYDASNLETGVPSRFSGSGSGTDFTFTITSLQPEDVATYYCQQSDNLPMYTFGQGTKLEIK

SEQ ID NO: 233 - protein, 121 aa, Artificial Sequence; identical to SEQ ID NO: 215
  Description of Artificial Sequence: Synthetic antibody

SEQ ID NO: 234 - protein, 109 aa, Artificial Sequence; identical to SEQ ID NO: 216
  Description of Artificial Sequence: Synthetic antibody

SEQ ID NO: 235-236 - skipped (2 entries)

SEQ ID NO: 237 - protein, 123 aa, Artificial Sequence
  Description of Artificial Sequence: Synthetic antibody
QVQLAQSGAEVKKPGASVKVSCKAAGYTFTSYDINWVRQATGQGLEWMGWMNSNSGNAGYAQKFQGRVTMTRDTSTSTAYMELSSLTSDDTAVYYCARMRTGWPTHGRPDDFWGRGTLVTVSS

SEQ ID NO: 238 - protein, 110 aa, Artificial Sequence
  Description of Artificial Sequence: Synthetic antibody
QSVLTQPPSASGTPGQRVTISCSGSNSNIGSYTVNWYQQFPGTAPKLLIYDNNQRTSGVPDRFSGSKSGTSASLAISGLQSEDEANYYCLVWDDSLNGLVFGGGTKLTVL

SEQ ID NO: 239 - protein, 122 aa, Artificial Sequence
  Description of Artificial Sequence: Synthetic antibody
EVQLVESGGGLVQPGGSLRLSCAASGFTVSSNYMSWVRQAPGKGLEWVSVIYPGGSAFYADSVKGRFTISRHNSNNTLCLQMNSLRTEDTAVYYCARSYDILTGYRDAFDIWGQGTMVTVSS

SEQ ID NO: 240 - protein, 112 aa, Artificial Sequence
  Description of Artificial Sequence: Synthetic antibody
QSVLTQPPSVSGAPGQRVTISCTGSSSNIGSGSDVHWYQQLPGTAPKLLIYGNTNRPSGVPDRFSGSKSGTSASLAITGLQAEDEADYYCQSYDSRLSGFVVFGGGTKLTVL

SEQ ID NO: 241 - protein, 119 aa, Artificial Sequence
  Description of Artificial Sequence: Synthetic antibody
EVQLVESGGGLVQPGGSLRLSCAASGLTVSSNYMSWVRQAPGKGLECVSVIYAGGNTYYADSVKGRFTISRDNSKNTLYLQMNSLRAEDTAVYYCARGDGGYYSPFDYWGQGTLVTVSS

SEQ ID NO: 242 - protein, 107 aa, Artificial Sequence
  Description of Artificial Sequence: Synthetic antibody
DIQMTQSPSSLSASVGDRVTITCRASQSISSYLNWYQQKPGKAPKVLIYAASTMQSGVPSRFRGSGSGTDFTLTISSLQLEDFATYYCQQSYSTPQTFGQGTKVEIK

SEQ ID NO: 243 - protein, 122 aa, Artificial Sequence
  Description of Artificial Sequence: Synthetic antibody
QVQLQQWGAGLLKPSETLSLTCAVSGGSFSAYYWSWIRQPPGKGLEWIGEINHSGSTNYNPSLRSRVTISVDTSKNQFSLKLSSVTAADTAVYYCARVGYSQGYYYYYMDVWGKGTTVTVSS

SEQ ID NO: 244 - protein, 107 aa, Artificial Sequence
  Description of Artificial Sequence: Synthetic antibody
DIQMTQSPSSLSASVGDRVTITCRASQSISNYLNWYQQKPGKAPKLLIYAASSLQSGVPSRFSGSGSGTDFSLTISSLQPEDFATYSCQQSYTTLLTFGGGTKVEIK

SEQ ID NO: 245-246 - skipped (2 entries)

SEQ ID NO: 247 - protein, 125 aa, Artificial Sequence
  Description of Artificial Sequence: Synthetic antibody
QVQLVQSGAEVKKPGSSVKVSCKASGGTFSSYSITWVRQAPGQGLEWMGRIIPVLGIANYAQKFQDRVTITADKSTSTAYMELSSLRSEDTAVYYCARVGVSGFKSGSNWYFDLWGRGTLVTVSS

SEQ ID NO: 248 - protein, 111 aa, Artificial Sequence
  Description of Artificial Sequence: Synthetic antibody
QSVLTQPPSVSGAPGQRVTLSCTGSNSNIGAGYDVHWYQQLPGTAPKLLIYGNSNRPSGVPDRFSGSKSGTSASLAITGLQAEDEADYYCQSYDSSLSDSVFGGGTKVTVL

SEQ ID NO: 249-250 - skipped (2 entries)

SEQ ID NO: 251 - protein, 122 aa, Artificial Sequence
  Description of Artificial Sequence: Synthetic antibody
QVQLVQSGAEVKKPGASVKVSCKASGYTFTSYYMHWVRQAPGQGLEWMGIINPGGGSTTYAQKFQGRVTMTSDTSTSTVYMELSSLRSEDTAMYYCARGAIPPNSRAEIDYWGQGTLVTVSS

SEQ ID NO: 252 - protein, 107 aa, Artificial Sequence
  Description of Artificial Sequence: Synthetic antibody
EIVMTQSPATLSVSPGERVTLSCRASQSVSSNLAWCQQKPGQAPRLLIYGASTRATGIPARFSGSGSGTEFTLTISSLQSEDFAVYYCQQYYNWPLTFGGGTKVEIK

SEQ ID NO: 253 - protein, 129 aa, Artificial Sequence
  Description of Artificial Sequence: Synthetic antibody
EVQLVESGGGVVRPGGSLRLSCAASGFIFDDYDMTWVRQAPGKGLEWVSGISWNGGNTGYADSVKGRFTISRDNAKNSLYLQMNSLRAEDTALYHCAVIMSPIPRYSGYDWAGGAFDIWGQGTMVTVSS

SEQ ID NO: 254 - protein, 108 aa, Artificial Sequence
  Description of Artificial Sequence: Synthetic antibody
SSELTQDPAVSVALGQTVRITCQGDSLRSYYASWYQQKPGQVPILVIYDKNNRPSGIPDRFSGSSSGNTASLTITGAQAEDEADYYCNSRDSSGNAVVFGGGTKLTVL

SEQ ID NO: 255 - protein, 127 aa, Artificial Sequence
  Description of Artificial Sequence: Synthetic antibody
QVQLVESGGGVVQPGRSLRLSCAASGFTFSNYGMHWVRQAPGKGLEWVAVMSYDGSNKYYADSVKGRFTISRDNSKNTLYLQMNSLRAEDTAVYYCAKNLGPYCSGGTCYSLVGDYWGQGTLVTVSS

SEQ ID NO: 256 - protein, 107 aa, Artificial Sequence
  Description of Artificial Sequence: Synthetic antibody
DIQMTQSPSSLSASVGDRVTITCQASQDISNYLNWYQQKPGKAPKLLIYDASNLETGVPSRFSGSGSGTDFTFTISSLQPEDIATYYCQQYANLPFTFGPGTKVDIK

SEQ ID NO: 257-260 - skipped (4 entries)

SEQ ID NO: 261 - protein, 121 aa, Artificial Sequence
  Description of Artificial Sequence: Synthetic antibody
EVQLVESGGGLVQPGRSLRLSCAASGFTFDDYAMHWVRQAPGKGLEWVSGISWNSGTIGYADSVKGRFIISRDNAKNSLYLQMNSLRPEDTALYYCAKDIIRQGEDGMDVWGQGTTVTVSS

SEQ ID NO: 262 - protein, 107 aa, Artificial Sequence
  Description of Artificial Sequence: Synthetic antibody
DIQMTQSPSSLSASVGDRVTITCRASQNIASYLNWYQQKPGKAPKLLIYAASSLQSGVPSRFSGSGSGTDFTLTISSLQPEEFATYYCQQSYSTPWTFGQGTKVEIK

SEQ ID NO: 263 - protein, 118 aa, Artificial Sequence
  Description of Artificial Sequence: Synthetic antibody
EVQLLESGGGLIQPGGSLRLSCAASGFTFSNYAMSWVRQAPGKGLEWVSGIISTSGGATYNADSVRGRFTTSRDNSKNILYLQMNSLRGEDTAVYYCVKGLFDWFPLWGQGTMVTVSS

SEQ ID NO: 264 - protein, 107 aa, Artificial Sequence
  Description of Artificial Sequence: Synthetic antibody
DIVMTQSPATLSVSPGERAILSCRASQSVRSNLAWYQQKPGQAPRLLISGASTRATAIPARFSGSGSGTEFTLTITSLQSEDCAVYYCHQYNNWPQTFGQGTKVEIK

SEQ ID NO: 265 - protein, 119 aa, Artificial Sequence
  Description of Artificial Sequence: Synthetic antibody
EVQLVESGGGLVQPGGSLRLSCAASGFTFSRHWMTWVRQAPGKGLEWVANIKQDGSEKYYVDSVKGRLTISRDNAKNSLYLQMNSLRAEDTAVYYCARLGFYYGGADYWGQGTLVTVSS

SEQ ID NO: 266 - protein, 112 aa, Artificial Sequence
  Description of Artificial Sequence: Synthetic antibody
NFMLTQPHSVSESPGKTVTISCTGSSGSIASNYVQWYQQRPGSAPTTVISEDNQRPSGVPDRFSGSIDSSSNSASLTISGLKTEDEADYYCQSYDGINRAWVFGGGTKLTVL

SEQ ID NO: 267 - protein, 122 aa, Artificial Sequence
  Description of Artificial Sequence: Synthetic antibody
NVQLVESGGGLVQPGGSLRLSCAASGFTFHHYAMHWVRQAPGKGLEWVSGISGSSDYRAYADSLKGRFTISRDYAKNSLWLQMNSLTSEDTAFYYCAKGVDYGGKLAYFDSWGQGTLVTVSS

SEQ ID NO: 268 - protein, 112 aa, Artificial Sequence
  Description of Artificial Sequence: Synthetic antibody
DIVMTQSPLSLPVTPGEPASISCRSSQSLLHSLGYNSLSWYLQKPGQSPHLLIYLGSNRASGVPDRFSGSGSATDFTLKISRLEAEDVGVYYCMQALQTPLTFGQGTRLEIK

SEQ ID NO: 269 - protein, 123 aa, Artificial Sequence
  Description of Artificial Sequence: Synthetic antibody
QVQLVESGGGVVQPGRSLRLSCAASGFSFSSYVMNWVRQAPGKGLEWVAVISYDGSSKYYADSVKGRFTISRDNSKNTLYLQMNSLRAEDTAVYYCARDIDSGYDPTPVFDYWGQGTLVTVSS

SEQ ID NO: 270 - protein, 108 aa, Artificial Sequence
  Description of Artificial Sequence: Synthetic antibody
DIQMTQSPSSLSACVGDRVTITCRASQSISSYLNWYQQKPGKGPKLLIYAASSLQSGVPSRFSGSGSGTDFTLTISSLQPEDFATYYCQQSYSSLSITFGQGTRLEIK

SEQ ID NO: 271 - protein, 122 aa, Artificial Sequence
  Description of Artificial Sequence: Synthetic antibody
EVQLVESGGGLVQPGGSLRLSCAASGFTFSSYDLHWVRQGTGKRLEWVSAIGTAGDTYYLGSVKGRFTISRENAKNSLYLQMNSLRAGDTAVYYCARVLYDSSGFYNWFDPWGQGTLVTVSS

SEQ ID NO: 272 - protein, 108 aa, Artificial Sequence
  Description of Artificial Sequence: Synthetic antibody
DIQMTQSPSSLSASVGDRVTITCRASQSISSYLNWYQQKPGKAPKLLIYAASSLESGVPSRFSGSGSGTDFTLTISSLQPEDFATYYCQQSYEIPPWTFGQGTKVEIK

SEQ ID NO: 273 - protein, 116 aa, Artificial Sequence
  Description of Artificial Sequence: Synthetic antibody
EVQLVESGGGLIQPGGSLRLSCAASEVTVSSNYMSWVRQAPGKGLEWVSLIYSGGTTYYADSVKGRFTISRDNSKNTLYLQMNSLRAEDTAVYYCARDFLRWHDLWGQGTLVTVSS

SEQ ID NO: 274 - protein, 107 aa, Artificial Sequence
  Description of Artificial Sequence: Synthetic antibody
DIQMTQSPSSLSASVGDRVTITCQASQDINNYLNWYQQKPGKAPKLLIYDASNLETGVPLRFSGSGSGTDFIFTISSLQPEDIATYYCQQYDNLPPVFGGGTKVEIK

SEQ ID NO: 275 - protein, 124 aa, Artificial Sequence; identical to SEQ ID NO: 231
  Description of Artificial Sequence: Synthetic antibody

SEQ ID NO: 276 - protein, 111 aa, Artificial Sequence
  Description of Artificial Sequence: Synthetic antibody
QSVLTQPPSVSGAPGQRVTISCTGSSSNIGAGYDVHWYQQLPETAPKLLIYANSNRPSGVPDRFSGSKSGTSASLAITGLQAEDEADYYCQSYDSSLSGSVFGGGTKLTVL

SEQ ID NO: 277 - protein, 122 aa, Artificial Sequence
  Description of Artificial Sequence: Synthetic antibody
QLQLQESGPGLVKPSETLSLTCTVSGGSISSSSYYWGWIRQPPGNGLEWIGSIYYSGSTYYNPSLKGRVSISVDTSKNQFSLKLSSVTAADTAVYYCARILVIFTLNWFDPWGQGTLVTVSS

SEQ ID NO: 278 - protein, 110 aa, Artificial Sequence
  Description of Artificial Sequence: Synthetic antibody
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSGNPIFGGGTKLTVL

SEQ ID NO: 279-280 - skipped (2 entries)

SEQ ID NO: 281 - protein, 116 aa, Artificial Sequence; identical to SEQ ID NO: 273
  Description of Artificial Sequence: Synthetic antibody

SEQ ID NO: 282 - protein, 107 aa, Artificial Sequence
  Description of Artificial Sequence: Synthetic antibody
DIQMTQSPSSLSASVGDRVTITCQASQDINNYLNWYQQKPGKAPKLLIYDASNLETGVPLRFSGSGSGTDFTFTISSLQPEDIATYYCQQYDNLPPVFGGGTKVEIK

SEQ ID NO: 283 - protein, 122 aa, Artificial Sequence
  Description of Artificial Sequence: Synthetic antibody
QVQLQESGPGLVKPSETLSLTCTVSGGSVSSGSYYWSWIRQPPGKGLECIGYIYYSGSSNYNPSLKSRVTISVDTSKNQFSLKMSSVTAADTAVYYCAGSPVPPTIVGASYWGQGTLVTVSS

SEQ ID NO: 284 - protein, 112 aa, Artificial Sequence
  Description of Artificial Sequence: Synthetic antibody
NFMLTQPHSVSESPGKTVTFSCTGSSGSIASNYVQWYQQRPGSAPTTVIYEDNQRPSGVPDRFSGSIDSSSNSASLTISGLKTEDEADYYCQSYDGINRWLVFGGGTKLTVL

SEQ ID NO: 285 - protein, 121 aa, Artificial Sequence
  Description of Artificial Sequence: Synthetic antibody
EVQLVQSGAEVKKPGESLKISCKGSGYSFTSYWIDWVRQMPGKGLEWMGIIYPGDSDTRYSPSFQGQVTISADKSTSTAYLQWSSLKASDTAMYYCARRGEAAGIWYFDLWGRGTLVTVSS

SEQ ID NO: 286 - protein, 117 aa, Artificial Sequence
  Description of Artificial Sequence: Synthetic antibody
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFEYVVFGGGTKLTVL

SEQ ID NO: 287 - protein, 124 aa, Artificial Sequence
  Description of Artificial Sequence: Synthetic antibody
EVQLVESGGGLVQPGGSLRLSCAASGFTVSSNYMSWVRQAPGKGLEWVSVIYSAGSTYYADSVKGRFSISRDKSKNTLYLQMNSLRAEDTAVYYCAKEGGSGSLRYYYYGMDVWGQGTTVTVSS

SEQ ID NO: 288 - protein, 110 aa, Artificial Sequence
  Description of Artificial Sequence: Synthetic antibody
QSVVTQPPSASGTPGQRVTISCSGSSSNIGYNIVNWYQQLPGTAPKLLIYSNNQRPSGVPDRFSGSKSGTSASLSISGLQSEDEADYYCAAWDDSLNGYVFGTGTKVTVL

SEQ ID NO: 289 - protein, 124 aa, Artificial Sequence
  Description of Artificial Sequence: Synthetic antibody
QVQLVESGGGVVQPGRSLRLSCAASGFTFSTYAMHWVRQAPGKGLEWVAVISYDGSNKYYADSVKGRFTISRDNSKNTLYLQMIGLRAEDTAVYYCARDWAPTYYDMPSAFDIWGQGTMVTVSS

SEQ ID NO: 290 - protein, 109 aa, Artificial Sequence
  Description of Artificial Sequence: Synthetic antibody
SYVLTQPPSVSVAPGKTARITCGGNNIGNKGVHWYQQKPGQAPVLVVDDDSDRPSGIPERFSGSNSGNTATLIISSVEVGDEADFYCQVWDSSSDHPGVFGGGTKLTVL

SEQ ID NO: 291 - protein, 124 aa, Artificial Sequence
  Description of Artificial Sequence: Synthetic antibody
EVQLVESGGGLVQPGGSLRLSCEASGFTFSSSEINWVRQAPGKGLEWVSHISSSGSIIYYADSVKGRFTISRDNAKNSLYLQMNSLRAEDTAVYYCARRSYRSSWYYYYGMDVWGQGTTVTVSS

SEQ ID NO: 292 - protein, 107 aa, Artificial Sequence
  Description of Artificial Sequence: Synthetic antibody
DIQLTQSPSFLSASVGDRVTITCRASQGISSYLAWYQQKPGKAPKLLIYAASTLQSGVPSRFSGSGSGTEFTLTISSLQPEDFATYYCQQLNSYPVTFGQGTKVEIK

SEQ ID NO: 293 - protein, 121 aa, Artificial Sequence
  Description of Artificial Sequence: Synthetic antibody
QVQLAESGGGVVQPGRSLRLSCAASGFTFSSYAMHWVRQAPGKGLEWVAVISYDGSNKYYADSVKGRFTISRDNSKNTLYLQMNSLRAEDTAVYYCARSTSGSYYYGMDVWGQGTTVTVSS

SEQ ID NO: 294 - protein, 112 aa, Artificial Sequence
  Description of Artificial Sequence: Synthetic antibody
QSALTQPASVSGSPGQSITISCTGTSSDVGDYNYVSWYQQHPGKAPKLMIYDVSNRPSGVSNRFSGSKSGNTASLTISGLQAEDEAEYYCSSYTSSSTLLYVFGTGTKVTVL

SEQ ID NO: 295 - protein, 119 aa, Artificial Sequence
  Description of Artificial Sequence: Synthetic antibody
QVQLVQSGAEVRKPGSSVKVSCKASGGTFSSYAISWVRQAPGQGLEWMGGIIPVFGTANYAQKFQGRVTITADKSTSTAFMELNSLRSEDTAVYYCARIGSYPEYFQHWGQGTLVTVSS

SEQ ID NO: 296 - protein, 108 aa, Artificial Sequence
  Description of Artificial Sequence: Synthetic antibody
EIVLTQSPATLSLSPGERATLSCRASQSVSSFLAWYQQKPGQAPRLLIYDASNRATGIPARFSGSGSGTDFTLTISSLEPEDFAVYYCHYRSNWPPVLTFGGGTKVEI

SEQ ID NO: 297 - protein, 122 aa, Artificial Sequence
  Description of Artificial Sequence: Synthetic antibody
QVQLVQSGAEVKKPGSSVKVSCKASGGTFSSYAIIWVRQAPGQGLEWMGGIIPIFGTTNYAQKFQGRVTITADESTSTAYVELSSLRSEDTAVYYCARIGHFDSSGYYLDYWGQGTLVTVSS

SEQ ID NO: 298 - protein, 109 aa, Artificial Sequence
  Description of Artificial Sequence: Synthetic antibody
EIVLTQSPATLSLSPGERATLSCRASQSVSSFLAWYQQKPGQAPRLLIYDASNRPTGIPARFTGSGSGTDFTLTISSLEPEDFAVYYCQHRTNWPPLFTFGPGTKVDIK

SEQ ID NO: 299 - protein, 121 aa, Artificial Sequence
  Description of Artificial Sequence: Synthetic antibody
QVQLVESGGGVVQPGRSLRLSCAASGFTFSSYALFWVRQAPGKGLEWVAVISYDGNNKYYADSVRGRFTISRDNSKNTLYLQMNSLRPEDTAVYYCARPYTGSYKSYMDVWGKGTTVTVSS

SEQ ID NO: 300 - protein, 113 aa, Artificial Sequence
  Description of Artificial Sequence: Synthetic antibody
DIVMTQSPDSLAVSLGERATINCKSSQSVLYSSNNKNSLAWYQQKPGQPPKLLIYWASTRESGVPDRFSGSGSGTDFTLTISSLQAEDVAVYYCQQYYSISWTFGQGTKVEIK

SEQ ID NO: 301 - protein, 121 aa, Artificial Sequence
  Description of Artificial Sequence: Synthetic antibody
QVQLQESGPGLVKPSQTLSLTCTVSGGSISSGGYFWSWIRQHPGKGLEWIGSIYYSGSTYYNPSLRSRITISVDTSKNQFSLKLSSVTAADTAVYYCARGGSGSYSLFDYWGQGTLVTVSS

SEQ ID NO: 302 - protein, 108 aa, Artificial Sequence
  Description of Artificial Sequence: Synthetic antibody
DIQMTQSPSSLSASVGDRVTITCQASQDITNYLNWYQQKPGKAPKLLIYDASNLETGVPSRFSGSGSGTDFTFTISSLQPEDFATYYCQQYDNLYSVHFGQGTKLEIK

SEQ ID NO: 303 - protein, 122 aa, Artificial Sequence
  Description of Artificial Sequence: Synthetic antibody
QVQLVESGGGVVQPGRSLRLSCAASGFTFSRHAMHWVRQAPGKGLEWVAVISYDGSNKYYADSVKGRFAISRDNSKNTLYLQMNSLRPEDTAVYYCARDPSPLVLITSIDYWGQGTLVTVSS

SEQ ID NO: 304 - protein, 108 aa, Artificial Sequence
  Description of Artificial Sequence: Synthetic antibody
SYELTQPPSVSVSPGQTARITCSGDALPRQYTYWYQQKPGQAPVLVIYKDSERPSGIPERFSGSSSGTTVTLTISGVQAEDEADYYCQSADTIGTYWVFGGGTKLTVL

SEQ ID NO: 305 - protein, 119 aa, Artificial Sequence
  Description of Artificial Sequence: Synthetic antibody
EVQLVESGGGLVQPGGSLRLSCAASGFTFSSYWMTWVRQAPGKGLEWVANIKQDGSEKYYVDSVKGRFTISRDNAKNSLSLQMNSLRVEDTAVYYCVRLGVSSWYFDYWGQGTLVTVSS

SEQ ID NO: 306 - protein, 106 aa, Artificial Sequence
  Description of Artificial Sequence: Synthetic antibody
SYELTQPPSVSVSPGQTASITCSGDKLGDKYACWYQQKPGQSPVLVIYQDTKRPSGIPERFSGSNSGNTATLTISGTQAMDEADYYCQAWGSSRGVFGGGTKLTVL

SEQ ID NO: 307 - protein, 122 aa, Artificial Sequence
  Description of Artificial Sequence: Synthetic antibody
QVQLVQSGAEVKKPGSSVKVSCKASGGTFSSYAISWVRQAPGQGLEWMGGIIPIFGAANYAQNFQGRVTITADESTSTGYMQLSSLRFEDTAVYYCARTSHYDSSGSYFEYWGQGTLVTVSS

SEQ ID NO: 308 - protein, 108 aa, Artificial Sequence
  Description of Artificial Sequence: Synthetic antibody
EIVLTQSPATLSLSPGERATLSCRASQSVSSYLAWYQQKPGQAPRLLIYDASNRATGIPARFSGSGSGTDFTLTISSLDPEDFAVYYCHKRSNWPPSLTFGGGTKVEI

SEQ ID NO: 309-311 - skipped (3 entries)

SEQ ID NO: 312 - protein, 8 aa, Artificial Sequence
  Description of Artificial Sequence: Synthetic antibody
GFTFSFYW

SEQ ID NO: 313 - protein, 8 aa, Artificial Sequence
  Description of Artificial Sequence: Synthetic antibody
IKQDGGEK

SEQ ID NO: 314 - protein, 12 aa, Artificial Sequence
  Description of Artificial Sequence: Synthetic antibody
ARLSGSSWDFDY

SEQ ID NO: 315 - protein, 8 aa, Artificial Sequence
  Description of Artificial Sequence: Synthetic antibody
GFTFDDYG

SEQ ID NO: 316 - protein, 8 aa, Artificial Sequence
  Description of Artificial Sequence: Synthetic antibody
INWNGGST

SEQ ID NO: 317 - protein, 22 aa, Artificial Sequence
  Description of Artificial Sequence: Synthetic antibody
ARRRSSSRYSSGWYMYYYYMDV

SEQ ID NO: 318 - protein, 10 aa, Artificial Sequence
  Description of Artificial Sequence: Synthetic antibody
GFSVSTSGEG

SEQ ID NO: 319 - protein, 7 aa, Artificial Sequence
  Description of Artificial Sequence: Synthetic antibody
IYWDDDK

SEQ ID NO: 320 - protein, 12 aa, Artificial Sequence
  Description of Artificial Sequence: Synthetic antibody
AHRLWFRDAFDI

SEQ ID NO: 321 - protein, 8 aa, Artificial Sequence
  Description of Artificial Sequence: Synthetic antibody
GFTFSTYW

SEQ ID NO: 322 - protein, 8 aa, Artificial Sequence
  Description of Artificial Sequence: Synthetic antibody
IKQDGSEK

SEQ ID NO: 323 - protein, 12 aa, Artificial Sequence
  Description of Artificial Sequence: Synthetic antibody
ARVGSSSWYFDY

SEQ ID NO: 324 - protein, 10 aa, Artificial Sequence
  Description of Artificial Sequence: Synthetic antibody
GGSISSGTYY

SEQ ID NO: 325 - protein, 7 aa, Artificial Sequence
  Description of Artificial Sequence: Synthetic antibody
TYYGGST

SEQ ID NO: 326 - protein, 15 aa, Artificial Sequence
  Description of Artificial Sequence: Synthetic antibody
ARRGNYYDSKNWFDP

SEQ ID NO: 327 - protein, 8 aa, Artificial Sequence
  Description of Artificial Sequence: Synthetic antibody
GFTFSSHG

SEQ ID NO: 328 - protein, 8 aa, Artificial Sequence
  Description of Artificial Sequence: Synthetic antibody
IWYDGSNK

SEQ ID NO: 329 - protein, 13 aa, Artificial Sequence
  Description of Artificial Sequence: Synthetic antibody
ARESADISSRLDY

SEQ ID NO: 330 - protein, 8 aa, Artificial Sequence
  Description of Artificial Sequence: Synthetic antibody
GFTFSSYA

SEQ ID NO: 331 - protein, 8 aa, Artificial Sequence
  Description of Artificial Sequence: Synthetic antibody
ISYDGSNK

SEQ ID NO: 332 - protein, 14 aa, Artificial Sequence
  Description of Artificial Sequence: Synthetic antibody
ARADTMVRGTYFEY

SEQ ID NO: 333 - protein, 10 aa, Artificial Sequence
  Description of Artificial Sequence: Synthetic antibody
GFSLSTSGVG

SEQ ID NO: 334 - protein, 7 aa, Artificial Sequence
  Description of Artificial Sequence: Synthetic antibody
IYWDDDK

SEQ ID NO: 335 - protein, 19 aa, Artificial Sequence
  Description of Artificial Sequence: Synthetic antibody
AHRLPTPQLLPSFENWFDP

SEQ ID NO: 336 - protein, 8 aa, Artificial Sequence
  Description of Artificial Sequence: Synthetic antibody
GFTFSSYW

SEQ ID NO: 337 - protein, 8 aa, Artificial Sequence
  Description of Artificial Sequence: Synthetic antibody
INSDGSST

SEQ ID NO: 338 - protein, 13 aa, Artificial Sequence
  Description of Artificial Sequence: Synthetic antibody
AREVEQLAHMVDY

SEQ ID NO: 339 - protein, 8 aa, Artificial Sequence
  Description of Artificial Sequence: Synthetic antibody
GFTFSSYA

SEQ ID NO: 340 - protein, 8 aa, Artificial Sequence
  Description of Artificial Sequence: Synthetic antibody
ISYDGINK

SEQ ID NO: 341 - protein, 14 aa, Artificial Sequence
  Description of Artificial Sequence: Synthetic antibody
ARPRSGSYYAYFDY

SEQ ID NO: 342 - protein, 8 aa, Artificial Sequence
  Description of Artificial Sequence: Synthetic antibody
GFTFSYYP

SEQ ID NO: 343 - protein, 8 aa, Artificial Sequence
  Description of Artificial Sequence: Synthetic antibody
TSYDGTNK

SEQ ID NO: 344 - protein, 10 aa, Artificial Sequence
  Description of Artificial Sequence: Synthetic antibody
ARGGATNFDY

SEQ ID NO: 345 - protein, 8 aa, Artificial Sequence
  Description of Artificial Sequence: Synthetic antibody
GFTFDDSA

SEQ ID NO: 346 - protein, 8 aa, Artificial Sequence
  Description of Artificial Sequence: Synthetic antibody
ISWNSGNV

SEQ ID NO: 347 - protein, 19 aa, Artificial Sequence
  Description of Artificial Sequence: Synthetic antibody
TKASRYCSSTICYWNWFDP

SEQ ID NO: 348 - protein, 8 aa, Artificial Sequence
  Description of Artificial Sequence: Synthetic antibody
GFTFSSYE

SEQ ID NO: 349 - protein, 8 aa, Artificial Sequence
  Description of Artificial Sequence: Synthetic antibody
ISSSGSAI

SEQ ID NO: 350 - protein, 19 aa, Artificial Sequence
  Description of Artificial Sequence: Synthetic antibody
AREARSRYFDWLPSYYFDY

SEQ ID NO: 351 - protein, 8 aa, Artificial Sequence
  Description of Artificial Sequence: Synthetic antibody
GYTFTTYA

SEQ ID NO: 352 - protein, 8 aa, Artificial Sequence
  Description of Artificial Sequence: Synthetic antibody
INTNTGNP

SEQ ID NO: 353 - protein, 18 aa, Artificial Sequence
  Description of Artificial Sequence: Synthetic antibody
ARDQDSGYPTYYYYYMDV

SEQ ID NO: 354 - protein, 8 aa, Artificial Sequence
  Description of Artificial Sequence: Synthetic antibody
GFTFSTYA

SEQ ID NO: 355 - protein, 8 aa, Artificial Sequence
  Description of Artificial Sequence: Synthetic antibody
ISYDGYNK

SEQ ID NO: 356 - protein, 14 aa, Artificial Sequence
  Description of Artificial Sequence: Synthetic antibody
ARGSAGNYYYGMDV

SEQ ID NO: 357 - protein, 8 aa, Artificial Sequence
  Description of Artificial Sequence: Synthetic antibody
GFTFSNYG

SEQ ID NO: 358 - protein, 8 aa, Artificial Sequence
  Description of Artificial Sequence: Synthetic antibody
ISNDEFNK

SEQ ID NO: 359 - protein, 20 aa, Artificial Sequence
  Description of Artificial Sequence: Synthetic antibody
AKGGDGSGWAWDGDNPPTDY

SEQ ID NO: 360-362 - skipped (3 entries)

SEQ ID NO: 363 - protein, 8 aa, Artificial Sequence
  Description of Artificial Sequence: Synthetic antibody
GYSFTSYW

SEQ ID NO: 364 - protein, 8 aa, Artificial Sequence
  Description of Artificial Sequence: Synthetic antibody
IYPGDSDT

SEQ ID NO: 365 - protein, 17 aa, Artificial Sequence
  Description of Artificial Sequence: Synthetic antibody
ARPDYSSGWFSYWYFDL

SEQ ID NO: 366 - protein, 8 aa, Artificial Sequence
  Description of Artificial Sequence: Synthetic antibody
GYTLTELS

SEQ ID NO: 367 - protein, 8 aa, Artificial Sequence
  Description of Artificial Sequence: Synthetic antibody
FDPEDAET

SEQ ID NO: 368 - protein, 14 aa, Artificial Sequence
  Description of Artificial Sequence: Synthetic antibody
ATGFAVFGRAAVPY

SEQ ID NO: 369 - protein, 8 aa, Artificial Sequence
  Description of Artificial Sequence: Synthetic antibody
GFTFSSYG

SEQ ID NO: 370 - protein, 8 aa, Artificial Sequence
  Description of Artificial Sequence: Synthetic antibody
ISYDGSNK

SEQ ID NO: 371 - protein, 17 aa, Artificial Sequence
  Description of Artificial Sequence: Synthetic antibody
AKDLTIVVIPAAPNFDY

SEQ ID NO: 372 - protein, 8 aa, Artificial Sequence
  Description of Artificial Sequence: Synthetic antibody
GYSFTSHW

SEQ ID NO: 373 - protein, 8 aa, Artificial Sequence
  Description of Artificial Sequence: Synthetic antibody
IYPGDSDT

SEQ ID NO: 374 - protein, 14 aa, Artificial Sequence
  Description of Artificial Sequence: Synthetic antibody
ASALRERGVQLWSV

SEQ ID NO: 375 - protein, 8 aa, Artificial Sequence
  Description of Artificial Sequence: Synthetic antibody
GFTFSSYA

SEQ ID NO: 376 - protein, 8 aa, Artificial Sequence
  Description of Artificial Sequence: Synthetic antibody
ISYDGSNK

SEQ ID NO: 377 - protein, 15 aa, Artificial Sequence
  Description of Artificial Sequence: Synthetic antibody
ARDQEWFRELFLFDY

SEQ ID NO: 378 - protein, 8 aa, Artificial Sequence
  Description of Artificial Sequence: Synthetic antibody
GFTFSTYG

SEQ ID NO: 379 - protein, 8 aa, Artificial Sequence
  Description of Artificial Sequence: Synthetic antibody
ISYDGSNK

SEQ ID NO: 380 - protein, 11 aa, Artificial Sequence
  Description of Artificial Sequence: Synthetic antibody
AKDGSIAAADY

SEQ ID NO: 381 - protein, 8 aa, Artificial Sequence
  Description of Artificial Sequence: Synthetic antibody
GFTFSSYS

SEQ ID NO: 382 - protein, 8 aa, Artificial Sequence
  Description of Artificial Sequence: Synthetic antibody
ISNSNSFI

SEQ ID NO: 383 - protein, 21 aa, Artificial Sequence
  Description of Artificial Sequence: Synthetic antibody
ARVNGNSNWNFGSYYYYYMDV

SEQ ID NO: 384 - protein, 8 aa, Artificial Sequence
  Description of Artificial Sequence: Synthetic antibody
GFTFSGYS

SEQ ID NO: 385 - protein, 8 aa, Artificial Sequence
  Description of Artificial Sequence: Synthetic antibody
ISSSSSYI

SEQ ID NO: 386 - protein, 17 aa, Artificial Sequence
  Description of Artificial Sequence: Synthetic antibody
ARWLQLRSDYYYFGMDV

SEQ ID NO: 387 - protein, 8 aa, Artificial Sequence
  Description of Artificial Sequence: Synthetic antibody
GGSISSYY

SEQ ID NO: 388 - protein, 7 aa, Artificial Sequence
  Description of Artificial Sequence: Synthetic antibody
IYYSGST

SEQ ID NO: 389 - protein, 17 aa, Artificial Sequence
  Description of Artificial Sequence: Synthetic antibody
ARAPRERLQWGEYYFDY

SEQ ID NO: 390 - protein, 8 aa, Artificial Sequence
  Description of Artificial Sequence: Synthetic antibody
GYSFTNSW

SEQ ID NO: 391 - protein, 8 aa, Artificial Sequence
  Description of Artificial Sequence: Synthetic antibody
IYPGDSDT

SEQ ID NO: 392 - protein, 14 aa, Artificial Sequence
  Description of Artificial Sequence: Synthetic antibody
ATHRCSGGFCYLAY

SEQ ID NO: 393 - protein, 8 aa, Artificial Sequence
  Description of Artificial Sequence: Synthetic antibody
GFTFSSYG

SEQ ID NO: 394 - protein, 8 aa, Artificial Sequence
  Description of Artificial Sequence: Synthetic antibody
IWYDGSKK

SEQ ID NO: 395 - protein, 21 aa, Artificial Sequence
  Description of Artificial Sequence: Synthetic antibody
ARDQSQGAYILTGYRGYGMDV

SEQ ID NO: 396 - protein, 8 aa, Artificial Sequence
  Description of Artificial Sequence: Synthetic antibody
GGSFSGHY

SEQ ID NO: 397 - protein, 7 aa, Artificial Sequence
  Description of Artificial Sequence: Synthetic antibody
INHSGST

SEQ ID NO: 398 - protein, 17 aa, Artificial Sequence
  Description of Artificial Sequence: Synthetic antibody
ARPPQAARIHYYYYMDV

SEQ ID NO: 399 - protein, 8 aa, Artificial Sequence
  Description of Artificial Sequence: Synthetic antibody
GFTFDDYA

SEQ ID NO: 400 - protein, 8 aa, Artificial Sequence
  Description of Artificial Sequence: Synthetic antibody
ISWNSDSI

SEQ ID NO: 401 - protein, 14 aa, Artificial Sequence
  Description of Artificial Sequence: Synthetic antibody
AKGRGAGYTSYMDV

SEQ ID NO: 402 - protein, 8 aa, Artificial Sequence
  Description of Artificial Sequence: Synthetic antibody
LFRSGGHW

SEQ ID NO: 403 - protein, 8 aa, Artificial Sequence
  Description of Artificial Sequence: Synthetic antibody
IYPGDSDT

SEQ ID NO: 404 - protein, 14 aa, Artificial Sequence
  Description of Artificial Sequence: Synthetic antibody
ASALRERGVQLWSV

SEQ ID NO: 405 - protein, 8 aa, Artificial Sequence
  Description of Artificial Sequence: Synthetic antibody
GFTFDDYA

SEQ ID NO: 406 - protein, 8 aa, Artificial Sequence
  Description of Artificial Sequence: Synthetic antibody
ISWNSGSI

SEQ ID NO: 407 - protein, 16 aa, Artificial Sequence
  Description of Artificial Sequence: Synthetic antibody
AKAHSTGHQYYYGMDV

SEQ ID NO: 408 - protein, 8 aa, Artificial Sequence
  Description of Artificial Sequence: Synthetic antibody
GFTFDDYA

SEQ ID NO: 409 - protein, 8 aa, Artificial Sequence
  Description of Artificial Sequence: Synthetic antibody
ISWNSGSI

SEQ ID NO: 410 - protein, 16 aa, Artificial Sequence
  Description of Artificial Sequence: Synthetic antibody
AKVSSITSLLGYYFDS

SEQ ID NO: 411 - protein, 8 aa, Artificial Sequence
  Description of Artificial Sequence: Synthetic antibody
GDTSSSYT

SEQ ID NO: 412 - protein, 8 aa, Artificial Sequence
  Description of Artificial Sequence: Synthetic antibody
IIPILGIA

SEQ ID NO: 413 - protein, 14 aa, Artificial Sequence
  Description of Artificial Sequence: Synthetic antibody
ARGVVAATPGWFDP

SEQ ID NO: 414 - protein, 8 aa, Artificial Sequence
  Description of Artificial Sequence: Synthetic antibody
GFTFSSYW

SEQ ID NO: 415 - protein, 8 aa, Artificial Sequence
  Description of Artificial Sequence: Synthetic antibody
INSDGSST

SEQ ID NO: 416 - protein, 12 aa, Artificial Sequence
  Description of Artificial Sequence: Synthetic antibody
AGSPWLRGDIDY

SEQ ID NO: 417 - protein, 8 aa, Artificial Sequence
  Description of Artificial Sequence: Synthetic antibody
GYTFTDYY

SEQ ID NO: 418 - protein, 8 aa, Artificial Sequence
  Description of Artificial Sequence: Synthetic antibody
INPNSRGT

SEQ ID NO: 419 - protein, 23 aa, Artificial Sequence
  Description of Artificial Sequence: Synthetic antibody
ARVVVLGYGRPNNYYDGRNVWDY

SEQ ID NO: 420 - protein, 10 aa, Artificial Sequence
  Description of Artificial Sequence: Synthetic antibody
GFSLGTSGMC

SEQ ID NO: 421 - protein, 7 aa, Artificial Sequence
  Description of Artificial Sequence: Synthetic antibody
IDWDDDK

SEQ ID NO: 422 - protein, 9 aa, Artificial Sequence
  Description of Artificial Sequence: Synthetic antibody
ARGVVTYDY

SEQ ID NO: 423 - protein, 8 aa, Artificial Sequence
  Description of Artificial Sequence: Synthetic antibody
GDTFSSYT

SEQ ID NO: 424 - protein, 8 aa, Artificial Sequence
  Description of Artificial Sequence: Synthetic antibody
IIPILGIP

SEQ ID NO: 425 - protein, 17 aa, Artificial Sequence
  Description of Artificial Sequence: Synthetic antibody
ARGRGYSNYGASYYMDV

SEQ ID NO: 426 - protein, 8 aa, Artificial Sequence
  Description of Artificial Sequence: Synthetic antibody
GFTFDDYA

SEQ ID NO: 427 - protein, 8 aa, Artificial Sequence
  Description of Artificial Sequence: Synthetic antibody
ISWNSDSI

SEQ ID NO: 428 - protein, 14 aa, Artificial Sequence
  Description of Artificial Sequence: Synthetic antibody
AKGRGAGYTSYMDV

SEQ ID NO: 429-431 - skipped (3 entries)

SEQ ID NO: 432 - protein, 8 aa, Artificial Sequence
  Description of Artificial Sequence: Synthetic antibody
GYTFTSYD

SEQ ID NO: 433 - protein, 8 aa, Artificial Sequence
  Description of Artificial Sequence: Synthetic antibody
MNSNSGNA

SEQ ID NO: 434 - protein, 16 aa, Artificial Sequence
  Description of Artificial Sequence: Synthetic antibody
ARMRTGWPTHGRPDDF

SEQ ID NO: 435 - protein, 8 aa, Artificial Sequence
  Description of Artificial Sequence: Synthetic antibody
GFTVSSNY

SEQ ID NO: 436 - protein, 7 aa, Artificial Sequence
  Description of Artificial Sequence: Synthetic antibody
IYPGGSA

SEQ ID NO: 437 - protein, 16 aa, Artificial Sequence
  Description of Artificial Sequence: Synthetic antibody
ARSYDILTGYRDAFDI

SEQ ID NO: 438 - protein, 8 aa, Artificial Sequence
  Description of Artificial Sequence: Synthetic antibody
GLTVSSNY

SEQ ID NO: 439 - protein, 7 aa, Artificial Sequence
  Description of Artificial Sequence: Synthetic antibody
IYAGGNT

SEQ ID NO: 440 - protein, 13 aa, Artificial Sequence
  Description of Artificial Sequence: Synthetic antibody
ARGDGGYYSPFDY

SEQ ID NO: 441 - protein, 8 aa, Artificial Sequence
  Description of Artificial Sequence: Synthetic antibody
GGSFSAYY

SEQ ID NO: 442 - protein, 7 aa, Artificial Sequence
  Description of Artificial Sequence: Synthetic antibody
INHSGST

SEQ ID NO: 443 - protein, 16 aa, Artificial Sequence
  Description of Artificial Sequence: Synthetic antibody
ARVGYSQGYYYYYMDV

SEQ ID NO: 444-446 - skipped (3 entries)

SEQ ID NO: 447 - protein, 8 aa, Artificial Sequence
  Description of Artificial Sequence: Synthetic antibody
GGTFSSYS

SEQ ID NO: 448 - protein, 8 aa, Artificial Sequence
  Description of Artificial Sequence: Synthetic antibody
IIPVLGIA

SEQ ID NO: 449 - protein, 18 aa, Artificial Sequence
  Description of Artificial Sequence: Synthetic antibody
ARVGVSGFKSGSNWYFDL

SEQ ID NO: 450-452 - skipped (3 entries)

SEQ ID NO: 453 - protein, 8 aa, Artificial Sequence
  Description of Artificial Sequence: Synthetic antibody
GYTFTSYY

SEQ ID NO: 454 - protein, 8 aa, Artificial Sequence
  Description of Artificial Sequence: Synthetic antibody
INPGGGST

SEQ ID NO: 455 - protein, 15 aa, Artificial Sequence
  Description of Artificial Sequence: Synthetic antibody
ARGAIPPNSRAEIDY

SEQ ID NO: 456 - protein, 8 aa, Artificial Sequence
  Description of Artificial Sequence: Synthetic antibody
GFIFDDYD

SEQ ID NO: 457 - protein, 8 aa, Artificial Sequence
  Description of Artificial Sequence: Synthetic antibody
ISWNGGNT

SEQ ID NO: 458 - protein, 22 aa, Artificial Sequence
  Description of Artificial Sequence: Synthetic antibody
AVIMSPIPRYSGYDWAGGAFDI

SEQ ID NO: 459 - protein, 8 aa, Artificial Sequence
  Description of Artificial Sequence: Synthetic antibody
GFTFSNYG

SEQ ID NO: 460 - protein, 8 aa, Artificial Sequence
  Description of Artificial Sequence: Synthetic antibody
MSYDGSNK

SEQ ID NO: 461 - protein, 20 aa, Artificial Sequence
  Description of Artificial Sequence: Synthetic antibody
AKNLGPYCSGGTCYSLVGDY

SEQ ID NO: 462-467 - skipped (6 entries)

SEQ ID NO: 468 - protein, 8 aa, Artificial Sequence
  Description of Artificial Sequence: Synthetic antibody
GFTFDDYA

SEQ ID NO: 469 - protein, 8 aa, Artificial Sequence
  Description of Artificial Sequence: Synthetic antibody
ISWNSGTI

SEQ ID NO: 470 - protein, 14 aa, Artificial Sequence
  Description of Artificial Sequence: Synthetic antibody
AKDIIRQGEDGMDV

SEQ ID NO: 471 - protein, 8 aa, Artificial Sequence
  Description of Artificial Sequence: Synthetic antibody
GFTFSNYA

SEQ ID NO: 472 - protein, 9 aa, Artificial Sequence
  Description of Artificial Sequence: Synthetic antibody
IISTSGGAT

SEQ ID NO: 473 - protein, 10 aa, Artificial Sequence
  Description of Artificial Sequence: Synthetic antibody
VKGLFDWFPL

SEQ ID NO: 474 - protein, 8 aa, Artificial Sequence
  Description of Artificial Sequence: Synthetic antibody
GFTFSRHW

SEQ ID NO: 475 - protein, 8 aa, Artificial Sequence
  Description of Artificial Sequence: Synthetic antibody
IKQDGSEK

SEQ ID NO: 476 - protein, 12 aa, Artificial Sequence
  Description of Artificial Sequence: Synthetic antibody
ARLGFYYGGADY

SEQ ID NO: 477 - protein, 8 aa, Artificial Sequence
  Description of Artificial Sequence: Synthetic antibody
GFTFHHYA

SEQ ID NO: 478 - protein, 8 aa, Artificial Sequence
  Description of Artificial Sequence: Synthetic antibody
ISGSSDYR

SEQ ID NO: 479 - protein, 15 aa, Artificial Sequence
  Description of Artificial Sequence: Synthetic antibody
AKGVDYGGKLAYFDS

SEQ ID NO: 480 - protein, 8 aa, Artificial Sequence
  Description of Artificial Sequence: Synthetic antibody
GFSFSSYV

SEQ ID NO: 481 - protein, 8 aa, Artificial Sequence
  Description of Artificial Sequence: Synthetic antibody
ISYDGSSK

SEQ ID NO: 482 - protein, 16 aa, Artificial Sequence
  Description of Artificial Sequence: Synthetic antibody
ARDIDSGYDPTPVFDY

SEQ ID NO: 483 - protein, 8 aa, Artificial Sequence
  Description of Artificial Sequence: Synthetic antibody
GFTFSSYD

SEQ ID NO: 484 - protein, 7 aa, Artificial Sequence
  Description of Artificial Sequence: Synthetic antibody
IGTAGDT

SEQ ID NO: 485 - protein, 16 aa, Artificial Sequence
  Description of Artificial Sequence: Synthetic antibody
ARVLYDSSGFYNWFDP

SEQ ID NO: 486 - protein, 8 aa, Artificial Sequence
  Description of Artificial Sequence: Synthetic antibody
EVTVSSNY

SEQ ID NO: 487 - protein, 7 aa, Artificial Sequence
  Description of Artificial Sequence: Synthetic antibody
IYSGGTT

SEQ ID NO: 488 - protein, 10 aa, Artificial Sequence
  Description of Artificial Sequence: Synthetic antibody
ARDFLRWHDL

SEQ ID NO: 489 - protein, 8 aa, Artificial Sequence
  Description of Artificial Sequence: Synthetic antibody
GDTFSSYT

SEQ ID NO: 490 - protein, 8 aa, Artificial Sequence
  Description of Artificial Sequence: Synthetic antibody
IIPILGIP

SEQ ID NO: 491 - protein, 17 aa, Artificial Sequence
  Description of Artificial Sequence: Synthetic antibody
ARGRGYSNYGASYYMDV

SEQ ID NO: 492 - protein, 10 aa, Artificial Sequence
  Description of Artificial Sequence: Synthetic antibody
GGSISSSSYY

SEQ ID NO: 493 - protein, 7 aa, Artificial Sequence
  Description of Artificial Sequence: Synthetic antibody
IYYSGST

SEQ ID NO: 494 - protein, 14 aa, Artificial Sequence
  Description of Artificial Sequence: Synthetic antibody
ARILVIFTLNWFDP

SEQ ID NO: 495-497 - skipped (3 entries)

SEQ ID NO: 498 - protein, 8 aa, Artificial Sequence
  Description of Artificial Sequence: Synthetic antibody
EVTVSSNY

SEQ ID NO: 499 - protein, 7 aa, Artificial Sequence
  Description of Artificial Sequence: Synthetic antibody
IYSGGTT

SEQ ID NO: 500 - protein, 10 aa, Artificial Sequence
  Description of Artificial Sequence: Synthetic antibody
ARDFLRWHDL

SEQ ID NO: 501 - protein, 10 aa, Artificial Sequence
  Description of Artificial Sequence: Synthetic antibody
GGSVSSGSYY

SEQ ID NO: 502 - protein, 7 aa, Artificial Sequence
  Description of Artificial Sequence: Synthetic antibody
IYYSGSS

SEQ ID NO: 503 - protein, 14 aa, Artificial Sequence
  Description of Artificial Sequence: Synthetic antibody
AGSPVPPTIVGASY

SEQ ID NO: 504 - protein, 8 aa, Artificial Sequence
  Description of Artificial Sequence: Synthetic antibody
GYSFTSYW

SEQ ID NO: 505 - protein, 8 aa, Artificial Sequence
  Description of Artificial Sequence: Synthetic antibody
IYPGDSDT

SEQ ID NO: 506 - protein, 14 aa, Artificial Sequence
  Description of Artificial Sequence: Synthetic antibody
ARRGEAAGIWYFDL

SEQ ID NO: 507 - protein, 8 aa, Artificial Sequence
  Description of Artificial Sequence: Synthetic antibody
GFTVSSNY

SEQ ID NO: 508 - protein, 7 aa, Artificial Sequence
  Description of Artificial Sequence: Synthetic antibody
IYSAGST

SEQ ID NO: 509 - protein, 18 aa, Artificial Sequence
  Description of Artificial Sequence: Synthetic antibody
AKEGGSGSLRYYYYGMDV

SEQ ID NO: 510 - protein, 8 aa, Artificial Sequence
  Description of Artificial Sequence: Synthetic antibody
GFTFSTYA

SEQ ID NO: 511 - protein, 8 aa, Artificial Sequence
  Description of Artificial Sequence: Synthetic antibody
ISYDGSNK

SEQ ID NO: 512 - protein, 17 aa, Artificial Sequence
  Description of Artificial Sequence: Synthetic antibody
ARDWAPTYYDMPSAFDI

SEQ ID NO: 513 - protein, 8 aa, Artificial Sequence
  Description of Artificial Sequence: Synthetic antibody
GFTFSSSE

SEQ ID NO: 514 - protein, 8 aa, Artificial Sequence
  Description of Artificial Sequence: Synthetic antibody
ISSSGSII

SEQ ID NO: 515 - protein, 17 aa, Artificial Sequence
  Description of Artificial Sequence: Synthetic antibody
ARRSYRSSWYYYYGMDV

SEQ ID NO: 516 - protein, 8 aa, Artificial Sequence
  Description of Artificial Sequence: Synthetic antibody
GFTFSSYA

SEQ ID NO: 517 - protein, 8 aa, Artificial Sequence
  Description of Artificial Sequence: Synthetic antibody
ISYDGSNK

SEQ ID NO: 518 - protein, 14 aa, Artificial Sequence
  Description of Artificial Sequence: Synthetic antibody
ARSTSGSYYYGMDV

SEQ ID NO: 519 - protein, 8 aa, Artificial Sequence
  Description of Artificial Sequence: Synthetic antibody
GGTFSSYA

SEQ ID NO: 520 - protein, 8 aa, Artificial Sequence
  Description of Artificial Sequence: Synthetic antibody
IIPVFGTA

SEQ ID NO: 521 - protein, 12 aa, Artificial Sequence
  Description of Artificial Sequence: Synthetic antibody
ARIGSYPEYFQH

SEQ ID NO: 522 - protein, 8 aa, Artificial Sequence
  Description of Artificial Sequence: Synthetic antibody
GGTFSSYA

SEQ ID NO: 523 - protein, 8 aa, Artificial Sequence
  Description of Artificial Sequence: Synthetic antibody
IIPIFGTT

SEQ ID NO: 524 - protein, 15 aa, Artificial Sequence
  Description of Artificial Sequence: Synthetic antibody
ARIGHFDSSGYYLDY

SEQ ID NO: 525 - protein, 8 aa, Artificial Sequence
  Description of Artificial Sequence: Synthetic antibody
GFTFSSYA

SEQ ID NO: 526 - protein, 8 aa, Artificial Sequence
  Description of Artificial Sequence: Synthetic antibody
ISYDGNNK

SEQ ID NO: 527 - protein, 14 aa, Artificial Sequence
  Description of Artificial Sequence: Synthetic antibody
ARPYTGSYKSYMDV

SEQ ID NO: 528 - protein, 10 aa, Artificial Sequence
  Description of Artificial Sequence: Synthetic antibody
GGSISSGGYF

SEQ ID NO: 529 - protein, 7 aa, Artificial Sequence
  Description of Artificial Sequence: Synthetic antibody
IYYSGST

SEQ ID NO: 530 - protein, 13 aa, Artificial Sequence
  Description of Artificial Sequence: Synthetic antibody
ARGGSGSYSLFDY

SEQ ID NO: 531 - protein, 8 aa, Artificial Sequence
  Description of Artificial Sequence: Synthetic antibody
GFTFSRHA

SEQ ID NO: 532 - protein, 8 aa, Artificial Sequence
  Description of Artificial Sequence: Synthetic antibody
ISYDGSNK

SEQ ID NO: 533 - protein, 15 aa, Artificial Sequence
  Description of Artificial Sequence: Synthetic antibody
ARDPSPLVLITSIDY

SEQ ID NO: 534 - protein, 8 aa, Artificial Sequence
  Description of Artificial Sequence: Synthetic antibody
GFTFSSYW

SEQ ID NO: 535 - protein, 8 aa, Artificial Sequence
  Description of Artificial Sequence: Synthetic antibody
IKQDGSEK

SEQ ID NO: 536 - protein, 12 aa, Artificial Sequence
  Description of Artificial Sequence: Synthetic antibody
VRLGVSSWYFDY

SEQ ID NO: 537 - protein, 8 aa, Artificial Sequence
  Description of Artificial Sequence: Synthetic antibody
GGTFSSYA

SEQ ID NO: 538 - protein, 8 aa, Artificial Sequence
  Description of Artificial Sequence: Synthetic antibody
IIPIFGAA

SEQ ID NO: 539 - protein, 15 aa, Artificial Sequence
  Description of Artificial Sequence: Synthetic antibody
ARTSHYDSSGSYFEY

SEQ ID NO: 540 - protein, 6 aa, Artificial Sequence
  Description of Artificial Sequence: Synthetic antibody
QSVSSY

SEQ ID NO: 541-542 - skipped (2 entries)

SEQ ID NO: 543 - protein, 6 aa, Artificial Sequence
  Description of Artificial Sequence: Synthetic antibody
KLGDKY

SEQ ID NO: 544 - protein, 3 aa, Artificial Sequence
  Description of Artificial Sequence: Synthetic antibody
QDS

SEQ ID NO: 545 - protein, 9 aa, Artificial Sequence
  Description of Artificial Sequence: Synthetic antibody
QAWDSSTGV

SEQ ID NO: 546 - protein, 6 aa, Artificial Sequence
  Description of Artificial Sequence: Synthetic antibody
QSVSTW

SEQ ID NO: 547 - protein, 3 aa, Artificial Sequence
  Description of Artificial Sequence: Synthetic antibody
EAS

SEQ ID NO: 548 - protein, 9 aa, Artificial Sequence
  Description of Artificial Sequence: Synthetic antibody
QQYNTYSGT

SEQ ID NO: 549 - protein, 6 aa, Artificial Sequence
  Description of Artificial Sequence: Synthetic antibody
QSISNY

SEQ ID NO: 550 - protein, 3 aa, Artificial Sequence
  Description of Artificial Sequence: Synthetic antibody
AAS

SEQ ID NO: 551 - protein, 9 aa, Artificial Sequence
  Description of Artificial Sequence: Synthetic antibody
QQTYSTFWT

SEQ ID NO: 552 - protein, 6 aa, Artificial Sequence
  Description of Artificial Sequence: Synthetic antibody
KLGDKY

SEQ ID NO: 553 - protein, 3 aa, Artificial Sequence
  Description of Artificial Sequence: Synthetic antibody
QDS

SEQ ID NO: 554 - protein, 9 aa, Artificial Sequence
  Description of Artificial Sequence: Synthetic antibody
QAWDSSTAV

SEQ ID NO: 555 - protein, 6 aa, Artificial Sequence
  Description of Artificial Sequence: Synthetic antibody
QSVSSN

SEQ ID NO: 556 - protein, 3 aa, Artificial Sequence
  Description of Artificial Sequence: Synthetic antibody
GAS

SEQ ID NO: 557 - protein, 11 aa, Artificial Sequence
  Description of Artificial Sequence: Synthetic antibody
QQYNNWPPMYT

SEQ ID NO: 558 - protein, 6 aa, Artificial Sequence
  Description of Artificial Sequence: Synthetic antibody
ALPTKY

SEQ ID NO: 559 - protein, 3 aa, Artificial Sequence
  Description of Artificial Sequence: Synthetic antibody
DDS

SEQ ID NO: 560 - protein, 9 aa, Artificial Sequence
  Description of Artificial Sequence: Synthetic antibody
YSTDSSGNV

SEQ ID NO: 561 - protein, 9 aa, Artificial Sequence
  Description of Artificial Sequence: Synthetic antibody
SSDVGGYNY

SEQ ID NO: 562 - protein, 3 aa, Artificial Sequence
  Description of Artificial Sequence: Synthetic antibody
DVS

SEQ ID NO: 563 - protein, 10 aa, Artificial Sequence
  Description of Artificial Sequence: Synthetic antibody
SSYTSSRAVL

SEQ ID NO: 564 - protein, 8 aa, Artificial Sequence
  Description of Artificial Sequence: Synthetic antibody
SSNIGNNA

SEQ ID NO: 565 - protein, 3 aa, Artificial Sequence
  Description of Artificial Sequence: Synthetic antibody
YDD

SEQ ID NO: 566 - protein, 11 aa, Artificial Sequence
  Description of Artificial Sequence: Synthetic antibody
ASWDDSLIGPV

SEQ ID NO: 567 - protein, 6 aa, Artificial Sequence
  Description of Artificial Sequence: Synthetic antibody
ALPNQY

SEQ ID NO: 568 - protein, 3 aa, Artificial Sequence
  Description of Artificial Sequence: Synthetic antibody
KDS

SEQ ID NO: 569 - protein, 11 aa, Artificial Sequence
  Description of Artificial Sequence: Synthetic antibody
QSADSSGTSWV

SEQ ID NO: 570 - protein, 6 aa, Artificial Sequence
  Description of Artificial Sequence: Synthetic antibody
QGISNY

SEQ ID NO: 571 - protein, 3 aa, Artificial Sequence
  Description of Artificial Sequence: Synthetic antibody
AAS

SEQ ID NO: 572 - protein, 9 aa, Artificial Sequence
  Description of Artificial Sequence: Synthetic antibody
QQYNSHPPT

SEQ ID NO: 573 - protein, 6 aa, Artificial Sequence
  Description of Artificial Sequence: Synthetic antibody
NIGRKS

SEQ ID NO: 574 - protein, 3 aa, Artificial Sequence
  Description of Artificial Sequence: Synthetic antibody
DDS

SEQ ID NO: 575 - protein, 13 aa, Artificial Sequence
  Description of Artificial Sequence: Synthetic antibody
QVWDSSSDHPEWV

SEQ ID NO: 576 - protein, 6 aa, Artificial Sequence
  Description of Artificial Sequence: Synthetic antibody
QSISSY

SEQ ID NO: 577 - protein, 3 aa, Artificial Sequence
  Description of Artificial Sequence: Synthetic antibody
GAS

SEQ ID NO: 578 - protein, 8 aa, Artificial Sequence
  Description of Artificial Sequence: Synthetic antibody
QQSYSTPT

SEQ ID NO: 579 - protein, 9 aa, Artificial Sequence
  Description of Artificial Sequence: Synthetic antibody
SSDIGGYNY

SEQ ID NO: 580 - protein, 3 aa, Artificial Sequence
  Description of Artificial Sequence: Synthetic antibody
DVS

SEQ ID NO: 581 - protein, 11 aa, Artificial Sequence
  Description of Artificial Sequence: Synthetic antibody
SSYTSSSTHVV

SEQ ID NO: 582 - protein, 11 aa, Artificial Sequence
  Description of Artificial Sequence: Synthetic antibody
QSLLHSDGKTY

SEQ ID NO: 583 - protein, 3 aa, Artificial Sequence
  Description of Artificial Sequence: Synthetic antibody
EVS

SEQ ID NO: 584 - protein, 9 aa, Artificial Sequence
  Description of Artificial Sequence: Synthetic antibody
MQSIQPPLT

SEQ ID NO: 585 - protein, 6 aa, Artificial Sequence
  Description of Artificial Sequence: Synthetic antibody
QTITNY

SEQ ID NO: 586 - protein, 3 aa, Artificial Sequence
  Description of Artificial Sequence: Synthetic antibody
AAS

SEQ ID NO: 587 - protein, 9 aa, Artificial Sequence
  Description of Artificial Sequence: Synthetic antibody
QQSYSTPYT

SEQ ID NO: 588 - protein, 12 aa, Artificial Sequence
  Description of Artificial Sequence: Synthetic antibody
QSVLYTPKNKNY

SEQ ID NO: 589 - protein, 3 aa, Artificial Sequence
  Description of Artificial Sequence: Synthetic antibody
WAS

SEQ ID NO: 590 - protein, 9 aa, Artificial Sequence
  Description of Artificial Sequence: Synthetic antibody
QQYYTAPLT

SEQ ID NO: 591-593 - skipped (3 entries)

SEQ ID NO: 594 - protein, 7 aa, Artificial Sequence
  Description of Artificial Sequence: Synthetic antibody
QSVSSNF

SEQ ID NO: 595 - protein, 3 aa, Artificial Sequence
  Description of Artificial Sequence: Synthetic antibody
GAS

SEQ ID NO: 596 - protein, 9 aa, Artificial Sequence
  Description of Artificial Sequence: Synthetic antibody
QQYGRSPIT

SEQ ID NO: 597 - protein, 6 aa, Artificial Sequence
  Description of Artificial Sequence: Synthetic antibody
KLGDKY

SEQ ID NO: 598 - protein, 3 aa, Artificial Sequence
  Description of Artificial Sequence: Synthetic antibody
QGA

SEQ ID NO: 599 - protein, 9 aa, Artificial Sequence
  Description of Artificial Sequence: Synthetic antibody
QAWDSSTVV

SEQ ID NO: 600 - protein, 9 aa, Artificial Sequence
  Description of Artificial Sequence: Synthetic antibody
SSDVGGYNY

SEQ ID NO: 601 - protein, 3 aa, Artificial Sequence
  Description of Artificial Sequence: Synthetic antibody
DVS

SEQ ID NO: 602 - protein, 11 aa, Artificial Sequence
  Description of Artificial Sequence: Synthetic antibody
SSYTSSSTPVV

SEQ ID NO: 603 - protein, 9 aa, Artificial Sequence
  Description of Artificial Sequence: Synthetic antibody
SSNIGAGYD

SEQ ID NO: 604 - protein, 3 aa, Artificial Sequence
  Description of Artificial Sequence: Synthetic antibody
INS

SEQ ID NO: 605 - protein, 10 aa, Artificial Sequence
  Description of Artificial Sequence: Synthetic antibody
QSYDSSLGAL

SEQ ID NO: 606 - protein, 6 aa, Artificial Sequence
  Description of Artificial Sequence: Synthetic antibody
QGISSW

SEQ ID NO: 607 - protein, 3 aa, Artificial Sequence
  Description of Artificial Sequence: Synthetic antibody
DAS

SEQ ID NO: 608 - protein, 9 aa, Artificial Sequence
  Description of Artificial Sequence: Synthetic antibody
QQANSFPPT

SEQ ID NO: 609 - protein, 12 aa, Artificial Sequence
  Description of Artificial Sequence: Synthetic antibody
QSVLHSSNNKDS

SEQ ID NO: 610 - protein, 3 aa, Artificial Sequence
  Description of Artificial Sequence: Synthetic antibody
WAS

SEQ ID NO: 611 - protein, 9 aa, Artificial Sequence
  Description of Artificial Sequence: Synthetic antibody
QQYYSTPWT

SEQ ID NO: 612 - protein, 6 aa, Artificial Sequence
  Description of Artificial Sequence: Synthetic antibody
QSVSSY

SEQ ID NO: 613 - protein, 3 aa, Artificial Sequence
  Description of Artificial Sequence: Synthetic antibody
DTS

SEQ ID NO: 614 - protein, 8 aa, Artificial Sequence
  Description of Artificial Sequence: Synthetic antibody
QQRGNWWT

SEQ ID NO: 615 - protein, 6 aa, Artificial Sequence
  Description of Artificial Sequence: Synthetic antibody
QSVSNN

SEQ ID NO: 616 - protein, 3 aa, Artificial Sequence
  Description of Artificial Sequence: Synthetic antibody
GAS

SEQ ID NO: 617 - protein, 10 aa, Artificial Sequence
  Description of Artificial Sequence: Synthetic antibody
QQCYNWPPWT

SEQ ID NO: 618 - protein, 9 aa, Artificial Sequence
  Description of Artificial Sequence: Synthetic antibody
SSDVGSYNL

SEQ ID NO: 619 - protein, 3 aa, Artificial Sequence
  Description of Artificial Sequence: Synthetic antibody
EVI

SEQ ID NO: 620 - protein, 11 aa, Artificial Sequence
  Description of Artificial Sequence: Synthetic antibody
CSYAVSTTYVI

SEQ ID NO: 621 - protein, 7 aa, Artificial Sequence
  Description of Artificial Sequence: Synthetic antibody
SGYSNYK

SEQ ID NO: 622 - protein, 8 aa, Artificial Sequence
  Description of Artificial Sequence: Synthetic antibody
VGTGGIVG

SEQ ID NO: 623 - protein, 14 aa, Artificial Sequence
  Description of Artificial Sequence: Synthetic antibody
GADHGSGSNFVFVV

SEQ ID NO: 624 - protein, 11 aa, Artificial Sequence
  Description of Artificial Sequence: Synthetic antibody
QSLLHSNGYNY

SEQ ID NO: 625 - protein, 3 aa, Artificial Sequence
  Description of Artificial Sequence: Synthetic antibody
LGS

SEQ ID NO: 626 - protein, 9 aa, Artificial Sequence
  Description of Artificial Sequence: Synthetic antibody
MQALQTPFT

SEQ ID NO: 627 - protein, 6 aa, Artificial Sequence
  Description of Artificial Sequence: Synthetic antibody
QSVSSN

SEQ ID NO: 628 - protein, 3 aa, Artificial Sequence
  Description of Artificial Sequence: Synthetic antibody
GAS

SEQ ID NO: 629 - protein, 10 aa, Artificial Sequence
  Description of Artificial Sequence: Synthetic antibody
QQYNYWPPLT

SEQ ID NO: 630 - protein, 6 aa, Artificial Sequence
  Description of Artificial Sequence: Synthetic antibody
NIGSKS

SEQ ID NO: 631 - protein, 3 aa, Artificial Sequence
  Description of Artificial Sequence: Synthetic antibody
DDS

SEQ ID NO: 632 - protein, 12 aa, Artificial Sequence
  Description of Artificial Sequence: Synthetic antibody
QVWDSSSDHHVV

SEQ ID NO: 633 - protein, 9 aa, Artificial Sequence
  Description of Artificial Sequence: Synthetic antibody
SSNIGAGYD

SEQ ID NO: 634 - protein, 3 aa, Artificial Sequence
  Description of Artificial Sequence: Synthetic antibody
INS

SEQ ID NO: 635 - protein, 10 aa, Artificial Sequence
  Description of Artificial Sequence: Synthetic antibody
QSYDSSLGAL

SEQ ID NO: 636 - protein, 6 aa, Artificial Sequence
  Description of Artificial Sequence: Synthetic antibody
QSISSF

SEQ ID NO: 637 - protein, 3 aa, Artificial Sequence
  Description of Artificial Sequence: Synthetic antibody
AAF

SEQ ID NO: 638 - protein, 9 aa, Artificial Sequence
  Description of Artificial Sequence: Synthetic antibody
QQSYNTPYT

SEQ ID NO: 639 - protein, 6 aa, Artificial Sequence
  Description of Artificial Sequence: Synthetic antibody
QSVSSY

SEQ ID NO: 640 - protein, 3 aa, Artificial Sequence
  Description of Artificial Sequence: Synthetic antibody
DAS

SEQ ID NO: 641 - protein, 11 aa, Artificial Sequence
  Description of Artificial Sequence: Synthetic antibody
QHRSNWPPRLT

SEQ ID NO: 642 - protein, 6 aa, Artificial Sequence
  Description of Artificial Sequence: Synthetic antibody
QSVSSN

SEQ ID NO: 643 - protein, 3 aa, Artificial Sequence
  Description of Artificial Sequence: Synthetic antibody
GAS

SEQ ID NO: 644 - protein, 8 aa, Artificial Sequence
  Description of Artificial Sequence: Synthetic antibody
QQYNNFLT

SEQ ID NO: 645 - protein, 8 aa, Artificial Sequence
  Description of Artificial Sequence: Synthetic antibody
SGSIASNY

SEQ ID NO: 646 - protein, 3 aa, Artificial Sequence
  Description of Artificial Sequence: Synthetic antibody
EDN

SEQ ID NO: 647 - protein, 11 aa, Artificial Sequence
  Description of Artificial Sequence: Synthetic antibody
QSYDGSNHAVV

SEQ ID NO: 648 - protein, 8 aa, Artificial Sequence
  Description of Artificial Sequence: Synthetic antibody
SSNIGSNT

SEQ ID NO: 649 - protein, 3 aa, Artificial Sequence
  Description of Artificial Sequence: Synthetic antibody
SNN

SEQ ID NO: 650 - protein, 11 aa, Artificial Sequence
  Description of Artificial Sequence: Synthetic antibody
AAWDDSLNALV

SEQ ID NO: 651 - protein, 6 aa, Artificial Sequence
  Description of Artificial Sequence: Synthetic antibody
QSIAGY

SEQ ID NO: 652 - protein, 3 aa, Artificial Sequence
  Description of Artificial Sequence: Synthetic antibody
GTT

SEQ ID NO: 653 - protein, 9 aa, Artificial Sequence
  Description of Artificial Sequence: Synthetic antibody
QQSYSTPGT

SEQ ID NO: 654 - protein, 6 aa, Artificial Sequence
  Description of Artificial Sequence: Synthetic antibody
QDINHY

SEQ ID NO: 655 - protein, 3 aa, Artificial Sequence
  Description of Artificial Sequence: Synthetic antibody
DAS

SEQ ID NO: 656 - protein, 10 aa, Artificial Sequence
  Description of Artificial Sequence: Synthetic antibody
QQSDNLPMYT

SEQ ID NO: 657 - protein, 6 aa, Artificial Sequence
  Description of Artificial Sequence: Synthetic antibody
NIGSKS

SEQ ID NO: 658 - protein, 3 aa, Artificial Sequence
  Description of Artificial Sequence: Synthetic antibody
DDS

SEQ ID NO: 659 - protein, 12 aa, Artificial Sequence
  Description of Artificial Sequence: Synthetic antibody
QVWDSSSDHHVV

SEQ ID NO: 660-662 - skipped (3 entries)

SEQ ID NO: 663 - protein, 8 aa, Artificial Sequence
  Description of Artificial Sequence: Synthetic antibody
NSNIGSYT

SEQ ID NO: 664 - protein, 3 aa, Artificial Sequence
  Description of Artificial Sequence: Synthetic antibody
DNN

SEQ ID NO: 665 - protein, 11 aa, Artificial Sequence
  Description of Artificial Sequence: Synthetic antibody
LVWDDSLNGLV

SEQ ID NO: 666 - protein, 9 aa, Artificial Sequence
  Description of Artificial Sequence: Synthetic antibody
SSNIGSGSD

SEQ ID NO: 667 - protein, 3 aa, Artificial Sequence
  Description of Artificial Sequence: Synthetic antibody
GNT

SEQ ID NO: 668 - protein, 12 aa, Artificial Sequence
  Description of Artificial Sequence: Synthetic antibody
QSYDSRLSGFVV

SEQ ID NO: 669 - protein, 6 aa, Artificial Sequence
  Description of Artificial Sequence: Synthetic antibody
QSISSY

SEQ ID NO: 670 - protein, 3 aa, Artificial Sequence
  Description of Artificial Sequence: Synthetic antibody
AAS

SEQ ID NO: 671 - protein, 9 aa, Artificial Sequence
  Description of Artificial Sequence: Synthetic antibody
QQSYSTPQT

SEQ ID NO: 672 - protein, 6 aa, Artificial Sequence
  Description of Artificial Sequence: Synthetic antibody
QSISNY

SEQ ID NO: 673 - protein, 3 aa, Artificial Sequence
  Description of Artificial Sequence: Synthetic antibody
AAS

SEQ ID NO: 674 - protein, 9 aa, Artificial Sequence
  Description of Artificial Sequence: Synthetic antibody
QQSYTTLLT

SEQ ID NO: 675-677 - skipped (3 entries)

SEQ ID NO: 678 - protein, 9 aa, Artificial Sequence
  Description of Artificial Sequence: Synthetic antibody
NSNIGAGYD

SEQ ID NO: 679 - protein, 3 aa, Artificial Sequence
  Description of Artificial Sequence: Synthetic antibody
GNS

SEQ ID NO: 680 - protein, 11 aa, Artificial Sequence
  Description of Artificial Sequence: Synthetic antibody
QSYDSSLSDSV

SEQ ID NO: 681-683 - skipped (3 entries)

SEQ ID NO: 684 - protein, 6 aa, Artificial Sequence
  Description of Artificial Sequence: Synthetic antibody
QSVSSN

SEQ ID NO: 685 - protein, 3 aa, Artificial Sequence
  Description of Artificial Sequence: Synthetic antibody
GAS

SEQ ID NO: 686 - protein, 9 aa, Artificial Sequence
  Description of Artificial Sequence: Synthetic antibody
QQYYNWPLT

SEQ ID NO: 687 - protein, 6 aa, Artificial Sequence
  Description of Artificial Sequence: Synthetic antibody
SLRSYY

SEQ ID NO: 688 - protein, 3 aa, Artificial Sequence
  Description of Artificial Sequence: Synthetic antibody
DKN

SEQ ID NO: 689 - protein, 11 aa, Artificial Sequence
  Description of Artificial Sequence: Synthetic antibody
NSRDSSGNAVV

SEQ ID NO: 690 - protein, 6 aa, Artificial Sequence
  Description of Artificial Sequence: Synthetic antibody
QDISNY

SEQ ID NO: 691 - protein, 3 aa, Artificial Sequence
  Description of Artificial Sequence: Synthetic antibody
DAS

SEQ ID NO: 692 - protein, 9 aa, Artificial Sequence
  Description of Artificial Sequence: Synthetic antibody
QQYANLPFT

SEQ ID NO: 693-698 - skipped (6 entries)

SEQ ID NO: 699 - protein, 6 aa, Artificial Sequence
  Description of Artificial Sequence: Synthetic antibody
QNIASY

SEQ ID NO: 700 - protein, 3 aa, Artificial Sequence
  Description of Artificial Sequence: Synthetic antibody
AAS

SEQ ID NO: 701 - protein, 9 aa, Artificial Sequence
  Description of Artificial Sequence: Synthetic antibody
QQSYSTPWT

SEQ ID NO: 702 - protein, 6 aa, Artificial Sequence
  Description of Artificial Sequence: Synthetic antibody
QSVRSN

SEQ ID NO: 703 - protein, 3 aa, Artificial Sequence
  Description of Artificial Sequence: Synthetic antibody
GAS

SEQ ID NO: 704 - protein, 9 aa, Artificial Sequence
  Description of Artificial Sequence: Synthetic antibody
HQYNNWPQT

SEQ ID NO: 705 - protein, 8 aa, Artificial Sequence
  Description of Artificial Sequence: Synthetic antibody
SGSIASNY

SEQ ID NO: 706 - protein, 3 aa, Artificial Sequence
  Description of Artificial Sequence: Synthetic antibody
EDN

SEQ ID NO: 707 - protein, 11 aa, Artificial Sequence
  Description of Artificial Sequence: Synthetic antibody
QSYDGINRAWV

SEQ ID NO: 708 - protein, 11 aa, Artificial Sequence
  Description of Artificial Sequence: Synthetic antibody
QSLLHSLGYNS

SEQ ID NO: 709 - protein, 3 aa, Artificial Sequence
  Description of Artificial Sequence: Synthetic antibody
LGS

SEQ ID NO: 710 - protein, 9 aa, Artificial Sequence
  Description of Artificial Sequence: Synthetic antibody
MQALQTPLT

SEQ ID NO: 711 - protein, 6 aa, Artificial Sequence
  Description of Artificial Sequence: Synthetic antibody
QSISSY

SEQ ID NO: 712 - protein, 3 aa, Artificial Sequence
  Description of Artificial Sequence: Synthetic antibody
AAS

SEQ ID NO: 713 - protein, 10 aa, Artificial Sequence
  Description of Artificial Sequence: Synthetic antibody
QQSYSSLSIT

SEQ ID NO: 714 - protein, 6 aa, Artificial Sequence
  Description of Artificial Sequence: Synthetic antibody
QSISSY

SEQ ID NO: 715 - protein, 3 aa, Artificial Sequence
  Description of Artificial Sequence: Synthetic antibody
AAS

SEQ ID NO: 716 - protein, 10 aa, Artificial Sequence
  Description of Artificial Sequence: Synthetic antibody
QQSYEIPPWT

SEQ ID NO: 717 - protein, 6 aa, Artificial Sequence
  Description of Artificial Sequence: Synthetic antibody
QDINNY

SEQ ID NO: 718 - protein, 3 aa, Artificial Sequence
  Description of Artificial Sequence: Synthetic antibody
DAS

SEQ ID NO: 719 - protein, 9 aa, Artificial Sequence
  Description of Artificial Sequence: Synthetic antibody
QQYDNLPPV

SEQ ID NO: 720 - protein, 9 aa, Artificial Sequence
  Description of Artificial Sequence: Synthetic antibody
SSNIGAGYD

SEQ ID NO: 721 - protein, 3 aa, Artificial Sequence
  Description of Artificial Sequence: Synthetic antibody
ANS

SEQ ID NO: 722 - protein, 11 aa, Artificial Sequence
  Description of Artificial Sequence: Synthetic antibody
QSYDSSLSGSV

SEQ ID NO: 723 - protein, 8 aa, Artificial Sequence
  Description of Artificial Sequence: Synthetic antibody
SGSIASNY

SEQ ID NO: 724 - protein, 3 aa, Artificial Sequence
  Description of Artificial Sequence: Synthetic antibody
EDN

SEQ ID NO: 725 - protein, 9 aa, Artificial Sequence
  Description of Artificial Sequence: Synthetic antibody
QSYDSGNPI

SEQ ID NO: 726-728 - skipped (3 entries)

SEQ ID NO: 729 - protein, 6 aa, Artificial Sequence
  Description of Artificial Sequence: Synthetic antibody
QDINNY

SEQ ID NO: 730 - protein, 3 aa, Artificial Sequence
  Description of Artificial Sequence: Synthetic antibody
DAS

SEQ ID NO: 731 - protein, 9 aa, Artificial Sequence
  Description of Artificial Sequence: Synthetic antibody
QQYDNLPPV

SEQ ID NO: 732 - protein, 8 aa, Artificial Sequence
  Description of Artificial Sequence: Synthetic antibody
SGSIASNY

SEQ ID NO: 733 - protein, 3 aa, Artificial Sequence
  Description of Artificial Sequence: Synthetic antibody
EDN

SEQ ID NO: 734 - protein, 11 aa, Artificial Sequence
  Description of Artificial Sequence: Synthetic antibody
QSYDGINRWLV

SEQ ID NO: 735 - protein, 7 aa, Artificial Sequence
  Description of Artificial Sequence: Synthetic antibody
SGYSNYK

SEQ ID NO: 736 - protein, 8 aa, Artificial Sequence
  Description of Artificial Sequence: Synthetic antibody
VGTGGIVG

SEQ ID NO: 737 - protein, 14 aa, Artificial Sequence
  Description of Artificial Sequence: Synthetic antibody
GADHGSGSNFEYVV

SEQ ID NO: 738 - protein, 8 aa, Artificial Sequence
  Description of Artificial Sequence: Synthetic antibody
SSNIGYNI

SEQ ID NO: 739 - protein, 3 aa, Artificial Sequence
  Description of Artificial Sequence: Synthetic antibody
SNN

SEQ ID NO: 740 - protein, 11 aa, Artificial Sequence
  Description of Artificial Sequence: Synthetic antibody
AAWDDSLNGYV

SEQ ID NO: 741 - protein, 6 aa, Artificial Sequence
  Description of Artificial Sequence: Synthetic antibody
NIGNKG

SEQ ID NO: 742 - protein, 3 aa, Artificial Sequence
  Description of Artificial Sequence: Synthetic antibody
DDS

SEQ ID NO: 743 - protein, 12 aa, Artificial Sequence
  Description of Artificial Sequence: Synthetic antibody
QVWDSSSDHPGV

SEQ ID NO: 744 - protein, 6 aa, Artificial Sequence
  Description of Artificial Sequence: Synthetic antibody
QGISSY

SEQ ID NO: 745 - protein, 3 aa, Artificial Sequence
  Description of Artificial Sequence: Synthetic antibody
AAS

SEQ ID NO: 746 - protein, 9 aa, Artificial Sequence
  Description of Artificial Sequence: Synthetic antibody
QQLNSYPVT

SEQ ID NO: 747 - protein, 9 aa, Artificial Sequence
  Description of Artificial Sequence: Synthetic antibody
SSDVGDYNY

SEQ ID NO: 748 - protein, 3 aa, Artificial Sequence
  Description of Artificial Sequence: Synthetic antibody
DVS

SEQ ID NO: 749 - protein, 12 aa, Artificial Sequence
  Description of Artificial Sequence: Synthetic antibody
SSYTSSSTLLYV

SEQ ID NO: 750 - protein, 6 aa, Artificial Sequence
  Description of Artificial Sequence: Synthetic antibody
QSVSSF

SEQ ID NO: 751 - protein, 3 aa, Artificial Sequence
  Description of Artificial Sequence: Synthetic antibody
DAS

SEQ ID NO: 752 - protein, 11 aa, Artificial Sequence
  Description of Artificial Sequence: Synthetic antibody
HYRSNWPPVLT

SEQ ID NO: 753 - protein, 6 aa, Artificial Sequence
  Description of Artificial Sequence: Synthetic antibody
QSVSSF

SEQ ID NO: 754 - protein, 3 aa, Artificial Sequence
  Description of Artificial Sequence: Synthetic antibody
DAS

SEQ ID NO: 755 - protein, 11 aa, Artificial Sequence
  Description of Artificial Sequence: Synthetic antibody
QHRTNWPPLFT

SEQ ID NO: 756 - protein, 12 aa, Artificial Sequence
  Description of Artificial Sequence: Synthetic antibody
QSVLYSSNNKNS

SEQ ID NO: 757 - protein, 3 aa, Artificial Sequence
  Description of Artificial Sequence: Synthetic antibody
WAS

SEQ ID NO: 758 - protein, 9 aa, Artificial Sequence
  Description of Artificial Sequence: Synthetic antibody
QQYYSISWT

SEQ ID NO: 759 - protein, 6 aa, Artificial Sequence
  Description of Artificial Sequence: Synthetic antibody
QDITNY

SEQ ID NO: 760 - protein, 3 aa, Artificial Sequence
  Description of Artificial Sequence: Synthetic antibody
DAS

SEQ ID NO: 761 - protein, 10 aa, Artificial Sequence
  Description of Artificial Sequence: Synthetic antibody
QQYDNLYSVH

SEQ ID NO: 762 - protein, 6 aa, Artificial Sequence
  Description of Artificial Sequence: Synthetic antibody
ALPRQY

SEQ ID NO: 763 - protein, 3 aa, Artificial Sequence
  Description of Artificial Sequence: Synthetic antibody
KDS

SEQ ID NO: 764 - protein, 11 aa, Artificial Sequence
  Description of Artificial Sequence: Synthetic antibody
QSADTIGTYWV

SEQ ID NO: 765 - protein, 6 aa, Artificial Sequence
  Description of Artificial Sequence: Synthetic antibody
KLGDKY

SEQ ID NO: 766 - protein, 3 aa, Artificial Sequence
  Description of Artificial Sequence: Synthetic antibody
QDT

SEQ ID NO: 767 - protein, 9 aa, Artificial Sequence
  Description of Artificial Sequence: Synthetic antibody
QAWGSSRGV

SEQ ID NO: 768 - protein, 6 aa, Artificial Sequence
  Description of Artificial Sequence: Synthetic antibody
QSVSSY

SEQ ID NO: 769 - protein, 3 aa, Artificial Sequence
  Description of Artificial Sequence: Synthetic antibody
DAS

SEQ ID NO: 770 - protein, 11 aa, Artificial Sequence
  Description of Artificial Sequence: Synthetic antibody
HKRSNWPPSLT

SEQ ID NO: 771 - DNA, 360 bp, Artificial Sequence
  Description of Artificial Sequence: Synthetic antibody
gaggtgcagctggtggagtctgggggagacttggtacagcctggggggtccctgagactctcctgtgcagcctctggattcacctttcgcagctatgccatgagctgggtccgccaggctccagggaaggggctggagtgggtctcagctattagtgataatgcttatagcacatactacgcagactccgtgaagggccggttcaccatctccagagacaattccaagaacactctgtatctgcaaatgaacagcctgagagccgaggacacggccatatattactgtgcgaagaatctttatagtggaaactccccatttgactactggggccagggaaccctggtcaccgtctcctca

SEQ ID NO: 772 - DNA, 317 bp, Artificial Sequence
  Description of Artificial Sequence: Synthetic antibody
  misc_feature (1)..(3): n is a, c, g, or t
  misc_feature (5)..(5): n is a, c, g, or t
nnntntgtgctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcactgccgtcttcggaactgggaccaaggtcaccgtcct

SEQ ID NO: 773 - DNA, 390 bp, Artificial Sequence
  Description of Artificial Sequence: Synthetic antibody
gaggtgcagctggtggagtctgggggaggcgtggtccagcctgggaggtccctgagactctcctgtgcagcgtctggattcaccttcagtagttatggcatgcattgggtccgccaggctccaggcaaggggctggagtgggtggcagttatatggcatgatggaagtaagaaatactatgcagactccgtgaagggccgattcaccatctccagagacaattccaagaacacgctgtatctgcaaatgaacagcctgagagccgaggacacggctgtgtattactgtgcgagagatcaaggtggatatgattacgtttgggggagttatcgatatacattttacgtctttgactactggggccagggaaccctggtcaccgtctcctca

SEQ ID NO: 774 - DNA, 390 bp, Artificial Sequence; identical to SEQ ID NO: 773
  Description of Artificial Sequence: Synthetic antibody

SEQ ID NO: 775 - DNA, 357 bp, Artificial Sequence
  Description of Artificial Sequence: Synthetic antibody
gaggtgcagctggtggagtctgggggaggcttggtccagcctggggggtccctgagactctcctgtgcagcctctggattcacctttagtttctattggatgagctgggtccgccaggctccagggaaggggctggagtgggtggccaacataaagcaagatggaagtgagaaatactatgtggactctgtgaagggccgattcagcatctccagagacaacgccaagaactcactgtatctgcaaatgaacagcctgagagccgaggacacggctgtgtattactgtgcgagagttggtagcagcagctggtattttgactactggggccagggaaccctggtcaccgtctcctca

SEQ ID NO: 776 - DNA, 317 bp, Artificial Sequence
  Description of Artificial Sequence: Synthetic antibody
  misc_feature (1)..(4): n is a, c, g, or t
nnnnttgtgctgactcagccaccctcagtgtccgtgtccccaggacagacagccagcatcacctgctctggagataaattgggggataaatatgcttgctggtatcagcagaagccaggccagtcccctgtgctggtcatctatcaagatatcaagcggccctcagggatccctgagcgattctctggctccaactctgggaacacagccactctgaccatcagcgggacccaggctatggatgaggctgactattactgtcaggcgtgggacagcaacactggagtgttcggcggagggaccaagctgaccgtcct

SEQ ID NO: 777 - DNA, 363 bp, Artificial Sequence
  Description of Artificial Sequence: Synthetic antibody
gaggtgcagctggtgcagtctggggctgaggtgaagaagcctggggcctcagtgaaggtctcctgcaaggcttctggatacaccctcaccaggtatgatatccactgggtgcgacaggccactggacaagggcttgagtggatgggatggttgaaccctaacggtggcaacacaggctatgcacagaagttccagggcagagtcaccatgaccagaaacaccgccataagcacagcctacatggagctgagcagcctgagatctgaggacgcggccgtgtattactgtgcgaggggtcagtgggaactagacgcttggtacttcgatctctggggccgtggcaccctggtcaccgtctcctca

SEQ ID NO: 778 - DNA, 329 bp, Artificial Sequence
  Description of Artificial Sequence: Synthetic antibody
  misc_feature (5)..(5): n is a, c, g, or t
cagtntgtgctgactcaggaccctgctgtgtctgtggccttgggacagacagtcaggatcacatgccaaggagacagcttcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattattgtaactcccgggacaccagtggtaaccatctagatgtggtattcggcggagggaccaagctgaccgtcct

SEQ ID NO: 779 - DNA, 363 bp, Artificial Sequence
  Description of Artificial Sequence: Synthetic antibody
gaggtgcagctggtgcagtctggggctgaggtgaagaagcctggggcctcactgaaggtctcctgcaaggcttctggatacaccttcacccgttatgatatcaactgggtgcgacaggccactggacaagggcttgagtggatgggatggatgaaccctaacagtgataacacaggctatgcacagaagttccaggacagagtcaccatgaccaggaacacctccataagcacagtctacatggagctgagcagcctgagatctgaggacacggccgtgtattactgtgcgaggggtcagtgggagctagacgtttggtacttcgatctctggggccgtggcaccctggtcaccgtctcctca

SEQ ID NO: 780 - DNA, 329 bp, Artificial Sequence
  Description of Artificial Sequence: Synthetic antibody
  misc_feature (4)..(5): n is a, c, g, or t
cagnntgtgctgactcaggaccctgctgtgtctgtggccttgggacagacactcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagcggtcaccatctagatgtggtattcggcggagggaccaagctgaccgtcct

SEQ ID NO: 781 - DNA, 369 bp, Artificial Sequence
  Description of Artificial Sequence: Synthetic antibody
caggtgcagctgcaggagtcgggcccaggactggtgaagccttcggagaccctgtccctcacctgcactgtctctggtggctccatcagtagttactactggagctggatccggcagcccccagggaagggactggagtggattgggtgtatctattacagtgggcgcaccaactacagcccctccctcaagagtcgagtcaccatatcagtagacacgtccaagaacctgttctccctgaagctgagctctgtgaccgctgcggacacggccgtgtattactgtgcgagaggaggccgccctggggctgaaggaccttatgatgcttttgatatctggggccaagggacaatggtcaccgtctcctca

SEQ ID NO: 782 - DNA, 323 bp, Artificial Sequence
  Description of Artificial Sequence: Synthetic antibody
  misc_feature (4)..(5): n is a, c, g, or t
cagnntgtgctgactcaggaccctgctgtgtctgtggccttgggacagacagtcaggatcacatgccaaggagacaacctcagaaggtattatgcaagctggtaccagcagaagccaggacaggcccctgtactagtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccacgctgaccgtcct

SEQ ID NO: 783 - DNA, 370 bp, Artificial Sequence
  Description of Artificial Sequence: Synthetic antibody
caggtgcagctggtgcaatctgggtctgagttgaagaagcctggggcctcagtgaaggtttcctgcaaggcttctggatacaccttcagtgactatgctatgaattgggtgcgacaggcccctggacaaggacttgagtggatgggatggatgaagtctaacagtggtaacacaggctatgcacagaagttccagggcagagtcactatgaccaggaacacctccataagtacagcctacatggagttgaccagcctgagatctgaggacacggccgtgtattactgtgcgagaatgcgcagtggctggcccacacatggccgcccggatgaccactggggccggggagccctggtcaccgtctcctcag

SEQ ID NO: 784 - DNA, 325 bp, Artificial Sequence
  Description of Artificial Sequence: Synthetic antibody
gacatccagatgacccagtctccatcctccctgtctgcatctgtgggagacagagtcaccatcacttgccgggcaagtcagagcattatcagctatttaaattggtatcaccagaaaccggggaaagcccctaaactcctgatctatgctgcatccagtttgcaaagtggggtcccatcaaggttcagtggcagtggatctgggacagattttactttcaccatcagcagcctgcagcctgaagatattgcaacatattactgtcaacagtctgataatctccccatgtacacttttggccaggggaccaagctggagatcaaac

SEQ ID NO: 785 - DNA, 387 bp, Artificial Sequence
  Description of Artificial Sequence: Synthetic antibody
caggtgcagctggtggagtctgggggaggcgtggtccagcctgggaggtccctgagactctcctgtgcagcctctggattcaccttcgttacctctggcatacactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaggagggccaaacaaggaagtactatatttcggggagttattggactacggtatggacgtctggggccaagggaccacggtcaccgtctcctca

SEQ ID NO: 786 - DNA, 343 bp, Artificial Sequence
  Description of Artificial Sequence: Synthetic antibody
gacatcgtgatgacccagtctccagactccctggctgtgtctctgggcgagagggccaccatcaactgcaagtccagccagagtgttttatacagttccgaaattaagaactacttagcttggtatcagcagaaaccaggacagcctcctaagctgctcatttactgggcatctacccgggaattcggggtccctgaccgattcagtggcagcgggtctgggacagatttcactctcaccatcagcagcctgcaggctgaagatgtggcagtttattactgtcagcaatattatagtggtcccctggacacttttggccaggggaccaagctggagatcaaac

SEQ ID NO: 787 - DNA, 366 bp, Artificial Sequence
  Description of Artificial Sequence: Synthetic antibody
caggtccagcttgtgcagtctggggctgaggtgaagaagcctggggcctcagtgaaggtttcctgcaaggtctctggatacaccttcactggctatgttgtacattgggtgcgccaggcccccggacaagaccttgagtggatgggatggatcaacactggctacggcaacacaaaatattcacagaagttccagggcagggtcactattagctgggacacatccgcgacgacagcctacatggagctgagcaacctgaaatctgaggacaaggctgtttattactgtgcgagtatgaaccggatgtcagagcaaacttactacggaatggacgtctggggccaggggaccacggtcaccgtctcc

SEQ ID NO: 788 - DNA, 316 bp, Artificial Sequence
  Description of Artificial Sequence: Synthetic antibody
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataagctcactctcggcggagggaccaaggtggagatcaaac

SEQ ID NO: 789 - DNA, 363 bp, Artificial Sequence
  Description of Artificial Sequence: Synthetic antibody
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaatccgtccctcaagagtcgagtcaccatatcagtggacacgtccaagaaccacttctccctgaaaatgaactctgtgaccgccgcggacacggctgtgtattactgtgcgagatgtcgccagatggggaacttctactactactacatggacgtctggggcaaggggaccacggtcaccgtctcc

SEQ ID NO: 790 - DNA, 334 bp, Artificial Sequence
  Description of Artificial Sequence: Synthetic antibody
cagtctgtgctgacgcagccgccctcagtgtctggggtcccagggcagagggtcaccgtctcctgcactgggagcagctccaacatcggggcaggttttgatgtatattggtaccagcagtttctaggaacagcccccaaactcctcatctatggcaacaacaatcggccctcaggcgtccctgaccgattctctgcctccaagtctggcacctcagcctccctggccatcactgggctccaggctgaagatgaggctgattattactgccagtcctttgacatcggccggggtggttggattttcggcggagggaccaagctgaccgtcctag

SEQ ID NO: 791 - DNA, 382 bp, Artificial Sequence
  Description of Artificial Sequence: Synthetic antibody
caggtgcacctacaggagtcgggcccaggactggtgaagccttcggagaccctgtccctcacctgcactgtctctggtggctccatcaataattattactggagctggatccggcagcccccggggaagggactggagtggattggggaaatccattacagtgggagcaccagctacagcccctccctcaagagtcgactcagcatatcagtagacaggtccaagaaccagttctccctgaagctggcctctgtgaccgctgcagacacggccgtgtattactgtgtgagggataattactttgataatagtggtcatcctgtgtatccggttccctggttcgacccctggggccagggaaccctggtcaccgtctcctcag

SEQ ID NO: 792 - DNA, 331 bp, Artificial Sequence
  Description of Artificial Sequence: Synthetic antibody
cagtctgtgctgacgcagccgccctcagtgtctggggtcccagggcagagggtcaccgtctcctgcactgggagcagctccaacatcggggcaggttttgatgtatattggtaccagcagtttctaggaacagcccccaaactcctcatctatggcaacaacaatcggccctcaggcgtccctgaccgattctctgcctccaagtctggcacctcagcctccctggccatcactgggctccaggctgaagatgaggctgattattactgccagtcctttgacatcggccggggtggttggattttcggcggagggaccaagctcaccgtcc

SEQ ID NO: 793 - DNA, 352 bp, Artificial Sequence
  Description of Artificial Sequence: Synthetic antibody
caggttcaggtggtgcagtctggagctgaggtaaagaagcctggggcctcagtgaaggtctcctgcaaggcttctggttacacctttaagaactatgggatcagttgggtgcgacaggcccctggacaagggcttgagtggatgggatggatcagcgcttacacaggaaacacaaactatgcacagaagttccagggcagaatgaccatgaccacagacacatccacgggaacaggttatatggaactgaggagcctgagatctgacgacacggccgtgtattactgtgcgagagtacaacgacgtcgacttgactactggggccagggaaccctggtcatcgtctcgtcag

SEQ ID NO: 794 - DNA, 331 bp, Artificial Sequence
  Description of Artificial Sequence: Synthetic antibody
gatattgtggtgacccagactccactctctctgtccgtcacccctggacagccggcctccatctcctgcaagtctagtgagaccctcctgcatagtgatggaaagacctatttgtcttggtatctgcagaagccaggccagcctccacagctcctgatctatgaagtttccaaccggttctctggagtgccagacaggttcagtggcagcgggtcaggaacagatttcacacttaaaatcggccgggtggaggctgaggatgttgggctttattactgcatgcaaagtatacagctcgccttcggccaagggacacgactggaaattgaac

SEQ ID NO: 795 - DNA, 372 bp, Artificial Sequence
  Description of Artificial Sequence: Synthetic antibody
gaggtgcagctggtggagtctgggggaggcgtggtccagcctgggaggtccctgagactctcctgtgcagcctctggattcaccttcagtacgtatgctatgcactgggtccgccaggctccaggcaaggggctggagtgggtggcagttatatcatatgatggaagcaataaatactacgcagactccgtgaagggccgattcaccatctccagagacaattccaagaacacactatatctgcaaatgatcggcctgagagctgaggacacggctgtgtattactgtgcgagagattgggcacctacgtactacgatatgccgagtgcttttgatatctggggccaagggacaatggtcaccgtctcttca

SEQ ID NO: 796 - DNA, 327 bp, Artificial Sequence
  Description of Artificial Sequence: Synthetic antibody
tcctatgagctgacacagccaccctcggtgtcagtggccccaggaaagacggccaggattacctgtgggggaaacaacattggaaataaaggtgtgcattggtaccagcagaagccaggccaggcccctgtgctggtcgtcgatgatgatagcgaccggccctcagggatccctgagcgattctctggctccaactctgggaacacggccaccctgatcatcagcagtgtcgaagtcggggatgaggccgacttttactgtcaggtgtgggatagtagtagtgatcatccgggggtgttcggcggagggaccaagctgaccgtccta

SEQ ID NO: 797 - DNA, 363 bp, Artificial Sequence
  Description of Artificial Sequence: Synthetic antibody
gaggtgcagctggtggagtctgggggaggcgtggtccagcctgggaggtccctgagactctcctgtgcagcctctggattcaccttcagtagctatgctatgcactgggtccgccaggctccaggcaaggggctggaatgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagaagcacgagtgggagctactactacggtatggacgtctggggccaagggaccacggtcaccgtctcctca

SEQ ID NO: 798 - DNA, 336 bp, Artificial Sequence; identical to SEQ ID NO: 140
  Description of Artificial Sequence: Synthetic antibody

SEQ ID NO: 799 - DNA, 363 bp, Artificial Sequence; identical to SEQ ID NO: 797
  Description of Artificial Sequence: Synthetic antibody

SEQ ID NO: 800 - DNA, 336 bp, Artificial Sequence; identical to SEQ ID NO: 140
  Description of Artificial Sequence: Synthetic antibody

SEQ ID NO: 801 - DNA, 366 bp, Artificial Sequence
  Description of Artificial Sequence: Synthetic antibody
gaggtgcagctggtgcagtctggggctgaggtgaagaagcctgggtcctcggtgaaggtctcctgcaaggcttctggaggcaccttcagcagctatgctatcatctgggtgcgacaggcccctggacaagggcttgagtggatgggagggatcatccctatctttggtacaacaaactacgcacagaagttccagggcagagtcacgattaccgcggacgaatccacgagcacagcctacgtggaactgagcagcctgagatctgaggacacggccgtgtattattgtgcgagaataggccattttgatagtagtggttattacttagactactggggccagggaaccctggtcaccgtctcctca

SEQ ID NO: 802 - DNA, 327 bp, Artificial Sequence; identical to SEQ ID NO: 144
  Description of Artificial Sequence: Synthetic antibody

SEQ ID NO: 803 - DNA, 366 bp, Artificial Sequence
  Description of Artificial Sequence: Synthetic antibody
gaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtgcagcaaactacgcacagaacttccagggcagagtcacgattaccgcggacgaatccacgagcacaggctacatgcaactgagcagcctgagatttgaggacacggccgtgtattactgtgcgagaacgtctcactatgatagtagtggttcctattttgaatactggggccagggaaccctggtcaccgtctcctca

SEQ ID NO: 804 - DNA, 327 bp, Artificial Sequence
  Description of Artificial Sequence: Synthetic antibody
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagaccctgaagattttgcagtttattactgtcacaagcgtagcaactggcctccttcgctcactttcggcggagggaccaaggtggaaatcaaa

SEQ ID NO: 805 - DNA, 357 bp, Artificial Sequence; identical to SEQ ID NO: 3
  Description of Artificial Sequence: Synthetic antibody

SEQ ID NO: 806 - DNA, 318 bp, Artificial Sequence
  Description of Artificial Sequence: Synthetic antibody
tcctatgagctgacacagccaccctcagtgtccgtgtccccaggacagacagccagcatcacctgctctggagataaattgggggataaatatgcttgttggtaccaacagaggccaggccagtcccctgtgctggtcatctatcaagatagtaagcggccctcagggatccctgagcgattctctggctccaactctgggaacacagccactctgaccatcagcgggacccaggctatggatgaggctgactattactgtcaggcgtgggacagcagcactggagtcttcggaactgggaccaaggtcaccgtccta

SEQ ID NO: 807 - DNA, 387 bp, Artificial Sequence; identical to SEQ ID NO: 5
  Description of Artificial Sequence: Synthetic antibody

SEQ ID NO: 808 - DNA, 321 bp, Artificial Sequence; identical to SEQ ID NO: 6
  Description of Artificial Sequence: Synthetic antibody

SEQ ID NO: 809 - DNA, 387 bp, Artificial Sequence; identical to SEQ ID NO: 5
  Description of Artificial Sequence: Synthetic antibody

SEQ ID NO: 810 - DNA, 321 bp, Artificial Sequence; identical to SEQ ID NO: 6
  Description of Artificial Sequence: Synthetic antibody

SEQ ID NO: 811 - DNA, 366 bp, Artificial Sequence; identical to SEQ ID NO: 803
  Description of Artificial Sequence: Synthetic antibody

SEQ ID NO: 812 - DNA, 327 bp, Artificial Sequence
  Description of Artificial Sequence: Synthetic antibody
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagaccctgaagattttgcagtttattactgtcacaagcgtagcaactggcctccttcgctcactttcggcggagggaccaaggtggagatcaaa

SEQ ID NO: 813 - DNA, 363 bp, Artificial Sequence
  Description of Artificial Sequence: Synthetic antibody
gaggtgcagctggtggagtctgggggaggcgtggtccagcctgggaggtccctgagactctcctgtgcagcctctggattcaccttcagtacctatgctatgcactgggtccgccaggctccaggcaaggggctggcgtgggtggcacttatatcatatgatggatataataaatactacgcagactccgtgaggggccgattcaccatctccagaatcaattccaagaacacgctgtctctgcagatgaacagcctgagagctgaggacacggctgtctattactgtgcgagagggtcagctggaaactactactacggtatggacgtctggggccaggggaccacggtcaccgtctcctca

SEQ ID NO: 814 - DNA, 321 bp, Artificial Sequence
  Description of Artificial Sequence: Synthetic antibody
gacatccagatgacccagtctccatcctccctgtctgcatctgtaggagacagagtcaccatcacttgccgggcaagtcagaccattaccaactatttaaattggtatcagctgaaatcagggagagcccccaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacgccgtacacttttggccaggggaccaaggtggagatcaaa

SEQ ID NO: 815 - DNA, 366 bp, Artificial Sequence; identical to SEQ ID NO: 801
  Description of Artificial Sequence: Synthetic antibody

SEQ ID NO: 816 - DNA, 327 bp, Artificial Sequence
  Description of Artificial Sequence: Synthetic antibody
gaaattgtgttgacacagtctccagccaccctgtctttgtctccaggagaaagagccaccctctcctgcagggccagtcagagtgttagcagcttcttagcctggtaccaacagaaacctggccaggctcccaggctcctcatctatgatgcatccaacaggcccactggcatcccagccaggttcactggcagtgggtctgggacagacttcactctcaccatcagcagcctagagcctgaagattttgcagtttattactgtcagcaccgtaccaactggcctcccttattcactttcggccctgggaccaaagtggatatcaaa

SEQ ID NO: 817 - DNA, 366 bp, Artificial Sequence; identical to SEQ ID NO: 803
  Description of Artificial Sequence: Synthetic antibody

SEQ ID NO: 818 - DNA, 327 bp, Artificial Sequence; identical to SEQ ID NO: 804
  Description of Artificial Sequence: Synthetic antibody

SEQ ID NO: 819 - DNA, 363 bp, Artificial Sequence
  Description of Artificial Sequence: Synthetic antibody
gaggtgcagctggtggagtctgggggaggcgtggtccagcctgggaggtccctgagactctcctgtgcagcctctggattcaccttcagtagttatgctctgttctgggtccgccaggctccaggcaaggggctggagtgggtggcagttatttcatatgatggaaataataaatactacgcagactccgtgaggggccgattcaccatctccagagacaattccaagaacacgctgtatctgcaaatgaacagcctgagacctgaggacacggctgtgtattactgtgcgagaccatatactgggagctacaagagctacatggacgtctggggcaaagggaccacggtcaccgtctcctca

SEQ ID NO: 820 - DNA, 339 bp, Artificial Sequence; identical to SEQ ID NO: 146
  Description of Artificial Sequence: Synthetic antibody

SEQ ID NO: 821 - DNA, 354 bp, Artificial Sequence
  Description of Artificial Sequence: Synthetic antibody
gaggtgcagctggtggagtccgggggaggcgtggtccagcctgggaggtccctgagactctcctgtgcagcctctggattcaccttcagtacctatggcatgcactgggtccgccaggctccaggcaaggggctggagtgggtggcagttatatcatatgatggaagtaataaatactatgcagactccgtgaagggccgattcaccatctccagagacaattccaagaacacactgtatctgcaaatgaacagcctgagagctgaggacacggctatgtattactgtgcgaaagatgggagtatagcagcagctgactactggggccagggaaccctggtcaccgtctcctca

SEQ ID NO: 822 - DNA, 339 bp, Artificial Sequence; identical to SEQ ID NO: 48
  Description of Artificial Sequence: Synthetic antibody

SEQ ID NO: 823 - DNA, 336 bp, Artificial Sequence
  Description of Artificial Sequence: Synthetic antibody
gaggtgcagctggtggagtctggaggaggcttgatccagcctggggggtccctgagactctcctgtgcagcctctgggttcatcgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtatattactgtgcgagagagagcacgcaatggggccagggaaccctggtcaccgtctcctca

SEQ ID NO: 824 - DNA, 321 bp, Artificial Sequence
  Description of Artificial Sequence: Synthetic antibody
gacatccagatgacccagtctccttccaccctgtctgcatctgtaggagacagagtcaccatcacttgccgggccagtcacagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatacttattctcaaacgttcggccaagggaccaaggtggagatcaaa

SEQ ID NO: 825 - DNA, 369 bp, Artificial Sequence
  Description of Artificial Sequence: Synthetic antibody
gaggtgcagctggtgcagtctgggcctgaggtgaagaagcctgggacctcagtgaaggtctcctgcaaggcttctggattcacctttatgagctctgctgtgcagtgggtgcgacaggctcgtggacaacgccttgagtggataggatggatcgtcattggcagtggtaacacaaactacgcacagaagttccaggaaagagtcaccattaccagggacatgtccacaagcacagcctacatggagctgagcagcctgagatccgaggacacggccgtgtattactgtgcggccccatattgtagtagtatcagctgcaatgatggttttgatatctggggccaagggacaatggtcaccgtctcttca

SEQ ID NO: 826 - DNA, 327 bp, Artificial Sequence
  Description of Artificial Sequence: Synthetic antibody
gaaattgtgttgacgcagtctccaggcaccctgtctttgtctccaggggagagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcactatggtagctcacggggttggacgttcggccaagggaccaaggtggaaatcaaa

SEQ ID NO: 827 - protein, 120 aa, Artificial Sequence
  Description of Artificial Sequence: Synthetic antibody
EVQLVESGGDLVQPGGSLRLSCAASGFTFRSYAMSWVRQAPGKGLEWVSAISDNAYSTYYADSVKGRFTISRDNSKNTLYLQMNSLRAEDTAIYYCAKNLYSGNSPFDYWGQGTLVTVSS

SEQ ID NO: 828 - protein, 106 aa, Artificial Sequence
  Description of Artificial Sequence: Synthetic antibody
  misc_feature (1)..(2): Xaa can be any naturally occurring amino acid
XXVLTQPPSVSVSPGQTASITCSGDKLGDKYACWYQQKPGQSPVLVIYQDSKRPSGIPERFSGSNSGNTATLTISGTQAMDEADYYCQAWDSSTAVFGTGTKVTVL

SEQ ID NO: 829 - protein, 130 aa, Artificial Sequence
  Description of Artificial Sequence: Synthetic antibody
EVQLVESGGGVVQPGRSLRLSCAASGFTFSSYGMHWVRQAPGKGLEWVAVIWHDGSKKYYADSVKGRFTISRDNSKNTLYLQMNSLRAEDTAVYYCARDQGGYDYVWGSYRYTFYVFDYWGQGTLVTVSS

SEQ ID NO: 830 - protein, 108 aa, Artificial Sequence
  Description of Artificial Sequence: Synthetic antibody
  misc_feature (1)..(2): Xaa can be any naturally occurring amino acid
XXVLTQDPAVSVALGQTVRITCQGDSLRNYYASWYQQKPGQAPVVVMYGKNNRPSGIPDRVSGSSSGNTASLTITGAQAEDEADYYCNSRDSSGNHLIFGGGTKLTVL

SEQ ID NO: 831 - protein, 119 aa, Artificial Sequence
  Description of Artificial Sequence: Synthetic antibody
EVQLVESGGGLVQPGGSLRLSCAASGFTFSFYWMSWVRQAPGKGLEWVANIKQDGSEKYYVDSVKGRFSISRDNAKNSLYLQMNSLRAEDTAVYYCARVGSSSWYFDYWGQGTLVTVSS

SEQ ID NO: 832 - protein, 106 aa, Artificial Sequence
  Description of Artificial Sequence: Synthetic antibody
  misc_feature (1)..(2): Xaa can be any naturally occurring amino acid
XXVLTQPPSVSVSPGQTASITCSGDKLGDKYACWYQQKPGQSPVLVIYQDIKRPSGIPERFSGSNSGNTATLTISGTQAMDEADYYCQAWDSNTGVFGGGTKLTVL

SEQ ID NO: 833 - protein, 121 aa, Artificial Sequence
  Description of Artificial Sequence: Synthetic antibody
EVQLVQSGAEVKKPGASVKVSCKASGYTLTRYDIHWVRQATGQGLEWMGWLNPNGGNTGYAQKFQGRVTMTRNTAISTAYMELSSLRSEDAAVYYCARGQWELDAWYFDLWGRGTLVTVSS

SEQ ID NO: 834 - protein, 110 aa, Artificial Sequence
  Description of Artificial Sequence: Synthetic antibody
  misc_feature (2)..(2): Xaa can be any naturally occurring amino acid
QXVLTQDPAVSVALGQTVRITCQGDSFRSYYASWYQQKPGQAPVLVIYGKNNRPSGIPDRFSGSSSGNTASLTITGAQAEDEADYYCNSRDTSGNHLDVVFGGGTKLTVL

SEQ ID NO: 835 - protein, 121 aa, Artificial Sequence
  Description of Artificial Sequence: Synthetic antibody
EVQLVQSGAEVKKPGASLKVSCKASGYTFTRYDINWVRQATGQGLEWMGWMNPNSDNTGYAQKFQDRVTMTRNTSISTVYMELSSLRSEDTAVYYCARGQWELDVWYFDLWGRGTLVTVSS

SEQ ID NO: 836 - protein, 110 aa, Artificial Sequence
  Description of Artificial Sequence: Synthetic antibody
  misc_feature (2)..(2): Xaa can be any naturally occurring amino acid
QXVLTQDPAVSVALGQTLRITCQGDSLRSYYASWYQQKPGQAPVLVIYGKNNRPSGIPDRFSGSSSGNTASLTITGAQAEDEADYYCNSRDSSGHHLDVVFGGGTKLTVL

SEQ ID NO: 837 - protein, 123 aa, Artificial Sequence
  Description of Artificial Sequence: Synthetic antibody
QVQLQESGPGLVKPSETLSLTCTVSGGSISSYYWSWIRQPPGKGLEWIGCIYYSGRTNYSPSLKSRVTISVDTSKNLFSLKLSSVTAADTAVYYCARGGRPGAEGPYDAFDIWGQGTMVTVSS

SEQ ID NO: 838 - protein, 108 aa, Artificial Sequence
  Description of Artificial Sequence: Synthetic antibody
  misc_feature (2)..(2): Xaa can be any naturally occurring amino acid
QXVLTQDPAVSVALGQTVRITCQGDNLRRYYASWYQQKPGQAPVLVIYGKNNRPSGIPDRFSGSSSGNTASLTITGAQAEDEADYYCNSRDSSGNHVVFGGGTTLTVL

SEQ ID NO: 839 - protein, 123 aa, Artificial Sequence
  Description of Artificial Sequence: Synthetic antibody
QVQLVQSGSELKKPGASVKVSCKASGYTFSDYAMNWVRQAPGQGLEWMGWMKSNSGNTGYAQKFQGRVTMTRNTSISTAYMELTSLRSEDTAVYYCARMRSGWPTHGRPDDHWGRGALVTVSS

SEQ ID NO: 840 - protein, 108 aa, Artificial Sequence
  Description of Artificial Sequence: Synthetic antibody
DIQMTQSPSSLSASVGDRVTITCRASQSIISYLNWYHQKPGKAPKLLIYAASSLQSGVPSRFSGSGSGTDFTFTISSLQPEDIATYYCQQSDNLPMYTFGQGTKLEIK

SEQ ID NO: 841 - protein, 129 aa, Artificial Sequence
  Description of Artificial Sequence: Synthetic antibody
QVQLVESGGGVVQPGRSLRLSCAASGFTFVTSGIHWVRQAPGKGLEWVAVISYDGSNKYYADSVKGRFTISRDNSKNTLYLQMNSLRAEDTAVYYCAKGGPNKEVLYFGELLDYGMDVWGQGTTVTVSS

SEQ ID NO: 842 - protein, 114 aa, Artificial Sequence
  Description of Artificial Sequence: Synthetic antibody
DIVMTQSPDSLAVSLGERATINCKSSQSVLYSSEIKNYLAWYQQKPGQPPKLLIYWASTREFGVPDRFSGSGSGTDFTLTISSLQAEDVAVYYCQQYYSGPLDTFGQGTKLEIK

SEQ ID NO: 843 - protein, 122 aa, Artificial Sequence
  Description of Artificial Sequence: Synthetic antibody
QVQLVQSGAEVKKPGASVKVSCKVSGYTFTGYVVHWVRQAPGQDLEWMGWINTGYGNTKYSQKFQGRVTISWDTSATTAYMELSNLKSEDKAVYYCASMNRMSEQTYYGMDVWGQGTTVTVS

SEQ ID NO: 844 - protein, 105 aa, Artificial Sequence
  Description of Artificial Sequence: Synthetic antibody
DIQMTQSPSSLSASVGDRVTITCQASQDISNYLNWYQQKPGKAPKLLIYDASNLETGVPSRFSGSGSGTDFTFTISSLQPEDIATYYCQQYDKLTLGGGTKVEIK

SEQ ID NO: 845 - protein, 121 aa, Artificial Sequence
  Description of Artificial Sequence: Synthetic antibody
QVQLQQWGAGLLKPSETLSLTCAVYGGSFSGYYWSWIRQPPGKGLEWIGEINHSGSTNYNPSLKSRVTISVDTSKNHFSLKMNSVTAADTAVYYCARCRQMGNFYYYYMDVWGKGTTVTVS

SEQ ID NO: 846 - protein, 111 aa, Artificial Sequence
  Description of Artificial Sequence: Synthetic antibody
QSVLTQPPSVSGVPGQRVTVSCTGSSSNIGAGFDVYWYQQFLGTAPKLLIYGNNNRPSGVPDRFSASKSGTSASLAITGLQAEDEADYYCQSFDIGRGGWIFGGGTKLTVL

SEQ ID NO: 847 - protein, 127 aa, Artificial Sequence
  Description of Artificial Sequence: Synthetic antibody
QVHLQESGPGLVKPSETLSLTCTVSGGSINNYYWSWIRQPPGKGLEWIGEIHYSGSTSYSPSLKSRLSISVDRSKNQFSLKLASVTAADTAVYYCVRDNYFDNSGHPVYPVPWFDPWGQGTLVTVSS

SEQ ID NO: 848 - protein, 110 aa, Artificial Sequence
  Description of Artificial Sequence: Synthetic antibody
QSVLTQPPSVSGVPGQRVTVSCTGSSSNIGAGFDVYWYQQFLGTAPKLLIYGNNNRPSGVPDRFSASKSGTSASLAITGLQAEDEADYYCQSFDIGRGGWIFGGGTKLTV

SEQ ID NO: 849 - protein, 117 aa, Artificial Sequence
  Description of Artificial Sequence: Synthetic antibody
QVQVVQSGAEVKKPGASVKVSCKASGYTFKNYGISWVRQAPGQGLEWMGWISAYTGNTNYAQKFQGRMTMTTDTSTGTGYMELRSLRSDDTAVYYCARVQRRRLDYWGQGTLVIVSS

SEQ ID NO: 850 - protein, 110 aa, Artificial Sequence
  Description of Artificial Sequence: Synthetic antibody
DIVVTQTPLSLSVTPGQPASISCKSSETLLHSDGKTYLSWYLQKPGQPPQLLIYEVSNRFSGVPDRFSGSGSGTDFTLKIGRVEAEDVGLYYCMQSIQLAFGQGTRLEIE

SEQ ID NO: 851 - protein, 124 aa, Artificial Sequence
  Description of Artificial Sequence: Synthetic antibody
EVQLVESGGGVVQPGRSLRLSCAASGFTFSTYAMHWVRQAPGKGLEWVAVISYDGSNKYYADSVKGRFTISRDNSKNTLYLQMIGLRAEDTAVYYCARDWAPTYYDMPSAFDIWGQGTMVTVSS

SEQ ID NO: 852 - protein, 109 aa, Artificial Sequence
  Description of Artificial Sequence: Synthetic antibody
SYELTQPPSVSVAPGKTARITCGGNNIGNKGVHWYQQKPGQAPVLVVDDDSDRPSGIPERFSGSNSGNTATLIISSVEVGDEADFYCQVWDSSSDHPGVFGGGTKLTVL

SEQ ID NO: 853 - protein, 121 aa, Artificial Sequence
  Description of Artificial Sequence: Synthetic antibody
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARSTSGSYYYGMDVWGQGTTVTVSS

SEQ ID NO: 854 - protein, 112 aa, Artificial Sequence; identical to SEQ ID NO: 294
  Description of Artificial Sequence: Synthetic antibody

SEQ ID NO: 855 - protein, 121 aa, Artificial Sequence; identical to SEQ ID NO: 853
  Description of Artificial Sequence: Synthetic antibody

SEQ ID NO: 856 - protein, 112 aa, Artificial Sequence; identical to SEQ ID NO: 294
  Description of Artificial Sequence: Synthetic antibody

SEQ ID NO: 857 - protein, 122 aa, Artificial Sequence
  Description of Artificial Sequence: Synthetic antibody
EVQLVQSGAEVKKPGSSVKVSCKASGGTFSSYAIIWVRQAPGQGLEWMGGIIPIFGTTNYAQKFQGRVTITADESTSTAYVELSSLRSEDTAVYYCARIGHFDSSGYYLDYWGQGTLVTVSS

SEQ ID NO: 858 - protein, 109 aa, Artificial Sequence; identical to SEQ ID NO: 298
  Description of Artificial Sequence: Synthetic antibody

SEQ ID NO: 859 - protein, 122 aa, Artificial Sequence
  Description of Artificial Sequence: Synthetic antibody
EVQLVQSGAEVKKPGSSVKVSCKASGGTFSSYAISWVRQAPGQGLEWMGGIIPIFGAANYAQNFQGRVTITADESTSTGYMQLSSLRFEDTAVYYCARTSHYDSSGSYFEYWGQGTLVTVSS

SEQ ID NO: 860 - protein, 109 aa, Artificial Sequence
  Description of Artificial Sequence: Synthetic antibody
EIVLTQSPATLSLSPGERATLSCRASQSVSSYLAWYQQKPGQAPRLLIYDASNRATGIPARFSGSGSGTDFTLTISSLDPEDFAVYYCHKRSNWPPSLTFGGGTKVEIK

SEQ ID NO: 861 - protein, 119 aa, Artificial Sequence; identical to SEQ ID NO: 157
  Description of Artificial Sequence: Synthetic antibody

SEQ ID NO: 862 - protein, 106 aa, Artificial Sequence; identical to SEQ ID NO: 158
  Description of Artificial Sequence: Synthetic antibody

SEQ ID NO: 863 - protein, 129 aa, Artificial Sequence; identical to SEQ ID NO: 159
  Description of Artificial Sequence: Synthetic antibody

SEQ ID NO: 864 - protein, 107 aa, Artificial Sequence; identical to SEQ ID NO: 160
  Description of Artificial Sequence: Synthetic antibody

SEQ ID NO: 865 - protein, 129 aa, Artificial Sequence; identical to SEQ ID NO: 159
  Description of Artificial Sequence: Synthetic antibody

SEQ ID NO: 866 - protein, 107 aa, Artificial Sequence; identical to SEQ ID NO: 160
  Description of Artificial Sequence: Synthetic antibody

SEQ ID NO: 867 - protein, 122 aa, Artificial Sequence; identical to SEQ ID NO: 859
  Description of Artificial Sequence: Synthetic antibody

SEQ ID NO: 868 - protein, 109 aa, Artificial Sequence; identical to SEQ ID NO: 860
  Description of Artificial Sequence: Synthetic antibody

SEQ ID NO: 869 - protein, 121 aa, Artificial Sequence
  Description of Artificial Sequence: Synthetic antibody
EVQLVESGGGVVQPGRSLRLSCAASGFTFSTYAMHWVRQAPGKGLAWVALISYDGYNKYYADSVRGRFTISRINSKNTLSLQMNSLRAEDTAVYYCARGSAGNYYYGMDVWGQGTTVTVSS

SEQ ID NO: 870 - protein, 107 aa, Artificial Sequence
  Description of Artificial Sequence: Synthetic antibody
DIQMTQSPSSLSASVGDRVTITCRASQTITNYLNWYQLKSGRAPKLLIYAASSLQSGVPSRFSGSGSGTDFTLTISSLQPEDFATYYCQQSYSTPYTFGQGTKVEIK

SEQ ID NO: 871 - protein, 122 aa, Artificial Sequence; identical to SEQ ID NO: 857
  Description of Artificial Sequence: Synthetic antibody

SEQ ID NO: 872 - protein, 109 aa, Artificial Sequence; identical to SEQ ID NO: 298
  Description of Artificial Sequence: Synthetic antibody

SEQ ID NO: 873 - protein, 122 aa, Artificial Sequence; identical to SEQ ID NO: 859
  Description of Artificial Sequence: Synthetic antibody

SEQ ID NO: 874 - protein, 109 aa, Artificial Sequence; identical to SEQ ID NO: 860
  Description of Artificial Sequence: Synthetic antibody

SEQ ID NO: 875 - protein, 121 aa, Artificial Sequence
  Description of Artificial Sequence: Synthetic antibody
EVQLVESGGGVVQPGRSLRLSCAASGFTFSSYALFWVRQAPGKGLEWVAVISYDGNNKYYADSVRGRFTISRDNSKNTLYLQMNSLRPEDTAVYYCARPYTGSYKSYMDVWGKGTTVTVSS

SEQ ID NO: 876 - protein, 113 aa, Artificial Sequence; identical to SEQ ID NO: 300
  Description of Artificial Sequence: Synthetic antibody

SEQ ID NO: 877 - protein, 118 aa, Artificial Sequence
  Description of Artificial Sequence: Synthetic antibody
EVQLVESGGGVVQPGRSLRLSCAASGFTFSTYGMHWVRQAPGKGLEWVAVISYDGSNKYYADSVKGRFTISRDNSKNTLYLQMNSLRAEDTAMYYCAKDGSIAAADYWGQGTLVTVSS

SEQ ID NO: 878 - protein, 113 aa, Artificial Sequence; identical to SEQ ID NO: 202
  Description of Artificial Sequence: Synthetic antibody

SEQ ID NO: 879 - protein, 112 aa, Artificial Sequence
  Description of Artificial Sequence: Synthetic antibody
EVQLVESGGGLIQPGGSLRLSCAASGFIVSSNYMSWVRQAPGKGLEWVSVIYSGGSTYYADSVKGRFTISRDNSKNTLYLQMNSLRAEDTAVYYCARESTQWGQGTLVTVSS

SEQ ID NO: 880 - protein, 107 aa, Artificial Sequence
  Description of Artificial Sequence: Synthetic antibody
DIQMTQSPSTLSASVGDRVTITCRASHSISSWLAWYQQKPGKAPKLLIYKASSLESGVPSRFSGSGSGTEFTLTISSLQPDDFATYYCQQYNTYSQTFGQGTKVEIK

SEQ ID NO: 881 - protein, 123 aa, Artificial Sequence
  Description of Artificial Sequence: Synthetic antibody
EVQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 882 - protein, 109 aa, Artificial Sequence
  Description of Artificial Sequence: Synthetic antibody
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 883 - protein, 8 aa, Artificial Sequence
  Description of Artificial Sequence: Synthetic antibody
GFTFRSYA

SEQ ID NO: 884 - protein, 8 aa, Artificial Sequence
  Description of Artificial Sequence: Synthetic antibody
ISDNAYST

SEQ ID NO: 885 - protein, 13 aa, Artificial Sequence
  Description of Artificial Sequence: Synthetic antibody
AKNLYSGNSPFDY

SEQ ID NO: 886 - protein, 8 aa, Artificial Sequence
  Description of Artificial Sequence: Synthetic antibody
GFTFSSYG

SEQ ID NO: 887 - protein, 8 aa, Artificial Sequence
  Description of Artificial Sequence: Synthetic antibody
IWHDGSKK

SEQ ID NO: 888 - protein, 23 aa, Artificial Sequence
  Description of Artificial Sequence: Synthetic antibody
ARDQGGYDYVWGSYRYTFYVFDY

SEQ ID NO: 889 - protein, 8 aa, Artificial Sequence
  Description of Artificial Sequence: Synthetic antibody
GFTFSFYW

SEQ ID NO: 890 - protein, 8 aa, Artificial Sequence
  Description of Artificial Sequence: Synthetic antibody
IKQDGSEK

SEQ ID NO: 891 - protein, 12 aa, Artificial Sequence
  Description of Artificial Sequence: Synthetic antibody
ARVGSSSWYFDY

SEQ ID NO: 892 - protein, 8 aa, Artificial Sequence
  Description of Artificial Sequence: Synthetic antibody
GYTLTRYD

SEQ ID NO: 893 - protein, 8 aa, Artificial Sequence
  Description of Artificial Sequence: Synthetic antibody
LNPNGGNT

SEQ ID NO: 894 - protein, 14 aa, Artificial Sequence
  Description of Artificial Sequence: Synthetic antibody
ARGQWELDAWYFDL

SEQ ID NO: 895 - protein, 8 aa, Artificial Sequence
  Description of Artificial Sequence: Synthetic antibody
GYTFTRYD

SEQ ID NO: 896 - protein, 8 aa, Artificial Sequence
  Description of Artificial Sequence: Synthetic antibody
MNPNSDNT

SEQ ID NO: 897 - protein, 14 aa, Artificial Sequence
  Description of Artificial Sequence: Synthetic antibody
ARGQWELDVWYFDL

SEQ ID NO: 898 - protein, 8 aa, Artificial Sequence
  Description of Artificial Sequence: Synthetic antibody
GGSISSYY

SEQ ID NO: 899 - protein, 7 aa, Artificial Sequence
  Description of Artificial Sequence: Synthetic antibody
IYYSGRT

SEQ ID NO: 900 - protein, 17 aa, Artificial Sequence
  Description of Artificial Sequence: Synthetic antibody
ARGGRPGAEGPYDAFDI

SEQ ID NO: 901 - protein, 8 aa, Artificial Sequence
  Description of Artificial Sequence: Synthetic antibody
GYTFSDYA

SEQ ID NO: 902 - protein, 8 aa, Artificial Sequence
  Description of Artificial Sequence: Synthetic antibody
MKSNSGNT

SEQ ID NO: 903 - protein, 16 aa, Artificial Sequence
  Description of Artificial Sequence: Synthetic antibody
ARMRSGWPTHGRPDDH

SEQ ID NO: 904 - protein, 8 aa, Artificial Sequence
  Description of Artificial Sequence: Synthetic antibody
GFTFVTSG

SEQ ID NO: 905 - protein, 8 aa, Artificial Sequence
  Description of Artificial Sequence: Synthetic antibody
ISYDGSNK

SEQ ID NO: 906 - protein, 22 aa, Artificial Sequence
  Description of Artificial Sequence: Synthetic antibody
AKGGPNKEVLYFGELLDYGMDV

SEQ ID NO: 907 - protein, 8 aa, Artificial Sequence
  Description of Artificial Sequence: Synthetic antibody
GYTFTGYV

SEQ ID NO: 908 - protein, 8 aa, Artificial Sequence
  Description of Artificial Sequence: Synthetic antibody
INTGYGNT

SEQ ID NO: 909 - protein, 16 aa, Artificial Sequence
  Description of Artificial Sequence: Synthetic antibody
ASMNRMSEQTYYGMDV

SEQ ID NO: 910 - protein, 8 aa, Artificial Sequence
  Description of Artificial Sequence: Synthetic antibody
GGSFSGYY

SEQ ID NO: 911 - protein, 7 aa, Artificial Sequence
  Description of Artificial Sequence: Synthetic antibody
INHSGST

SEQ ID NO: 912 - protein, 16 aa, Artificial Sequence
  Description of Artificial Sequence: Synthetic antibody
ARCRQMGNFYYYYMDV

SEQ ID NO: 913 - protein, 8 aa, Artificial Sequence
  Description of Artificial Sequence: Synthetic antibody
GGSINNYY

SEQ ID NO: 914 - protein, 7 aa, Artificial Sequence
  Description of Artificial Sequence: Synthetic antibody
IHYSGST

SEQ ID NO: 915 - protein, 21 aa, Artificial Sequence
  Description of Artificial Sequence: Synthetic antibody
VRDNYFDNSGHPVYPVPWFDP

SEQ ID NO: 916 - protein, 8 aa, Artificial Sequence
  Description of Artificial Sequence: Synthetic antibody
GYTFKNYG

SEQ ID NO: 917 - protein, 8 aa, Artificial Sequence
  Description of Artificial Sequence: Synthetic antibody
ISAYTGNT

SEQ ID NO: 918 - protein, 10 aa, Artificial Sequence
  Description of Artificial Sequence: Synthetic antibody
ARVQRRRLDY

SEQ ID NO: 919 - protein, 8 aa, Artificial Sequence
  Description of Artificial Sequence: Synthetic antibody
GFTFSTYA

SEQ ID NO: 920 - protein, 8 aa, Artificial Sequence
  Description of Artificial Sequence: Synthetic antibody
ISYDGSNK

SEQ ID NO: 921 - protein, 17 aa, Artificial Sequence
  Description of Artificial Sequence: Synthetic antibody
ARDWAPTYYDMPSAFDI

SEQ ID NO: 922 - protein, 8 aa, Artificial Sequence
  Description of Artificial Sequence: Synthetic antibody
GFTFSSYA

SEQ ID NO: 923 - protein, 8 aa, Artificial Sequence
  Description of Artificial Sequence: Synthetic antibody
ISYDGSNK

SEQ ID NO: 924 - protein, 14 aa, Artificial Sequence
  Description of Artificial Sequence: Synthetic antibody
ARSTSGSYYYGMDV

SEQ ID NO: 925 - protein, 8 aa, Artificial Sequence
  Description of Artificial Sequence: Synthetic antibody
GFTFSSYA

SEQ ID NO: 926 - protein, 8 aa, Artificial Sequence
  Description of Artificial Sequence: Synthetic antibody
ISYDGSNK

SEQ ID NO: 927 - protein, 14 aa, Artificial Sequence
  Description of Artificial Sequence: Synthetic antibody
ARSTSGSYYYGMDV

SEQ ID NO: 928 - protein, 8 aa, Artificial Sequence
  Description of Artificial Sequence: Synthetic antibody
GGTFSSYA

SEQ ID NO: 929 - protein, 8 aa, Artificial Sequence
  Description of Artificial Sequence: Synthetic antibody
IIPIFGTT

SEQ ID NO: 930 - protein, 15 aa, Artificial Sequence
  Description of Artificial Sequence: Synthetic antibody
ARIGHFDSSGYYLDY

SEQ ID NO: 931 - protein, 8 aa, Artificial Sequence
  Description of Artificial Sequence: Synthetic antibody
GGTFSSYA

SEQ ID NO: 932 - protein, 8 aa, Artificial Sequence
  Description of Artificial Sequence: Synthetic antibody
IIPIFGAA

SEQ ID NO: 933 - protein, 15 aa, Artificial Sequence
  Description of Artificial Sequence: Synthetic antibody
ARTSHYDSSGSYFEY

SEQ ID NO: 934 - protein, 8 aa, Artificial Sequence
  Description of Artificial Sequence: Synthetic antibody
GFTFSFYW

SEQ ID NO: 935 - protein, 8 aa, Artificial Sequence
  Description of Artificial Sequence: Synthetic antibody
IKQDGGEK

SEQ ID NO: 936 - protein, 12 aa, Artificial Sequence
  Description of Artificial Sequence: Synthetic antibody
ARLSGSSWDFDY

SEQ ID NO: 937 - protein, 8 aa, Artificial Sequence
  Description of Artificial Sequence: Synthetic antibody
GFTFDDYG

SEQ ID NO: 938 - protein, 8 aa, Artificial Sequence
  Description of Artificial Sequence: Synthetic antibody
INWNGGST

SEQ ID NO: 939 - protein, 22 aa, Artificial Sequence
  Description of Artificial Sequence: Synthetic antibody
ARRRSSSRYSSGWYMYYYYMDV

SEQ ID NO: 940 - protein, 8 aa, Artificial Sequence
  Description of Artificial Sequence: Synthetic antibody
GFTFDDYG

SEQ ID NO: 941 - protein, 8 aa, Artificial Sequence
  Description of Artificial Sequence: Synthetic antibody
INWNGGST

SEQ ID NO: 942 - protein, 22 aa, Artificial Sequence
  Description of Artificial Sequence: Synthetic antibody
ARRRSSSRYSSGWYMYYYYMDV

SEQ ID NO: 943 - protein, 8 aa, Artificial Sequence
  Description of Artificial Sequence: Synthetic antibody
GGTFSSYA

SEQ ID NO: 944 - protein, 8 aa, Artificial Sequence
  Description of Artificial Sequence: Synthetic antibody
IIPIFGAA

SEQ ID NO: 945 - protein, 15 aa, Artificial Sequence
  Description of Artificial Sequence: Synthetic antibody
ARTSHYDSSGSYFEY

SEQ ID NO: 946 - protein, 8 aa, Artificial Sequence
  Description of Artificial Sequence: Synthetic antibody
GFTFSTYA

SEQ ID NO: 947 - protein, 8 aa, Artificial Sequence
  Description of Artificial Sequence: Synthetic antibody
ISYDGYNK

SEQ ID NO: 948 - protein, 14 aa, Artificial Sequence
  Description of Artificial Sequence: Synthetic antibody
ARGSAGNYYYGMDV

SEQ ID NO: 949 - protein, 8 aa, Artificial Sequence
  Description of Artificial Sequence: Synthetic antibody
GGTFSSYA

SEQ ID NO: 950 - protein, 8 aa, Artificial Sequence
  Description of Artificial Sequence: Synthetic antibody
IIPIFGTT

SEQ ID NO: 951 - protein, 15 aa, Artificial Sequence
  Description of Artificial Sequence: Synthetic antibody
ARIGHFDSSGYYLDY

SEQ ID NO: 952 - protein, 8 aa, Artificial Sequence
  Description of Artificial Sequence: Synthetic antibody
GGTFSSYA

SEQ ID NO: 953 - protein, 8 aa, Artificial Sequence
  Description of Artificial Sequence: Synthetic antibody
IIPIFGAA

SEQ ID NO: 954 - protein, 15 aa, Artificial Sequence
  Description of Artificial Sequence: Synthetic antibody
ARTSHYDSSGSYFEY

SEQ ID NO: 955 - protein, 8 aa, Artificial Sequence
  Description of Artificial Sequence: Synthetic antibody
GFTFSSYA

SEQ ID NO: 956 - protein, 8 aa, Artificial Sequence
  Description of Artificial Sequence: Synthetic antibody
ISYDGNNK

SEQ ID NO: 957 - protein, 14 aa, Artificial Sequence
  Description of Artificial Sequence: Synthetic antibody
ARPYTGSYKSYMDV

SEQ ID NO: 958 - protein, 8 aa, Artificial Sequence
  Description of Artificial Sequence: Synthetic antibody
GFTFSTYG

SEQ ID NO: 959 - protein, 8 aa, Artificial Sequence
  Description of Artificial Sequence: Synthetic antibody
ISYDGSNK

SEQ ID NO: 960 - protein, 11 aa, Artificial Sequence
  Description of Artificial Sequence: Synthetic antibody
AKDGSIAAADY

SEQ ID NO: 961 - protein, 8 aa, Artificial Sequence
  Description of Artificial Sequence: Synthetic antibody
GFIVSSNY

SEQ ID NO: 962 - protein, 7 aa, Artificial Sequence
  Description of Artificial Sequence: Synthetic antibody
IYSGGST

SEQ ID NO: 963 - protein, 6 aa, Artificial Sequence
  Description of Artificial Sequence: Synthetic antibody
ARESTQ

SEQ ID NO: 964 - protein, 8 aa, Artificial Sequence
  Description of Artificial Sequence: Synthetic antibody
GFTFMSSA

SEQ ID NO: 965 - protein, 8 aa, Artificial Sequence
  Description of Artificial Sequence: Synthetic antibody
IVIGSGNT

SEQ ID NO: 966 - protein, 16 aa, Artificial Sequence
  Description of Artificial Sequence: Synthetic antibody
AAPYCSSISCNDGFDI

SEQ ID NO: 967 - protein, 6 aa, Artificial Sequence
  Description of Artificial Sequence: Synthetic antibody
KLGDKY

SEQ ID NO: 968 - protein, 3 aa, Artificial Sequence
  Description of Artificial Sequence: Synthetic antibody
QDS

SEQ ID NO: 969 - protein, 9 aa, Artificial Sequence
  Description of Artificial Sequence: Synthetic antibody
QAWDSSTAV

SEQ ID NO: 970 - protein, 6 aa, Artificial Sequence
  Description of Artificial Sequence: Synthetic antibody
SLRNYY

SEQ ID NO: 971 - protein, 3 aa, Artificial Sequence
  Description of Artificial Sequence: Synthetic antibody
GKN

SEQ ID NO: 972 - protein, 11 aa, Artificial Sequence
  Description of Artificial Sequence: Synthetic antibody
NSRDSSGNHLI

SEQ ID NO: 973 - protein, 6 aa, Artificial Sequence
  Description of Artificial Sequence: Synthetic antibody
KLGDKY

SEQ ID NO: 974 - protein, 3 aa, Artificial Sequence
  Description of Artificial Sequence: Synthetic antibody
QDI

SEQ ID NO: 975 - protein, 9 aa, Artificial Sequence
  Description of Artificial Sequence: Synthetic antibody
QAWDSNTGV

SEQ ID NO: 976 - protein, 6 aa, Artificial Sequence
  Description of Artificial Sequence: Synthetic antibody
SFRSYY

SEQ ID NO: 977 - protein, 3 aa, Artificial Sequence
  Description of Artificial Sequence: Synthetic antibody
GKN

SEQ ID NO: 978 - protein, 13 aa, Artificial Sequence
  Description of Artificial Sequence: Synthetic antibody
NSRDTSGNHLDVV

SEQ ID NO: 979 - protein, 6 aa, Artificial Sequence
  Description of Artificial Sequence: Synthetic antibody
SLRSYY

SEQ ID NO: 980 - protein, 3 aa, Artificial Sequence
  Description of Artificial Sequence: Synthetic antibody
GKN

SEQ ID NO: 981 - protein, 13 aa, Artificial Sequence
  Description of Artificial Sequence: Synthetic antibody
NSRDSSGHHLDVV

SEQ ID NO: 982 - protein, 6 aa, Artificial Sequence
  Description of Artificial Sequence: Synthetic antibody
NLRRYY

SEQ ID NO: 983 - protein, 3 aa, Artificial Sequence
  Description of Artificial Sequence: Synthetic antibody
GKN

SEQ ID NO: 984 - protein, 11 aa, Artificial Sequence
  Description of Artificial Sequence: Synthetic antibody
NSRDSSGNHVV

SEQ ID NO: 985 - protein, 6 aa, Artificial Sequence
  Description of Artificial Sequence: Synthetic antibody
QSIISY

SEQ ID NO: 986 - protein, 3 aa, Artificial Sequence
  Description of Artificial Sequence: Synthetic antibody
AAS

SEQ ID NO: 987 - protein, 10 aa, Artificial Sequence
  Description of Artificial Sequence: Synthetic antibody
QQSDNLPMYT

SEQ ID NO: 988 - protein, 12 aa, Artificial Sequence
  Description of Artificial Sequence: Synthetic antibody
QSVLYSSEIKNY

SEQ ID NO: 989 - protein, 3 aa, Artificial Sequence
  Description of Artificial Sequence: Synthetic antibody
WAS

SEQ ID NO: 990 - protein, 10 aa, Artificial Sequence
  Description of Artificial Sequence: Synthetic antibody
QQYYSGPLDT

SEQ ID NO: 991 - protein, 6 aa, Artificial Sequence
  Description of Artificial Sequence: Synthetic antibody
QDISNY

SEQ ID NO: 992 - protein, 3 aa, Artificial Sequence
  Description of Artificial Sequence: Synthetic antibody
DAS

SEQ ID NO: 993 - protein, 7 aa, Artificial Sequence
  Description of Artificial Sequence: Synthetic antibody
QQYDKLT

SEQ ID NO: 994 - protein, 9 aa, Artificial Sequence
  Description of Artificial Sequence: Synthetic antibody
SSNIGAGFD

SEQ ID NO: 995 - protein, 3 aa, Artificial Sequence
  Description of Artificial Sequence: Synthetic antibody
GNN

SEQ ID NO: 996 - protein, 11 aa, Artificial Sequence
  Description of Artificial Sequence: Synthetic antibody
QSFDIGRGGWI

SEQ ID NO: 997 - protein, 9 aa, Artificial Sequence
  Description of Artificial Sequence: Synthetic antibody
SSNIGAGFD

SEQ ID NO: 998 - protein, 3 aa, Artificial Sequence
  Description of Artificial Sequence: Synthetic antibody
GNN

SEQ ID NO: 999 - protein, 11 aa, Artificial Sequence
  Description of Artificial Sequence: Synthetic antibody
QSFDIGRGGWI

SEQ ID NO: 1000 - protein, 11 aa, Artificial Sequence
  Description of Artificial Sequence: Synthetic antibody
ETLLHSDGKTY

SEQ ID NO: 1001 - protein, 3 aa, Artificial Sequence
  Description of Artificial Sequence: Synthetic antibody
EVS

SEQ ID NO: 1002 - protein, 7 aa, Artificial Sequence
  Description of Artificial Sequence: Synthetic antibody
MQSIQLA

SEQ ID NO: 1003 - protein, 6 aa, Artificial Sequence
  Description of Artificial Sequence: Synthetic antibody
NIGNKG

SEQ ID NO: 1004 - protein, 3 aa, Artificial Sequence
  Description of Artificial Sequence: Synthetic antibody
DDS

SEQ ID NO: 1005 - protein, 12 aa, Artificial Sequence
  Description of Artificial Sequence: Synthetic antibody
QVWDSSSDHPGV

SEQ ID NO: 1006 - protein, 9 aa, Artificial Sequence
  Description of Artificial Sequence: Synthetic antibody
SSDVGDYNY

SEQ ID NO: 1007 - protein, 3 aa, Artificial Sequence
  Description of Artificial Sequence: Synthetic antibody
DVS

SEQ ID NO: 1008 - protein, 12 aa, Artificial Sequence
  Description of Artificial Sequence: Synthetic antibody
SSYTSSSTLLYV

SEQ ID NO: 1009 - protein, 9 aa, Artificial Sequence
  Description of Artificial Sequence: Synthetic antibody
SSDVGDYNY

SEQ ID NO: 1010 - protein, 3 aa, Artificial Sequence
  Description of Artificial Sequence: Synthetic antibody
DVS

SEQ ID NO: 1011 - protein, 12 aa, Artificial Sequence
  Description of Artificial Sequence: Synthetic antibody
SSYTSSSTLLYV

SEQ ID NO: 1012 - protein, 6 aa, Artificial Sequence
  Description of Artificial Sequence: Synthetic antibody
QSVSSF

SEQ ID NO: 1013 - protein, 3 aa, Artificial Sequence
  Description of Artificial Sequence: Synthetic antibody
DAS

SEQ ID NO: 1014 - protein, 11 aa, Artificial Sequence
  Description of Artificial Sequence: Synthetic antibody
QHRTNWPPLFT

SEQ ID NO: 1015 - protein, 6 aa, Artificial Sequence
  Description of Artificial Sequence: Synthetic antibody
QSVSSY

SEQ ID NO: 1016 - protein, 3 aa, Artificial Sequence
  Description of Artificial Sequence: Synthetic antibody
DAS

SEQ ID NO: 1017 - protein, 11 aa, Artificial Sequence
  Description of Artificial Sequence: Synthetic antibody
HKRSNWPPSLT

SEQ ID NO: 1018 - protein, 6 aa, Artificial Sequence
  Description of Artificial Sequence: Synthetic antibody
KLGDKY

SEQ ID NO: 1019 - protein, 3 aa, Artificial Sequence
  Description of Artificial Sequence: Synthetic antibody
QDS

SEQ ID NO: 1020 - protein, 9 aa, Artificial Sequence
  Description of Artificial Sequence: Synthetic antibody
QAWDSSTGV

SEQ ID NO: 1021 - protein, 6 aa, Artificial Sequence
  Description of Artificial Sequence: Synthetic antibody
QSVSTW

SEQ ID NO: 1022 - protein, 3 aa, Artificial Sequence
  Description of Artificial Sequence: Synthetic antibody
EAS

SEQ ID NO: 1023 - protein, 9 aa, Artificial Sequence
  Description of Artificial Sequence: Synthetic antibody
QQYNTYSGT

SEQ ID NO: 1024 - protein, 6 aa, Artificial Sequence
  Description of Artificial Sequence: Synthetic antibody
QSVSTW

SEQ ID NO: 1025 - protein, 3 aa, Artificial Sequence
  Description of Artificial Sequence: Synthetic antibody
EAS

SEQ ID NO: 1026 - protein, 9 aa, Artificial Sequence
  Description of Artificial Sequence: Synthetic antibody
QQYNTYSGT

SEQ ID NO: 1027 - protein, 3 aa, Artificial Sequence
  Description of Artificial Sequence: Synthetic antibody
DAS

SEQ ID NO: 1028 - protein, 11 aa, Artificial Sequence
  Description of Artificial Sequence: Synthetic antibody
HKRSNWPPSLT

SEQ ID NO: 1029 - protein, 6 aa, Artificial Sequence
  Description of Artificial Sequence: Synthetic antibody
QTITNY

SEQ ID NO: 1030 - protein, 3 aa, Artificial Sequence
  Description of Artificial Sequence: Synthetic antibody
AAS

SEQ ID NO: 1031 - protein, 9 aa, Artificial Sequence
  Description of Artificial Sequence: Synthetic antibody
QQSYSTPYT

SEQ ID NO: 1032 - protein, 6 aa, Artificial Sequence
  Description of Artificial Sequence: Synthetic antibody
QSVSSF

SEQ ID NO: 1033 - protein, 3 aa, Artificial Sequence
  Description of Artificial Sequence: Synthetic antibody
DAS

SEQ ID NO: 1034 - protein, 11 aa, Artificial Sequence
  Description of Artificial Sequence: Synthetic antibody
QHRTNWPPLFT

SEQ ID NO: 1035 - protein, 6 aa, Artificial Sequence
  Description of Artificial Sequence: Synthetic antibody
QSVSSY

SEQ ID NO: 1036 - protein, 3 aa, Artificial Sequence
  Description of Artificial Sequence: Synthetic antibody
DAS

SEQ ID NO: 1037 - protein, 11 aa, Artificial Sequence
  Description of Artificial Sequence: Synthetic antibody
HKRSNWPPSLT

SEQ ID NO: 1038 - protein, 12 aa, Artificial Sequence
  Description of Artificial Sequence: Synthetic antibody
QSVLYSSNNKNS

SEQ ID NO: 1039 - protein, 3 aa, Artificial Sequence
  Description of Artificial Sequence: Synthetic antibody
WAS

SEQ ID NO: 1040 - protein, 9 aa, Artificial Sequence
  Description of Artificial Sequence: Synthetic antibody
QQYYSISWT

SEQ ID NO: 1041 - protein, 12 aa, Artificial Sequence
  Description of Artificial Sequence: Synthetic antibody
QSVLHSSNNKDS

SEQ ID NO: 1042 - protein, 3 aa, Artificial Sequence
  Description of Artificial Sequence: Synthetic antibody
WAS

SEQ ID NO: 1043 - protein, 9 aa, Artificial Sequence
  Description of Artificial Sequence: Synthetic antibody
QQYYSTPWT

SEQ ID NO: 1044 - protein, 6 aa, Artificial Sequence
  Description of Artificial Sequence: Synthetic antibody
HSISSW

SEQ ID NO: 1045 - protein, 3 aa, Artificial Sequence
  Description of Artificial Sequence: Synthetic antibody
KAS

SEQ ID NO: 1046 - protein, 9 aa, Artificial Sequence
  Description of Artificial Sequence: Synthetic antibody
QQYNTYSQT

SEQ ID NO: 1047 - protein, 7 aa, Artificial Sequence
  Description of Artificial Sequence: Synthetic antibody
QSVSSSY

SEQ ID NO: 1048 - protein, 3 aa, Artificial Sequence
  Description of Artificial Sequence: Synthetic antibody
GAS

SEQ ID NO: 1049 - protein, 10 aa, Artificial Sequence
  Description of Artificial Sequence: Synthetic antibody
QHYGSSRGWT

SEQ ID NO: 1050 - protein, 108 aa, Artificial sequence
  Description of Artificial Sequence: Synthetic antibody
EIVLTQSPGTLSLSPGERATLSCRASQSVSSNFLAWYQQKPGQAPRLLIYGASSRATGIPDRFSGSGSGTDFTLTISRLEPEDFAVYYCQQYGSSPWTFGQGTKVEIK

SEQ ID NO: 1051 - protein, 109 aa, Artificial sequence
  Description of Artificial Sequence: Synthetic antibody
EIVLTQSPGTLSLSPGERATLSCRASQSVSSNFLAWYQQKPGQAPRLLIYGASSRATGIPDRFSGSGSGTDFTLTISRLEPEDFAVYYCQQYGSSPPWTFGQGTKVEIK

SEQ ID NO: 1052 - protein, 108 aa, Artificial sequence
  Description of Artificial Sequence: Synthetic antibody
EIVLTQSPGTLSLSPGERATLSCRASQSVSSNFLAWYQQKPGQAPRLLIYGASSRATGIPDRFSGSGSGTDFTLTISRLEPEDFAVYYCQQYGSSLWTFGQGTKVEIK

SEQ ID NO: 1053 - protein, 108 aa, Artificial sequence
  Description of Artificial Sequence: Synthetic antibody
EIVLTQSPGTLSLSPGERATLSCRASQSVSSNFLAWYQQKPGQAPRLLIYGASSRATGIPDRFSGSGSGTDFTLTISRLEPEDFAVYYCQQYGSSSWTFGQGTKVEIK

SEQ ID NO: 1054 - protein, 109 aa, Artificial sequence
  Description of Artificial Sequence: Synthetic antibody
EIVLTQSPGTLSLSPGERATLSCRASQSVSSNFLAWYQQKPGQAPRLLIYGASSRATGIPDRFSGSGSGTDFTLTISRLEPEDFAVYYCQQYGSSPTWTFGQGTKVEIK

SEQ ID NO: 1055 - protein, 8 aa, Artificial sequence
  Description of Artificial Sequence: Synthetic antibody
IGWIVCAA

SEQ ID NO: 1056 - protein, 10 aa, Artificial sequence
  Description of Artificial Sequence: Synthetic antibody
GYCSSTSCYA

SEQ ID NO: 1057 - protein, 10 aa, Artificial sequence
  Description of Artificial Sequence: Synthetic antibody
GYCTNGVCYA

SEQ ID NO: 1058 - protein, 22 aa, Artificial sequence
  Description of Artificial Sequence: Synthetic antibody
GYCSGGSCYSSSYLAWQYGSSP

SEQ ID NO: 1059 - protein, 10 aa, Artificial sequence
  Description of Artificial Sequence: Synthetic antibody
DAFDIWWTFG

SEQ ID NO: 1060 - protein, 40 aa, Artificial sequence
  Description of Artificial Sequence: Synthetic antibody
IGWIVCAAPYCSSISCNDGFDIWSSYLAWHYGSSRGRWFG

SEQ ID NO: 1061 - protein, 40 aa, Artificial sequence
  Description of Artificial Sequence: Synthetic antibody
IGWIQCAAPYCSRTSCHDAFDIWSSYLAWHFGSSSQWTFG

SEQ ID NO: 1062 - protein, 39 aa, Artificial sequence
  Description of Artificial Sequence: Synthetic antibody
IGWIVCAAPHCNRTSCYDAFDLWSSYLGWQYGSSPWTFG

SEQ ID NO: 1063 - protein, 40 aa, Artificial sequence
  Description of Artificial Sequence: Synthetic antibody
IGWIVCAAPRCSSGGSCYDGFDIWSSYLAWQYGSSPWTFG

SEQ ID NO: 1064 - protein, 39 aa, Artificial sequence
  Description of Artificial Sequence: Synthetic antibody
IGWIVCAAPYCSSTRCYDAFDIWSSYLAWQYGRSPWTFG

SEQ ID NO: 1065 - protein, 39 aa, Artificial sequence
  Description of Artificial Sequence: Synthetic antibody
IGWIVCAAPHCSSTICYDGFDIWSSYLAWQYGSSPWTFG

SEQ ID NO: 1066 - protein, 40 aa, Artificial sequence
  Description of Artificial Sequence: Synthetic antibody
VGWIVCASPYCSGGSCSDGFDIWSSYLAWQYVGLTGWTFG

SEQ ID NO: 1067 - protein, 39 aa, Artificial sequence
  Description of Artificial Sequence: Synthetic antibody
IGWIVCAAPHCSSTSCGDAFDIWSSYLAWQYGNSPWTFG

SEQ ID NO: 1068 - protein, 39 aa, Artificial sequence
  Description of Artificial Sequence: Synthetic antibody
IGWIVCAAPYCSGGSCSDAFDIWSSYLAWQYGSSPWTFG

SEQ ID NO: 1069 - protein, 39 aa, Artificial sequence
  Description of Artificial Sequence: Synthetic antibody
IGWIVCAAVDCNSTSCYDAFDIWSSYLAWQYDISPWTFG

SEQ ID NO: 1070 - protein, 39 aa, Artificial sequence
  Description of Artificial Sequence: Synthetic antibody
IGWIVCAAPHCSGGSCLDAFDIWSSYLAWQYGSSPWTFG

SEQ ID NO: 1071 - protein, 39 aa, Artificial sequence
  Description of Artificial Sequence: Synthetic antibody
IGWIVCAAPHCSGGSCYDAFDIWSSYLAWQYGSSPWTFG

SEQ ID NO: 1072 - protein, 40 aa, Artificial sequence
  Description of Artificial Sequence: Synthetic antibody
IGWIVCAANHCSSGGSCYDGFDIWSSYLAWQYGSSPWMFG

SEQ ID NO: 1073 - protein, 40 aa, Artificial sequence
  Description of Artificial Sequence: Synthetic antibody
IGWIVCAAPYCSSGGSCNDAFDIWSSYLAWQYGSSPWTFG